(19) United States
(12) Patent Application Publication
(10) Pub. No.: US 2024/0122926 A1
(43) Pub. Date: Apr. 18, 2024

(54) APELIN RECEPTOR MODULATORS FOR TREATING MUSCLE CONDITIONS

(71) Applicant: BioAge Labs, Inc., Richmond, CA (US)

(72) Inventors: Kristen Patricia FORTNEY, Derry, NH (US); Eric Kim MORGEN, Toronto (CA); Justin REBO, Derry, NH (US); Robert HUGHES, Berkeley, CA (US); Fred ASWAD, Berkeley, CA (US); Peng Khun LEONG, San Francisco, CA (US); Sashanaz H. IGDARI, San Ramon, CA (US); Paul David RUBIN, Henderson, NV (US); Rusty Lee MONTGOMERY, Longmont, CO (US)

(21) Appl. No.: 18/373,880

(22) Filed: Sep. 27, 2023

Related U.S. Application Data
(60) Provisional application No. 63/410,576, filed on Sep. 27, 2022.
(60) Provisional application No. 63/413,436, filed on Oct. 5, 2022.
(60) Provisional application No. 63/428,405, filed on Nov. 28, 2022.
(60) Provisional application No. 63/429,946, filed on Dec. 2, 2022.
(60) Provisional application No. 63/478,291, filed on Jan. 3, 2023.
(60) Provisional application No. 63/478,302, filed on Jan. 3, 2023.
(60) Provisional application No. 63/478,474, filed on Jan. 4, 2023.
(60) Provisional application No. 63/489,935, filed on Mar. 13, 2023.
(60) Provisional application No. 63/493,987, filed on Apr. 3, 2023.
(60) Provisional application No. 63/512,888, filed on Jul. 10, 2023.
(60) Provisional application No. 63/517,584, filed on Aug. 3, 2023.
(60) Provisional application No. 63/520,333, filed on Aug. 17, 2023.

(51) Int. Cl. A61K 31/506; A61P 21/00

(52) U.S. Cl. CPC A61K 31/506; A61P 21/00

(57) ABSTRACT

Apelin receptor modulators can improve physical performance, slow progression of age-related frailty, and can reduce age-related muscle weakness in human patients. This disclosure provides methods for treating muscle conditions using a particular class of apelin receptor modulators (e.g., agonists). The muscle condition can be an age-related muscle condition. Also provided is a method for maintaining and/or increasing muscle mass, muscle function, and/or muscle strength in an elderly subject by administration of the apelin receptor modulator. In some embodiments, the apelin receptor modulator (e.g., agonist) is BGE-105, or a pharmaceutically acceptable salt thereof.

### 1. CROSS-REFERENCE TO RELATED APPLICATIONS

This application claims the benefit of U.S. Provisional Application Nos. 63/410,576, filed Sep. 27, 2022; 63/413,436, filed Oct. 5, 2022; 63/428,405, filed Nov. 28, 2022; 63/429,946, filed Dec. 2, 2022; 63/478,291, filed Jan. 3, 2023; 63/478,302, filed Jan. 3, 2023; 63/478,474, filed Jan. 4, 2023; 63/489,935, filed Mar. 13, 2023; 63/493,987, filed Apr. 3, 2023; 63/512,888, filed Jul. 10, 2023; 63/517,584, Aug. 3, 2023; 63/520,333, filed Aug. 17, 2023, each of which is incorporated in its entirety by reference.

### 2. BACKGROUND

As people age, they accumulate physiologic and pathophysiologic changes; these accumulated age-related changes predispose a person to death from various external and internal stressors. Frailty is highly prevalent in old age and considered synonymous with disability, comorbidity, and other characteristics that confer high risk for falls, disability, nursing home admission, hospitalization, and mortality. Frailty is considered a clinical syndrome which can be characterized according to indices of frailty that are composite measures of such age-related changes. As the median age of the population increases, there is an increasing need for drugs that reduce or counteract the accumulation of age-related deficits including frailty in elderly individuals.

### 3. SUMMARY

This disclosure provides methods for treating muscle conditions using a particular class of apelin receptor modulators, and in particular treatment for a variety of age-related muscle conditions. In some embodiments, the apelin receptor modulator is an apelin receptor agonist.

We applied bioinformatic and machine learning approaches to analyze human data using survival predictor models and discovered an association of apelin protein levels with future aging outcomes. We discovered that higher circulating levels of apelin are associated with reduced all-cause mortality (p=0.0002)—that is, greater longevity. In addition, our analyses demonstrated that higher levels of apelin are associated with better future physical function, and measures of frailty.

Based on this discovery, we tested a modulator of the apelin receptor, BGE-105, for its effect on aged mice in models of frailty. BGE-105 has the structure shown below:

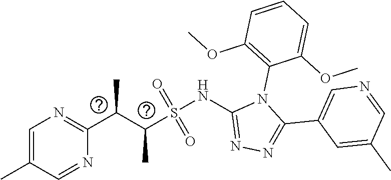

BGE-105 (also referred to as AMG-986) is known to activate the apelin receptor and induces a cardiovascular response in rats (Ason et al., JCI Insight. 5(8):1-16(2020)). Clinical trials were performed with AMG-986 to study the safety, tolerability, and pharmacokinetics in healthy subjects and heart failure subjects (NCT03276728) those with impaired renal function (NCT03318809). Nevertheless, the compound's effect on muscle loss and function in elderly individuals is unknown.

In a first set of experiments, we demonstrated that aged mice (24-month-old) treated with BGE-105 exhibit a statistically significant increase in voluntary motor activity (p=0.00228) and a statistically significant improvement in grip strength (p=0.04) as compared to age-matched controls, indicating improved physical health and increased muscle strength.

In addition, aged mice (18-month-old) first injected with a cardiotoxin and then treated with BGE-105 showed significantly higher levels of several mRNA transcripts which are indicative of muscle regeneration.

Third, immortalized muscle precursor cells from human patients showed a dose-dependent relationship between cell growth and differentiation, and concentration of BGE-105.

Further, immobilized aged mice (20-months-old) that were orally dosed with BGE-105 displayed significantly reduced muscle atrophy as compared to immobilized mice that were injected with vehicle.

A Phase 1 clinical study was performed and BGE-105 was shown to prevent or attenuate muscle atrophy in human patients on bed rest. Extensive assessments of several muscle dynamics, patient serum biomarkers and proteomics analyses were performed that are consistent with demonstrations of efficacy in clinical studies.

These analyses further support the use of an apelin receptor modulator such as BGE-105 for treating or preventing various critical muscle conditions or chronic muscle conditions, including, but not limited to diaphragm atrophy, critical care myopathy, frailty, COPD-associated muscle dysfunction, sarcopenia, or other muscle conditions (e.g., as described herein).

Thus, an apelin receptor modulator such as BGE-105 can increase physical performance, counteract age-related frailty, and can reduce age-related muscle weakness.

Accordingly, a first aspect of the present disclosure provides a method for treating or preventing a muscle condition in a subject, the method including administering to a subject in need thereof an effective dose of an apelin receptor modulator. In some aspects of the invention the modulator is an apelin receptor agonist, such as an apelin receptor agonist of formula (I) or (II) as described herein. In some embodiments, the muscle condition is an age-related muscle condition. In some embodiments, the apelin receptor agonist is BGE-105, or a pharmaceutically acceptable salt thereof.

In another aspect, the present disclosure provides a method for maintaining and/or increasing muscle mass, muscle function, and/or muscle strength in an subject. In some embodiments, the subject is an elderly human. The method can include administering to a subject in need thereof an effective dose of an apelin receptor agonist, such as an apelin receptor agonist of formula (I) or (II) as described herein. In some embodiments, the apelin receptor agonist is BGE-105, or a pharmaceutically acceptable salt thereof.

In some embodiments of the methods of this disclosure, the subject is human and has, or is identified as having, one or more of low muscle strength, low muscle force, low muscle mass, low muscle volume. In some embodiments, the muscle is skeletal muscle. In some embodiments, the muscle is the diaphragm, tibialis anterior, tibialis posterior, gastrocnemius, sartorius, vastus intermedius, vastus laterals, vastus medialis, soleus, rectus femorus, or extensor digitorum longus.

In some embodiments of the methods of this disclosure, the subject is human and has, or is identified as having, one or more of diabetes mellitus, insulin insensitivity or resistance, cardiovascular disease, neurologic disease, and chronic obstructive pulmonary disease (COPD).

In some embodiments of the methods of this disclosure, the subject is human and has low muscle strength, low muscle force, low muscle mass, and/or low muscle volume due to disuse atrophy after immobilization.

In some embodiments of the methods of this disclosure, the subject is human and has diaphragm dysfunction and/or diaphragm atrophy. In some embodiments of the methods of this disclosure, the subject is human and has critical illness myopathy.

### 4. BRIEF DESCRIPTION OF THE DRAWINGS

These and other features, aspects, and advantages of the present invention will become better understood with regard to the following description, and accompanying drawings, where:

FIG. 1 shows the structure of BGE-105.

FIGS. 2A-2D graph results from a bioinformatic survival model examining the relationship between serum levels of a given protein and future risk of all-cause mortality (i.e. longevity) or retaining full mobility in human healthy aging cohorts, using non-public clinical outcome data and proteomics data generated on archived samples. FIG. 2A shows a Kaplan-Meier curve of survival probability for humans in the top 20% (upper 20%) versus bottom 20% (lower 20%) of apelin protein levels, demonstrating that in humans higher circulating levels of apelin were associated with decreased all-cause mortality (p=0.0002). FIG. 2B shows a similar model of full mobility by protein levels, where higher circulating levels of apelin were associated with increased retention of full mobility (p=0.0082). The hazard ratio for apelin was 0.88 in FIG. 2A and 0.89 in FIG. 2B. In both cases, the hazard ratio given is for the continuous Cox proportional hazards analysis, which is fitting to the entire distribution of apelin measurements. P-values in FIGS. 2A and 2B were calculated for these hazard ratios, based on testing the null hypothesis that the hazard ratio in each case equals 1. FIG. 2C, shows the serum abundance of the apelin protein module (highlighted by the oval) in the Honolulu Heart Study (HHS) cohort. Each node represents a protein, and the edges between the nodes represent significant correlations. FIG. 2D shows the first principal component of the apelin protein module and death rate. The relative death rate (log; y-axis) was derived from the multivariate Cox regression model for the first principal component (PC1) after adjusting for age, smoking pack years, and alcohol status. The reference used was the median value of PC1.

FIGS. 3A-3I show the effect of BGE-105 on activity and muscle strength of 24-month-old C57BL/6 mice. FIGS. 3A and 3D show the results of repeated experiments on C57BL/6 mice on an activity wheel in their cages, with the readout being km/day. In FIGS. 3A and 3D the solid dots, and associated line, are for mice that were treated with BGE-105, whereas the open dots, and associated line, are for mice that were not treated with BGE-105. Kendall rank correlation tau p=0.00228 and 1.14e-04, respectively. In FIGS. 3B and 3C the open dots are for mice that were treated with BGE-105, whereas the solid dots are for mice that were treated with vehicle only, it shows that on average there was an increase in the latency to fall in the grid hang test (a measure of increased muscle strength) with mice that were treated with BGE-105 as opposed to vehicle. At the end of the study, the BGE-105-treated mice had significantly higher tissue wet weight in the tibialis anterior (TA) (FIG. 3F), and a trend toward higher tissue wet weight in the gastrocnemius and quadriceps (FIGS. 3G and 3H), and no difference in heart (FIG. 3I). The BGE-105-treated mice also had higher body weights (FIG. 3E), although the increase was not significant, just at p-value cutoff.

FIGS. 4A-4D depict increased levels of pAMPK (FIG. 4A-4B) and pAkt (FIGS. 4C-4D) in BGE-105-treated mice vs. vehicle-treated mice.

FIGS. 4E-4F depict that per unit mass, the soleus contained about half as much APLNR receptor as the heart, potentially explaining the stronger response in heart tissue.

FIGS. 5A-5B depict the levels of apelin receptor protein found in rat tissue. FIGS. 5C-5D depict oral dosing of rats with BGE-105 for 5 consecutive days induced phosphorylation of Akt in the TA in a dose-dependent manner, with 50 mg/kg BID eliciting the strongest response. FIGS. 5E-5F depict the same for Erk. FIGS. 5G-5I depict the effect of chronic administration of BGE-105 on apelin receptor protein levels in the TA.

FIGS. 6A-6B show that in cells stably expressing human apelin receptor, BGE-105 was 10-fold more potent than Pyr¹-Apelin-13 and in cells stably expressing mouse apelin receptor, BGE-105 was 30-fold more potent than Pyr¹-Apelin-13.

FIGS. 7A-7F show the effect of administering PBS, Pyr¹-Apelin-13 (apelin) (0.5 mol/kg/day), BA1 (BGE-105 50 mg/kg/day), or BA2 (BGE-105 200 mg/kg/day) on transcript levels in the tibialis anterior of aged (18-month-old) mice either 3 days or 7 days post injection of cardiotoxin.

FIGS. 7G-7L show the effect of administering PBS, Pyr¹-Apelin-13 (apelin) (0.5 mol/kg/day), BA1 (BGE-105 50 mg/kg/day), or BA2 (BGE-105 200 mg/kg/day) on transcript levels in the gastrocnemius of aged (18-month-old) mice either 3 days or 7 days post injection with cardiotoxin.

FIGS. 7M-7R show the effect of administering PBS, Pyr¹-Apelin-13 (apelin) (0.5 μmol/kg/day), BA1 (BGE-105 50 mg/kg/day), or BA2 (BGE-105 200 mg/kg/day) on transcript levels in the tibialis of young (3-month-old) mice either 3 days or 7 days post injection with cardiotoxin.

FIGS. 7S-7X show the effect of administering PBS, Pyr¹-Apelin-13 (apelin) (0.5 mol/kg/day), BA1 (BGE-105 50 mg/kg/day), or BA2 (BGE-105 200 mg/kg/day) on transcript levels in the gastrocnemius of young (3-month-old) mice either 3 days or 7 days post injection with cardiotoxin.

FIGS. 7Y-7Z show the cross-sectional area of the tibialis at day 3 and day 7 post injection of cardiotoxin after treatment with PBS, Pyr¹-Apelin-13 (apelin), BA1 (BGE-105 50 mg/kg/d), and BA2 (BGE-105 200 mg/kg/d).

FIGS. 7AA-7BB show the amount of centrally nucleated fibers (CNM) as part of the regenerative process after cardiotoxin injection for PBS, Pyr¹-Apelin-13 (apelin), BA1 (BGE-105 50 mg/kg/day), and BA2 (BGE-105 200 mg/kg/day) treatments. Mice were 18-months old. FIG. 7AA shows representative distribution of DAPI stained nuclei and positively stained eMHC fibers. FIG. 7BB shows the quantification of the amount of centrally nucleated myofibers (CNM).

FIGS. 8A-8C show the ability of BGE-105 to increase the proliferation of immortalized human muscle cells from both younger (25-years-old) and older (79-years-old) subjects.

FIGS. 8D-8K show the levels of PAX7, MYf5, MyoD and MyoG expression in immortalized muscle cells from older (79-years old) and younger (25-years-old) subjects after incubation with DMSO (0.1%), Pyr¹-Apelin-13 (Apelin/Ape) (1 nM), or BGE-105 (BA) at 0.05, 0.5, 5, or 50 nM. FIG. 8D shows the levels of PAX7 in the younger cells. FIG. 8E shows the levels of MYf5 in the younger cells after treatment. FIG. 8F shows the MyoD expression levels after treatment in the younger cells. FIG. 8G shows the levels of MyoG expression after treatment in the younger cells. FIG. 8H shows PAX7 levels after treatment in older cells. FIG. 8I shows the levels of Myf5 expression after treatment in cells derived from the older donor. FIG. 8J shows the levels of MyoD after treatment cells derived from the older donor. FIG. 8K shows the levels of MyoG after treatment in the older cells.

FIGS. 9A-9M show effects of BGE-105 in preventing disuse-induced muscle atrophy in aged mice.

FIG. 10 provides a study dosing outline for BGE-105 in a Phase 1 clinical study with single ascending dose (SAD) cohorts 1-3, or multiple doses (MD) cohorts 1A-1C of Example 9.

FIG. 11 provides a screening and pre-treatment outline of SAD cohorts of Part A in Example 9. Abbreviations: AE=adverse event; ECG=electrocardiogram; HbsAg=hepatitis B surface antigen; HCV=hepatitis C virus; HIV=human immunodeficiency virus; PD=pharmacodynamic; SAD=single ascending dose TBD=to be determined.

FIG. 12 provides a treatment and follow up outline of SAD cohorts of Part A in Example 9. Abbreviations: AE=adverse event; ECG=electrocardiogram; EOS=end of study; EOT=end of treatment; FU=follow-up; IP=investigational product; IV=intravenous; LD=loading dose; MD=maintenance dose; PD=pharmacodynamic; PK=pharmacokinetic; SAD=single ascending dose; TBD=to be determined.

FIG. 13 provides a screening and pre-treatment outline of MD cohorts of Part B in Example 9. Abbreviations: AP=anterior-posterior; BMR=basal metabolic rate; D3-Cr=deuterated creatine; ECG=electrocardiogram; FSR=fractional synthetic rate; HbsAg=hepatitis B surface antigen; HCV=hepatitis C virus; HIV=human immunodeficiency virus; PD=pharmacodynamic; TBD=to be determined.

FIG. 14 provides a treatment and follow up outline of MD cohorts of Part B in Example 9. Abbreviations: AP=anterior-posterior; BMR=basal metabolic rate; D3-Cr=deuterated creatine; ECG=electrocardiogram; FSR=fractional synthetic rate; HbsAg=hepatitis B surface antigen; HCV=hepatitis C virus; HIV=human immunodeficiency virus; PD=pharmacodynamic; TBD=to be determined.

FIG. 15 shows Preliminary PK data from the 3 SAD cohorts demonstrating dose proportionality. The Cₘₐₓ remained within the expected range and for the highest dose (240 mg/1440 mg) the AUCₗₐₛₜ was 1062 μg*hr/mL.

FIG. 16 shows average of percent change of HOMA-IR from baseline.

FIG. 17 shows mouse apelin receptor (APJ)/APLNR expression levels in the heart, diaphragm, tibialis anterior (TA), and the brain of adult mice tissue. As shown, APLNR is highly expressed in the diaphragm, in line with canonical apelin target tissues: skeletal and cardiac muscles. Tissues were harvested from a 7 month-old mouse and homogenized. Western blots were performed using 10 μg protein and probed for APJ (Invitrogen #5H5L9) and loading control GAPDH (Abcam #EPR1689). Bands were quantitated using a BioRad ChemiDoc XRS+Molecular Imager. APJ signal was normalized to GAPDH.

FIGS. 18A-18B show the effects of BGE-105 on rest-induced reduction in thigh circumference, Vastus Lateralis diameter % (thickness), Vastus Lateralis cross sectional area (CSA) %, muscle degeneration, and cumulative protein synthesis rate % (as measured from a biopsy). FIG. 18B shows thigh circumference of patients received placebo (Cohort 1A of MD study of Example 9) and BGE-105 treatment (Cohort 1B of MD study of Example 9) period is measured and presented as percent change from baseline over the course (middle panel) or after (left panel) of the 10-day bed rest duration. p=0.0004.

FIG. 19 shows effects of patients treated with BGE-105 on muscle diameter and cross-sectional area of the vastus lateralis via ultrasound (left panel) after 10-days of bed rest. The vastus lateralis diameter (middle panel) or vastus lateralis cross sectional area (left panel) of patients treated with BGE-105 or placebo is presented as a mean percent change from baseline. p=0.0297.

FIGS. 20A-20C show fatty degeneration of the vastus lateralis via echo density as measured using an ultrasound muscle quality grading scale (panels A-B). FIG. 20A is a diagram illustrating a grade of 1 in normal muscle (1, open box), FIG. 20B illustrates a grade 2 of muscle containing some fatty streaks (2, striped box). Shown are muscle quality at baseline and after 10 days of bed rest observed in each of placebo and BGE-105 group (FIG. 20C) (p=0.0019).

FIG. 21 shows rate of muscle protein synthesis in the vastus lateralis, measured via microbiopsy in individual subjects in the BGE-105 treated group and the placebo group. Muscle protein synthesis is measured after 10 days bed rest and normalized fraction synthetic rate by subject is presented. p=0.0043.

FIGS. 22A-22B illustrate a timeline of the phase 2 POC trial in ICU diaphragm atrophy or critical illness myopathy. FIG. 22B illustrates design of the phase 2 POC trial in mechanically ventilated patients.

FIGS. 23A-23B show healthy volunteer characteristics and of the Phase 1b bed rest atrophy of the MD study and incidence of treatment emergent adverse (Part B of Example 9). The Placebo group is represented as Cohort 1A of the MD, Part B study, and the BGE-105 treated group is represented as Cohort 1B of the MD, Part B study of Example 9.

FIG. 24 shows effect of BGE-105 on rest-induced reduction in thigh circumference of subjects participating in Phase 1b, MD, Part B clinical trial of Example 9. Thigh circumference was measured in patients receiving placebo or BGE-105 treatment and presented as percent change from baseline. Subjects received BGE-105 fully preserve or show increased thigh circumference over 10 days of bed rest. Measurements are made 15 cm superior of the mid patella. P=0.0004.

FIGS. 25A-25B show effect of BGE-105 on rest-induced reduction in calf circumference of subjects participating in Phase 1b, MD, Part B clinical trial of Example 9. FIG. 25A shows graph of calf circumferences of patients receiving placebo or BGE-105 treatment is measured and presented as percent change from baseline over the course or after the 10-day bed rest duration. Calf circumferences are presented as percent change from baseline to day 10 (right panel). p=0.0024. Measurements are made 15 cm superior of the mid patella. FIG. 25B shows the percentage change in calf circumference exhibited by various subjects from baseline to day 10.

FIG. 26 shows the effects of BGE-105 on gastrocnemius diameter and gastrocnemius cross-sectional area via ultrasound after 10-days of bed rest. The gastrocnemius diameter (middle panel, p=0.836) or gastrocnemius cross sectional area (left panel, p=0.278) of patients treated with BGE-105 or placebo is presented as a mean percent change from baseline. As shown, there is a larger decrease in gastrocnemius diameter (10.982% decrease) and cross-sectional area (15.72% decrease) in the placebo group as compared to the diameter (2.942 decrease) and cross-sectional area (7.758% decrease) in the BGE-105 treated group.

FIGS. 27A-27B show effects of BGE-105 in thigh and calf circumference, vastus lateralis cross sectional area, and gastroenemius cross sectional area measurements in female subjects. Thigh circumference (p=0.0033), calf circumference (p=0.0146), vastus lateralis cross sectional area (p=0.066), and gastrocnemius cross sectional area (p=0.4381) in subjects receiving placebo or BGE-105 were measured and presented as percent change from baseline. As shown, there is a larger decrease in thigh circumference (6.06% decrease) and calf circumference (5.486% decrease) in the placebo group as compared to the thigh circumference (1.674% decrease) and calf circumference (1.166% decrease) in the BGE-105 treated group (FIG. 27A). As shown, there is a larger decrease in vastus lateralis cross sectional area (23.67% decrease) and gastrocnemius cross sectional area (15.32% decrease) in the placebo group as compared to the vastus lateralis cross sectional area (9.676% decrease) and gastrocnemius cross sectional area (5.558% decrease) in the BGE-105 treated group (FIG. 27B).

FIG. 28 shows synthetic rate of muscle myofibril proteins in the vastus lateralis in BGE-105 treated and placebo treated patients. Average vastus lateralis muscle myofibril synthesis rate is measured and presented as change from baseline (left panel). Average myofibril FSR/day is presented as percent change from baseline (right panel). As shown, there is a larger decrease in the synthetic rate of muscle myofibril proteins in placebo (58% decrease) vs BGE-105 treated (36% decrease) group.

FIG. 29 illustrates measurements taken during the Phase 1b bed rest atrophy study used for assessment of several muscle dynamics as shown in Example 9.

FIG. 30 illustrates description of enabling modalities used to assess muscle dynamics in Phase 1b bed rest atrophy study of Example 9.

FIG. 31 illustrates the site and method of ultrasound measurements of leg muscle. Ultrasound measures skeletal muscle circumference, cross-sectional area, color flow analysis, anterior-posterior diameter, and echo density of the vastus lateralis and gastrocnemius.

FIG. 32 illustrates incorporation of deuterated water into muscle proteins for calculation of the fractional synthetic rate. Deuterium can be measured both invasively (via tissue biopsy) and noninvasively (via, blood and urine—a virtual biopsy).

FIG. 33 shows microbiopsy of the vastus lateralis performed at several time points (at baseline day zero of bedrest, at day 5 of bedrest, and at the end of the 10-day bed rest period) for measuring the fractional synthetic rate of hundreds of muscle proteins. Biopsy performed with microneedle was collected at three timepoints: baseline, midway through the treatment period, and end of the treatment period.

FIG. 34 depicts a flowchart for assessing total muscle mass using a D3-creatine tracer during the phase 1b study of Example 9. The data is validated against MRI muscle volume in humans (r=0.87, p<0.01).

FIG. 35 shows correlation of atrophy of skeletal muscle and decrease in muscle protein synthesis in older patients (>65 years old). Muscle atrophy was presented as change in lean leg mass (g) (left panel). Muscle fractional synthetic rate is measured before and after the 10 day bed rest period in patients treated with BGE-105 or placebo, and is presented as percent change (right panel).

FIG. 36A shows the effect of BGE-105 on muscle protein synthesis in vastus lateralis microbiopsies in subjects treated with BGE-105 or placebo. Shown is ratio of protein FSR BGE-105 over placebo after 10 days of bed rest (middle panel). Shown is comparison of cumulative protein synthesis in placebo and BGE-105 treated subjects at after 5 day bed rest and after 10 day bed rest (right panel). FIG. 36B shows the fractional synthesis of BGE-105 treated patients and placebo treated patients on day 11 of bedrest. As shown, BGE-105 resulted in higher muscle protein synthesis in the vastus lateralis compared to placebo-treated patients, measured via microbiopsy.

FIG. 37 shows the step ratio of patients who wore a wearable activity device during Day 10 through Day 60 (post-bedrest time period) of the Phase 1b, MD, Part B clinical trial of Example 9.

FIGS. 38A-38D show proteomic profiling analysis performed on serum collected from subjects of the phase 1B clinical trial of Example 9. 11 treated and 11 placebo subject's serum levels were profiled for their proteomics collected at day −1 (baseline), day 5 and day 11. FIG. 38A shows the number of proteins associated with frailty (functionality), walk speed, instrumental activities of daily living (IADL) (functional instrument), and grip strength that are changed in patients treated with BGE-105 in the phase 1b clinical trial of Example 9. FIG. 38B shows that BGE-105 shifted the serum proteome towards a healthier state, recapitulating the benefits of naturally high apelin levels in subjects treated with BGE-105. FIG. 38C shows changes in baseline energy expenditure in BGE-105 treated and placebo treated subjects using a SomaSignal test on the proteomic data. FIG. 38D shows changes in cardiorespiratory fitness (VO₂) max and basal metabolic rate in BGE-105 treated and placebo treated subjects using a SomaSignal test on the proteomic data.

FIG. 39 provides the clinical outline of a Phase 2 clinical study for treatment of patients with ICU diaphragm atrophy.

FIGS. 40-62 shows details and results of snRNAseq analyses of human muscle tissue samples from a Phase 1b clinical study of BGE-105 for treating muscle atrophy.

FIG. 40 illustrates workflow of a nucleic isolation kit used in BGE105 clinical trial streamlined sample preparation. 10× Genomics single cell library technology is used for assessing tissue samples.

FIG. 41 shows 11 cell types were identified, consistent with published muscle atlas. Two methods and two annotations were used. Top 20 variable genes within clusters were used as markers for cell type annotation.

FIG. 42 shows that there is consistency between the top 20 cell-type specific expressed genes and known cell type markers. Plot shows log 2(fold change) in expression in a specific cell-type compared to rest for the top 20 cell-type specific expressed genes. Analysis was performed using all samples.

FIG. 43 shows that differentially expressed genes associated with BGE105 were identified for each cell types.

FIG. 44 shows that signaling pathways that control muscle loss and promote muscle growth were enriched in BGE105 treatment associated genes in fast skeletal muscle. Genes with notable significance (padj<0.1) were underscored.

FIG. 45 shows that treatment association of most of significant genes (padj<0.1) in muscle growth/loss related signaling pathways were in the anticipated direction. Shown are detection of genes associated with fast skeletal muscle treatment (padj<0.001) and slow skeletal muscle (Type I) treatment.

FIG. 46 shows that bulk expression level of VEGFA, PPRGC1A and COL1A1 were higher in treated group than that in placebo group on day 11.

FIG. 47 shows that bulk expression level of TNNC1 and MYH7 were higher in treated compared to placebo on day 11. Shown is bulk expression level: mean of gene across all cells for each patient. Cell types were ignored. TNNC1 and MYH7 are both slow skeletal muscle cell markers.

FIG. 48 shows that for fast skeletal muscle: 10 groups of 5+ enriched pathways were identified.

FIG. 49 shows that for slow skeletal muscle: 13 groups of 5+ enriched pathways were identified.

FIG. 50 shows cell type-specific patterns of differential gene expression associated with BGE-105 treatment identified in muscle biopsies.

FIG. 51 shows that differentially regulated pathways suggest BGE-105 beneficial effects on key muscle and adipocyte processes.

FIG. 52 shows BGE-105 prevents bed rest-induced downregulation of muscle contractile proteins in fast and slow skeletal muscles. p values reflect change after 10 days of bedrest, compared to baseline.

FIG. 53 shows BGE-105 prevents bed rest-induced downregulation of mitochondrial biogenic regulator PGC-1α and all respiratory complexes. Shown are representative genes.

FIG. 54 shows BGE-105 prevented detrimental expression level of genes involved in muscle metabolic processes. Shown are differential gene expression of insulin signaling pathway (p=2.61E-03), AMPK signaling pathway (p=5.04E-03), and glucagon signaling (p=2.61E-03).

FIG. 55 shows BGE-105 prevents bed rest-induced upregulation of genes involved in triglyceride storage and fatty acid metabolism, potential mechanism for promoting fat loss.

FIG. 56 shows APLNR was expressed in more endothelial cells in the treated group. A small proportion of cells expressed APLNR (272 cells). 46% of these cells were within the endothelial cells. T-test of APLNR expression level in detected cells between treated and placebo groups: Day6_t.stat=2.02, Day6_p.val=0.04, Day11_t.stat=2.33, Day111p.val=0.02.

FIG. 57 Cell differentiation trajectory and pseudotime inference.

FIG. 58 shows a plot indicating cells were less differentiated with treatment for Fast/Slow skeletal muscle, macrophages, T/NK cells and muscle stem cell. Baseline muscle stem cells (treated and placebo) were set as the root.

FIGS. 59-61 illustrate the results of secondary analyses. Evaluation of signal from protein synthesis rate analysis. Investigation of aging and muscle signatures from published research.

FIG. 59 shows BGE-105 resulted in relatively higher muscle protein synthesis in the vastus lateralis, measured via microbiopsy (p<0.005). Shown is muscle protein synthetic rate after 10 days of bed rest. Asterisks indicate 10 significant genes: TPM2, PYGM, MYH2, TNNI2, TNNC2, TNNC1, ENO3, ALDOA, ATP 5F1B, MDH2.

FIG. 60 shows the validation of muscle protein synthesis assay results in snRNA-seq analysis. 15 out of 18 muscle proteins show statistically highly expressed in fast skeletal muscle in treated compared to the placebo group. 13 myofibril proteins (5 proteins were significant in FSR analysis: TPM2, TNNC1, TNNC2, TNNI2, MYH2) and 5 significant non-myofibril proteins in FSR analysis (5 proteins: ENO3, PYGM, ALDOA, MDH2 ATP5F1B). All 38 proteins in the heavy-water were also measured in snRNA-seq data.

FIG. 61 shows that BGE-105 treatment shifts the transcriptome of fast/slow skeletal muscles towards a state linked to younger muscle. Transcripts negatively associated with BGE-105 treatment shows enrichment for the muscle aging signature. See e.g., Perez et al. (2022), Aging.

FIG. 62 shows graph that indicate percentage of cells that have Mitochondria reads more than 5% within a sample. Y-axis is the percentage of cells that MT reads >5% within a sample (62 samples=21 patients*3 time points) compared to that in baseline. P values of T-test are day 6, p=0.08; day 11, p=0.04. Cells that have low qualities have been removed before calculating the proportion of cells that have more than 5% Mitochondrial reads for each patient (cells with less than 200 genes or less than 500 reads were removed, total remaining MT cells (>5%).

FIG. 63 shows BGE-105 preserves synthesis rate of structural proteins to maintain muscle mass. Muscle protein fractional synthesis rate (FSR) were determined for skeletal muscle proteins detected in vastus lateralis muscle tissue (collected by microneedle biopsy). FSRs were scaled across subjects within each protein to account for different baseline rates of fractional synthesis. A two-sample t-test comparing values between groups is statistically significant at p<0.005.

FIG. 64 shows BGE-105 prevent bed rest-induced downregulation of mitochondrial biogenic regulator PGC-1α and mitochondrial respiratory complexes. A representative gene is shown from each respiratory complex.

FIG. 65, panels A-B, show BGE-105 treatment shift the proteome towards an estimated higher basal metabolic rate and VO2 max by SomaSignal tests. Panel A: The resting energy expenditure test was validated by SomaLogic using plasma proteomics in comparison to measured resting energy expenditure using indirect calorimetry and the Abbreviated Weir Formula. Plasma samples were derived from UK-based adults aged 29-64 years (N=9022). Panel B: The VO₂ max test was validated by SomaLogic using plasma proteomics in comparison to VO₂ max values derived from oxygen consumption at maximal exercise (in ml·kg/min) during a standard graded cardiopulmonary exercise test (CPET). Plasma samples were derived from North American adults aged 15-65 years old (N=743).

FIG. 66 shows BGE-105 improves post-bedrest recovery by wearable accelerometer. Day 1 to Day 10: treatment and bed rest period. Normal activities resume on Day 11. Both groups show a steep recovery in step count immediately after the bedrest period, but this was more pronounced in the azelaprag group, leading to separations between the curves between approximately Day 15 and Day 36, after which the curves became indistinguishable. Spearman correlation coefficient used to test for increasing separation between the curves; p-values of 0.028, 0.00092, and 0.00002 at the ends of the first, second, and third weeks post-bedrest, respectively.

FIG. 67 illustrates the flowchart of a single-center, double-blind phase 2A, randomized control trial (RCT), BGE-105 clinical trial, investigating effects on muscle mass (primary outcome), muscle strength, and frailty outcomes.

FIG. 68 shows BGE-105 prevented bed rest-induced downregulation of muscle contractile proteins in fast and slow skeletal muscle. Shown are single-nucleic transcriptomics data of percentage change from baseline for calcium-ATPase type 2 in the sarco-/endoplasmic reticulum (SERCA2) and myosin light chain 3 (MYL3). p values reflect change after 10 days of bedrest, compared to baseline.

FIGS. 69A-69B show BGE-105 prevented bed rest-induced downregulation of mitochondrial biogenic regulator PGC-1α and all respiratory complexes. FIG. 69A shows a summary of downregulation of respiratory complexes. FIG. 69B shows representative genes. p values reflect change after 10 days of bedrest, compared to baseline.

FIGS. 70A-70B show BGE-105 preserved gene expression involved in glucose metabolism. p values reflect change after 10 days of bedrest, compared to baseline. FIG. 70A shows a summary of preserved gene expression in glucagon, insulin and AMPK pathways. FIG. 70B shows insulin signaling pathway differential expression (p=2.61E-03) and AMPK signaling pathway differential expression (p=5.04E-03).

### 5. DETAILED DESCRIPTION OF THE INVENTION

### 5.1. Apelin Receptor Modulators and Frailty

5.1.1. Survival Predictability Model

The present disclosure describes a bioinformatics model that generally relates to building of survival predictor models that output a survival metric. Such survival metrics may relate to survival related observables, such as survival expectancy and/or risk of death. Survival predictor models may be built by selecting observables that relate to survival periods (“aging indicator”). Such aging indicators may comprise variables that correlate with all-cause mortality, such as certain clinical factors. Survival predictor models can utilize one or a plurality of survival biomarkers together with one or more aging indicators to generate a survival metric.

In some embodiments, a survival predictor model of the present disclosure examines the relationship between serum levels of apelin, and future risk of all-cause mortality in human healthy aging cohorts, with clinical outcome data proprietary to those cohorts and proteomics data generated on archived samples, based on survival modeling. Additionally, the relationship between apelin and mobility decline events (e.g., a decrease in ability of walking, stair-climbing, or transferring activities as shown by self-reported difficulty of these activities) is examined using a Cox proportional hazards model, with a hazard ratio and associated p-value generated for apelin.

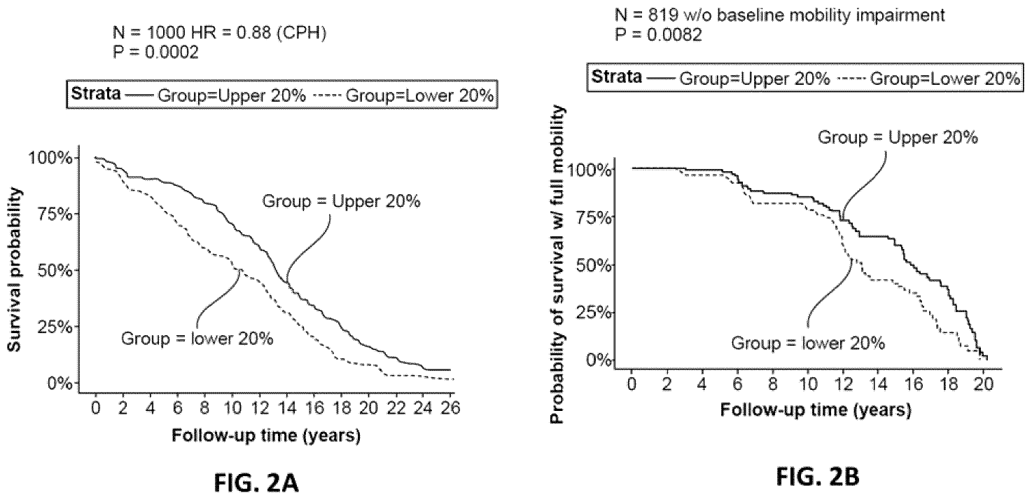

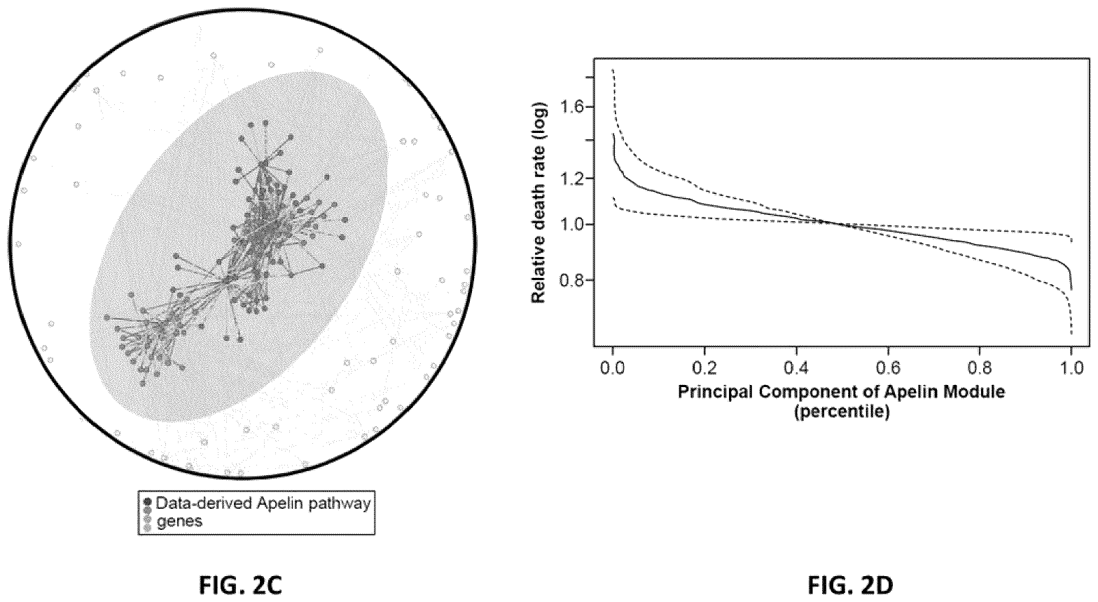

We applied such bioinformatic and machine learning approaches to analyze human data using survival predictor models and discovered an association of apelin receptor levels with future aging outcomes. We discovered that higher circulating levels of apelin are associated with decreased all-cause mortality (p=0.0002)—that is, greater longevity. See, e.g., FIG. 2A. In addition, our analyses demonstrated that higher levels of apelin are associated with better future physical function in healthy aging human subjects. FIG. 2B shows a similar model that indicates higher circulating levels of apelin are associated with increased retention of full mobility (p=0.0082).

5.1.2. Apelin Receptor Expression in Aged Subjects

There is also a demonstrated relationship between the age of mice or humans and apelin receptor expression. The expression of apelin receptor decreases with age in skeletal muscle. Samples taken from frail older patients showed an even larger decrease in apelin receptor levels. Further details are provided in the experimental section, see, e.g., Example 1 and FIGS. 2A-2D.

5.1.3. Aged Mouse Study

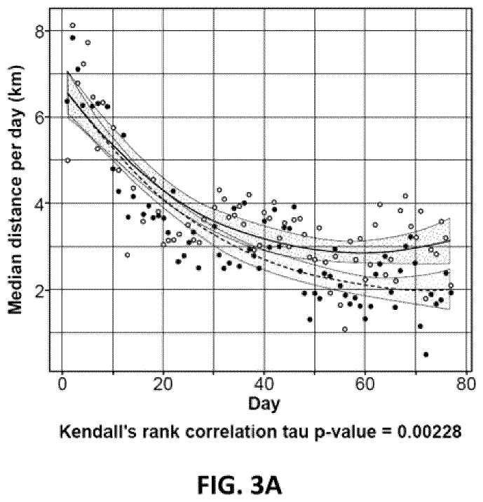

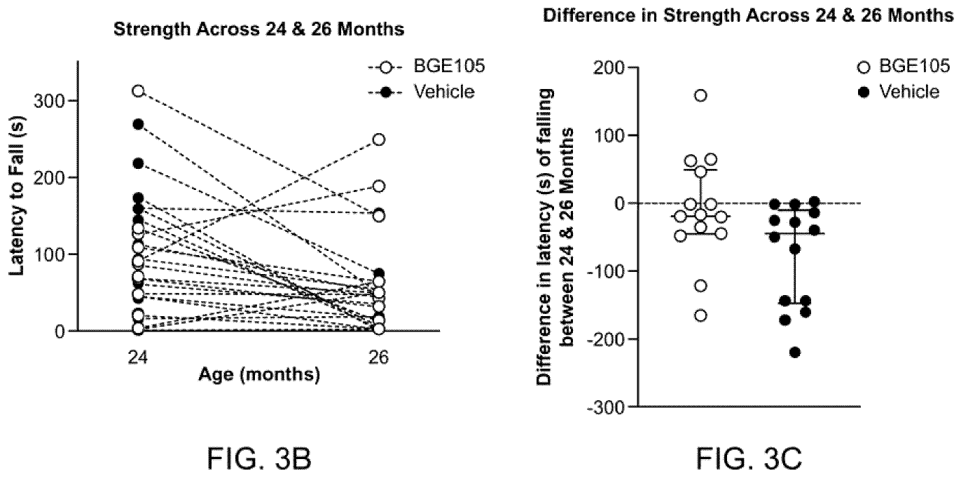

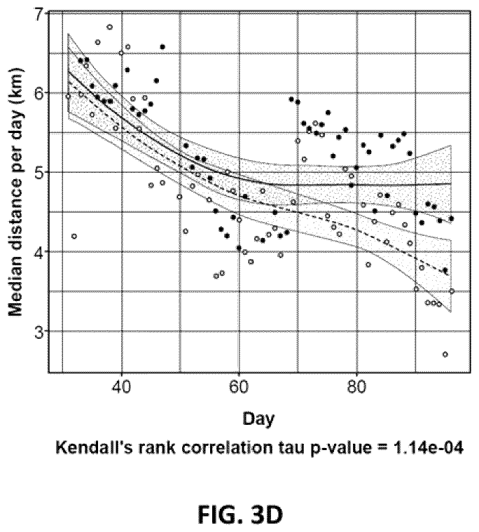

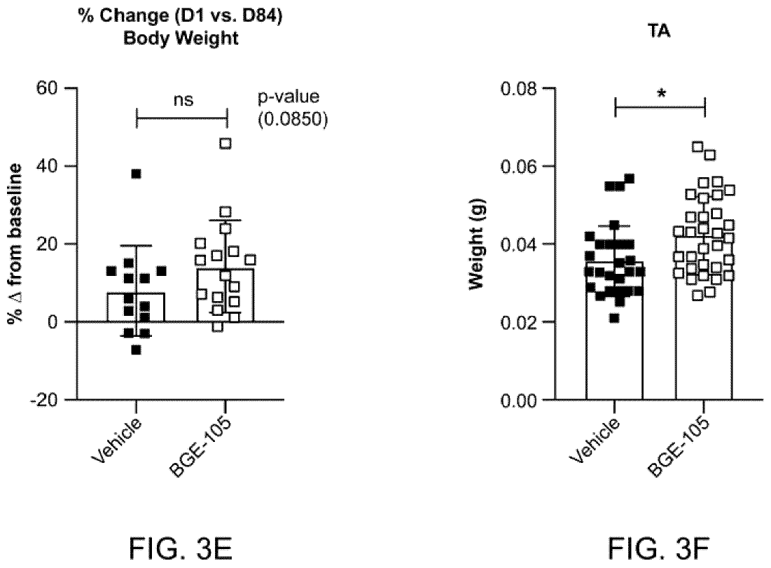

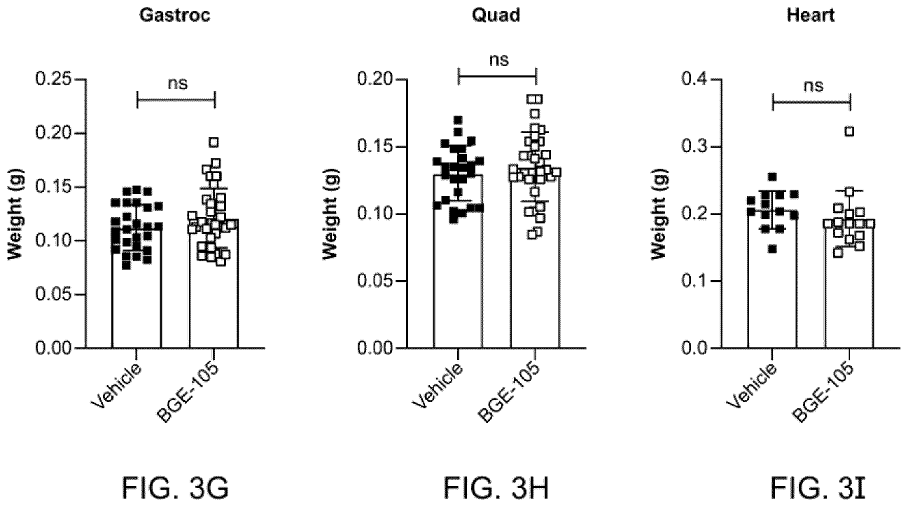

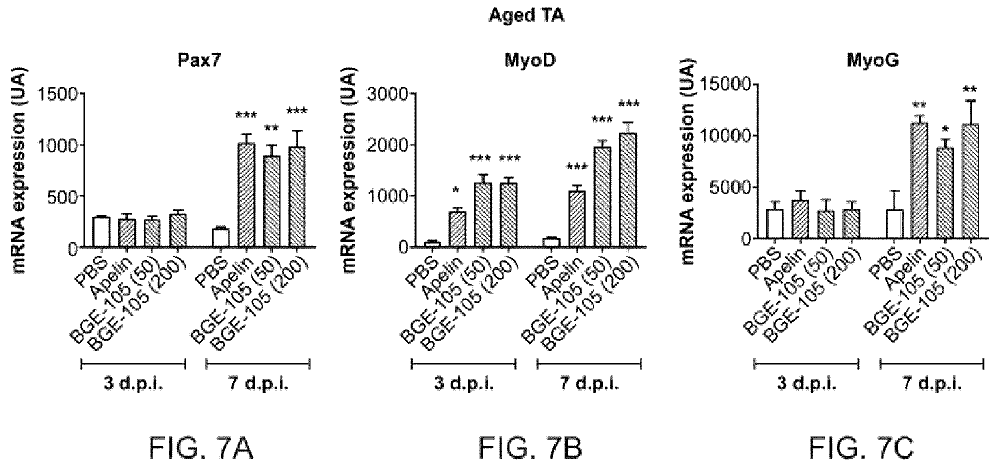

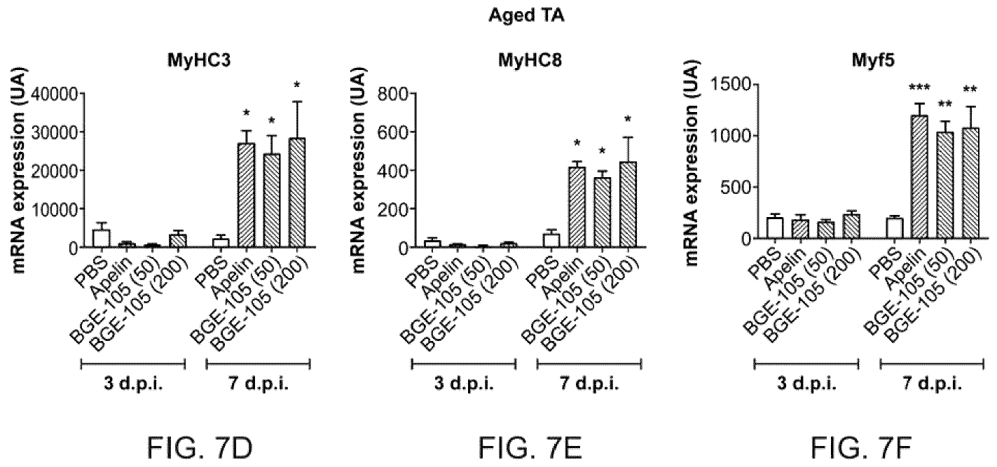

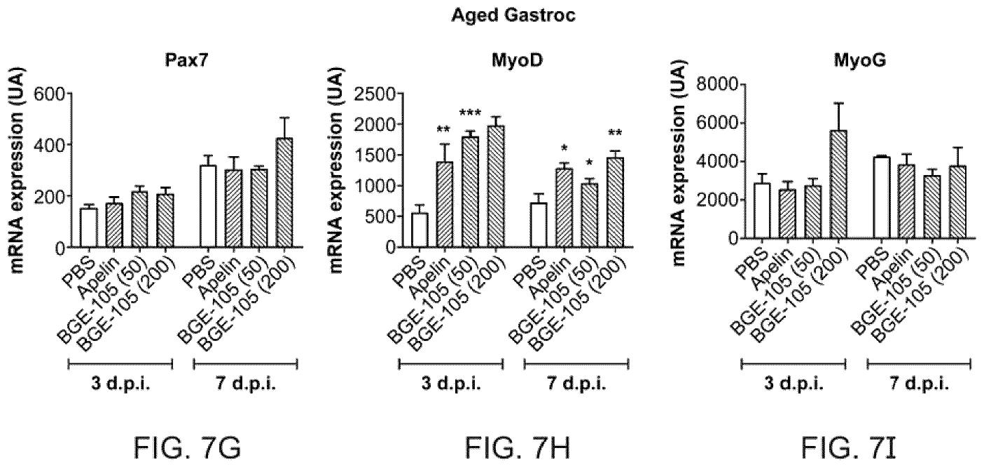

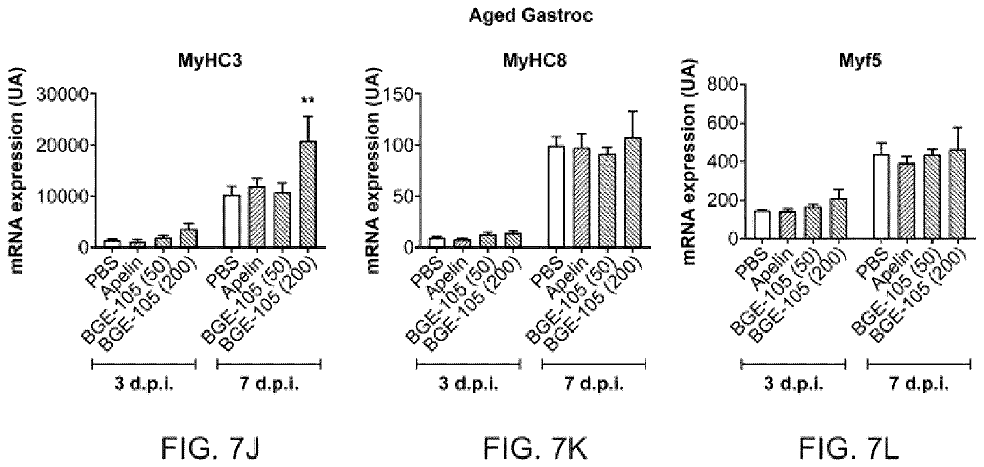

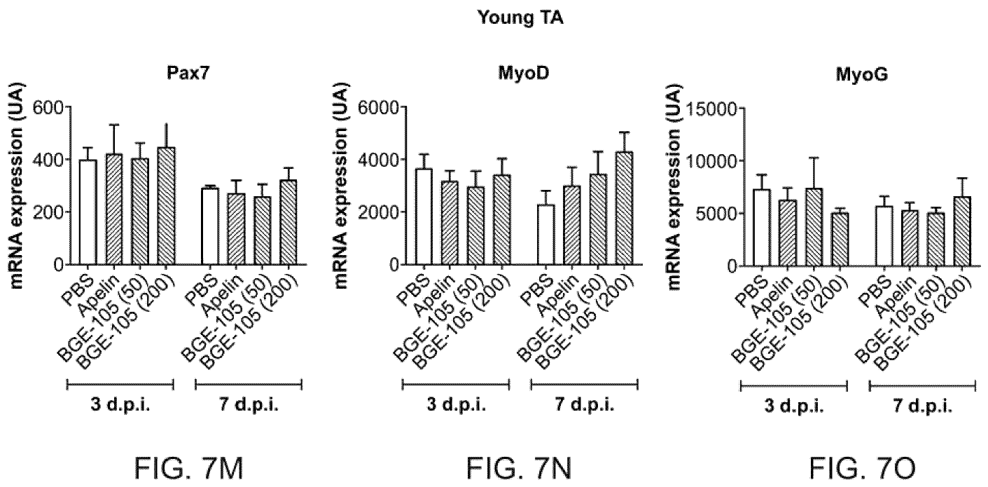

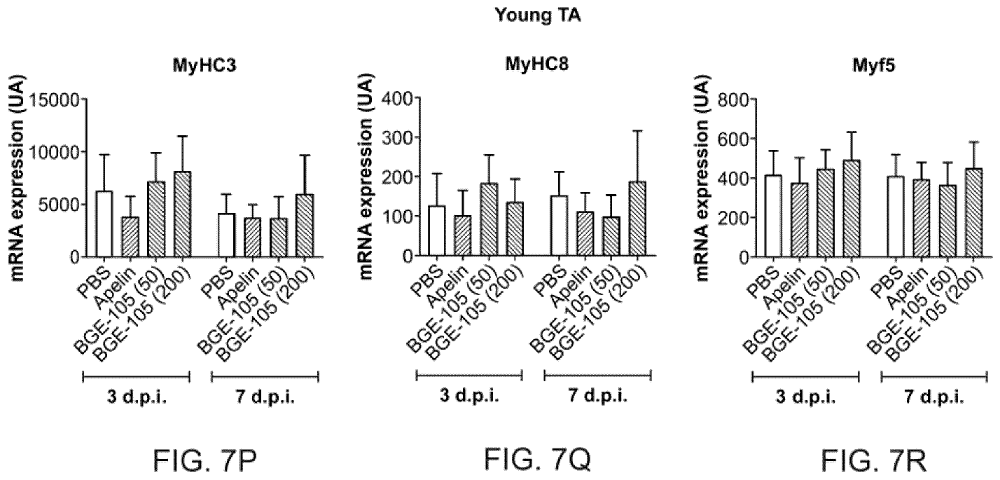

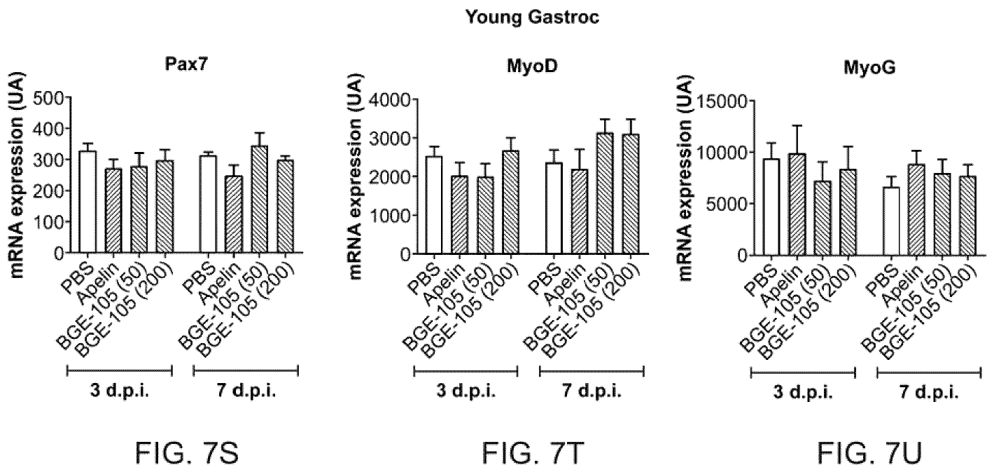

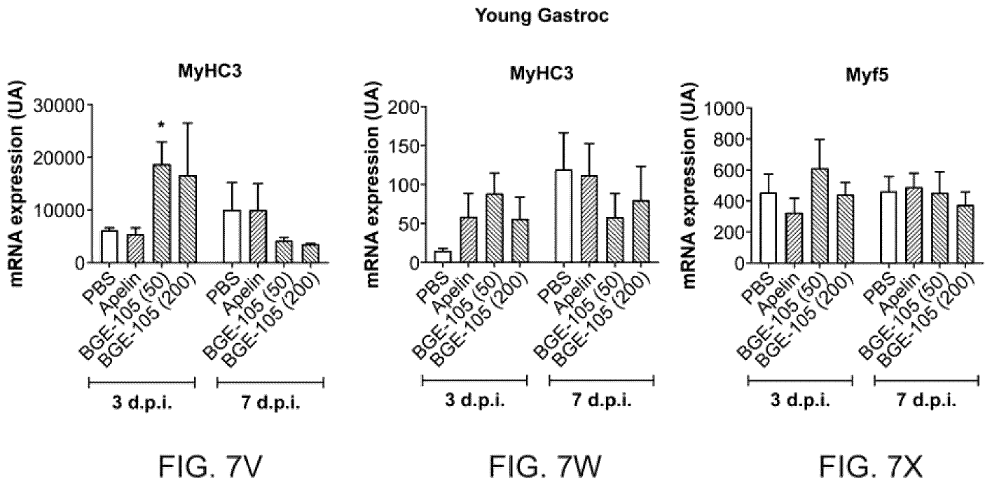

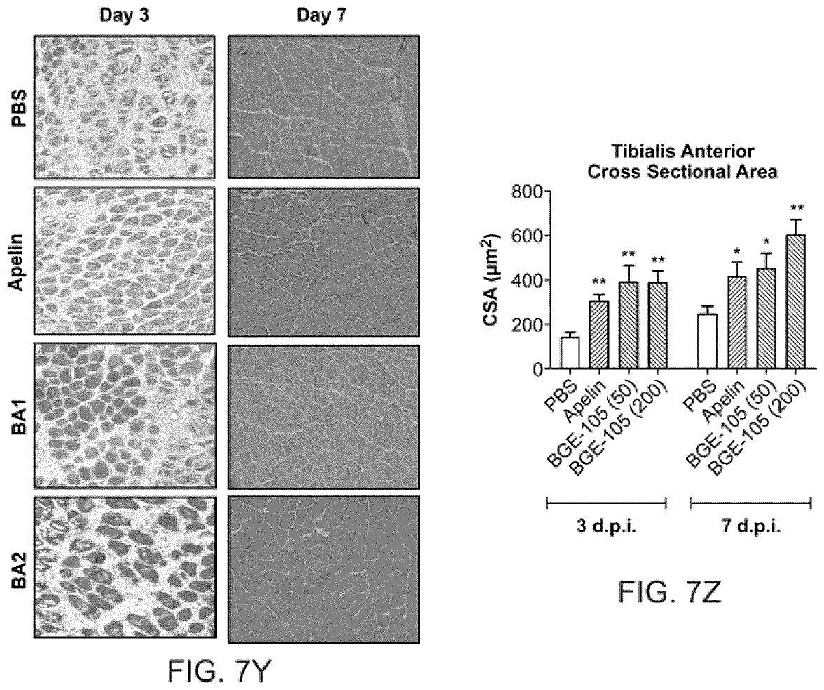

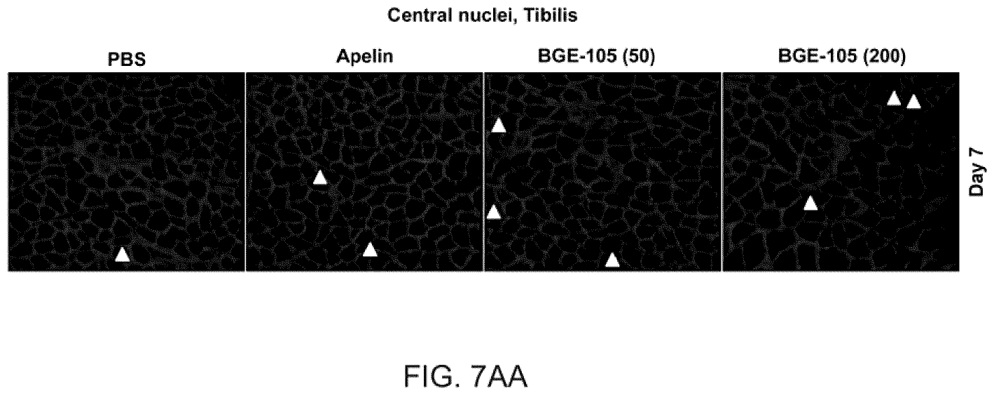

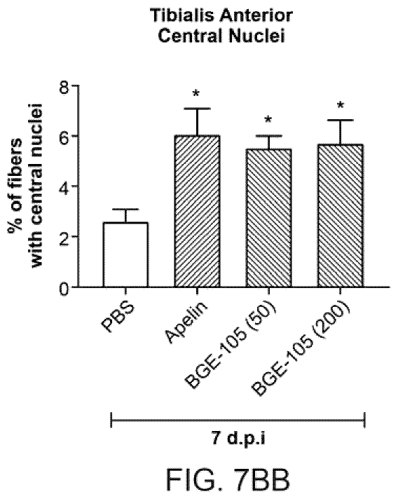

We demonstrated that aged mice (24-months old) treated with BGE-105 exhibit a statistically significant increase in voluntary activity (p=0.002) and an improvement in grip strength (p=0.04) as compared to age-matched controls, indicating improved physical health and increased muscle strength (FIGS. 3A-3I). In addition, aged mice (18-months old) first injected with a cardiotoxin and then treated with BGE-105 showed significantly higher levels of several transcripts which are indicative of muscle regeneration (FIGS. 7A-7X). Cells from these mice were frozen and histological sections were stained. The sections showed a dose-dependent increase in central nucleated fibers (indicative of muscle growth) (FIGS. 7AA-7BB). Further details are provided in the experimental section, see, e.g., Examples 2-5 and FIGS. 3-7.

5.1.4. BGE-105 Activates the Apelin Pathway In Vitro

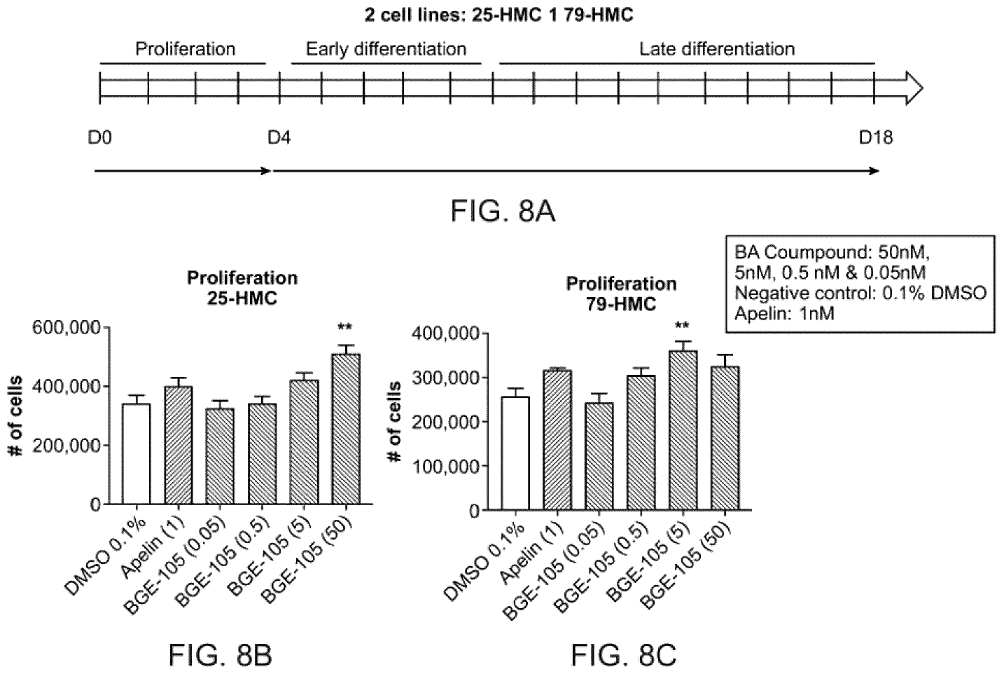

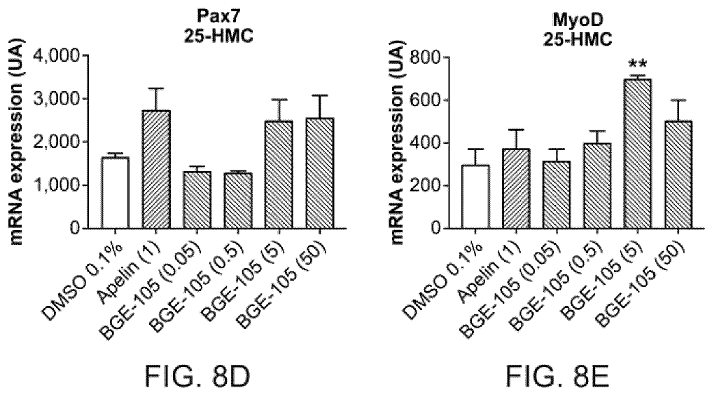

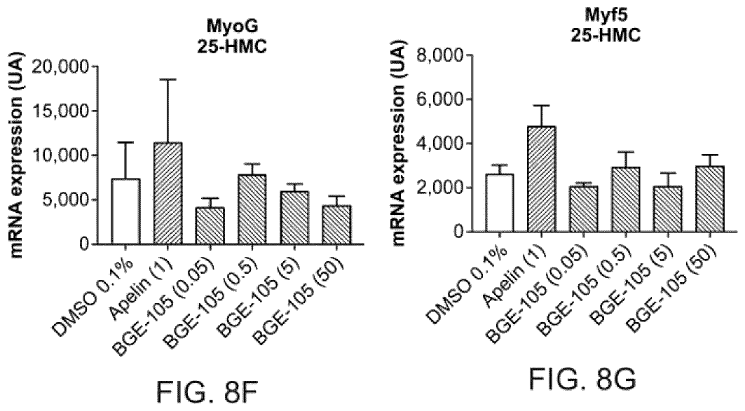

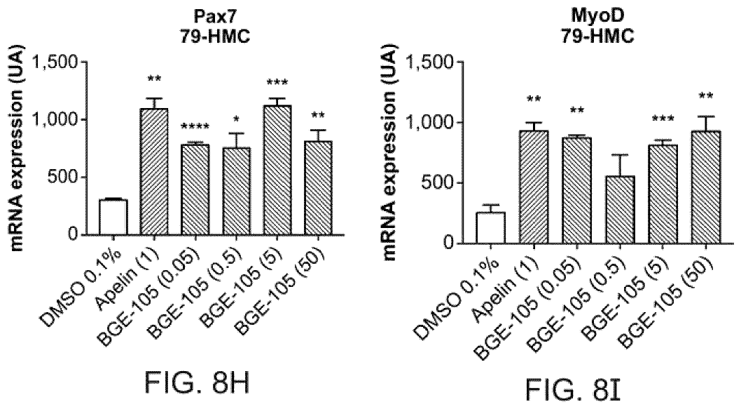

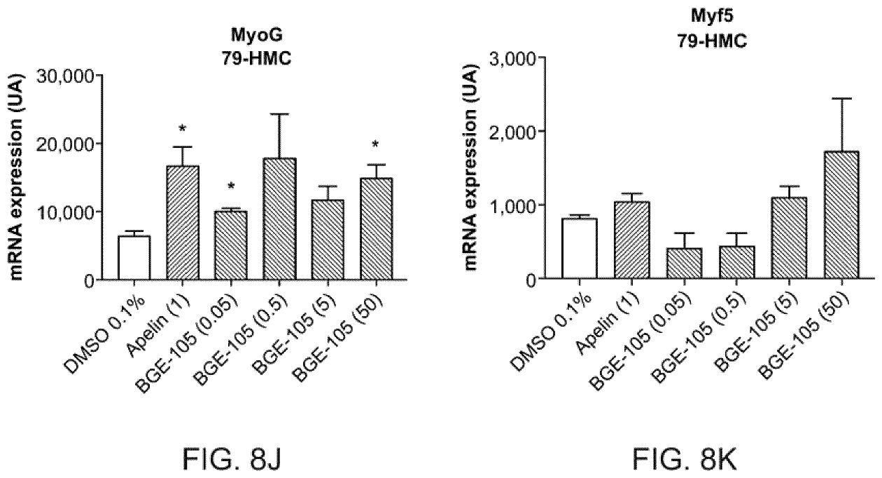

We demonstrated that immortalized human muscles from younger and older patients showed increased proliferation after treatment with increased dosages of BGE-105 (FIGS. 8B-8C). The younger cells showed a significant increase in cell proliferation at 50 nM, and the older cells had a significant increase in cell proliferation at 5 nM. Further details are provided in the experimental section, see, e.g., Example 6 and FIG. 8.

5.1.5. BGE-105 Prevents Atrophy in Immobilized Mouse Muscle

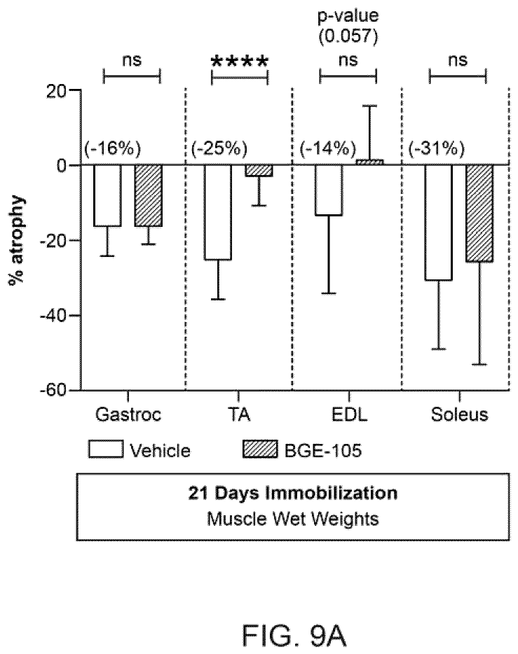

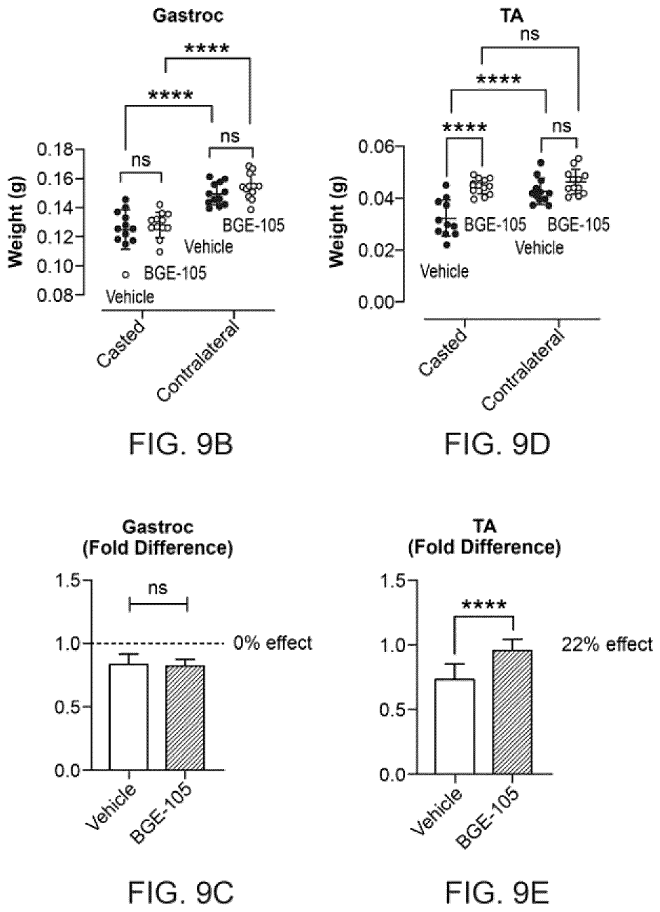

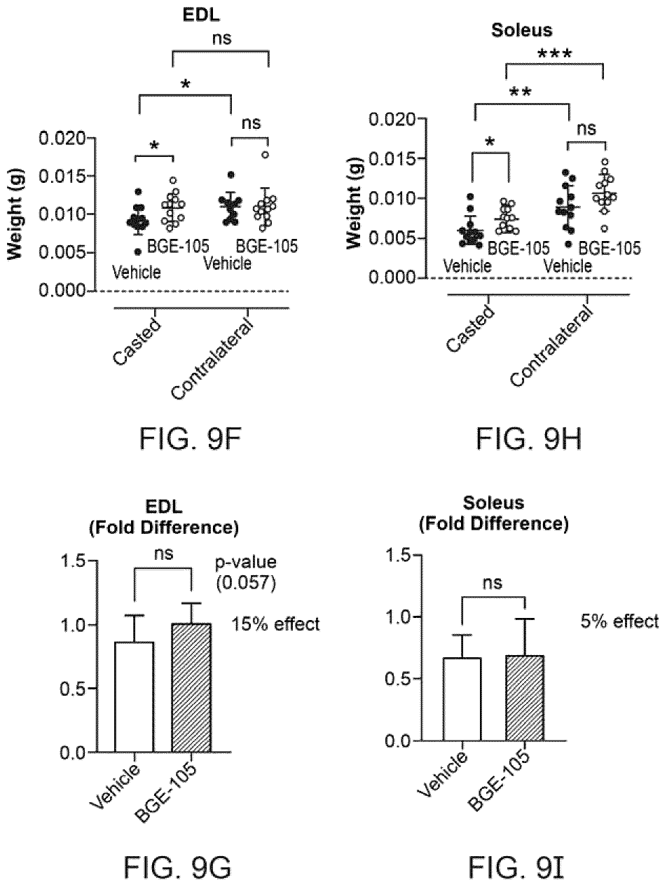

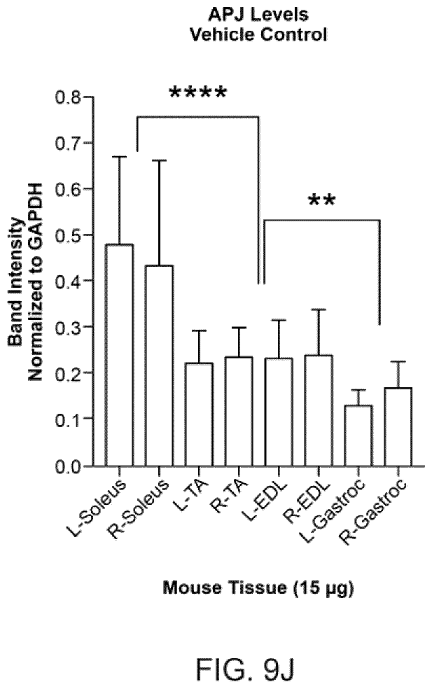

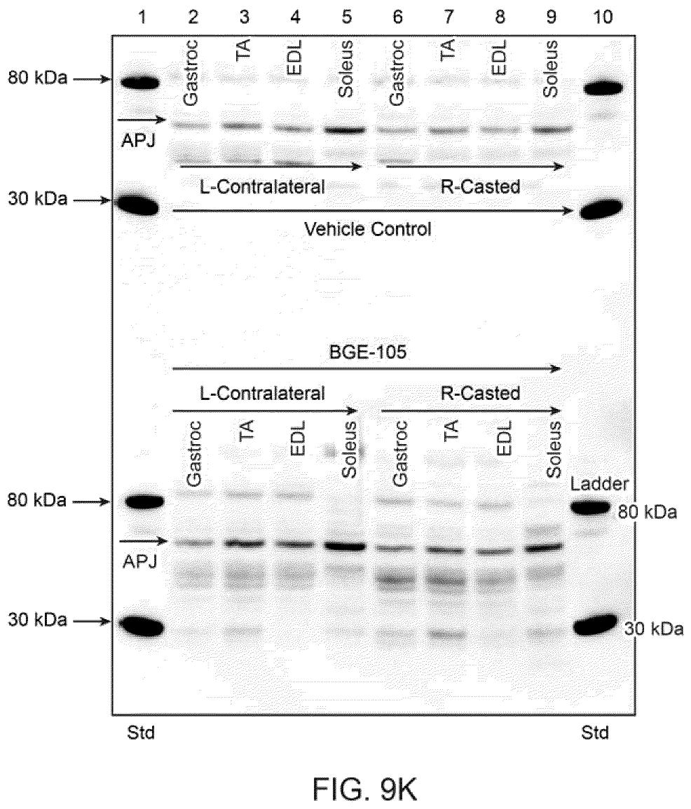

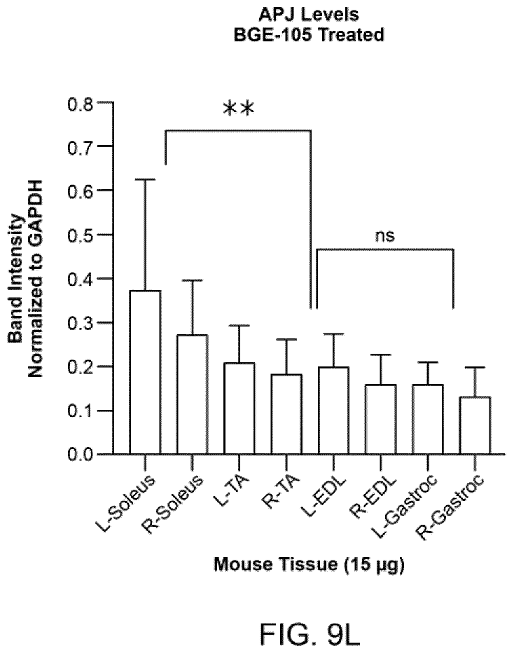

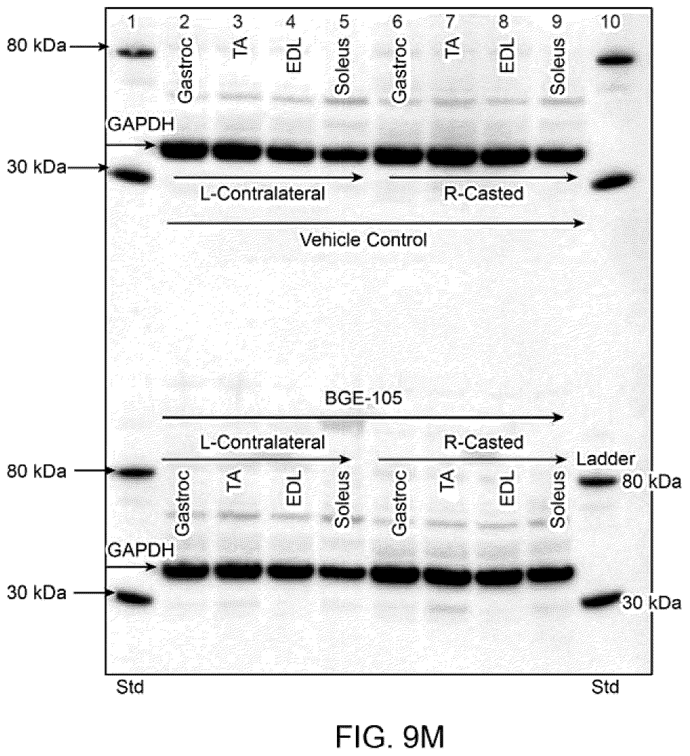

We demonstrated that 20-month-old mice which were immobilized and treated with BGE-105 showed a significant improvement in maintaining muscle weight in the tibialis anterior as compared to vehicle-treated controls. (FIGS. 9D and 9E). There was also a near significant improvement in muscle weight as compared to vehicle in the extensor digitorum longus (FIGS. 9F and 9G) and in the soleus (FIGS. 9H and 9I) muscle as compared to vehicle-treated controls.

There was a significant decrease in the percent atrophy in the tibialis anterior muscle, a near significant decrease in the percent atrophy in the extensor digitorum longus, a marginal improvement in the percent atrophy in the soleus, and no improvement in the gastrocnemius (FIG. 9A). Further details are provided in the experimental section, see, e.g., Example 7 and FIG. 9.

### 5.2. Methods of Treating Muscle Conditions

Clinical studies are described herein which shown that BGE-105 prevent or attenuate muscle atrophy in human patients, including patients on bed rest. Extensive assessments of several muscle dynamics, patient serum biomarkers and proteomics analyses were also performed that are consistent with our demonstrations of clinical efficacy in humans.

Accordingly, in a first aspect the present disclosure provides a method of treating a subject for a muscle condition, such as a muscle condition associated with aging, using an apelin receptor modulator. The method includes administering to a subject a therapeutically effective amount of an apelin receptor modulator of formula (I) or (II) (e.g., as described herein). In some embodiments, the subject is an elderly human subject. In some embodiments, the subject is human and not elderly.

The “muscle condition associated with aging” (referred to interchangeably herein as an “age-related muscle condition”) refers to a degenerative disease or condition or impairment associated with muscle in a mammalian subject. In some embodiments, the muscle is skeletal muscle. Skeletal muscle is considered an organ of the muscular system. Skeletal muscle can include muscle tissues responsible for skeletal movement. For example, skeletal muscle can include muscles under conscious or voluntary control, such as striated muscles.

In some embodiments, other parts of the mammal can be affected by an age-related muscle condition, such as blood vessels (e.g., arteries), nerves, bones, or skin. In some embodiments, the age-related muscle condition is associated with inflammation and/or impairment of mitochondrial function.

Examples of muscle conditions that can be targeted for treatment according to the methods of this disclosure include, but are not limited to, sarcopenia, frailty, muscle weakness due to hip fracture, reduction in risk of hip fracture, ICU associated muscle weakness, muscle atrophy, diaphragm disfunction, diaphragm atrophy, ventilator-induced diaphragmatic dysfunction (VIDD), immobilization associated muscle weakness, immobility associated muscle weakness, recovery from muscle injury, muscle wasting, and critical illness myopathy. In certain embodiments, the muscle condition is acute muscle atrophy (e.g., patients who are on bedrest). In certain embodiments, the muscle condition is chronic muscle loss. In certain embodiments, the muscle condition is ICU diaphragm atrophy. In certain embodiments, the condition is critical illness myopathy.

In some embodiments, the muscle condition is sarcopenia. Sarcopenia is a condition characterized by loss of skeletal muscle mass and function. When this condition is associated with aging, it can also be referred to as age-related sarcopenia. Diagnosis of sarcopenia can be achieved via an assessment of low muscle mass plus the presence of low muscle function (low muscle strength/weakness or low physical performance) (see e.g., Cruz-Jentoft et al., (2010) Sarcopenia: European consensus on definition and diagnosis Report of the European Working Group on Sarcopenia in Older People. Age and Ageing; 39: 412-423; Muscaritoli et al., (2010) Consensus definition of sarcopenia, cachexia and pre-cachexia: joint document elaborated by Special Interest Groups (SIG) “cachexia-anorexia in chronic wasting diseases” and “nutrition in geriatrics”. Clin Nutr. April, 29(2):154-9; Fielding et al. (2011) Sarcopenia: An Undiagnosed Condition in Older Adults. Current Consensus Definition: Prevalence, Etiology, and Consequences. International Working Group on Sarcopenia. J Am Med Dir Assoc, 12: 249-256; and Studenski et al. (2014) The FNIH Sarcopenia Project: Rationale, study description, conference recommendations and final estimates. J Gerontol A Biol Sci Med Sci 69(5): 547-558).

Frailty is a geriatric condition characterized by an increased vulnerability to external stressors. It is strongly linked to adverse outcomes, including mortality, nursing home admission, and falls. In some embodiments, the muscle condition is a condition associated with one or more characteristic measures of frailty. In some embodiments, the subject is classified as frail. In some embodiments, the subject is classified as pre-frail, and is at a high risk or progression to being frail. Frailty can be diagnosed and/or characterized according to various indices of frailty that are composite measures of age-related changes indices of frailty, such as methods based on the Fried's frailty scale (see e.g., Fried, et al., Frailty in older adults: evidence for a phenotype. J Gerontol A Biol Sci Med Sci. 2001, 56: M146-M156) and/or the Mitnitski's Frailty Index (see e.g., Mitnitski et al., Frailty, fitness and late-life mortality in relation to chronological and biological age. BMC Geriatr. 2002, 2: 1-10).

In some embodiments, the muscle condition is muscle atrophy. Muscle atrophy refers to any wasting or loss of muscle tissue resulting from lack of use. Muscle atrophy can lead to muscle weakness and cause disability. In some embodiments, the muscle condition is immobilization-associated muscle weakness, which refers to any wasting or loss of muscle tissue resulting from immobilization, e.g., for medical reasons.

In some embodiments, the muscle condition is muscle weakness, also referred to as muscle fatigue, which refers to a condition characterized by the subject's inability to exert force with skeletal muscles. Muscle weakness often follows muscle atrophy.

Muscle atrophy was measured using various endpoints, such as skeletal muscle protein fractional synthetic rate (FSR) in a liquid biopsy. Other measurements of muscle atrophy include diaphragm thickness, echo-density (e.g. of vastus lateralis), muscle circumference (of muscles such as the thigh/vastus lateralis), muscle cross-sectional area, and the like. Detection of muscle circumference can be measured using ultrasound. Ultrasound can be used to assess diaphragm dysfunction, predict extubating success or failure, quantify respiratory effort, and detect atrophy in, for example, mechanically ventilated subjects.

In some embodiments, the muscle condition is a skeletal muscle condition. In some embodiments, the muscle condition is not a cardiovascular condition. In some embodiments, the subject is not suffering from, or identified as having, a cardiovascular disease or condition. In some embodiments, the subject is not suffering from, or at risk of, a heart failure.

In some embodiments the age-related muscle condition is associated with the loss-of-function, decrease in the ability to regenerate, or heal after injury of skeletal muscle. In some embodiments the age-related muscle condition is associated with the loss-of-function of muscle stem cells.

In some embodiments, the muscle condition is due to insulin insensitivity associated with muscle atrophy. Type 2 diabetes mellitus can be associated with an accelerated muscle loss during aging, decreased muscle function, and increased disability.

5.2.1. Patient Age

In some embodiments of the method of treating a subject for a muscle condition, the subject has, or is suspected of having, an age-related muscle condition.

In some embodiments, the subject is human. The subject can be a human patient suffering from, or a risk of, an age-related muscle condition. In some embodiments, the patient is at least 40-years-old. In some embodiments, the patient is at least 50-years-old. In some embodiments, the patient is at least 60-years-old. In some embodiments, the patient is at least 65-years-old. In some embodiments, the patient is at least 70-years-old. In some embodiments, the patient is at least 75-years-old. In some embodiments, the patient is at least 80-years-old. In some embodiments, the patient is at least 85-years-old. In some embodiments, the patient is at least 90-years-old. In certain embodiments, the patient is 40-50 years old, 50-60 years old, 60-70 years old, 70-80 years old, or 80-90 years old.

5.2.2. Assessment of Patients

A subject can be identified as in need of treatment according to the methods of this disclosure, using a variety of different assessment methods.

A sarcopenia diagnosis can be determined or confirmed by the presence of low muscle quantity or quality. When low muscle strength or force, low muscle quantity/quality and low physical performance are all detected, sarcopenia is considered severe. In some embodiments, the patient has low muscle quantity or quality as compared to criteria representative of a healthy human subject, e.g., a subject of the same age or younger.

Low muscle mass can be assessed using appendicular lean body mass (ALBM). In some embodiments, low muscle mass is indicated by an ALBM adjusted for body mass index (BMI) of <0.789 kg for men or <0.512 kg for women, where ALBM can be measured by dual energy X-ray absorptiometry (DXA).

Low muscle mass can be assessed by the appendicular skeletal muscle index (ASMI). In some low muscle mass is indicated by an appendicular skeletal muscle index (ASMI) of less than 7.26 kg/m² for men, or less than 5.5 kg/m² for women, said ASMI being defined as appendicular skeletal muscle mass divided by the square of height, said ASMI being measured by dual energy X-ray absorptiometry (DXA).

Low muscle strength can include low grip strength, and be determined using a handgrip strength test. In some embodiments, low grip strength is assessed by measuring the amount of static force that the hand can squeeze around a handgrip dynamometer, e.g., as indicated by a value of less than 30 kg, such as less than 26 kg for men, or less than 20 kg for women, such as less than 16 kg, in the handgrip strength test.

In some embodiments, the human subject has, or is identified as having, low muscle strength. In some embodiments, the human subject has, or is identified as having, low muscle force.

In some embodiments, the human subject has, or is identified as having, low lower limb muscle mass. In some embodiments, the human subject has, or is identified as having, low upper limb muscle mass. In some embodiments, the human subject has, or is identified as having, decreased muscle function. In some embodiments, the human subject has, or is identified as having, decreased muscle strength.

In some embodiments, the human subject has, or is identified as having, low muscle volume. In some embodiments, the muscle volume is skeletal muscle volume. In some embodiments, the muscle is skeletal muscle. In some embodiments, the muscle is diaphragm, tibialis anterior, tibialis posterior, gastrocnemius, sartorius, vastus intermedius, vastus laterals, vastus medialis, soleus, rectus femorus, or extensor digitorum longus.

In some embodiments, the muscle volume is the muscle volume of one or more upper limb muscles selected from the group consisting of: shoulder abductors, shoulder adductors, elbow flexors, elbow extensors, wrist flexors, and wrist extensors.

In some embodiments, muscle mass is assessed after the dosing. In some embodiments, muscle mass is assessed at least one day after dosing. In some embodiments, the muscle mass is assessed at least one week after dosing. In some embodiments, the muscle mass is assessed at least one month after dosing.

In some embodiments, the muscle condition is a skeletal muscle condition. In some embodiments, the skeletal muscle expresses the apelin receptor and administration of the apelin receptor modulator activates the apelin/APJ system (APLNR gene) in the muscle tissue of the subject. The muscle of interest expresses the apelin receptor, and in some embodiments, the level of expression of the apelin receptor can be assessed or determined in a muscle tissue of the subject prior to and/or after treatment. In some embodiments, the subject has, or is identified as having, a low circulating level of apelin. Apelin circulating levels can be assessed in a biological sample obtained from the subject, e.g., using a quantitative assay (e.g., ELISA assay, or LC/MS) for determining the amount of an apelin peptide in a sample.

In some embodiments, the muscle condition is a diaphragmatic muscle condition. In some embodiments, the diaphragmatic muscle condition is diaphragm atrophy. In some embodiments, the diaphragmatic muscle condition is diaphragm dysfunction. Dysfunction of the diaphragm ranges from a partial loss of the ability to generate pressure (weakness) to a complete loss of diaphragmatic function (paralysis). Patients with bilateral diaphragmatic paralysis or severe diaphragmatic weakness are likely to have dyspnea or recurrent respiratory failure. They can have considerable dyspnea at rest, when supine, with exertion, or when immersed in water above their waist. Further, patients with bilateral diaphragmatic paralysis are at an increased risk for sleep fragmentation and hypoventilation during sleep.

In some embodiments of the methods of this disclosure, the subject is human and has, or is identified as having, one or more of diabetes mellitus, insulin insensitivity, cardiovascular disease, and neurologic disease.

In some embodiments, the subject is human and has, or is identified to have diaphragm atrophy. In certain embodiments, the subject is human is undergoing mechanical ventilation (e.g. is mechanically ventilated at time of diagnosis). In certain embodiments, the subject is human and has, or is identified to have diaphragm atrophy caused by mechanical ventilation. In some embodiments, the subject is human and is on a ventilator (e.g. mechanical ventilatory). In some embodiments, the subject is human and has lost muscle mass during periods of immobilization and bed rest. In some embodiments, the subject is human and has acute myopathy. In some embodiments, the subject is human, hospitalized, and has acute myopathy. In some embodiments, the subject has a chronic medical condition related to muscle aging.

In certain embodiments, the patient is on bedrest. In certain embodiments, the subject has chronic obstructive pulmonary disease (COPD). In certain embodiments, the subject who has COPD is on bedrest and has bedrest-induced muscle atrophy/muscle loss. In certain embodiments, the subject who has COPD is on bedrest and has bedrest-induced muscle degradation. In certain embodiments, the subject who has COPD is on bedrest and has muscle loss associated with myopathy. In certain embodiments, the subject who has COPD has severe or acute muscle loss.

In some embodiments of the methods of this disclosure, the subject is human and has, or is identified as having, hypoxic respiratory failure. Hypoxic respiratory failure can be measured by stratifying diaphragm thickness.

Muscle atrophy can be measured using various endpoints, such as skeletal muscle protein fractional synthetic rate (FSR) in a liquid biopsy. Other measurements of muscle atrophy include diaphragm thickness, echo-density (e.g. of vastus lateralis), muscle circumference (of muscles such as the thigh/vastus lateralis), muscle cross-sectional area, and the like. Detection of muscle circumference can be measured using ultrasound. Ultrasound can be used to assess diaphragm dysfunction, predict extubating success or failure, quantify respiratory effort, and detect atrophy in, for example, mechanically ventilated subjects.

Diaphragm atrophy can be measured by a change in diaphragm thickness. For example, diaphragmatic thickness can be measured in subjects that are mechanically ventilated before ventilation, at the time of ventilation, after a number of days on a ventilator, after treatment, and the like (see e.g., Schepens et al., (2015) Crit Care; 19: 422)

### 5.3. Methods of Maintaining Muscle Mass or Muscle Strength

Aspects of this disclosure include a method for maintaining and/or increasing muscle mass and/or muscle strength in subject in need. In some embodiment the subject is an elderly human. In various embodiments, an apelin receptor modulator (e.g., as described herein) is administered to the elderly subject to maintain or increase muscle mass and/or muscle strength in skeletal muscle of the subject. In some embodiments, the apelin receptor modulator is an apelin receptor agonist.

In some embodiments, the elderly subject is human and at least 60-years-old. In some embodiments, the patient is at least 65-years-old. In some embodiments, the patient is at least 70-years-old. In some embodiments, the patient is at least 75-years-old. In some embodiments, the patient is at least 80-years-old. In some embodiments, the patient is at least 85-years-old. In some embodiments, the patient is at least 90-years-old. In certain embodiments, the patient is 60-70 years old, 70-80 years old, or 80-90 years old.

The muscle mass and/or muscle strength of a subject can be monitored during treatment and compared to a baseline assessment performed prior to dosing with the apelin receptor modulator. In some embodiments, the apelin receptor modulator is an apelin receptor agonist. In some embodiments, the muscle mass or muscle strength of a subject is at least maintained at baseline levels during treatment. In some embodiments, the subject is one who has suffered from declining muscle mass and/or muscle strength over time, and administration of the apelin receptor modulator according to methods of this disclosure reverses and/or ameliorates the decline. In some embodiments, the apelin receptor modulator is an apelin receptor agonist.

Low muscle mass can be assessed using appendicular lean body mass (ALBM). In some embodiments, low muscle mass is indicated by an ALBM adjusted for body mass index (BMI) of <0.789 kg for men or <0.512 kg for women, where ALBM can be measured by dual energy X-ray absorptiometry (DXA).

Low muscle mass can be assessed by the appendicular skeletal muscle index (ASMI). In some low muscle mass is indicated by an appendicular skeletal muscle index (ASMI) of less than 7.26 kg/m² for men, or less than 5.5 kg/m² for women, said ASMI being defined as appendicular skeletal muscle mass divided by the square of height, said ASMI being measured by dual energy X-ray absorptiometry (DXA).

Low muscle strength can be determined using a handgrip strength test. In some embodiments, low muscle strength is indicated by a value of less than 30 kg, such as less than 26 kg for men, or less than 20 kg for women, such as less than 16 kg, in the handgrip strength test.

In some embodiments, muscle mass is assessed before and after the dosing of the apelin receptor agonist. In some embodiments, the muscle mass is assessed at least one day after dosing. In some embodiments, the muscle mass is assessed at least one week after dosing. In some embodiments, the muscle mass is assessed at least one month after dosing.

In some embodiments, muscle strength is assessed before and after the dosing of the apelin receptor agonist. In some embodiments, the muscle strength is assessed at least one day after dosing. In some embodiments, the muscle strength is assessed at least one week after dosing. In some embodiments, the muscle strength is assessed at least one month after dosing.

In some embodiments, the subject has, or is identified as having, a low circulating level of apelin. Apelin circulating levels can be assessed in a biological sample obtained from the subject.

In some embodiments, the subject has, or is identified as having, altered levels of the serum proteins that are associated in BioAge's longitudinal aging cohort data with frailty, sarcopenia, muscle atrophy, or muscle weakness.

### 5.4. Apelin Receptor Modulators

Apelin is the endogenous ligand for the apelin receptor (also referred to as APJ, or APLNR). The apelin receptor is a member of the rhodopsin-like G protein-coupled receptor (GPCR) family. The apelin/APJ system is distributed in diverse periphery organ tissues and can play various roles in the physiology and pathophysiology of many organs. The apelin/APJ system participates in various cell activities such as proliferation, migration, apoptosis or inflammation. An apelin receptor modulators can activate the APJ system directly or indirectly, competitively, or non-competitively. Accordingly, an apelin receptor modulator of this disclosure can be referred to as an apelin receptor agonist.

As further described below, in some embodiments of the methods of this disclosure, the apelin receptor modulator (e.g., apelin receptor agonist) is a compound described in U.S. Pat. No. 9,573,936 or 9,868,721, the disclosures of which are herein incorporated by reference in their entirety.

As known by those skilled in the art, certain compounds of this disclosure may exist in one or more tautomeric forms. Because one chemical structure may only be used to represent one tautomeric form, it will be understood that for convenience, referral to a compound of a given structural formula includes tautomers of the structure represented by the structural formula.

In some embodiments, the apelin receptor modulator is a compound of formula (I) or (II):

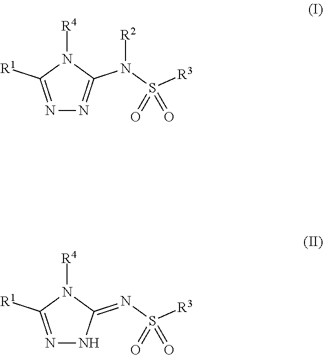

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

In some embodiments, the apelin receptor modulator is a compound of formula (I) or (II):

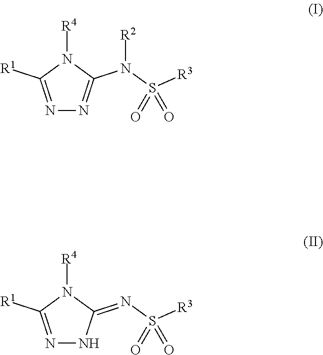

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, or S;
R² is selected from —H, or C₁-C₄ alkyl or is absent in the compounds of Formula II;
R³ is a group of formula —(CR^{3d}R^{3e})—(CR^{3f}R^{3g})-Q;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), or —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), or —N(C₁-C₆ alkyl)₂;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, or a heteroaryl group, and the Q heterocyclyl group may be substituted with 1 oxo substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, or S, or a monocyclic or bicyclic heterocyclyl group with 5 to 10 ring members containing 1, 2, 3, or 4 heteroatoms independently selected from N, O, or S, wherein the C₆-C₁₀ aryl group, the heteroaryl group, or the heterocyclyl group are unsubstituted or are substituted with 1, 2, or 3 R^{4a} substituents; and
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), or —C(═O)N(C₁-C₆ alkyl)₂, and the heterocyclyl R⁴ group may be further substituted with 1 oxo substituent.

As noted above, apelin receptor agonist compounds of this disclosure may exist in multiple tautomeric forms. This is particularly true in compounds of Formula I where R² is H. These forms are illustrated below as Tautomer A and Tautomer B:

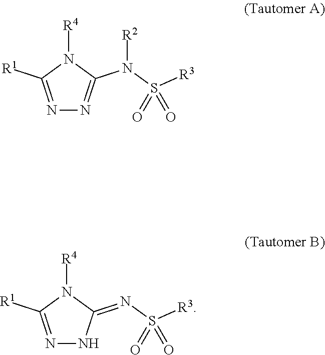

Apelin receptor agonist compounds of this disclosure are depicted structurally and generally named as compounds in the “Tautomer A” form. However, it is specifically contemplated and known that the compounds exist in “Tautomer B” form and thus compounds in “Tautomer B” form are expressly considered to be part of this disclosure. For this reason, the claims refer to compounds of Formula I and Formula II. Depending on the compound, some compounds may exist primarily in one form more than another. Also, depending on the compound and the energy required to convert one tautomer to the other, some compounds may exist as mixtures at room temperature whereas others may be isolated in one tautomeric form or the other.

In some embodiments of formula (I) and (II), R¹ is an unsubstituted pyridyl or is a pyridyl substituted with 1 or 2 R^{1a} substituents.

In some embodiments of formula (I) and (II), R^{1a} in each instance is independently selected from —CH₃, —CH₂CH₃, —F, —Cl, —Br, —CN, —CF₃, —CH═CH₂, —C(═O)NH₂, —C(═O)NH(CH₃), —C(═O)N(CH₃)₂, —C(═O)NH(CH₂CH₃), —OH, —OCH₃, —OCHF₂, —OCH₂CH₃, —OCH₂CF₃, —OCH₂CH₂OH, —OCH₂C(CH₃)₂OH, —OCH₂C(CF₃)₂OH, —OCH₂CH₂OCH₃, —NH₂, —NHCH₃, —N(CH₃)₂, phenyl, and a group of formula

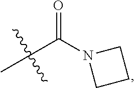 [SMILES: *C(=O)N1CCC1]

wherein the symbol [symbol] , when drawn across a bond, indicates the point of attachment to the rest of the molecule.

In some embodiments of formula (I) and (II), R¹ is selected from

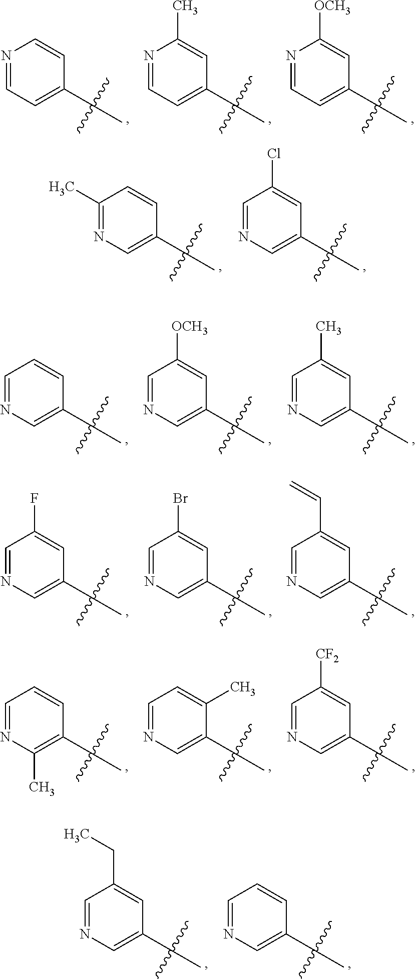

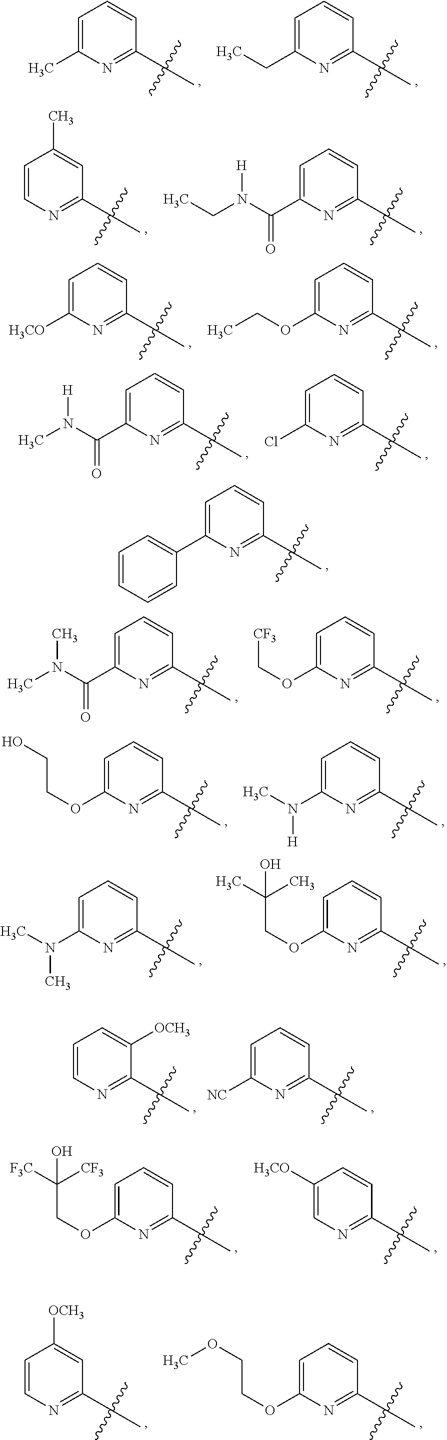

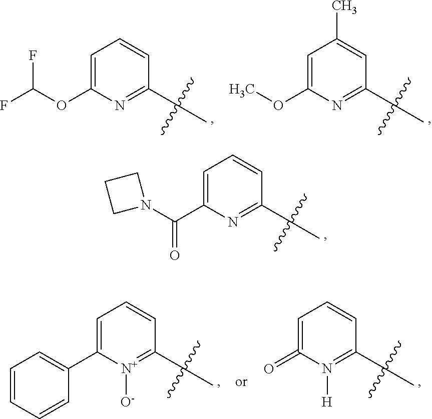

wherein the symbol [symbol] , when drawn across a bond, indicates the point of attachment to the rest of the molecule.

In some embodiments of formula (I) and (II), R¹ is selected from

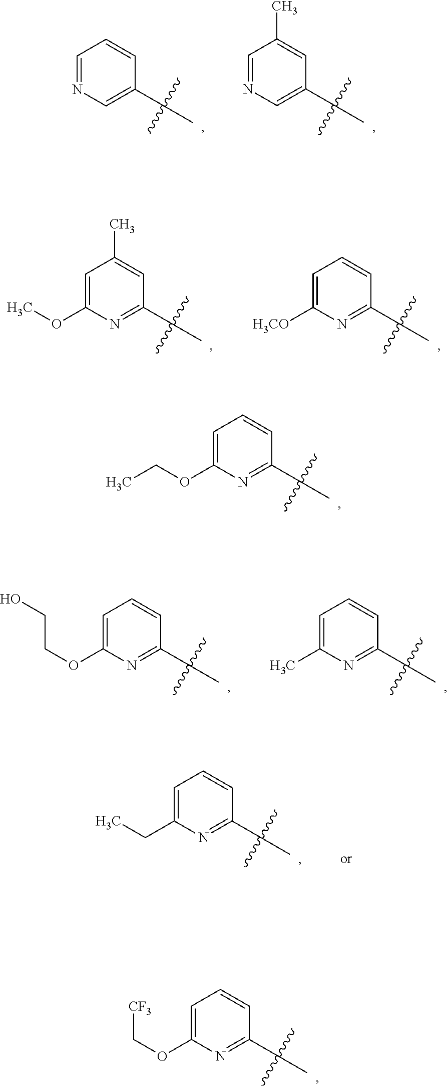

wherein the symbol [symbol] , when drawn across a bond, indicates the point of attachment to the rest of the molecule.

In some embodiments of formula (I) and (II), R² is —H.

In some embodiments of formula (I) and (II), R⁴ is a phenyl, pyridyl, pyrimidinyl, isoxazolyl, indolyl, naphthyl, or pyridinyl any of which may be unsubstituted or substituted with 1, 2, or 3 R^{4a} substituents. In some embodiments of formula (I) and (II), R⁴ is a phenyl substituted with 1 or 2 R^{4a} substituents. In some embodiments of formula (I) and (II), the 1 or 2 R^{4a} substituents are —O—(C₁-C₂ alkyl) groups.

In some embodiments of formula (I) and (II), R^{4a} is in each instance independently selected from —CH₃, —F, —Cl, —Br, —CN, —CF₃, —OCH₃, —OCHF₂, —OCH₂CH₃, —C(═O)OCH₃, —C(═O)CH₃, or —N(CH₃)₂.

In some embodiments of formula (I) and (II), R⁴ is selected from:

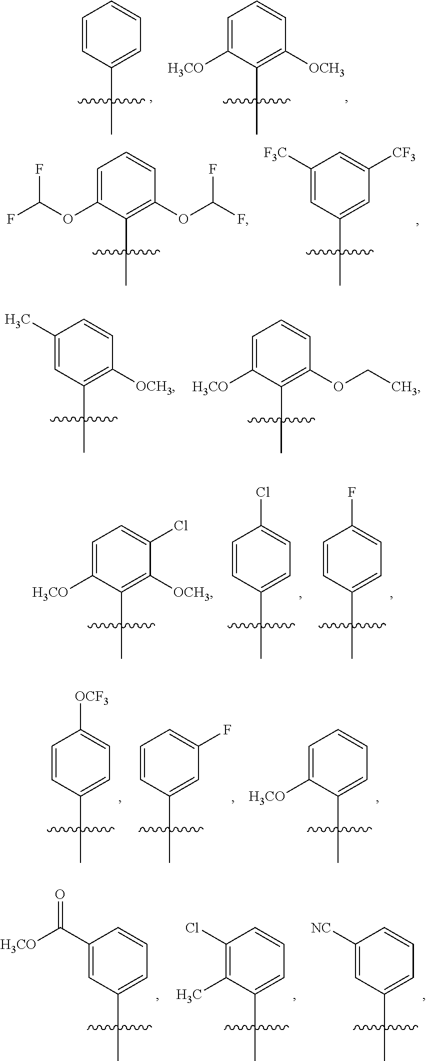

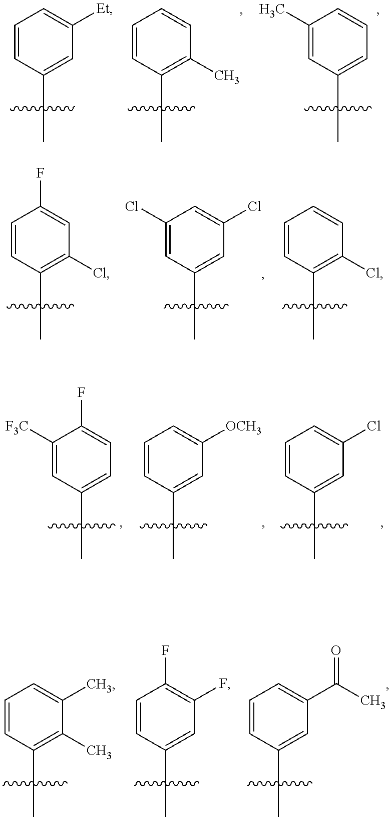

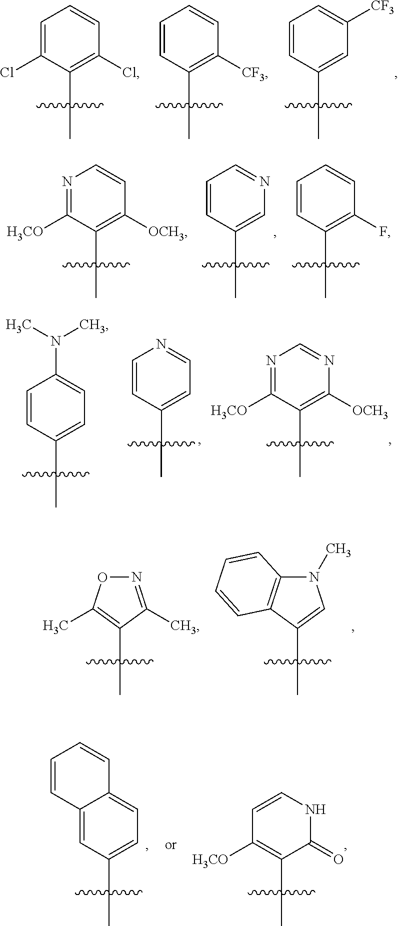

wherein the symbol [symbol] , when drawn across a bond, indicates the point of attachment to the rest of the molecule.

In some embodiments of formula (I) and (II), R³ is selected from a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, or a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

In some embodiments of formula (I) and (II), Q is selected from pyrimidinyl, pyridyl, isoxazolyl, thiazolyl, imidazolyl, phenyl, tetrahydropyrimidinonyl, cyclopropyl, cyclobutyl, cyclohexyl, morpholinyl, pyrrolidinyl, pyrazinyl, imidazo[1,2-a]pyridinyl, pyrazolyl, or oxetanyl any of which may be unsubstituted or substituted with 1, 2, or 3, R^{Q} substituents.

In some embodiments of formula (I) and (II), Q is a monocyclic heteroaryl group with 5 or 6 ring members containing 1 or 2 heteroatoms selected from N, O, or S and Q is unsubstituted or is substituted with 1 or 2 R^{Q} substituents.

In some embodiments of formula (I) and (II), Q is selected from

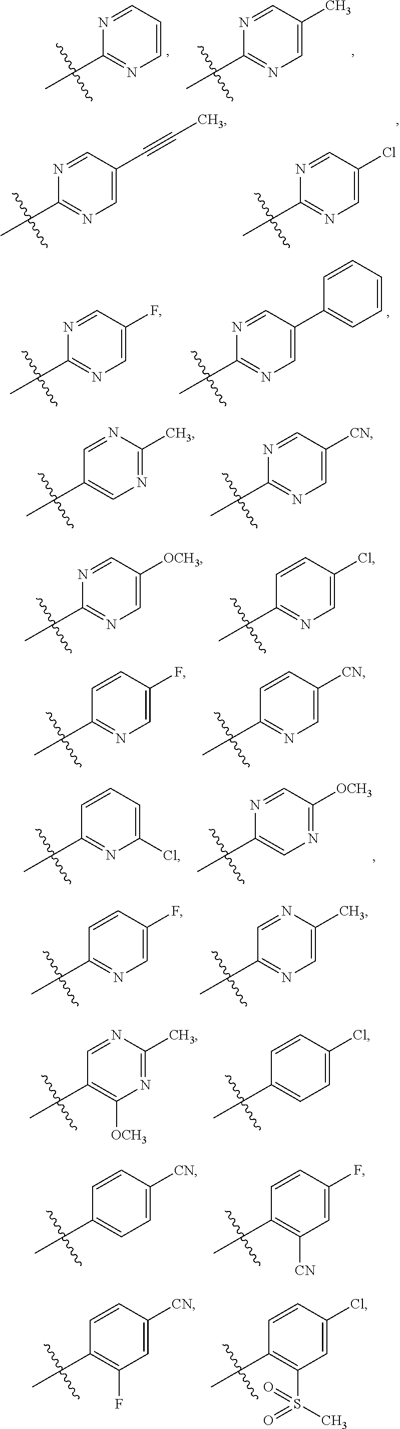

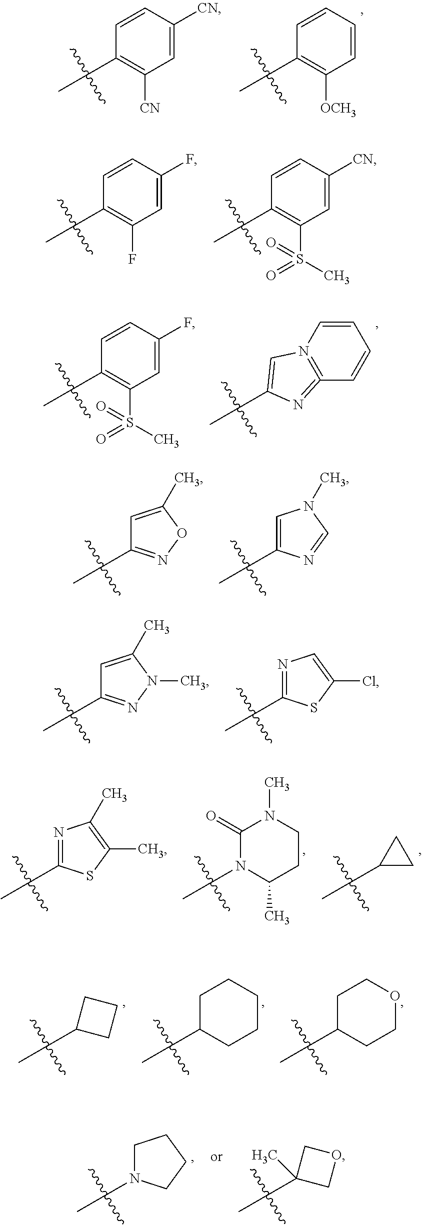

wherein the symbol [symbol] , when drawn across a bond, indicates the point of attachment to the rest of the molecule.

In some embodiments of formula (I) and (II), R³ is a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

In some embodiments of formula (I) and (II), R³ is a group of formula —(CR^{3d}R^{3e})—(CR^{3f}R^{3g})-Q.

In some embodiments of formula (I) and (II), R³ has the formula

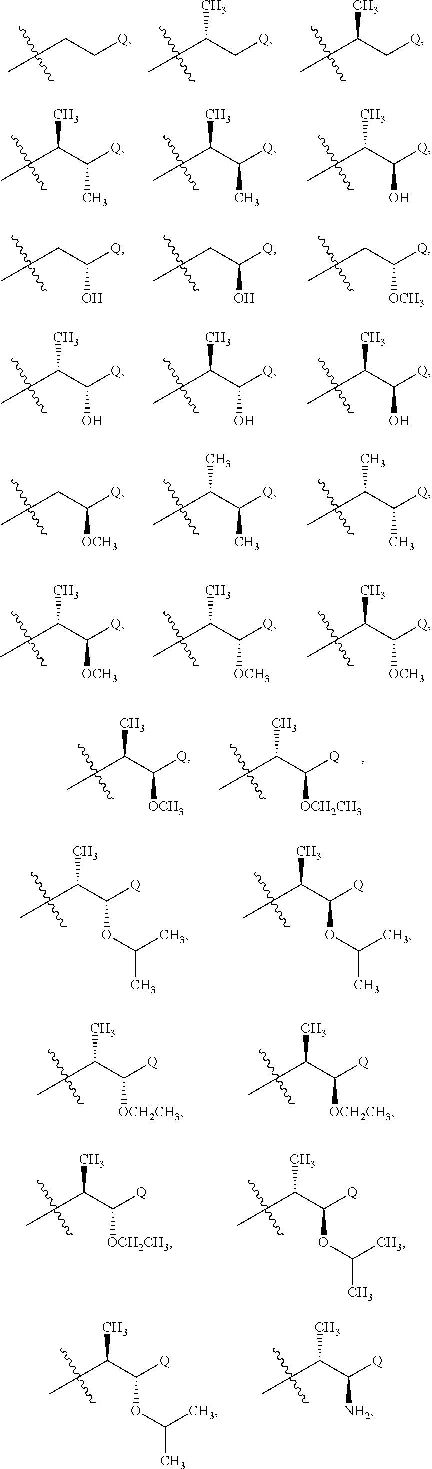

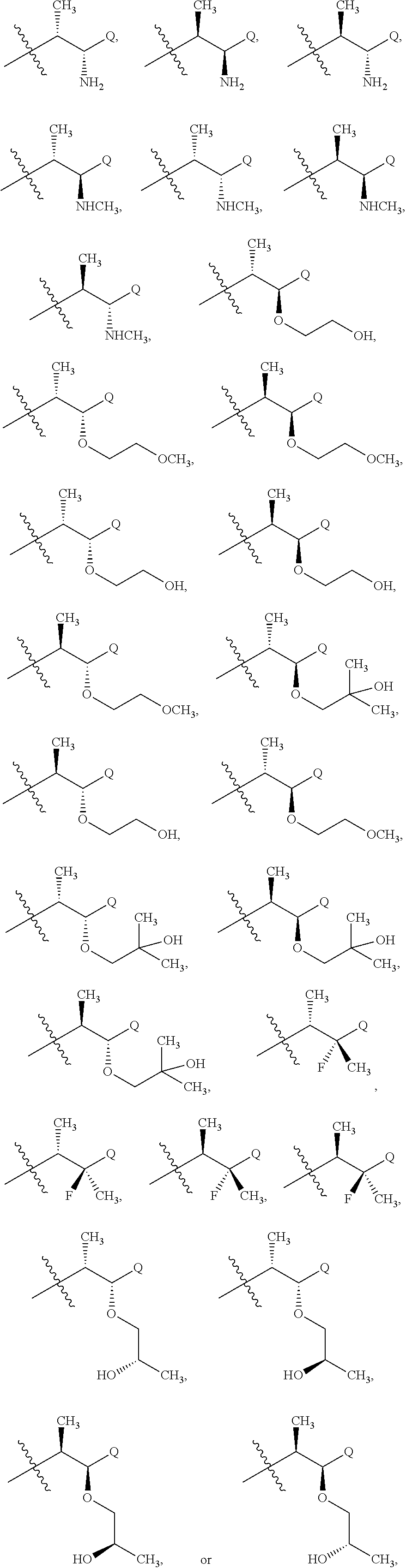

wherein the symbol [symbol] , when drawn across a bond, indicates the point of attachment to the rest of the molecule.

In some embodiments of formula (I) and (II), R³ has the formula

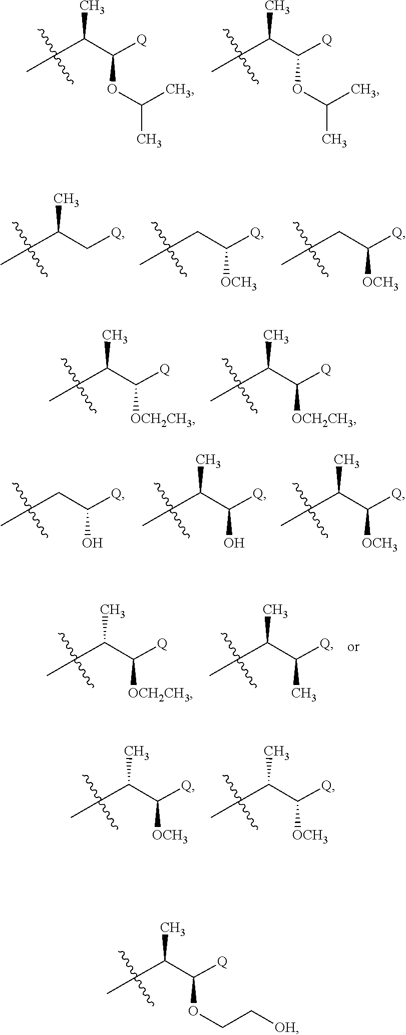

wherein the symbol [symbol] , when drawn across a bond, indicates the point of attachment to the rest of the molecule.

In particular embodiments of formula (I) and (II), the apelin receptor agonist is
(1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide;
(2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide;
(1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide;
(1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide;
(1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide;
(1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide;
(1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide;
(2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide;
(1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide;
(1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide;
(1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide;
(1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide;
(1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide;
(2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide;
(1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide;
(1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide;
(1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide;
(1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide;
(1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide;
(2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide;
(1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide;
(1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide;
(1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide; or
(1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide.

In a particular embodiment of formula (I) and (II), the apelin receptor agonist is (1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide or the pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide or the pharmaceutically acceptable salt thereof.

In a particular embodiment of formula (I) and (II), the apelin receptor agonist is (1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide or the pharmaceutically acceptable salt thereof.

In a particular embodiment of formula (I) and (II), the apelin receptor agonist is (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propane sulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide or the pharmaceutically acceptable salt thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide or the pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide or the pharmaceutically acceptable salt thereof.

In a particular embodiment of formula (I) and (II), the apelin receptor agonist is (1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide or the pharmaceutically acceptable salt thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide or the pharmaceutically acceptable salt thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide or the pharmaceutically acceptable salt thereof.

In a particular embodiment of formula (I) and (II), the apelin receptor agonist is (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide or the pharmaceutically acceptable salt thereof.

In a particular embodiment of formula (I) and (II), the apelin receptor agonist is (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide or the pharmaceutically acceptable salt thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide or the pharmaceutically acceptable salt thereof.

In a particular embodiment of formula (I) and (II), the apelin receptor agonist is (1R,2S)—N-(4-(2,6-dimethoxyphenyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)-1-(5-chloro-2-pyrimidinyl)-N-(4-(2,6-dimethoxyphenyl)-5-(5-methyl-3-pyridinyl)-4H-1,2,4-triazol-3-yl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)—N-(4-(2,6-dimethoxyphenyl)-5-(5-methyl-3-pyridinyl)-4H-1,2,4-triazol-3-yl)-1-methoxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-hydroxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)—N-(4-(2,6-dimethoxyphenyl)-5-(3-pyridinyl)-4H-1,2,4-triazol-3-yl)-1-methoxy-1-(5-methyl-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(6-methyl-2-pyridinyl)-4H-1,2,4-triazol-3-yl)-1-hydroxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(5-fluoro-2-pyrimidinyl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrazinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(3-pyridinyl)-4H-1,2,4-triazol-3-yl)-1-ethoxy-1-(5-fluoro-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-(1-methylethoxy)-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2R)-1-(5-chloro-2-pyrimidinyl)-N-(4-(2,6-dimethoxyphenyl)-5-(3-pyridinyl)-4H-1,2,4-triazol-3-yl)-1-methoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-methoxy-1-(5-methoxy-2-pyrazinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(3-pyridinyl)-4H-1,2,4-triazol-3-yl)-3-(5-methyl-2-pyrazinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-dimethoxyphenyl)-5-(5-methyl-3-pyridinyl)-4H-1,2,4-triazol-3-yl)-1-ethoxy-1-(5-fluoro-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(4,6-dimethoxy-5-pyrimidinyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2R)-1-(5-chloro-2-pyrimidinyl)-N-(4-(2,6-dimethoxyphenyl)-5-(3-pyridinyl)-4H-1,2,4-triazol-3-yl)-1-ethoxy-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-ethoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(2,6-difluorophenyl)-5-(6-methoxy-2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1R,2S)—N-(4-(4,6-dimethoxy-5-pyrimidinyl)-5-(2-pyridinyl)-4H-1,2,4-triazol-3-yl)-1-methoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is N-(4-(2,6-dimethoxyphenyl)-5-(5-methyl-3-pyridinyl)-4H-1,2,4-triazol-3-yl)-1-isopropoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (1S,2S)—N-(4-(2,6-dimethoxyphenyl)-5-(5-methyl-3-pyridinyl)-4H-1,2,4-triazol-3-yl)-1-isopropoxy-1-(5-methyl-2-pyrimidinyl)-2-propanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

In a particular embodiment of formula (I) and (II), the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide (BGE-105) or a pharmaceutically acceptable salt thereof.

In a particular embodiment of formula (I) and (II), the apelin receptor agonist is

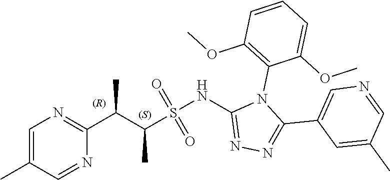

(BGE-105) or a pharmaceutically acceptable salt thereof.

U.S. Pat. Nos. 9,573,936, 9,868,721, 9,745,286, 9,656,997, 9,751,864, 9,656,998, 9,845,310, 10,058,550, 10,221,162, and 10,344,016, the disclosures of which are incorporated herein by reference in their entirety, describe apelin receptor agonists of formula (I) or (II), and methods of synthesizing such triazole agonists of the apelin receptor, including BGE-105. See e.g., Example 263.0 of U.S. Pat. No. 9,573,936.

If any variable occurs more than one time in a chemical formula, its definition on each occurrence is independent of its definition at every other occurrence. If the chemical structure and chemical name conflict, the chemical structure is determinative of the identity of the compound. The compounds of this disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

Certain compounds of this disclosure may possess asymmetric carbon atoms (optical centers) or double bonds; the racemates, enantiomers, diastereomers, geometric isomers and individual isomers are all intended to be encompassed within the scope of the invention. Furthermore, atropisomers and mixtures thereof such as those resulting from restricted rotation about two aromatic or heteroaromatic rings bonded to one another are intended to be encompassed within the scope of the invention. For example, when R⁴ is a phenyl group and is substituted with two groups bonded to the C atoms adjacent to the point of attachment to the N atom of the triazole, then rotation of the phenyl may be restricted. In some instances, the barrier of rotation is high enough that the different atropisomers may be separated and isolated.

Unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. If the stereochemistry of a structure or a portion of a structure is not indicated with, for example, bold or dashed lines, the structure or portion of the structure is to be interpreted as encompassing all stereoisomers of it. A bond drawn with a wavy line indicates that both stereoisomers are encompassed.

Various compounds of this disclosure contain one or more chiral centers, and can exist as racemic mixtures of enantiomers, mixtures of diastereomers or enantiomerically or optically pure compounds. This invention encompasses the use of stereomerically pure forms of such compounds, as well as the use of mixtures of those forms. For example, mixtures comprising equal or unequal amounts of the enantiomers of a particular compound of the invention may be used in methods and compositions of the invention. These isomers may be asymmetrically synthesized or resolved using standard techniques such as chiral columns or chiral resolving agents.

Compounds of the present disclosure include, but are not limited to, compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. The term “compound” encompasses not only the compound itself, but also a pharmaceutically acceptable salt thereof, a solvate thereof, a chelate thereof, a non-covalent complex thereof, a prodrug thereof, and mixtures of any of the foregoing. In some embodiments, the term “compound” encompasses the compound itself, pharmaceutically acceptable salts thereof, tautomers of the compound, pharmaceutically acceptable salts of the tautomers, and ester prodrugs such as (C₁-C₄)alkyl esters. In other embodiments, the term “compound” encompasses the compound itself, pharmaceutically acceptable salts thereof, tautomers of the compound, pharmaceutically acceptable salts of the tautomers.

The term “solvate” refers to the compound formed by the interaction of a solvent and a compound. Suitable solvates are pharmaceutically acceptable solvates, such as hydrates, including monohydrates and hemi-hydrates.

The compounds of this disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., assay reagents, and diagnostic agents, e.g., in vivo imaging agents. All isotopic variations of the compounds of the invention, whether radioactive or not, are intended to be encompassed within the scope of the invention. For example, if a variable is said or shown to be H, this means that variable may also be deuterium (D) or tritium (T).

The term “pharmaceutically acceptable salt” refers to a salt that is acceptable for administration to a subject. Examples of pharmaceutically acceptable salts include, but are not limited to: mineral acid salts such as hydrochloride, hydrobromide, hydroiodide, phosphate, sulfate, and nitrate; sulfonic acid salts such as methanesulfonate, ethanesulfonate, benzenesulfonate, p-toluenesulfonate, and trifluoromethanesulfonate; organic acid salts such as oxalate, tartrate, citrate, maleate, succinate, acetate, trifluoroacetate, benzoate, mandelate, ascorbate, lactate, gluconate, and malate; amino acid salts such as glycine salt, lysine salt, arginine salt, ornithine salt, glutamate, and aspartate; inorganic salts such as lithium salt, sodium salt, potassium salt, calcium salt, and magnesium salt; and salts with organic bases such as ammonium salt, triethylamine salt, diisopropylamine salt, and cyclohexylamine salt. The term “salt(s)” as used herein encompass hydrate salt(s).

Other examples of pharmaceutically salts include anions of the compounds of the present disclosure compounded with a suitable cation. For therapeutic use, salts of the compounds of the present disclosure can be pharmaceutically acceptable. However, salts of acids and bases that are non-pharmaceutically acceptable may also find use, for example, in the preparation or purification of a pharmaceutically acceptable compound.

Compounds included in the present compositions and methods that are basic in nature are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions, including but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

Compounds included in the present compositions and methods that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

Furthermore, if the compounds of the present invention or salts thereof form hydrates or solvates, these are also included in the scope of the compounds of the present invention or salts thereof.

Compounds included in the present compositions and methods that include a basic or acidic moiety can also form pharmaceutically acceptable salts with various amino acids. The compounds of the disclosure can contain both acidic and basic groups; for example, one amino and one carboxylic acid group. In such a case, the compound can exist as an acid addition salt, a zwitterion, or a base salt.

5.4.1. Pharmaceutical Composition

The apelin receptor agonist compounds used in the methods described herein can be formulated in any appropriate pharmaceutical composition for administration by any suitable route of administration. The pharmaceutical compositions can include the compound or the pharmaceutically acceptable salt thereof, the tautomer thereof, the pharmaceutically acceptable salt of the tautomer, the stereoisomer of any of the foregoing, or the mixture thereof according to any one of the embodiments described herein and at least one pharmaceutically acceptable excipient, carrier or diluent. In some such embodiments, the compound or the pharmaceutically acceptable salt thereof, the tautomer thereof, the pharmaceutically acceptable salt of the tautomer, the stereoisomer of any of the foregoing, or the mixture thereof according to any one of the embodiments is present in an amount effective for the treatment of a muscle condition (e.g., as described herein), for activating the APJ receptor.

Suitable routes of administration include, but are not limited to, oral, topical, and intravenous routes of administration. Suitable routes also include pulmonary administration, including by oral inhalation. The most suitable route may depend upon the condition and disorder of the recipient. The formulations may conveniently be presented in unit dosage form and may be prepared by any of the methods known in the art of pharmacy.

In some embodiments, the pharmaceutical composition is formulated for oral delivery whereas in other embodiments, the pharmaceutical composition is formulated for intravenous delivery. In some embodiments, the pharmaceutical composition is formulated for oral administration once a day or QD, and in some such formulations is a tablet where the effective amount of the active ingredient ranges from 5 mg to 60 mg, from 6 mg to 58 mg, from 10 mg to 40 mg, from 15 mg to 30 mg, from 16 mg to 25 mg, or from 17 mg to 20 mg. In some such compositions, the amount of active ingredient is 17 mg.

All methods include the step of bringing into association an apelin agonist, or a salt thereof, with the carrier which constitutes one or more excipients. In general, the formulations are prepared by uniformly and intimately bringing into association the active ingredient with liquid carriers or finely divided solid carriers or both and then, if necessary, shaping the product into the desired formulation.

In certain embodiments, the route of administration for use in the methods described herein is parenteral administration. In certain embodiments, the route of administration for use in the methods described herein is intravenous administration (e.g., intravenous infusion). In certain embodiments, the route of administration for use in the methods described herein is oral administration. In certain embodiments, the route of administration for use in the methods described herein is constant intravenous infusion.

Formulations of the present methods suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations for parenteral administration include aqueous and non-aqueous sterile injection solutions which may contain antioxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

The pharmaceutical composition may comprise one or more pharmaceutical excipients. Any suitable pharmaceutical excipient may be used, and one of ordinary skill in the art is capable of selecting suitable pharmaceutical excipients. Pharmaceutical excipients include, for example, those described in the Handbook of Pharmaceutical Excipients, 8th Revised Ed. (2017).

5.4.2. Dosage Regimens

In various embodiments, the apelin receptor agonist (e.g., as described herein) is administered at a dose sufficient to treat an age-related muscle condition (e.g., as described herein).

In various embodiments, the apelin receptor agonist (e.g., as described herein) is administered in a method for maintaining and/or increasing muscle mass and/or muscle strength in an elderly subject. In some embodiments, the elderly subject is human and at least 50 years old, at least 55 years old, at least 60-years-old, or at least 65 years old.

In various embodiments, the dose of the apelin receptor agonist is at least 0.01 mg/kg, such as at least 0.5 mg/kg, or at least 1 mg/kg. In certain embodiments, the dose is 25 mg/kg to 1,000 mg/kg per day. In certain embodiments, the dose is 25 mg/kg to 1,500 mg/kg per day.

In some embodiments, the apelin receptor agonist is administered in a dose that is independent of patient weight or surface area (flat dose).

In various embodiments, the dose is 1-5000 mg. In various embodiments, the dose is 25-2000 mg. In some embodiments, the dose is at least 60 mg, at least 100 mg, at least 120 mg, at least 140 mg, at least 160 mg, at least 180 mg, at least 200 mg, at least 220 mg, at least 240 mg, at least 260 mg, at least 280 mg, at least 300 mg, at least 320 mg, at least 340 mg, at least 360 mg, at least 380 mg, at least 400 mg, at least 420 mg, at least 440 mg, at least 460 mg, at least 480 mg, at least 500 mg, at least 520 mg, at least 550 mg, at least 580 mg, at least 600 mg, at least 650 mg, at least 700 mg, at least 750 mg, at least 800 mg, at least 850 mg, at least 900 mg, at least 950 mg, at least 1000 mg, at least 1100 mg, at least 1200 mg, at least 1300 mg, at least 1400 mg, at least 1440 mg, or at least 100 mg. In various embodiments, the dose is 25-2000 mg. In some embodiments, the dose is at least 200 mg. In some embodiments, the dose is at least 240 mg. In some embodiments, the dose is at least 60 mg. In some embodiments, the dose is at least 360 mg. In some embodiments, the dose is at least 120 mg. In some embodiments, the dose is at least 720 mg.

The apelin receptor agonist can be administered in a single dose or in multiple doses.

In some embodiments, the dose is administered daily. In some embodiments, the dose is a single ascending dose (SAD). In some embodiments, the single ascending dose comprises a first dose of at least 60 mg, and a second dose of at least 360 mg. In some embodiments, the dose is a single ascending dose (SAD). In some embodiments, the single ascending dose comprises a first dose of at least 120 mg, and a second dose of at least 720 mg. In some embodiments, the dose is a single ascending dose (SAD). In some embodiments, the single ascending dose comprises a first dose of at least 240 mg, and a second dose of at least 1440 mg. In some embodiments, the dose is a single ascending dose (SAD). In some embodiments, the single ascending dose comprises a first dose of at least 20 mg, at least 30 mg, at least 40 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 170 mg, at least 180 mg, at least 190 mg, at least 200 mg, at least 210 mg, at least 220 mg, at least 230 mg, at least 240 mg, at least 250 mg, at least 260 mg, at least 270 mg, or at least 280 mg; and a second dose of at least of at least 20 mg, at least 30 mg, at least 40 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 170 mg, at least 180 mg, at least 190 mg, at least 200 mg, at least 210 mg, at least 220 mg, at least 230 mg, at least 240 mg, at least 250 mg, at least 260 mg, at least 270 mg, at least 280 mg, at least 290 mg, at least 300 mg, at least 310 mg, at least 320 mg, at least 330 mg, at least 340 mg, at least 350 mg, at least 360 mg, at least 400 mg, at least 450 mg, at least 500 mg, at least 550 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, or at least 2000 mg.

In some embodiments, the dose is administered as a plurality of equally or unequally divided sub-doses. In some embodiments, the dose is administered as multiple doses. In some embodiments, the dose is administered intravenously for 1 hour every day for at least 2 days, at least 3 days, at least 4 days, at least 5 days, at least 6 days, at least 7 days, at least 8 days, at least 9 days, or at least 10 days.

In certain embodiments, the dose is administered continuously (e.g., IV infusion) for a period of time. In certain embodiments, the dose is administered as a loading intravenous infusion dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours). In certain embodiments, following the loading dose, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a loading dose and a 24 hour or 48-hour washout period, the dose is administered as an intravenous infusion maintenance dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours). In certain embodiments, following a first loading dose and a 24 hour or 48-hour washout period, the dose is administered as an intravenous infusion loading dose for a period of time (e.g., 10 minutes, 20 minutes, 30 minutes, 40 minutes, 50 minutes, 1 hour, 2 hours, 3 hours, 5 hours, 6 hours, 7 hours, 8 hours, 9 hours, or 10 hours), followed by a maintenance dose for a period of time (e.g., 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or 48 hours).

In certain embodiments, the apelin receptor agonist is administered as follows: a loading dose for 1 hour via intravenous infusion, a 48-hour wash-out period, and a 1-hour loading dose via intravenous infusion followed by a 22-hour maintenance dose via intravenous infusion. In certain embodiments, the apelin receptor agonist is administered as follows: a loading dose for 1 hour via intravenous infusion, followed by a 119-hour maintenance dose via intravenous infusion.

In some embodiments, the apelin receptor agonist is administered orally, intravenously, intranasally, or intramuscularly. In some embodiments, the apelin receptor agonist is administered orally.

In some embodiments, the apelin receptor agonist is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In some embodiments, the apelin receptor agonist is administered continuously for at least 10 minutes, at least 20 minutes, at least 30 minutes, at least 40 minutes, at least 50 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 48 hours, at least 72 hours, at least 100 hours, at least 110 hours, at least 115 hours, at least 120 hours, or at least 125 hours.

5.4.3. Dosage Form

In some embodiments, an apelin receptor modulator or salt thereof is administered in a suspension. In other embodiments, an apelin receptor modulator or salt thereof is administered in a solution. In some embodiments, an apelin receptor modulator or salt thereof is administered in a solid dosage form. In particular embodiments, the solid dosage form is a capsule. In particular embodiments, the solid dosage form is a tablet. In specific embodiments, an apelin receptor modulator is in a crystalline or amorphous form. In particular embodiments, an apelin receptor modulator is in amorphous form. In some embodiments, the apelin receptor modulator is an apelin receptor agonist.

In one aspect of the methods, the apelin receptor modulator, or the pharmaceutical composition including same, is administered intravenously, topically, orally, by inhalation, by infusion, by injection, intraperitoneally, intramuscularly, subcutaneously, intra-aurally, by intra-articular administration, by intra-mammary administration, by topical administration or by absorption through epithelial or mucocutaneous linings. In certain embodiments, the apelin receptor modulator, or the pharmaceutical composition including same, is administered via intravenous infusion, in a capsule, or as a tablet.

### 5.5. Definitions

Unless defined otherwise, all technical and scientific terms used herein have the meaning commonly understood by a person skilled in the art to which this invention belongs.

The terms “individual,” “host,” and “subject” are used interchangeably, and refer to an animal to be treated, including but not limited to humans and non-human primates; rodents, including rats and mice; bovines; equines; ovines; felines; and canines. “Mammal” means a member or members of any mammalian species. Non-human animal models, i.e., mammals, non-human primates, murines, lagomorpha, etc. may be used for experimental investigations. The term “patient” refers to a human subject.

The term “modulator” refers to a compound or composition that modulates the level of a target, or the activity or function of a target, which may be, but is not limited to, apelin receptor. In some embodiments, the modulator compound can agonize or activate the target, such as apelin receptor. An agonist or activator of a target can increase the level of activity or signaling associated with the target.

The terms “treating,” “treatment,” and grammatical variations thereof are used in the broadest sense understood in the clinical arts. Accordingly, the terms do not require cure or complete remission of disease, and the terms encompass obtaining any clinically desired pharmacologic and/or physiologic effect, including improvement in physiologic measures associated with “normal”, non-pathologic, aging. Unless otherwise specified, “treating” and “treatment” do not encompass prophylaxis.

The phrase “therapeutically effective amount” refers to the amount of a compound that, when administered to a mammal or other subject for treating or preventing a disease, condition, or disorder, is sufficient to effect treatment of the disease, condition, or disorder. The “therapeutically effective amount” may vary depending on the compound, the disease and its severity and the age, weight, etc., of the subject to be treated.

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1 to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

Unless specifically stated or otherwise apparent from context, as used herein the term “about” is understood as within range of normal tolerance in the art, for example within 2 standard deviations of the mean, and is meant to encompass variations of ±20% or ±10%, more preferably ±5%, even more preferably ±1%, and still more preferably ±0.1% from the stated value. Where a percentage is provided with respect to an amount of a component or material in a composition, the percentage should be understood to be a percentage based on weight, unless otherwise stated or understood from the context.

It should be understood that the order of steps or order for performing certain actions is immaterial so long as the present disclosure remain operable. Moreover, two or more steps or actions can be conducted simultaneously.

The terms “pharmaceutically acceptable excipient,” “pharmaceutically acceptable diluent,” “pharmaceutically acceptable carrier,” and “pharmaceutically acceptable adjuvant” are used interchangeably and refer to an excipient, diluent, carrier, or adjuvant that is useful in preparing a pharmaceutical composition that is generally safe, non-toxic and neither biologically nor otherwise undesirable, and include an excipient, diluent, carrier, and adjuvant that is acceptable for veterinary use as well as human pharmaceutical use. The phrase “pharmaceutically acceptable excipient” includes both one and more than one such excipient, diluent, carrier, and/or adjuvant.

“Alkyl” refers to a saturated branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl and propan-2-yl, butyls such as butan-1-yl, butan-2-yl, 2-methyl-propan-1-yl, 2-methyl-propan-2-yl, tert-butyl, and the like. In certain embodiments, an alkyl group comprises 1 to 20 carbon atoms. In some embodiments, alkyl groups include 1 to 10 carbon atoms or 1 to 6 carbon atoms whereas in other embodiments, alkyl groups include 1 to 4 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl group and an alkyl group having 1 to 4 carbon atoms may be referred to as a (C₁-C₄)alkyl. This nomenclature may also be used for alkyl groups with differing numbers of carbon atoms. The term “alkyl may also be used when an alkyl group is a substituent that is further substituted in which case a bond between a second hydrogen atom and a C atom of the alkyl substituent is replaced with a bond to another atom such as, but not limited to, a halogen, or an O, N, or S atom. For example, a group —O—(C₁-C₆ alkyl)-OH will be recognized as a group where an —O atom is bonded to a C₁-C₆ alkyl group and one of the H atoms bonded to a C atom of the C₁-C₆ alkyl group is replaced with a bond to the O atom of an —OH group. As another example, a group —O—(C₁-C₆ alkyl)-O—(C₁-C₆ alkyl) will be recognized as a group where an —O atom is bonded to a first C₁-C₆ alkyl group and one of the H atoms bonded to a C atom of the first C₁-C₆ alkyl group is replaced with a bond to a second O atom that is bonded to a second C₁-C₆ alkyl group.

“Alkenyl” refers to an unsaturated branched or straight-chain hydrocarbon group having at least one carbon-carbon double bond derived by the removal of one hydrogen atom from a single carbon atom of a parent alkene. The group may be in either the Z- or E-form (cis or trans) about the double bond(s). Typical alkenyl groups include, but are not limited to, ethenyl; propenyls such as prop-1-en-1-yl, prop-1-en-2-yl, prop-2-en-1-yl (allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl; and the like. In certain embodiments, an alkenyl group has 2 to 20 carbon atoms and in other embodiments, has 2 to 6 carbon atoms. An alkenyl group having 2 to 6 carbon atoms may be referred to as a (C₂-C₆)alkenyl group.

“Alkynyl” refers to an unsaturated branched or straight-chain hydrocarbon having at least one carbon-carbon triple bond derived by the removal of one hydrogen atom from a single carbon atom of a parent alkyne. Typical alkynyl groups include, but are not limited to, ethynyl; propynyl; butynyl, 2-pentynyl, 3-pentynyl, 2-hexynyl, 3-hexynyl and the like. In certain embodiments, an alkynyl group has 2 to 20 carbon atoms and in other embodiments, has 2 to 6 carbon atoms. An alkynyl group having 2 to 6 carbon atoms may be referred to as a —(C₂-C₆)alkynyl group.

“Alkoxy” refers to a radical —OR where R represents an alkyl group as defined herein. Representative examples include, but are not limited to, methoxy, ethoxy, propoxy, butoxy, cyclohexyloxy, and the like. Typical alkoxy groups include 1 to 10 carbon atoms, 1 to 6 carbon atoms or 1 to 4 carbon atoms in the R group. Alkoxy groups that include 1 to 6 carbon atoms may be designated as —O—(C₁-C₆) alkyl or as —O—(C₁-C₆ alkyl) groups. In some embodiments, an alkoxy group may include 1 to 4 carbon atoms and may be designated as —O—(C₁-C₄) alkyl or as —O—(C₁-C₄ alkyl) groups group.

“Aryl” refers to a monovalent aromatic hydrocarbon group derived by the removal of one hydrogen atom from a single carbon atom of a parent aromatic ring system. Aryl encompasses monocyclic carbocyclic aromatic rings, for example, benzene. Aryl also encompasses bicyclic carbocyclic aromatic ring systems where each of the rings is aromatic, for example, naphthalene. Aryl groups may thus include fused ring systems where each ring is a carbocyclic aromatic ring. In certain embodiments, an aryl group includes 6 to 10 carbon atoms. Such groups may be referred to as C₆-C₁₀ aryl groups. Aryl, however, does not encompass or overlap in any way with heteroaryl as separately defined below. Hence, if one or more carbocyclic aromatic rings is fused with an aromatic ring that includes at least one heteroatom, the resulting ring system is a heteroaryl group, not an aryl group, as defined herein.

“Carbonyl” refers to the radical —C(O) or —C(═O) group.

“Carboxy” refers to the radical —C(O)OH.

“Cyano” refers to the radical —CN.

“Cycloalkyl” refers to a saturated cyclic alkyl group derived by the removal of one hydrogen atom from a single carbon atom of a parent cycloalkane. Typical cycloalkyl groups include, but are not limited to, groups derived from cyclopropane, cyclobutane, cyclopentane, cyclohexane, cycloheptane, cyclooctane, and the like. Cycloalkyl groups may be described by the number of carbon atoms in the ring. For example a cycloalkyl group having 3 to 7 ring members may be referred to as a (C₃-C₇)cycloalkyl and a cycloalkyl group having 4 to 7 ring members may be referred to as a (C₄-C₇)cycloalkyl. In certain embodiments, the cycloalkyl group can be a (C₃-C₁₀)cycloalkyl, a (C₃-C₈)cycloalkyl, a (C₃-C₇)cycloalkyl, a (C₃-C₆)cycloalkyl, or a (C₄-C₇)cycloalkyl group and these may be referred to as C₃-C₁₀ cycloalkyl, C₃-C₈ cycloalkyl, C₃-C₇ cycloalkyl, C₃-C₆ cycloalkyl, or C₄-C₇ cycloalkyl groups using alternative language.

“Heterocyclyl” refers to a cyclic group that includes at least one saturated or unsaturated, but non-aromatic, cyclic ring. Heterocyclyl groups include at least one heteroatom as a ring member. Typical heteroatoms include O, S and N and are independently chosen. Heterocyclyl groups include monocyclic ring systems and bicyclic ring systems. Bicyclic heterocyclyl groups include at least one non-aromatic ring with at least one heteroatom ring member that may be fused to a cycloalkyl ring or may be fused to an aromatic ring where the aromatic ring may be carbocyclic or may include one or more heteroatoms. The point of attachment of a bicyclic heterocyclyl group may be at the non-aromatic cyclic ring that includes at least one heteroatom or at another ring of the heterocyclyl group. For example, a heterocyclyl group derived by removal of a hydrogen atom from one of the 9 membered heterocyclic compounds shown below may be attached to the rest of the molecule at the 5-membered ring or at the 6-membered ring.

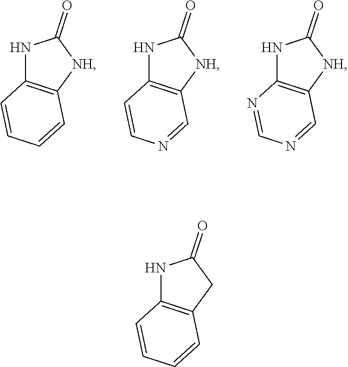 [SMILES: O=c1[nH]c2ccccc2[nH]1 ; O=c1[nH]c2ccncc2[nH]1 ; O=c1[nH]c2cncnc2[nH]1 ; O=C1Cc2ccccc2N1]

In some embodiments, a heterocyclyl group includes 5 to 10 ring members of which 1, 2, 3 or 4 or 1, 2, or 3 are heteroatoms independently selected from O, S, or N. In other embodiments, a heterocyclyl group includes 3 to 7 ring members of which 1, 2, or 3 heteroatoms are independently selected from O, S, or N. In such 3-7 membered heterocyclyl groups, only 1 of the ring atoms is a heteroatom when the ring includes only 3 members and includes 1 or 2 heteroatoms when the ring includes 4 members. In some embodiments, a heterocyclyl group includes 3 or 4 ring members of which 1 is a heteroatom selected from O, S, or N. In other embodiments, a heterocyclyl group includes 5 to 7 ring members of which 1, 2, or 3 are heteroatoms independently selected from O, S, or N. Typical heterocyclyl groups include, but are not limited to, groups derived from epoxides, aziridine, azetidine, imidazolidine, morpholine, piperazine, piperidine, hexahydropyrimidine, 1,4,5,6-tetrahydropyrimidine, pyrazolidine, pyrrolidine, quinuclidine, tetrahydrofuran, tetrahydropyran, benzimidazolone, pyridinone, and the like. Substituted heterocyclyl also includes ring systems substituted with one or more oxo (═O) or oxide (—O⁻) substituents, such as piperidinyl N-oxide, morpholinyl-N-oxide, 1-oxo-1-thiomorpholinyl, pyridinonyl, benzimidazolonyl, benzo[d]oxazol-2(3H)-only, 3,4-dihydroisoquinolin-1(2H)-only, indolin-only, 1H-imidazo[4,5-c]pyridin-2(3H)-only, 7H-purin-8(9H)-only, imidazolidin-2-only, 1H-imidazol-2(3H)-only, 1,1-dioxo-1-thiomorpholinyl, and the like.

“Halo” or “halogen” refers to a fluoro, chloro, bromo, or iodo group.

“Haloalkyl” refers to an alkyl group in which at least one hydrogen is replaced with a halogen. Thus, the term “haloalkyl” includes monohaloalkyl (alkyl substituted with one halogen atom) and polyhaloalkyl (alkyl substituted with two or more halogen atoms). Representative “haloalkyl” groups include difluoromethyl, 2,2,2-trifluoroethyl, 2,2,2-trichloroethyl, and the like. The term “perhaloalkyl” means, unless otherwise stated, an alkyl group in which each of the hydrogen atoms is replaced with a halogen atom. For example, the term “perhaloalkyl”, includes, but is not limited to, trifluoromethyl, pentachloroethyl, 1,1,1-trifluoro-2-bromo-2-chloroethyl, and the like.

“Heteroaryl” refers to a monovalent heteroaromatic group derived by the removal of one hydrogen atom from a single atom of a parent heteroaromatic ring system. Heteroaryl groups typically include 5- to 14-membered, but more typically include 5- to 10-membered aromatic, monocyclic, bicyclic, and tricyclic rings containing one or more, for example, 1, 2, 3, or 4, or in certain embodiments, 1, 2, or 3, heteroatoms chosen from O, S, or N, with the remaining ring atoms being carbon. In monocyclic heteroaryl groups, the single ring is aromatic and includes at least one heteroatom. In some embodiments, a monocyclic heteroaryl group may include 5 or 6 ring members and may include 1, 2, 3, or 4 heteroatoms, 1, 2, or 3 heteroatoms, 1 or 2 heteroatoms, or 1 heteroatom where the heteroatom(s) are independently selected from O, S, or N. In bicyclic aromatic rings, both rings are aromatic. In bicyclic heteroaryl groups, at least one of the rings must include a heteroatom, but it is not necessary that both rings include a heteroatom although it is permitted for them to do so. For example, the term “heteroaryl” includes a 5- to 7-membered heteroaromatic ring fused to a carbocyclic aromatic ring or fused to another heteroaromatic ring. In tricyclic aromatic rings, all three of the rings are aromatic and at least one of the rings includes at least one heteroatom. For fused, bicyclic and tricyclic heteroaryl ring systems where only one of the rings contains one or more heteroatoms, the point of attachment may be at the ring including at least one heteroatom or at a carbocyclic ring. When the total number of S and O atoms in the heteroaryl group exceeds 1, those heteroatoms are not adjacent to one another. In certain embodiments, the total number of S and O atoms in the heteroaryl group is not more than 2 In certain embodiments, the total number of S and O atoms in the aromatic heterocycle is not more than 1 Heteroaryl does not encompass or overlap with aryl as defined above. Examples of heteroaryl groups include, but are not limited to, groups derived from acridine, carbazole, cinnoline, furan, imidazole, indazole, indole, indolizine, isobenzofuran, isochromene, isoindole, isoquinoline, isothiazole, 2H-benzo[d][1,2,3]triazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and the like. In certain embodiments, the heteroaryl group can be between 5 to 20 membered heteroaryl, such as, for example, a 5 to 14 membered or 5 to 10 membered heteroaryl. In certain embodiments, heteroaryl groups can be those derived from thiophene, pyrrole, benzothiophene, 2H-benzo[d][1,2,3]triazole benzofuran, indole, pyridine, quinoline, imidazole, benzimidazole, oxazole, tetrazole, and pyrazine.

As described herein, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### 6. ADDITIONAL EMBODIMENTS

Aspects of this disclosure are further described in the following numbered clauses.

Clause 1. A method of treating a muscle condition in a subject, the method comprising administering to a subject in need thereof an effective dose of an apelin receptor agonist of formula (I) or (II):

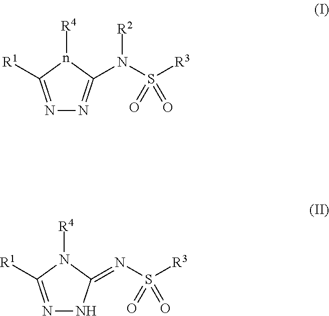

or a pharmaceutically acceptable salt thereof, a tautomer thereof, a pharmaceutically acceptable salt of the tautomer, a stereoisomer of any of the foregoing, or a mixture thereof,
wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{a1} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

Clause 2. The method of clause 1, wherein the muscle condition is an age-related muscle condition.

Clause 3. The method of any one of clauses 1 to 2, wherein the subject is human and at least 40-years-old.

Clause 4. The method of clause 3, wherein the subject is at least 50-years-old.

Clause 5. The method of clause 4, wherein the subject is at least 60-years-old.

Clause 6. The method of clause 5, wherein the subject is at least 65-years-old.

Clause 7. The method of clause 6, wherein the subject is at least 70-years-old.

Clause 8. The method of clause 7, wherein the subject is at least 75-years-old.

Clause 9. The method of clause 8, wherein the subject is at least 80-years-old.

Clause 10. The method of any one of clauses 1 to 9, wherein the muscle condition is a skeletal muscle condition.

Clause 11. The method of clause 10, wherein the muscle expresses the apelin receptor and administration of the apelin receptor agonist activates the apelin/APJ (APLNR) system in the muscle tissue of the subject.

Clause 12. The method of any one of clauses 1 to 11, wherein the muscle condition is not a cardiovascular condition.

Clause 13. The method of any one of clauses 1 to 11, wherein the subject is not suffering from, or at risk of, a heart failure.

Clause 14. The method of any one of clauses 1 to 13, wherein the age-related muscle condition is associated with inflammation and/or impairment of mitochondrial function.

Clause 15. The method of any of one of clauses 1 to 14, wherein the age-related muscle condition is associated with a loss-of-function of muscle, decrease in the ability to regenerate muscle, or decrease in the ability to heal after injury of muscle.

Clause 16. The method of any of one of clauses 1 to 15, wherein the age-related muscle condition is associated with the loss-of-function of muscle stem cells.

Clause 17. The method of any one of clauses 1 to 16, wherein the age-related muscle condition is selected from sarcopenia, frailty, hip fracture, ICU associated muscle weakness, mechanical ventilation-related muscle weakness, immobilization associated muscle weakness, recovery from muscle injury, muscle atrophy, diaphragm atrophy, critical illness myopathy, and muscle wasting.

Clause 18. The method of any of one of clauses 1 to 17, wherein the age-related muscle condition is associated with insulin insensitivity or resistance, or Type 2 diabetes mellitus.

Clause 19. The method of any one of clauses 1 to 18, wherein the human subject has, or is identified as having, low muscle strength or low muscle force.

Clause 20. The method of any one of clauses 17 to 19, wherein the human subject has, or is identified as having, chronic obstructive pulmonary disease (COPD).

Clause 21. The method of any one of clauses 1 to 20, wherein the human subject has, or is identified as having, low lower limb muscle mass.

Clause 22. The method of any one of clauses 1 to 21, wherein the human subject has, or is identified as having, low upper limb muscle mass.

Clause 23. The method of any one of clauses 1 to 22, wherein the human subject has, or is identified as having, low muscle volume.

Clause 24. The method of clause 23, wherein the muscle volume is muscle volume.

Clause 25. The method of clause 24, wherein the muscle is tibialis anterior, tibialis posterior, gastrocnemius, sartorius, vastus intermedius, vastus laterals, vastus medialis, soleus, rectus femorus, extensor digitorum longus, or diaphragm.

Clause 26. The method of any one of clauses 1 to 25, wherein the apelin receptor agonist is administered orally, intravenously, intranasally, or intramuscularly.

Clause 27. The method of any one of clauses 1 to 26, wherein the dose is administered daily.

Clause 28. The method of any one of clauses 1 to 27, wherein the dose is administered as a plurality of equally or unequally divided sub-doses.

Clause 29. The method of any one of clauses 1 to 28, wherein the dose is administered at varying dosing intervals.

Clause 30. The method of any one of clauses 1 to 29, wherein the dose is 200 mg.

Clause 31. The method of clause one of clauses 1 to 30, further comprising, assessing muscle mass after the dosing.

Clause 32. The method of clause 31, wherein the muscle mass is assessed at least one day after dosing.

Clause 33. The method of clause 32, wherein the muscle mass is assessed at least one week or at least two weeks after dosing.

Clause 34. The method of clause 33, wherein the muscle mass is assessed at least one month after dosing.

Clause 35. The method of any of clauses 1-34, wherein the subject has a low circulating level of apelin.

Clause 36. A method for maintaining and/or increasing muscle mass and/or muscle strength in an elderly subject, the method comprising administering to a subject in need thereof an effective dose of an apelin receptor agonist of formula (I) or (II):

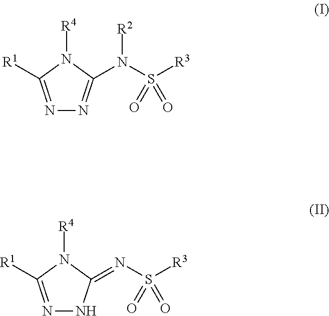

or a pharmaceutically acceptable salt thereof, a tautomer thereof, a pharmaceutically acceptable salt of the tautomer, a stereoisomer of any of the foregoing, or a mixture thereof, wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{3a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{3a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups

Clause 37. The method of clause 36, wherein the subject is at least 60-years-old.

Clause 38. The method of clause 37, wherein the subject is at least 65-years-old.

Clause 39. The method of clause 38, wherein the subject is at least 70-years-old.

Clause 40. The method of clause 39, wherein the subject is at least 75-years-old.

Clause 41. The method of clause 40, wherein the subject is at least 80-years-old.

Clause 42. The method of any one of clauses 36 to 41, wherein the human subject has, or is identified as having, low muscle strength.

Clause 43. The method of any one of clauses 36 to 42, wherein the human subject has, or is identified as having, low muscle force.

Clause 44. The method of any one of clauses 36 to 43, wherein the human subject has, or is identified as having, low lower limb muscle mass.

Clause 45. The method of any one of clauses 36 to 44, wherein the human subject has, or is identified as having, low upper limb muscle mass.

Clause 46. The method of any one of clauses 36 to 45, wherein the human subject has, or is identified as having, low muscle volume.

Clause 47. The method of clause 46, wherein the muscle volume is muscle volume.

Clause 48. The method of clause 47, wherein the muscle is diaphragm, tibialis anterior, tibialis posterior, gastrocnemius, sartorius, vastus intermedius, vastus laterals, vastus medialis, soleus, rectus femorus, or extensor digitorum longus.

Clause 49. The method of any one of clauses 47 to 48, wherein the muscle is a skeletal muscle.

Clause 50. The method of any one of clauses 36 to 49, wherein the human subject is mechanically ventilated.

Clause 51. The method of any one of clauses 36 to 50, wherein the human subject has, or is identified as having reduced diaphragm thickness as compared to a human subject that is not mechanically ventilated.

Clause 52. The method of any one of clauses 36 to 51, wherein the human subject has, or is identified as having diaphragm atrophy.

Clause 53. The method of any one of clauses 36 to 52, wherein the human subject has, or is identified as having ventilator-induced diaphragmatic dysfunction (VIDD).

Clause 54. The method of any one of clauses 36 to 53, wherein the human subject has, or is identified as having hypoxic respiratory failure.

Clause 55. The method of any one of clauses 48 to 54, wherein the muscle expresses the apelin receptor.

Clause 56. The method of any of clauses 36 to 55, wherein the human subject has a low circulating apelin level.

Clause 57. The method of any one of clauses 36 to 56, wherein the apelin receptor agonist is administered orally, intravenously, intranasally, or intramuscularly.

Clause 58. The method of any one of clauses 36 to 57, wherein the dose is administered daily.

Clause 59. The method of any one of clauses 36 to 58, wherein the dose is administered as a plurality of equally or unequally divided sub-doses.

Clause 60. The method of any one of clauses 36-59, wherein the dose is administrated intravenously.

Clause 61. The method of clause 60, wherein the dose is administered intravenously at a loading dose of at least 60 mg followed by a maintenance dose of at least 360 mg.

Clause 62. The method of clause 60, wherein the dose is administered intravenously at a loading dose of at least 120 mg followed by a maintenance dose of at least 720 mg.

Clause 63. The method of clause 60, wherein the dose is administered intravenously at a loading dose of at least 240 mg followed by a maintenance dose of at least 1440 mg.

Clause 64. The method of any one of clauses 61 to 63, wherein the loading dose is administered for at least 1 hour.

Clause 65. The method of any one of clauses 61 to 64, wherein the maintenance dose is administered for at least 20 hours.

Clause 66. The method of any one of clauses 61 to 64, wherein the maintenance dose is administered for at least 22 hours.

Clause 67. The method of any one of clauses 61 to 64, wherein the maintenance dose is administered for at least 100 hours.

Clause 68. The method of any one of clauses 36 to 60, wherein the dose is at least 60 mg.

Clause 69. The method of any one of clauses 36 to 60, wherein the dose is at least 75 mg.

Clause 70. The method of any one of clauses 36 to 60, wherein the dose is at least 120 mg.

Clause 71. The method of any one of clauses 36 to 60, wherein the dose is at least 240 mg.

Clause 72. The method of any one of clauses 36 to 60, wherein the dose is at least 150 mg.

Clause 73. The method of any one of clauses 36 to 60, wherein the dose is at least 300 mg.

Clause 74. The method of any one of clauses 36 to 60, wherein the dose is at least 375 mg.

Clause 75. The method of any one of clauses 36 to 60, wherein the dose is 75-150 mg.

Clause 76. The method of any one of clauses 36 to 60, wherein the dose is 150-300 mg.

Clause 77. The method of any one of clauses 36 to 60, wherein the dose is 240-1440 mg.

Clause 78. The method of any one of clauses 36 to 60, wherein the dose is 75 mg.

Clause 79. The method of any one of clauses 36 to 60, wherein the dose is 150 mg.

Clause 80. The method of any one of clauses 36 to 60, wherein the dose is 240 mg.

Clause 81. The method of any one of clauses 36 to 60, wherein the dose is 300 mg.

Clause 82. The method of any one of clauses 36 to 60, wherein the dose is 375 mg.

Clause 83. The method of any one of clauses 36 to 60, wherein the dose is 450 mg.

Clause 84. The method of any one of clauses 36 to 60, wherein the dose is a single ascending dose of 60 mg/360 mg.

Clause 85. The method of any one of clauses 36 to 60, wherein the dose is a single ascending dose of 120 mg/720 mg.

Clause 86. The method of any one of clauses 36 to 60, wherein the dose is a single ascending dose of 240 mg/1440 mg.

Clause 87. The method of any one of clauses 36 to 86, further comprising, assessing muscle mass or muscle thickness after the dosing.

Clause 88. The method of clause 87, wherein the muscle mass is assessed at least one day after dosing.

Clause 89. The method of clause 87, wherein the muscle mass is assessed at least one week after dosing.

Clause 90. The method of clause 88, wherein the muscle mass is assessed at least one month after dosing.

Clause 91. The method of any one of clauses 1 to 90, wherein R¹ is an unsubstituted pyridyl or is a pyridyl substituted with 1 or 2 R^{1a} substituents.

Clause 92. The method of any one of clauses 1 to 91, wherein R^{1a} in each instance is independently selected from —CH₃, —CH₂CH₃, —F, —Cl, —Br, —CN, —CF₃, —CH═CH₂, —C(═O)NH₂, —C(═O)NH(CH₃), —C(═O)N(CH₃)₂, —C(═O)NH(CH₂CH₃), —OH, —OCH₃, —OCHF₂, —OCH₂CH₃, —OCH₂CF₃, —OCH₂CH₂OH, —OCH₂C(CH₃)₂OH, —OCH₂C(CF₃)₂OH, —OCH₂CH₂OCH₃, —NH₂, —NHCH₃, —N(CH₃)₂, phenyl, and a group of formula

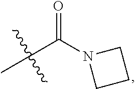 [SMILES: *C(=O)N1CCC1]

wherein the symbol [symbol] , when drawn across a bond, indicates the point of attachment to the rest of the molecule.

Clause 93. The method of any one of clauses 1 to 92, wherein R¹ is selected from

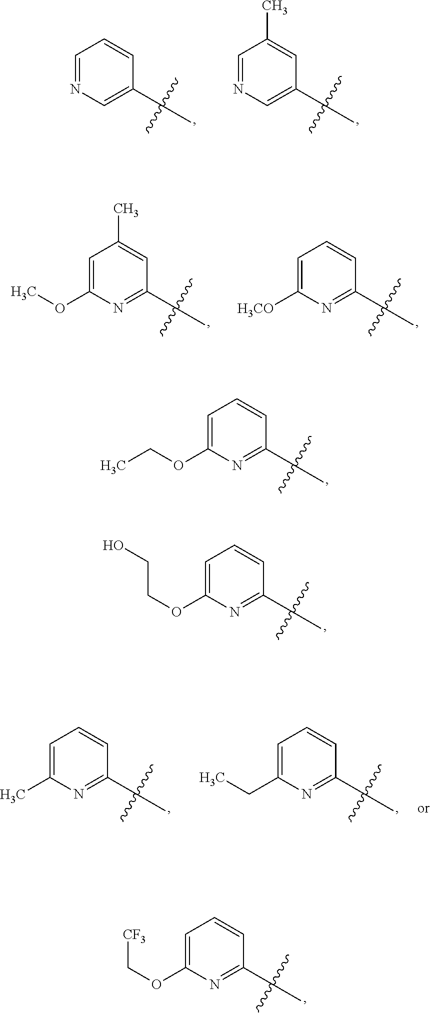

wherein the symbol [symbol] , when drawn across a bond, indicates the point of attachment to the rest of the molecule.

Clause 94. The method of any one of clauses 1 to 93, wherein R² is —H.

Clause 95. The method of any one of clauses 1 to 94, wherein R⁴ is a phenyl, pyridyl, pyrimidinyl, isoxazolyl, indolyl, naphthyl, or pyridinyl any of which may be unsubstituted or substituted with 1, 2, or 3 R^{4a} substituents.

Clause 96. The method of clause 95, wherein R⁴ is a phenyl substituted with 1 or 2 R^{4a} substituents.

Clause 97. The method of clause 96, wherein the 1 or 2 R^{4a} substituents are —O—(C₁-C₂ alkyl) groups.

Clause 98. The method of any one of clauses 1 to 97, wherein R^{4a} is in each instance independently selected from —CH₃, —F, —Cl, —Br, —CN, —CF₃, —OCH₃, —OCHF₂, —OCH₂CH₃, —C(═O)OCH₃, —C(═O)CH₃, or —N(CH₃)₂.

Clause 99. The method of any one of clauses 1 to 83, wherein R³ is selected from a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3b}═CR^{3c})-Q, or a group of formula -(heterocyclyl)-Q, wherein the heterocyclyl of the -(heterocyclyl)-Q has 5 to 7 ring members of which 1, 2, or 3 are heteroatoms selected from N, O, or S and is unsubstituted or is substituted with 1, 2, or 3 R^{3h} substituents.

Clause 100. The method of any one of clauses 1 to 98, wherein Q is selected from pyrimidinyl, pyridyl, isoxazolyl, thiazolyl, imidazolyl, phenyl, tetrahydropyrimidinonyl, cyclopropyl, cyclobutyl, cyclohexyl, morpholinyl, pyrrolidinyl, pyrazinyl, imidazo[1,2-a]pyridinyl, pyrazolyl, or oxetanyl any of which may be unsubstituted or substituted with 1, 2, or 3, R^{Q} substituents.

Clause 101. The method of any one of clauses 1 to 99, wherein Q is a monocyclic heteroaryl group with 5 or 6 ring members containing 1 or 2 heteroatoms selected from N, O, or S and Q is unsubstituted or is substituted with 1 or 2 R^{Q} substituents.

Clause 102. The method of any one of clauses 1 to 101, wherein R³ is a group of formula —(CR^{3d}R^{3e})—(CR^{3f}R^{3g})-Q.

Clause 103. The method of any one of clauses 1 to 102, wherein R³ has the formula

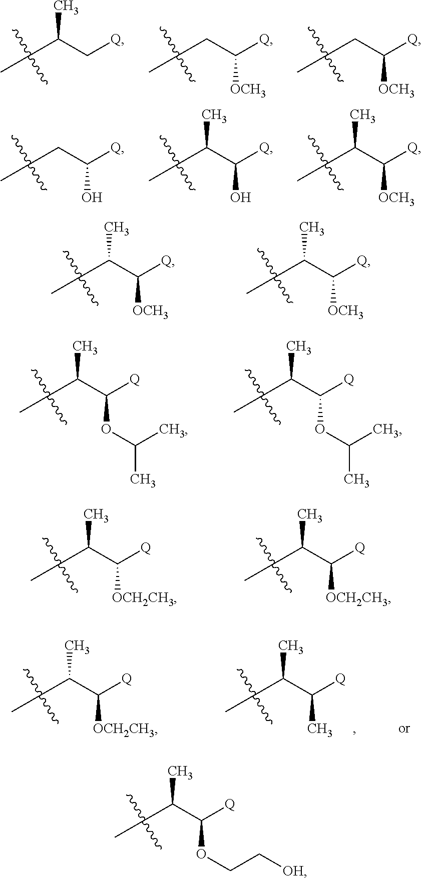

wherein the symbol [symbol] when drawn across a bond, indicates the point of attachment to the rest of the molecule.

Clause 104. The method of any one of clauses 1 to 103, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide, or a pharmaceutically acceptable salt thereof, a tautomer thereof, a pharmaceutically acceptable salt of the tautomer, a stereoisomer of any of the foregoing, or a mixture thereof.

Clause 105. The method of clause 104, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide or a pharmaceutically acceptable salt thereof.

Clause 106. The method of clause 104, wherein the apelin receptor agonist is BGE-105.

### 7. EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature

### 7.1. Example 1: Bioinformatic Analyses Identify Relationships Between Apelin and all-Cause Mortality and Mobility Decline Events in Human Healthy Aging Cohorts

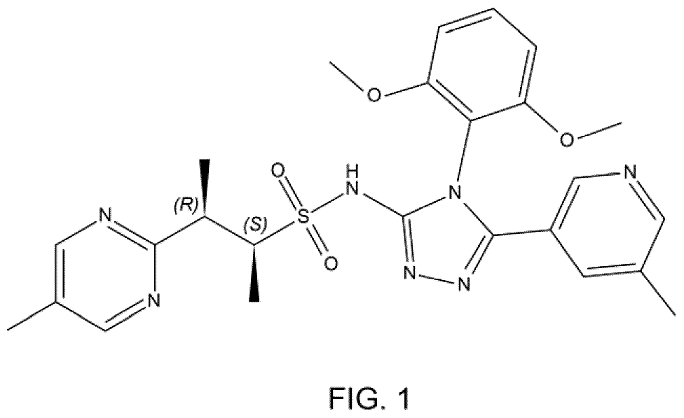

A survival predictor model was used to examine the relationship between serum levels of apelin and future risk of all-cause mortality in human healthy aging cohorts, using unpublished clinical outcome data and proteomics data generated on archived samples, based on survival modeling. By merging the proteomic data with participant phenotype and clinical data, “muscle aging scores” were calculated for all measured proteins that reflected a composite of hazard ratios associated with longitudinal grip strength decline and mortality. The analysis identified apelin (APLN), an exerkine secreted by skeletal muscle in response to exercise, as a target for muscle aging. Additionally, in the cohort, Apelin protein levels were associated with increased probabilities of both longevity (living to ≥90 yrs) and preservation of grip strength (living to ≥90 without grip strength decline from baseline of ≥10 kg) (FIG. 1b).

Additionally, the relationship between apelin levels and mobility decline events (e.g., a decrease in walking, stair-climbing, or transferring activities indicated by self-reported difficulty of these activities) was examined. A Cox proportional hazards model was used, with a hazard ratio and associated p-value generated for apelin.

As shown in FIG. 2A, a Kaplan-Meier curve of survival probability was generated for humans in the top 20% (blue) versus bottom 20% (red) of apelin protein levels. In humans, we have discovered that higher circulating levels of apelin are associated with decreased all-cause mortality (p=0.0002). FIG. 2B shows a similar model, where we discovered that higher circulating levels of apelin are associated with increased mobility (p=0.0082). The hazard ratio for apelin (0.88 in FIG. 2A and 0.89 in FIG. 2B) was generated using a Cox proportional hazards model. In both cases, the hazard ratio given is for the continuous Cox proportional hazards analysis, which is fitting to the entire distribution of apelin measurements. p-values in FIGS. 2A and 2B were calculated for these hazard ratios, based on testing the null hypothesis that the hazard ratio in each case equals 1.

Next, the protein levels were subjected to a rank-based inverse normalization and calculated pairwise Spearman correlations coefficient between normalized levels of all 4,575 proteins. Among the 590 proteins that were significantly correlated (Benjamini-Hochberg FDR<0.05) with apelin (referred to as the apelin protein module; FIG. 2C), a hypergeometric test revealed significant enrichment of proteins associated with all-cause mortality (p=1.04E-10).

A multivariate Cox regression model was then used to test for association between the first principal component (PC1) of the apelin protein module and death rate after adjusting for age, pack-years smoked, and monthly alcohol consumption. The contribution of PC1 to the relative death rate in this model (i.e., the termplot), with the median PC1 value used as a reference, ranged from 1.43 to 0.77 (FIG. 2D).

FIG. 2C, shows the serum abundance of the apelin protein module (highlighted by the green oval) in the HHS cohort. Each node represents a protein, and the edges between the nodes represent significant correlations. FIG. 2D shows the first principal component of the apelin protein module and death rate. The relative death rate (log; y-axis) was derived from the multivariate Cox regression model for the PC1 after adjusting for age, smoking pack years, and alcohol status. The reference used was the median value of PC1.

### 7.2. Example 2: BGE-105 Improves Activity Levels in Old Mice (Frailty Study)

Based on the discovery of the association of baseline apelin and apelin receptor protein levels with future aging outcomes in otherwise healthy, aged, humans as described in Example 1, an agonist of apelin receptors was administered to elderly mice to assess the effects of the agonist on voluntary physical activity as compared to age-matched controls.

BGE-105 has the structure shown below (FIG. 1):

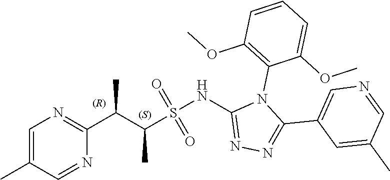

BGE-105 is known to activate the apelin receptor and it induces a cardiovascular response in rats (Ason et al., JCI Insight. 5(8):1-16(2020)). Clinical trials were also done with BGE-105 to study the safety, tolerability, and pharmacokinetics in healthy subjects and those with suffering impaired renal function (NCT03318809) or heart failure (NCT03276728).

In the current study, aged (24-month-old) mice were treated with BGE-105 daily (in water ad libitum) for 2 months. The animals were housed with access to voluntary running wheels that wirelessly transmit running data to a computer for analysis. Voluntary running wheel activity levels were measured daily, and body weights were measured every 2 weeks. The effects of BGE-105 on the prevention of frailty in mice were examined.

The formal test involve d calculating a Spearman correlation coefficient between these daily differences and the day number (e.g., days 1, 2, 3, etc. of the experiment) and testing the null hypothesis that this correlation coefficient equals 0.

The first day of the study (Study Day 1) started with animal acclimation, followed by the BGE-105 treatment start date on Study Day 19 (Phase Day 1). The study concluded on Study Day 83. Activity wheel monitoring started on Study Day 1 and ended on Study Day 83 (Phase Day 64). The data was analyzed at the end of the study. The total duration of activity monitoring after BGE-105 treatment initiation was 64 days. For the frailty portion of the study, mice were assessed using an activity monitoring wheel which was monitored passively with a computer monitoring system.

As shown in Table 1, the study included 23-24-month-old mice from strain C57BL/6. It is known that mice ranging from 18-24 months of age correlate with humans ranging from 56-69 years of age, with mice older than 24 months correlating with humans beyond 69 years old (Flurkey, Currer, and Harrison, 2007. “The mouse in biomedical research” in James G. Fox (ed.), American College of Laboratory Animal Medicine series, Elsevier, AP: Amsterdam; Boston). This age range meets the definition of “old,” defined as the presence of senescent changes in biomarkers in animals.

TABLE 1
Test Articles

| | | | | Number of | Dose Regimen | Dose | Dose | |
|---|---|---|---|---|---|---|---|---|
| Cohort | Strain | Category | Age | Animals | and Route | Volume | Concentration | |
| BGE- | C57bl/6 | E | 23-24 | 20 ± 0 | In Drinking | ad libitum | 275 | ug/mL |
| 105 | | | Months | | 8.5 pH Water | | | |
| Vehicle | C57bl/6 | E | 23-24 | 20 ± 0 | In Drinking | ad libitum | 0 | ug/mL |
| | | | Months | | 8.5 pH Water | | | |

Mice were treated with BGE-105 at a dose concentration of 275 ug/mL. BGE-105 was dissolved in deionized water at 275 ug/mL. BGE-105 was administered in drinking water consumed ad libitum. The compound is mildly acidic when dissolved, resulting in a pH 4.5 solution. The deionized water was adjusted to pH 8.5 by adding 1N NaOH. The vehicle control group consumed water (ad libitum) of the same pH without drug.

The study parameters for Groups 1-2 are provided in Table 2. The study parameters for mice in Groups 1-2 included animal acclimation, animal welfare, such as checking the weight of the animal, clinical examination, administering the treatment, activity monitoring, and blood collection, on the particular Study Days and/or Phase Days.

TABLE 2
Procedure Schedule

| | | |
|---|---|---|
| Phase Day (PD)/ | Experimental | |
| Study Day (SD) | Arm/Group | Description |

Acclimation

| | | |
|---|---|---|
| SD 1-7 | Acclimation-1: | Weigh mice at beginning and |
| | All Groups | end of week |
| | | Weigh control water |
| | | Clinical Examination |
| | | Single house in Mouse Cages |
| | | with Activity Monitoring Wheel |
| | | Exclude Nestlets, Innodome, |
| | | Innowheel |
| SD 8-19 | Acclimation-2: | Weigh mice |
| | All Groups | Clinical Examination |
| | | Insert a bottle of control water |
| | | with a pH of 8.5 |
| | | Weigh water bottle once every |
| | | 48 hours |
| | | Activity Monitoring Wheel |
| | | Begin monitoring 24-hour |
| | | activity monitoring wheel |
| | | Exclude Nestlets, Innodome, |
| | | Innowheel |
| SD 19 | Active: | Grid Hang Test |
| | All Groups | 1 trial (baseline values before |
| | | treatment begins) |

Treatment

| | | |
|---|---|---|
| PD 1-7/SD 20-26 | Active: | Randomize animals with |
| | All Groups | consideration of behavior tests, |
| | | Smart Wheel data and Body |
| | | Weights. |
| | | [0001] Weigh mice |
| | | once every week for |
| | | the first 2 weeks, |
| | | then on an as needed |
| | | basis |
| | | [0002] Clinical |
| | | Examination |
| | | Begin treating the experimental |
| | | group with BGE-105 treated water |
| | | for the duration of the study at a pH |
| | | of 8.5. |
| | | Control group continues to drink pH |
| | | matched control water. |
| | | weigh water bottle once every 48 |
| | | hours |
| | | Check the pH of BGE-105 and |
| | | vehicle when prepared fresh and |
| | | after 2 weeks at RT before replacing |
| | | with freshly prepared bottles |
| PD 8-65/SD 26-83 | Active: | Weigh mice once every week |
| | All Groups | Clinical Examination |
| | | Monitor the amount of food in each |
| | | dish once per day |
| | | Activity Monitoring Wheel: |
| | | Continue activity monitoring |
| | | Continue BGE-105 vs control |
| | | treatment as above |
| | | Weigh water bottle once every week |
| | | Monitor mice daily through cage for |
| | | clinical signs and to ensure wheel is |
| | | not blocked by debris |
| | | Minimize interaction with mice and |
| | | allow them to express their natural |
| | | activity |
| PD all/SD all | Active: | Change cages as necessary |
| | All Groups | |
| PD 65/SD 80 | Active: | Activity Monitoring Wheel: |
| | All Groups | End activity monitoring. |
| | | Grid Hang Test |
| | | 1 trial (final values) |

| | |
|---|---|
| Humane Endpoint: | If animal meets humane endpoint: |
| (criteria for euthanasia): | Notified study director and take |
| 1.Moribund | note of time and cage number and |
| a. Indicated by: BCS = 1 for 48 | euthanize animal as appropriate |
| consecutive hours OR 25% weight | |
| loss OR severely hunched posture | |
| with squinting eyes OR inability | |
| to self-right | |
| 2. Agonal breathing/cyanosis | |
| 3. Inability to eat or drink | |
| 4. Severe diarrhea | |
| 5. Severe bleeding from any orifice | |
| 6. Severe convulsions from which | |
| the animal does not recover | |
| (e.g. status epilepticus) | |
| 7. Self-induced trauma | |
| (e.g. autotomy) | |
| Animal Found Dead: | If an animal is found dead: |
| | 1. NOTIFIED STUDY |
| | DIRECTOR AND TAKE |
| | NOTE OF TIME AND CAGE |
| | NUMBER |

Activity Monitoring Wheel Test

The activity-monitoring wheel is a running disk that monitors rotations. The wheel is capable of monitoring voluntary wheel running 24 hours a day. Activity was monitored passively and wirelessly with a computer monitoring system. Running wheel activity levels were monitored daily. The wheel data was reported as the daily median rotations in each group (BGE-105 treated vs. controls). Mouse activity levels were measured as the number of wheel revolutions per day for each mouse and converted into a daily count of kilometers run using the diameter of the wheel. Within each experimental group, the daily median value for activity was calculated. For each experiment, a baseline period before experiment start was used to calculate median baseline activity levels for each mouse. These baselines were subtracted from future measurements for the same mouse. The resulting daily-corrected medians during the experiment were plotted for each day of the experiment and a smoothed curve was drawn using local regression (LOESS). The daily differences between the distances run in each group were calculated and tested for an increasing trend using Kendall's rank correlation tau.

Study Results

As shown in FIG. 3A, BGE-105-treated mice were significantly more active than controls (Kendall rank correlation tau p-value=0.00228). This study was performed twice, yielding similar results in each replicate (FIG. 3D, Kendall rank correlation tau p-value=1.14e-04).

Activity levels decreased in both groups of elderly mice over the course of these experiments (FIG. 3A and FIG. 3D), but decreased significantly less in the treated group, resulting in progressive divergence of the activity curves for the drug-treated and vehicle-treated group. In both experiments, by the end of the experimental period, BGE-105-treated mice ran on average at least 1 km/day more than mice treated with vehicle.

Grid Hang Test

Four 20-gallon plastic buckets were used to suspend a three-by-three-foot metal grid suspended approximately three feet from the ground. The ground just below the grid was padded with soft material. The metal grid was placed on its side so that it was perpendicular to the buckets' surface. The mouse was placed on the grid and carefully lowered so that the mouse began to hang. Once the grid was completely parallel with the horizontal plane (i.e. the floor), the timer was started. The timer was stopped when the mouse fell onto the padded floor and the time to fall was recorded and graphed.

Study Results

To determine whether increased wheel activity was accompanied by an increase in muscle strength, near the end of the frailty study we subjected mice to a grid hang test, which measures forearm grip strength. The mice were tested at 24 months and again at 26 months after 64 days of treatment with BGE-105 or vehicle. Average latency to fall increased in the BGE-105-treated mice (p=0.04, Mann-Whitney U test) (FIGS. 3B and 3C). Thus, the increase in running activity observed in BGE-105-treated mice were accompanied by improved forearm grip strength. At the end of the study, the BGE-105-treated mice had significantly higher tissue wet weight in the TA (FIG. 3F), and a trend toward higher tissue wet weight in the gastrocnemius and quadriceps (FIGS. 3G and 3H), and no difference in heart (FIG. 3I). The BGE-105-treated mice also had higher body weights (FIG. 3E), although the increase was not significant, just at p-value cutoff (0.0850).

### 7.3. Example 3: BGE-105 Activates Apelin Receptor Signaling Pathways

Administration of an apelin receptor agonist can induce the phosphorylation and activation of AMPK in heart tissue. Tissue samples were lysed using T-PER tissue protein extraction reagent (Thermo Fisher Scientific #78510) containing EDTA and protease/phosphatase inhibitors on the Omni Bead Ruptor 12 Homogenizer. Total protein was extracted then quantified using Pierce™ BCA Protein Assay Kit. Loaded equal amounts of total protein per lane on a 4-12% SDS-PAGE gel and transferred to PVDF membrane. Membranes were blocked and blotted with anti-phospho-AMPKα-Thr172 (Cell Signaling Technology, CST #2535), total-AMPKα (CST #2532), anti-phospho-Akt-Ser473 (CST #4060), total-Akt (CST #4685), anti-phospho-ERK-1/2-Thr202/Tyr204 of Erk1 and Thr185/Tyr187 of Erk2 (CST #4370), total-ERK-1/2 (CST #9107) or anti-APLNR receptor (abcam, ab214369) antibodies. Band intensities were normalized to loading control anti-3-Actin (CST #3700) or anti-GAPDH (abcam, ab181602) antibodies. Immunoreactive proteins were detected using SuperSignal™ West Femto Substrate (Thermo Fisher Scientific #34095) and quantified by Image Lab™ software (Bio-Rad Laboratories, Inc.).

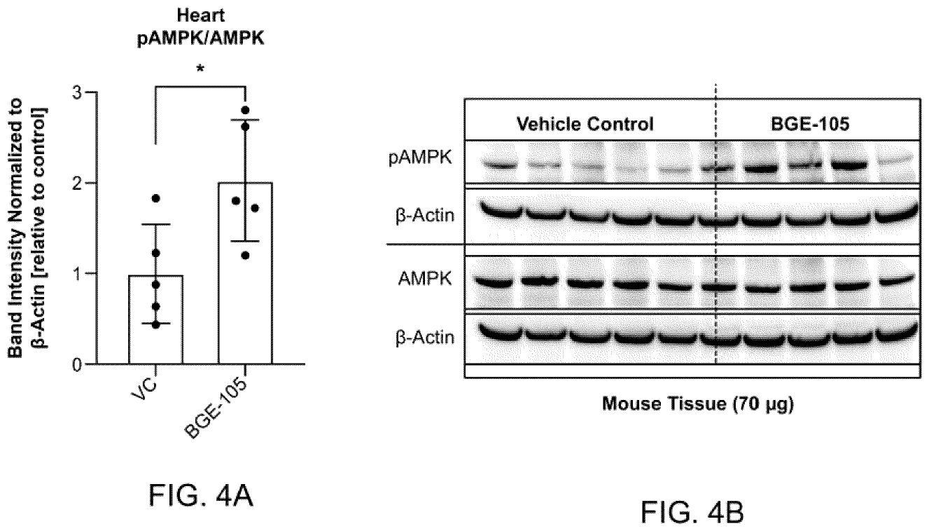

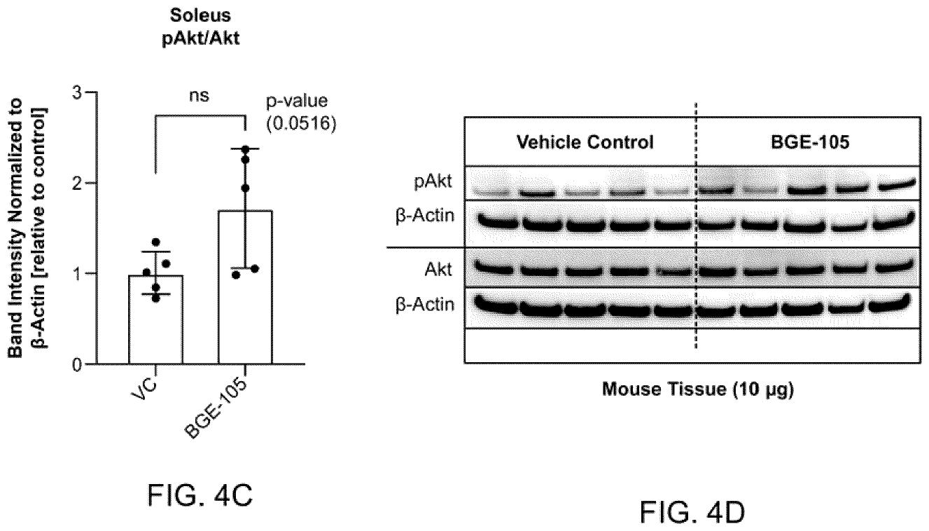

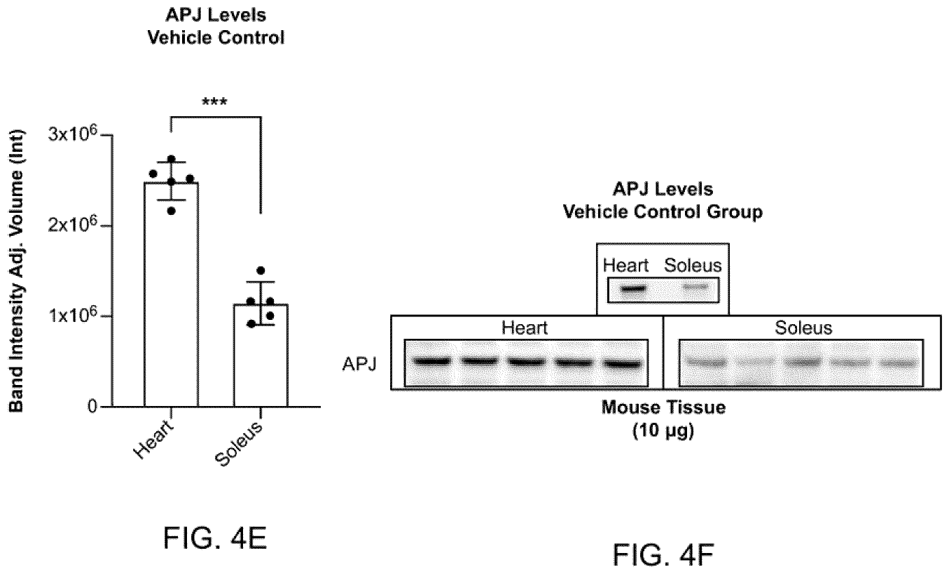

Following oral administration of 45 mg/kg BGE-105 or vehicle to mice, pAMPK levels in the heart were significantly higher in the BGE-105-treated group than in the vehicle control group, FIGS. 4A-4B. We also assessed the effect of BGE-105 in skeletal muscle. Apelin can induce the phosphorylation of Akt in the soleus muscle and improve glucose homeostasis. In the BGE-105-treated group, we observed a marginally significant increase in pAkt levels in the soleus, (p=0.0516), FIGS. 4C-4D. Beta-actin served as the internal control. Per unit mass, the soleus contained about half as much apelin receptor as the heart, FIGS. 4E-4F, potentially explaining the stronger response in heart tissue.

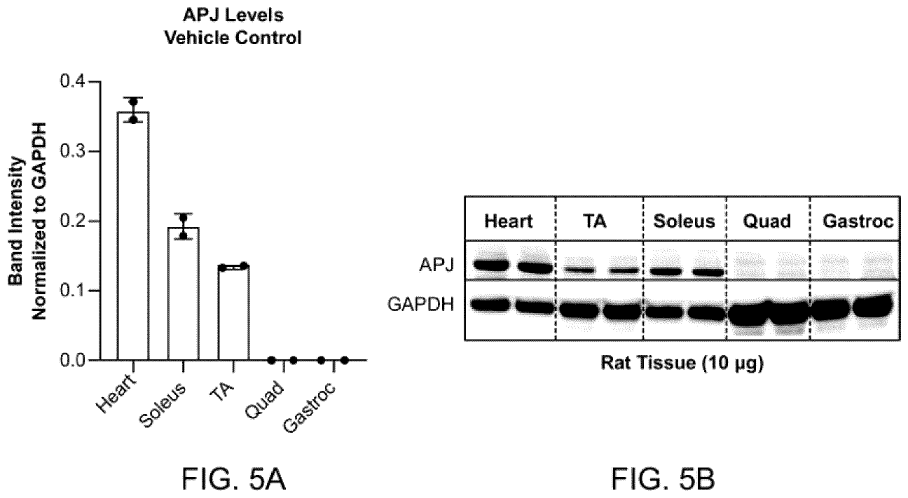

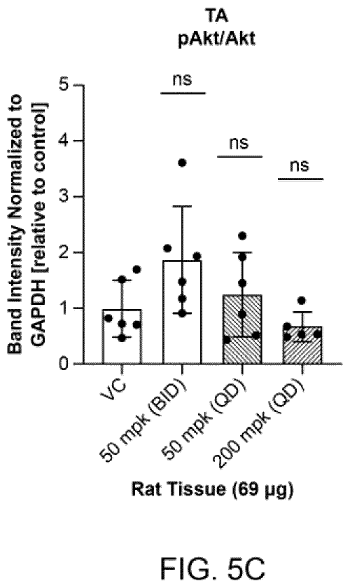

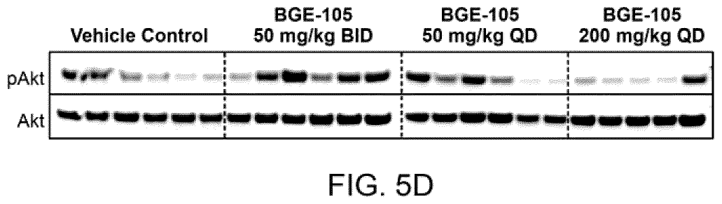

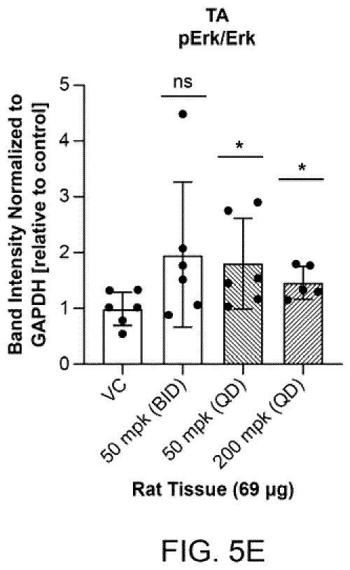

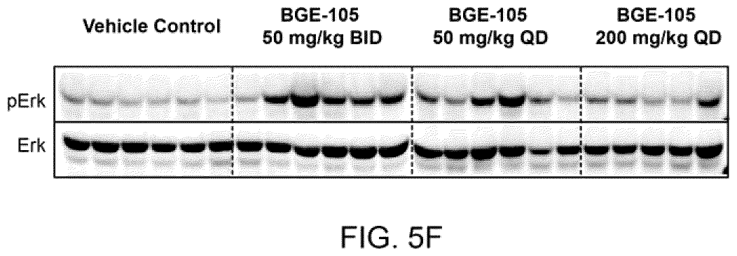

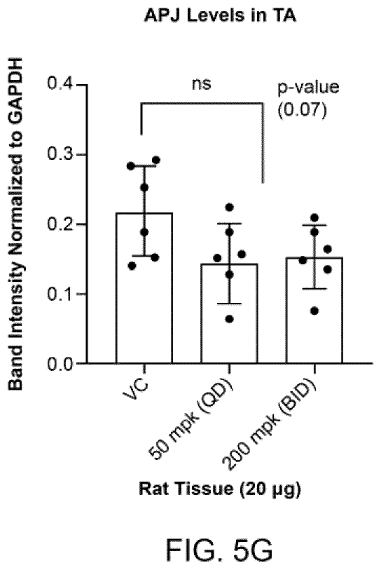

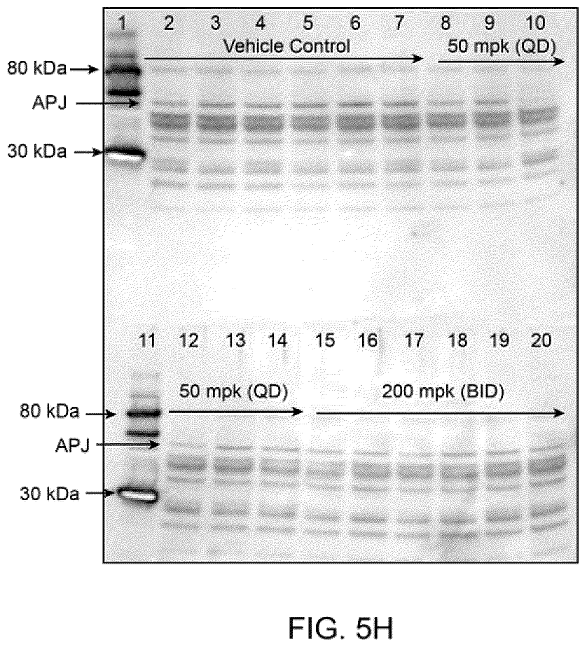

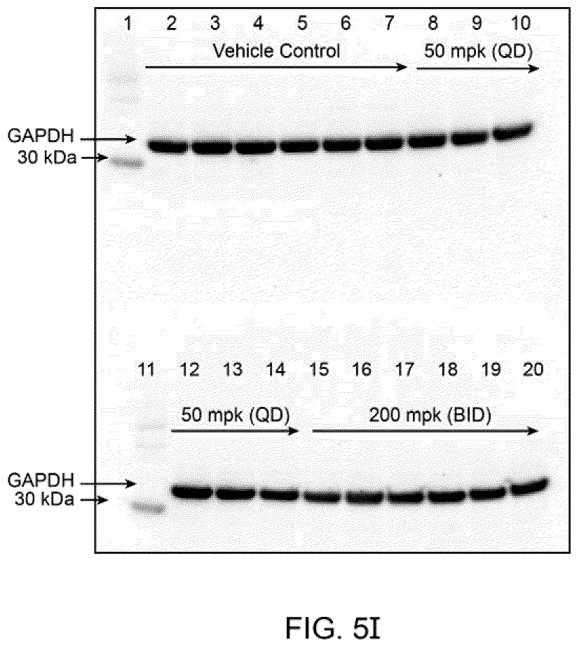

The difference between tissues was conserved among rodent species: In rats, as in mice, apelin receptor levels in rat tissue were 2-fold higher in heart than in soleus, FIGS. 5A-5B. Rat tibialis anterior (TA) had apelin receptor levels similar to those of soleus, and quadriceps and gastrocnemius had apelin receptor levels below the limit of detection in our western blot assay. Oral dosing of rats with BGE-105 for 5 consecutive days induced phosphorylation of Akt in TA in a dose-dependent manner, with 50 mg/kg BID eliciting the strongest response, FIGS. 5C-5D. A similar trend was observed for phosphorylation of Erk, FIGS. 5E-5F. Given that pErk is downstream of pAkt, this observation is consistent with a known signal transduction pathway. Next, we assessed the effect of chronic administration of BGE-105 on apelin receptor levels. BGE-105 elicited a modest but non-significant decrease in apelin receptor levels in the TA after 5 consecutive days of oral dosing at the lowest and highest doses tested, 50 mg/kg QD and 200 mg/kg BID respectively, FIGS. 5G-5I.

### 7.4. Example 4: BGE-105 Activates the Apelin Receptor in a Manner Similar to Apelin

We compared the abilities of BGE-105 and Pyr¹-Apelin-13 to activate APLNR receptor and recruit β-arrestin using the PathHunter β-arrestin assay.

The EC50 of BGE-105 was compared to Pyr¹-Apelin-13 on recruiting β-arrestin by either mouse or human APLNR using the PathHunter β-arrestin eXpress GPCR Assay. APLNR activation was determined by β-arrestin recruitment as measured by the ProLink 3-gal complementation technology (93-0001, DiscoveRx). In brief, CHO cells stably expressing APLNR were seeded and incubated overnight at 37° C. The compounds were tested in duplicate and diluted to obtain a 10-point curve with 3-fold serial dilutions (<1% DMSO). The compounds and cells were incubated for 3 hours at 37° C. After the incubation period the detection reagents were added and the plate chemiluminescent signal was measured after 30 min at RT.

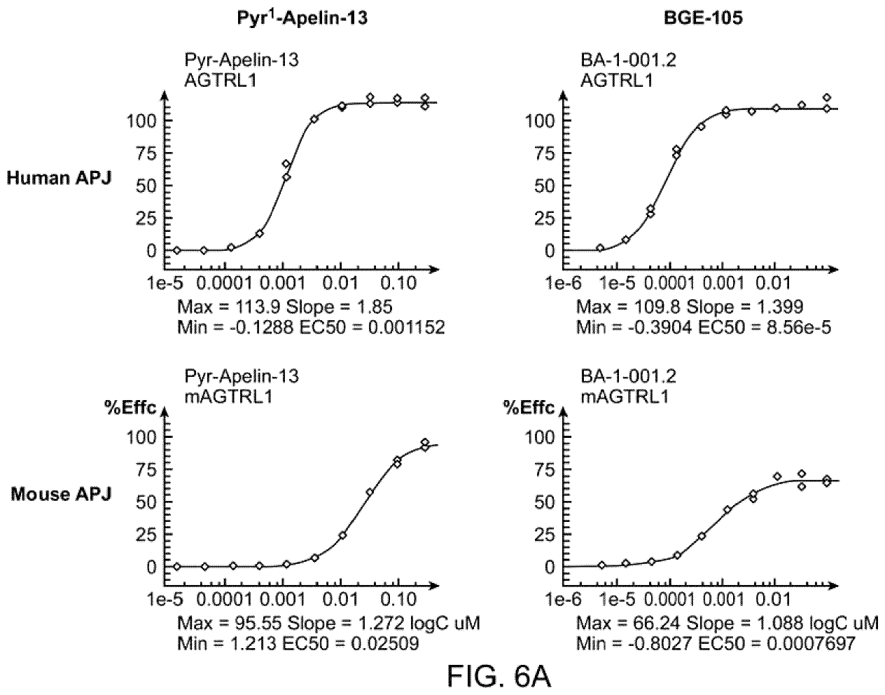

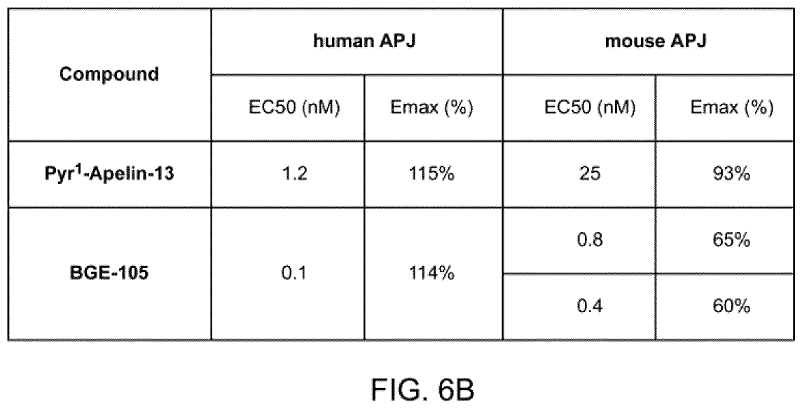

In cells stably expressing human APLNR, BGE-105 was 10-fold more potent than Pyr¹-Apelin-13: BGE-105, EC50=0.1 nM; Pyr¹-Apelin-13, EC50=1.2 nM, FIG. 6A. In cells stably expressing mouse APLNR, BGE-105 was 30-fold more potent than Pyr¹-Apelin-13: BGE-105, EC50=0.8 nM; Pyr¹-Apelin-13 (EC50=25 nM), FIG. 6A.

Although the increases in potency were comparable between human and mouse APLNR, the maximum effect (Emax) was not: human APLNR, Emax=114%; mouse APLNR, Emax=65%. Replication of the mouse APLNR β-arrestin assay with a fresh preparation of BGE-105 yielded similar data: Emax=60%, FIG. 6B. Thus, the potency of BGE-105 as an APLNR agonist may be higher in humans than in mice.

### 7.5. Example 5: BGE-105 Accelerates Regeneration of CTX-Induced Muscle Injury in Aged Mice

Impairment of muscle regeneration can contribute to age-related muscle weakness. This is particularly true in aged individuals who engage in physical activity. Exercise-induced muscle hypertrophy is linked to the capacity of muscle stem cells to be activated and promote regeneration. We evaluated the effects of oral treatment with BGE-105 during muscle regenerative processes (FIGS. 7A-7BB). To that end, we injected 18-month-old male (and 3-month old male) mice with cardiotoxin (CTX) in the left tibialis and gastrocnemius and then administered an oral bolus of BGE-105 (50 or 200 mg/kg/day) for 3 or 7 consecutive days.

Mice were i.p injected with buprenorphine (Centravet, 0.1 mg/kg) 30 minutes before injury and the day after. The day of the injury, mice were anesthetized with isoflurane inhalation and hindlimbs were shaved. Then, 10 μM of cardiotoxin (CTX, Latoxan, #L8102) was injected through two injections of 25 μl into the left tibialis muscle and two injections of 50 μl into the left gastrocnemius muscle, using a 22-gauge needle (Hamilton). Mice were euthanized 3 and 7 days after injury by cervical dislocation, muscles (PBS- and CTX-injected) were cut in two parts, one being snap frozen into liquid nitrogen for total RNA extraction and the other part being embedded into OCT, frozen in isopentane cooled with liquid nitrogen for histological analysis.

Mouse muscle samples were dissected and cryopreserved in OCT frozen in liquid nitrogen cooled isopentane. Samples were then sectioned at 10 μm on a cryostat and post-fixed with 4% Paraformaldehyde (PFA) for 15 min at room temperature. Muscle frozen sections (10 μm) were stained by helaun/eosin or immune-labeled for laminin (Abcam) and embryonic myosin. Briefly, sections were blocked 1 h in PBS plus 4% BS(a), 2% goat serum, 0.01% Triton X-100. Sections were then incubated overnight with primary antibodies. After washes in PBS, sections were incubated 1 h with secondary antibodies anti-Ig2b AF 488 (Life Technology). Slides were finally mounted in ProLong Gold antifade Reagent (Molecular probes by Life Technology) with DAPI. Images were captured using a digital camera (Nanozoomer, Hamamatsu) attached to a motorized fluorescence microscope or using Olympus VS120 Virtual Microscopy Slide Scanning System. The area covered by eMHC-positive fibers and degenerated area was determined manually across the entire sections using the VS-ASW FL software measurement tools. The size of myofibers with central nuclei was calculated from laminin/DAPI staining on all fibers of the section and area determination were performed across the entire sections, using an automated image processing algorithm developed internally using the MetaXpress software (Molecular Devices).

Study Results

FIGS. 7A-7F show the effect of administering PBS, Pyr¹-Apelin-13 (apelin) (0.5 mol/kg/day), BA1 (BGE-105 50 mg/kg/day), or BA2 (BGE-105 200 mg/kg/day) on transcript levels in the tibialis anterior of aged (18-month-old) mice either 3 days or 7 days post injection of cardiotoxin. FIG. 7A shows a significant increase in Pax7 levels for apelin (injection) and both BGE-105 (P.O.) dosages 7 days post administration. FIG. 7B shows a significant increase in the levels of MyoD at both 3 and 7 days post injection for apelin and both BGE-105 dosages. FIG. 7C shows a significant increase in MyoG levels 7 days post administration for apelin and both BGE-105 dosages. FIG. 7D shows a significant increase in MyHC3 levels 7 days post injection for apelin and BGE-105 (P.O.) at both dosages. FIG. 7E shows a significant change in the MyHC8 levels 7 days post injection for apelin and both BGE-105 dosages. FIG. 7F shows a significant change in the Myf5 levels 7 days post injection for apelin and both BGE-105 dosages.

FIGS. 7G-7L show the effect of administering PBS, Pyr¹-Apelin-13 (apelin) (0.5 mol/kg/day), BA1 (BGE-105 50 mg/kg/day), or BA2 (BGE-105 200 mg/kg/day) on transcript levels in the gastrocnemius of aged (18-month-old) mice either 3 days or 7 days post injection with cardiotoxin. FIG. 7G shows no significant change in Pax7 levels. FIG. 7H shows a significant increase in the levels of MyoD at both 3 and 7 days post injection for apelin and both BGE-105 dosages. FIG. 7I shows no significant change in MyoG levels. FIG. 7J shows a significant increase in MyHC3 levels only at 7 days post injection for the larger BGE-105 injection. FIG. 7K does not show a change in the MyHC8 levels at any time point for any injection. FIG. 7L shows no significant change in Myf5 levels.

FIGS. 7M-7R show the effect of administering PBS, Pyr¹-Apelin-13 (apelin) (0.5 μmol/kg/day), BA1 (BGE-105 50 mg/kg/day), or BA2 (BGE-105 200 mg/kg/day) on transcript levels in the tibialis of young (3-month-old) mice either 3 days or 7 days post injection with cardiotoxin. FIG. 7M shows no change in Pax7 levels. FIG. 7N shows no significant increase in the MyoD levels. FIG. 7O shows no difference in MyoG levels. FIG. 7P shows no significant change in the MyHC3 levels. FIG. 7Q shows no significant change in the MyHC8 levels. FIG. 7R shows no difference in Myf5 levels.

FIGS. 7S-7X show the effect of administering PBS, Pyr¹-Apelin-13 (apelin) (0.5 mol/kg/day), BA1 (BGE-105 50 mg/kg/day), or BA2 (BGE-105 200 mg/kg/day) on transcript levels in the gastrocnemius of young (3-month-old) mice either 3 days or 7 days post injection with cardiotoxin. FIG. 7S shows no change in Pax7 levels. FIG. 7T shows no change in the levels of MyoD. FIG. 7U shows no change in MyoG levels. FIG. 7V shows an increase in MyHC3 levels only 3 days post injections for the smaller BGE-105 dosage. FIG. 7W shows a non-significant increase in MyHC8 levels 3 days post injections for apelin and both BGE-105 dosages. FIG. 7X shows no change in Myf5 levels.

FIGS. 7Y-7Z show the cross-sectional area of the tibialis at day 3 and day 7 post injection of cardiotoxin after treatment with PBS, Pyr¹-Apelin-13 (apelin), BA1 (BGE-105 50 mg/kg/d), and BA2 (BGE-105 200 mg/kg/d). FIG. 7Y shows representative histological cross-sectional slices of the tibialis 3 and 7 days post injection with treatment of PBS, apelin, BA1, or BA2. FIG. 7Z shows the quantification of the cross-sectional histological slides, which shows a significant increase in cross sectional area for apelin, BA1, and BA2 at both 3 and 7 days post injection.

FIGS. 7AA-7BB show the amount of centrally nucleated fibers (CNM) as part of the regenerative process after cardiotoxin injection for PBS, Pyr¹-Apelin-13 (apelin), BA1 (BGE-105 50 mg/kg/day), and BA2 (BGE-105 200 mg/kg/day) treatments. Mice were 18-months old. FIG. 7AA shows representative distribution of DAPI stained nuclei and positively-stained eMHC fibers. FIG. 7BB shows the quantification of the amount of centrally nucleated myofibers (CNM). There was a significant increase in the amount of CNM after apelin and the higher of the two BGE-105 treatments.

The results presented in FIGS. 7A-7F demonstrate that, as previously demonstrated with apelin-13, BGE-105 treatment enhanced regeneration of tibialis 7 days after CTX injection. Indeed, the expression levels of genes involved in muscle regeneration (Pax7, MyoD, MyoG, Myf5, MyHC3, and MyHC8) were dramatically higher in BGE-105-treated animals than in their PBS-treated littermates. This effect was not as prominent in the gastrocnemius (FIGS. 7G-7L) suggesting that BGE-105 is more efficacious in muscle types with high receptor density, such as the tibialis. Moreover, BGE-105 was not as effective in young mice (FIGS. 7M-7X), suggesting that it is most efficacious in aged muscle with compromised repair capacity. Analysis of muscle size (cross-sectional area) confirmed the positive effect of BGE-105 on muscle regeneration in tibialis of aged mice (FIGS. 7Y-7Z) 3 and 7 days after CTX injection. Finally, BGE-105 treatment promoted accumulation of central nuclei in regenerating fibers (FIGS. 7AA-7BB), indicating that this compound can have a beneficial effect on regeneration.

Overall, the effect was less pronounced in the gastrocnemius, suggesting that an APJ agonist is most efficacious in tissues with high APJ receptor density e.g. tibialis anterior. It was also less effective in young mice suggesting that an APJ agonist is most efficacious in aged muscle with compromised repair capacity.

### 7.6. Example 6: BGE-105 Promotes Early Proliferation and Differentiation in Human Myoblast Cells

Immortalized human cells from male donors aged 25 years old (25-HMC) and 79 years old (79-HMC) are grown from the proliferation stage until they become 80% confluent, differentiate, and become myotubes. The cells were treated from day 1 to day 4 with either Pyr¹-Apelin-13 at 1 nM, BGE-105 at 0.05, 0.5, 5, 50 nM, or vehicle (<0.1% DMSO) (FIG. 8A to 8K). Early proliferation markers (Pax7, Myf5, MyoD, and MyoG were assessed via RT-PCR (FIG. 8D to 8K).

Study Results

Short-term (from day 0 to day 4 post seeding) BGE-105 treatment induced a significant increase of cell proliferation in cells from both young and aged donors (FIGS. 8B-8C). BGE-105 treatment also increased the expression of muscle cell differentiation markers such as Pax7 and MyoD in young donor cells (FIGS. 8D-8G) and Pax7, Myf5, MyoD, and MyoG in aged-donor cells (FIGS. 8H-8K). Altogether, these results support the use of BGE-105 in human muscle physiology in aged populations.

FIGS. 8A-8C show the ability of BGE-105 to increase the proliferation of immortalized human muscle cells from both younger (25-years-old) and older (79-years-old) subjects. FIG. 8A shows the experimental protocol with an initial incubation (Treatment #1) during in vitro proliferation of cells (days 0-4) with a second incubation (Treatment #2) during the differentiation stage into myotubes (days 4-18). The treatments were DMSO (0.1%), Pyr¹-Apelin-13 (1 nM), or BA (BGE-105) at 0.05, 0.5, 5, or 50 nM. FIG. 8B shows the proliferation of the cells (measured at day 4) from the younger subject with an increase at 5 nM and a significant increase at 50 nM of BGE-105 treatment. FIG. 8C shows the proliferation of the cells from the older subject with a significant increase at 5 nM of BGE-105 and an increase at 50 nM of BGE-105 treatment.

FIGS. 8D-8K show the levels of PAX7, MYf5, MyoD and MyoG expression in immortalized muscle cells from older (79-years old) and younger (25-years-old) subjects after incubation with DMSO (0.1%), Pyr¹-Apelin-13 (Apelin/Ape) (1 nM), or BGE-105 (BA) at 0.05, 0.5, 5, or 50 nM. FIG. 8D shows the levels of PAX7 in the younger cells. BGE-105 at 5 and at 50 nM recapitulated the levels of apelin. FIG. 8E shows the levels of MYf5 in the younger cells after treatment. There was no change in the level of MYf5 relative to the control for any amount of BGE-105. FIG. 8F shows the MyoD expression levels after treatment in the younger cells. There was a very significant increase of MyoD expression at 5 nM of BGE-105. FIG. 8G shows the levels of MyoG expression after treatment in the younger cells. There was no significant change after treatment. FIG. 8H shows PAX7 levels after treatment in older cells. There was a significant increase at all treatment doses as compared to control. FIG. 8I shows the levels of Myf5 expression after treatment in cells derived from the older donor. At 5 and 50 nM the levels were equal to apelin treatment. FIG. 8J shows the levels of MyoD after treatment cells derived from the older donor. There was an increase at all treatment levels with the higher dosages, approaching the levels of expression caused by apelin. FIG. 8K shows the levels of MyoG after treatment in the older cells. There was an increase at all treatment levels approaching the levels of expression caused by apelin.

### 7.7. Example 7: BGE-105 Prevents Disuse-Induced Muscle Atrophy in Aged Mice

BGE-105 activates pathways that benefit skeletal muscle physiology, notably the pAkt/pErk pathway, which plays a pivotal role in regulating muscle mass. Limb immobilization causes a loss of gross skeletal muscle mass accompanied by a significant decrease in apelin transcript levels. Hence, we tested whether BGE-105 rescues muscle atrophy induced by chronic immobilization. Because skeletal muscle atrophy caused by disuse is exaggerated during aging, we evaluated the effects of BGE-105 on maintenance of muscle mass in aged mice subjected to immobilization of the plantar flexor group (soleus, TA, EDL, gastrocnemius). Animals were orally administered vehicle or BGE-105 at 50 mg/kg BID; 1 week into treatment, the right hindlimb was immobilized by casting and the muscles were allowed to atrophy over 21 days.

Twenty-month-old male C57/Bl6 mice (n=10/group) were administered P.O. vehicle or BGE-105 at 50 mg/kg BID at ZT1 and ZT11.5. One week into the treatment, mice underwent modified hindlimb casting on one limb. Mice were anesthetized with isoflurane inhalation and the hindlimb wiped with povidone-iodine, then ethanol, and loosely wrapped in surgical gauze. A custom-made plastic immobilization device was placed on the limb, with the foot in full extension, so as to result in the maximal in vivo unloading of the plantarflexor group. The device was fixed to the hindlimb using Vetbond and the animal returned to its cage. After 3 weeks of treatment following casting, mice were euthanized 1 hour after the final ZT1 dose, and tissues isolated, weighed, then flash frozen in liquid nitrogen for subsequent western blot analysis.

Study Results

Immobilization caused significant atrophy in the casted limb of the vehicle-treated group for all muscle types, FIG. 9A. Notably, wet weight of tibialis anterior (TA) muscle decreased by 25% in the placebo group, but by only 3% in the BGE-105 group, FIGS. 9D-9E. Similarly, extensor digitorum longus (EDL) atrophied by 14% in the placebo group but did not measurably atrophy in the BGE-105 group, FIGS. 9F-9G. A modest rescue in atrophy was observed in soleus, although this effect was not significant, FIGS. 9H-9I.

In these animals, gastrocnemius contained significantly less apelin receptor density than the other muscles, FIGS. 9J-9K, potentially explaining why BGE-105 is less effective in this muscle. FIGS. 9L-9M shows apelin receptor levels after one month of BGE-105 treatment at 50 mg/kg BID. Again, we observed that chronic activation of the apelin receptor by BGE-105 had a noticeable but non-significant drop in apelin levels. Thus, although BGE-105 did downregulate the apelin receptor after chronic treatment, the effect was not significant at the dose tested.

Our data demonstrate that aged mice treated with BGE-105 were protected against some loss of muscle mass induced by immobilization. Thus, BGE-105 may have clinical benefits to protect against disuse atrophy in humans.

### 7.8. Example 8: BGE-105 Prevents Atrophy in Immobilized Human Muscles

Two groups of healthy older adult humans (e.g., N=10 per group) who are moderately active remain in bed continuously for 10 days, except for toileting, and they consume a eucaloric diet providing the recommended dietary allowance for protein (0.8 g/kg of protein per day). One group is given 200 mg of BGE-105 a day, while the other receives a placebo. Measurements before and after bed rest include muscle function and protein synthesis.

BGE-105 is shown to prevent or attenuate muscle atrophy in immobilized human muscles during periods of disuse.

### 7.9. Example 9: Safety, Tolerability, Pharmacokinetics, and Pharmacodynamics of BGE-105 in Healthy Adult Patients (Phase 1b Clinical Study)

The study described herein characterizes the apelin effects of BGE-105 with both single and multiple doses. The target indication includes treatment to improve frailty and to improve muscle function in the elderly. Two groups (Group A “Part A”, single-ascending dose (SAD), and Group B “Part B”, multiple dose (MD)) of healthy older adults participate in the study for approximately 42 days including a Screening/Pre-Treatment Period of up to 14 days, a Treatment Period of 5 days for Part A and 7 days for Part B, and a Follow-up Visit 27 days after the first administration of study drug (BGE-105 or placebo).

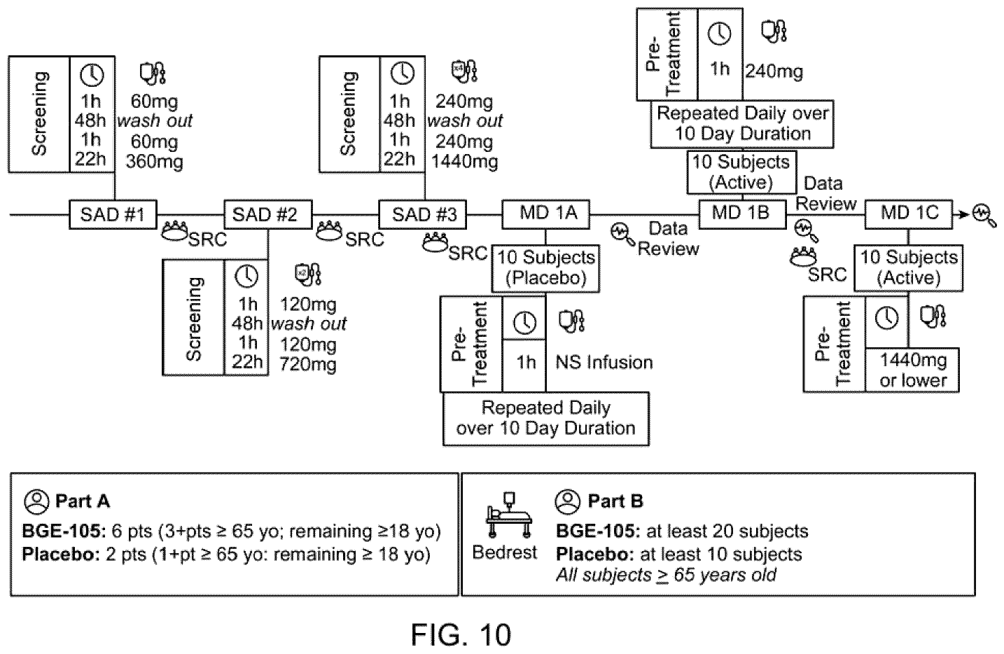

In Part A, of 24 subjects enrolled (3 SAD cohorts, 8 subjects each), a total of at least 12 subjects are ≥65 years of age (≥4 subjects in each cohort). The remaining subjects are ≥18 years old. In each cohort, 6 subjects receive BGE-105 and 2 subjects receive placebo for a total of 18 BGE-105-treated subjects and 6 placebo treated subjects, for a total of 24 subjects. In addition to characterizing the PD effects associated with acute BGE-105 exposure, the use of a 48-hour “drug holiday” (wash out period in FIG. 10) between first and second doses in the SAD cohorts, infusions permit gathering information related to tachyphylaxis and durability of apelin-like effects.

In Part B, of the 30 subjects enrolled, all of which are ≥65 years of age. The 30 subjects who qualify are enrolled to receive treatment in either Cohort 1A (placebo), Cohort 1B (240 mg BGE-105 daily), or Cohort 1C (240 mg/1440 mg). Cohort 1A includes 10 subjects receiving placebo normal saline (NS), Cohort 1B includes 10 subjects each who receives BGE-105, and Cohort 1C includes up to 10 subjects each who receives BGE-105. Subjects participating in this study for approximately 81 days including a screening period of up to 16 days, an Outpatient Pre-Treatment Period of 5 days (Day −5 to Day −1) with heavy water and D3-creatine (D3-Cr), and a Treatment period of 10 days on bed rest with heavy water/D3-Cr and BGE-105 or Placebo, and a post-dose Follow-Up Period (Days 11 to Day 60) that includes 2 follow-up visits on Days 11, 12, 13, 14, 21, 30, and 60 days after the first administration of study drug (BGE-105 or Placebo). In the multi-dose cohorts (1A, 1B, 1C), PD parameters for effects on changes in insulin sensitivity and muscle indices during a period of bed rest are measured and evaluated to better inform decisions regarding choice of dose and direction of further development.

### Objects and Endpoints

Primary Objective. To evaluate the safety and tolerability of single ascending doses and multiple ascending doses of BGE-105 in healthy adult subjects (≥18 years of age in Part A, ≥65 years of age in Part B) with an emphasis on older subjects (≥65 years of age in Part A) after administration of BGE-105 by intravenous (IV) infusion.

Safety Assessments. Safety assessments are summarized in Table 6.

TABLE 6
Safety assessments for Phase 1b trial in healthy adult patients

Assessment of treatment emergent adverse events (TEAEs)
Assessment of the use of concomitant medications
Clinical laboratory assessments including complete blood count, clinical
chemistries, and urinalysis
Additional physical examinations including vital signs ECG assessments,
including continuous telemetry monitoring

Additional physical examinations include vital signs. ECG assessments, include continuous telemetry monitoring.

### Secondary Objectives and Endpoints

Secondary Objectives. Secondary objectives include: to characterize the pharmacodynamic (PD) effects of BGE-105 after IV infusion in healthy adult subjects; to characterize the pharmacokinetics (PK) of BGE-105 after IV infusion in healthy adult subjects; and to characterize the PK/PD relationships of BGE-105 on predefined biomarkers (including, but not limited to, glucose, insulin, and insulin sensitivity) and PD variables (such as changes in systolic and diastolic blood pressure, heart rate), and in the multiple dose cohorts (Part B), measurement of muscle protein synthesis rate from administration of heavy water and a micro-(small needle) biopsy of the vastus lateralis, D3-creatine (D3-Cr) total skeletal muscle mass from urine samples, and muscle circumference, cross-sectional area, color flow analysis, anterior-posterior (AP) diameter, and echo density by ultrasound of the vastus lateralis and the gastrocnemius muscles.

Secondary Endpoints. Secondary Endpoints are summarized in Table 7.

TABLE 7
Secondary endpoints of Phase 1a trial in healthy adult patients
PK parameters of BGE-105 including, but not limited to:

Area under the serum concentration time curve (AUC) over the 24-hour
dosing interval and from time 0 to the time of the last observed serum
concentration following the final dose (AUC0-last)
AUC from time 0 to infinity (AUC0-inf)
Maximum observed serum concentration (Cₘₐₓ)
Time to reach Cₘₐₓ (Tₘₐₓ)
Terminal elimination half-life (T½)
Evaluation of steady-state and time to reach steady-state by
evaluation of trough BGE-105 concentrations
Total body clearance (CL) and volume of distribution (Vz)
Assessment of attainment of steady-state by comparison of:
up through 96-hour concentrations for Part A and
up through 336-hour concentrations for Part B
PD parameters including, but not limited to changes in:
Systolic and diastolic BP
Pulse rate and heart rate
Physical Function Tests Day 1 and Day 11 (48 hours after the start
of the last infusion), and at a post-dose
follow-up visits through Day 60. Physical Function Tests
include assessment/rehabilitation of 1) sit to stand test, 2) short physical
performance battery (SPPB), and 3) Tinetti Performance Oriented
Mobility Assessment (POMA).
Biomarkers including, but not limited to changes in:
Plasma glucose
Plasma insulin
Insulin resistance (HOMA-IR)
Multi-dose (Part B, MD cohorts) only PD parameters assessed
before and after bedrest:
Skeletal muscle circumference, cross-sectional area, color flow
analysis, AP diameter, and echo density
D3-creatine total skeletal muscle mass
Muscle protein synthesis rate (contractile and sarcoplasmic) after Heavy
Water administration in blood, urine and saliva samples
Micro (fine needle) skeletal muscle biopsy (of the vastus lateralis)
to determine “flux” proteomics, the rate of synthesis
of hundreds of muscle proteins including
contractile, mitochondrial, sarcoplasmic, membrane-bound, and matrix.

This study is a randomized, placebo controlled, double blind, single ascending dose (SAD) and a single-blind, multiple dose (MD) study, in up to 72 healthy adult subjects. There are 6 cohorts: 3 cohorts (8 subjects each) in Part A and 3 cohorts (10 subjects each) in Part B. Cohorts can be split and dosing staggered by 1 day to facilitate collection of data on heavy procedure days (e.g., the 10 subjects in the MD cohort can be split into groups of 5, staggered 1 day apart). The study design is provided as FIG. 10.

In each of the 3 cohorts in Part A (SAD cohorts), there are a total of 6 healthy male or female receiving BGE-105 (at least 3 subjects ≥65 years old, remaining subjects ≥18 years old) and 2 Healthy male or female receiving placebo (at least 1 subject ≥65 years old, remaining subject ≥18 years old) via IV infusion.

After a baseline period of 24 hours in the clinic, all subjects receive a LD, 1-hour infusion on Day 1. After a 48-hour wash-out period, in which PK and PK/PD data is collected, subjects receive a 23-hour infusion (1-hour LD followed by a 22-hour maintenance dose). See FIG. 10 for the different dose levels in each SAD cohort. See e.g., Table 3 below. Part A is double-blinded. The cohorts of Part A is conducted sequentially starting with SAD cohort 1.

In each of the 3 cohorts in Part B, 30 total subjects are enrolled to receive treatment in either Cohort 1A, Cohort 1B, or Cohort 1C. Cohort 1A includes 10 subjects who all receive placebo NS over a 1 hour infusion for 10 days on Days 1 through Day 10. Cohort 1B includes 10 subjects each who receive BGE-105, 240 mg over 1 hour infusion, up to 10 days on Days 1 through 10. Cohort 1C includes up to 10 subjects each who receive BGE-105 at a dose not to exceed 1400 mg over 24 hours up to 10 days. Patients are at bed rest for the entire 10-day duration of treatment.

The dose of BGE-105 for Cohort 1C is a dose not to exceed 1440 mg over 24 hours which was the highest dose given in the SAD and was well tolerated. The dosing regimen over the 10 days is guided by HOMA-IR data as required from Cohort 1B and the dose, dose regimen, or both may be changed.

The Pre-Treatment Period for all 3 cohorts starts on Day −5 and continue through Day −1. All subjects in each cohort are admitted to the unit on Day −2. All subjects start on bed rest on Day 1 and continue to Day 10.

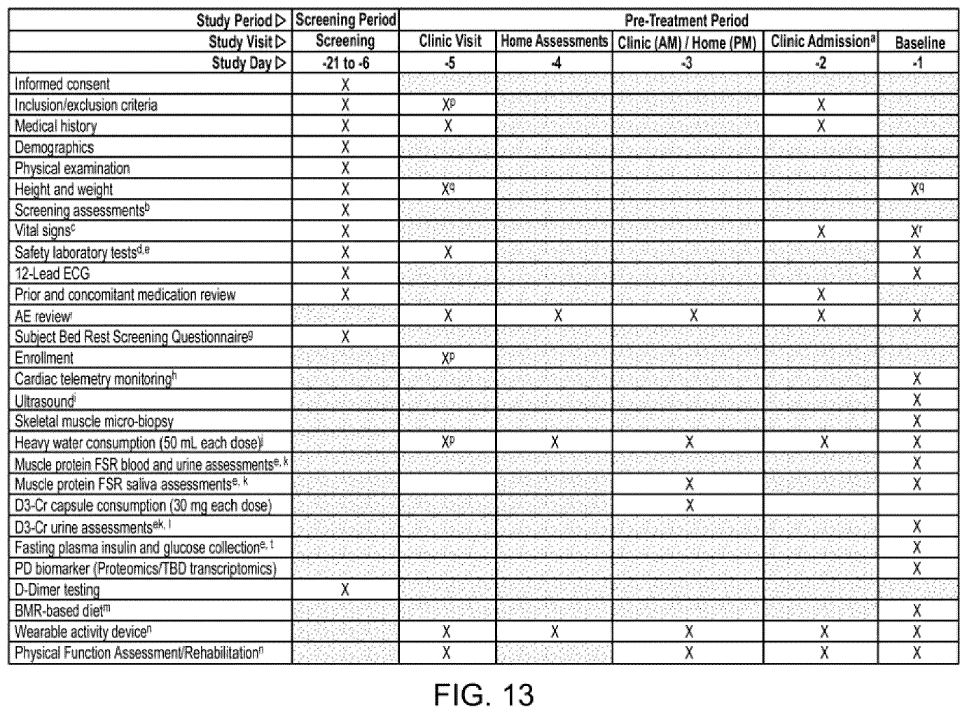

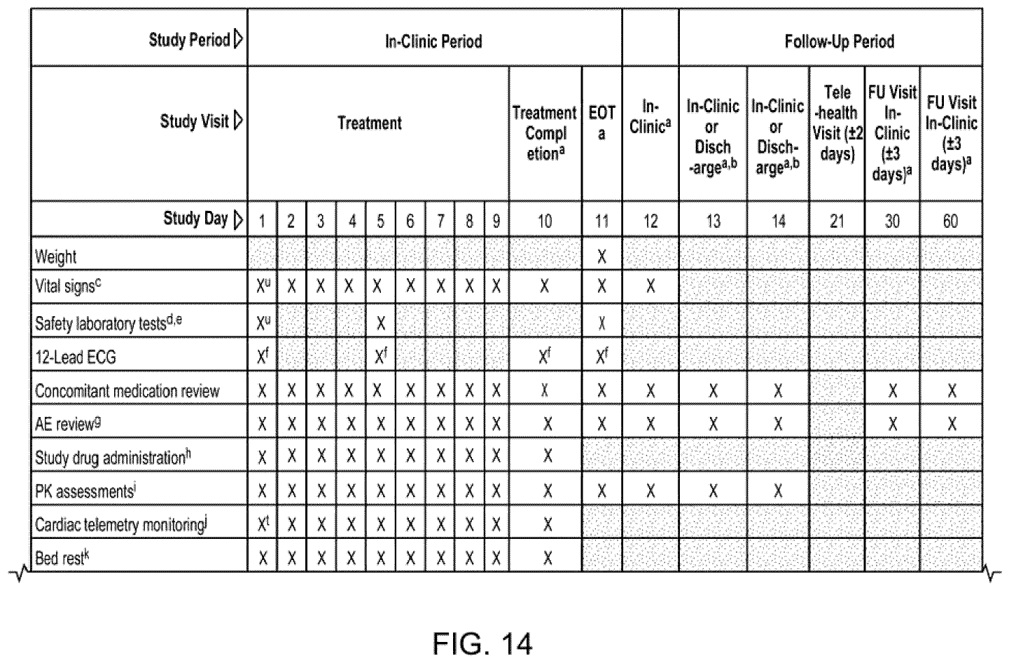

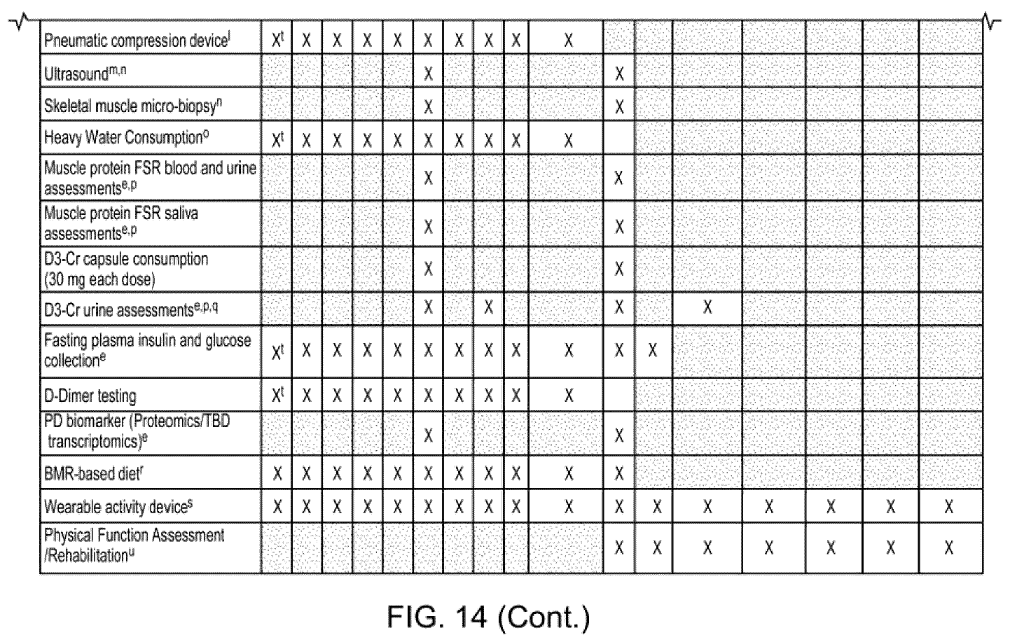

All multiple dose (Part B) cohort subjects receive heavy water and D3-Cr starting on Day −5 and continue through Day 10 according to FIGS. 13-14. FIG. 13 provides a screening and pre-treatment outline of MD cohorts. FIG. 14 provides a treatment and follow up outline of MD cohorts.

Subjects receive heavy water starting on Day −5 and D3-Cr starting on Day −3 and both continue through Day 10 according to FIGS. 13-14.

After the completion of multiple dose Cohort 1A, a pharmacodynamic evaluation is performed by the Sponsor of the results from samples collected from the skeletal muscle biopsy, blood, urine, and saliva to measure the effects on skeletal muscle. HOMA-IR is evaluated to assess insulin resistance. The data is unblinded to the Sponsor and is used to confirm the number of days of bed rest for Cohort 1B and 1C (e.g., 10 days or less). In addition, between Cohort 1B and Cohort 1C, unblinded HOMA-IR is evaluated by the Sponsor to confirm the dose and dosing regimen for Cohort 1C. Data from other measures such as ultrasound measurements, pharmacokinetic data and the data for muscle mass and muscle protein synthetic rates was reviewed by the Sponsor in an unblinded manner after each cohort.

Subjects have additional in-clinic and home assessments during the Pre-Treatment Period and an extended Follow-Up Period through Day 60. See FIGS. 13-14.

For the multiple dose cohorts (Part B), Cohort 1A is conducted to confirm the effects on skeletal muscle induced by bed rest as well as the effect on HOMA-IR. The data from Cohort 1A is used to confirm 10 days of bed rest is sufficient to characterize the effect on muscle protein synthesis rate after administration of heavy water and via micro-(fine needle) skeletal muscle biopsy of the vastus lateralis, D3-Cr total skeletal muscle mass determination from a fasting urine sample, and muscle circumference, cross-sectional area, color flow analysis, anterior-posterior (AP) diameter, and echo density by ultrasound of the vastus lateralis and the gastrocnemius. Cohorts 1B and 1C proceeded with 10 days of bed rest or less if evidence a shorter period can elicit measurable effects on skeletal muscle.

The cohorts of Part A and the cohorts of Part B was conducted sequentially starting with SAD Cohort 1. This process was repeated for each cohort in Parts A and B of the study.

Dosage Rationale. Based on preliminary results from SAD cohorts (BGE-105-101), an IV dose of BGE-105 up to 4-fold higher than the maximum dose studied in the previous, completed Phase 1 trials (BGE-105-101) was proposed.

Given the good tolerability, lack of human safety findings and lack of dose limiting toxicities in both tox species (NOAEL is maximum dose tested in both rat and dog), this proposed dose increase is justified as long as predicted human exposures are not expected to exceed tox species exposures. The updated exposure margins considering the highest maximum exposures in rats and dogs are calculated as follows: maximum exposure in the rat (male) was in the 14-day IV study and at the 300 mg/kg dose, the AUCₗₐₛₜ is 1070 μg*hr/mL; maximum exposure in dogs occurred at the 300 mg/kg dose in the 9-month oral toxicity study and the AUCₗₐₛₜ is 1310*μg·hr/mL.

Therefore, based on the PK of the single dose of 240 mg loading dose followed by 1440 mg IV for 22 hours, multiple dosing with this dose (the highest proposed dose for use in this study) the predicted human exposure margins would be: Human:Rat=1036/1070=0.97; Human:Dog=1036/1310=0.79.

Thus, all available safety data continues to support use of doses up to 240/1440 mg and human exposures do not exceed toxicology coverage, even at the highest proposed dose.

TABLE 3
Cohorts, Doses, and Dosing Regimens

| | | | |
|---|---|---|---|
| | Planned | Planned IV infusion | Planned number of |
| Cohort | Doses | Dosing Regimen | subjects |

Part A SAD (24 subjects)

| | | | |
|---|---|---|---|
| Cohort 1: | 60 mg/ | LD 60 mg/1 h | Total: 8 subjects |
| | 360 mg | 48-hour washout period | BGE-105: 6 (at least 3 ≥ |
| | | LD 60 mg/1 h + | 65 yo, remaining ≥ 18 yo) |
| | | MD 360 mg/22 h | Placebo: 2 (at least 1 ≥ 65 |
| | | | yo, remaining ≥ 18 yo) |
| Cohort 2: | 120 mg/ | LD 120 mg/1 h | Total: 8 subjects |
| | 720 mg | 48-hour washout period | BGE-105: 6 (at least 3 ≥ |
| | | LD 120 mg/1 h + MD | 65 yo, remaining ≥ 18 yo) |
| | | 720 mg/22 h | Placebo: 2 (at least 1 ≥ 65 |
| | | | yo, remaining ≥ 18 yo) |
| Cohort 3: | 240 mg/ | LD 240 mg/1 h | Total: 8 subjects |
| | 1440 mg | 48-hour washout | BGE-105: 6 (at least 3 ≥ |
| | | period 240 mg/1 h + | 65 yo, remaining ≥ 18 yo) |
| | | MD 1440 mg/22 h | Placebo: 2 (at least 1 ≥ 65 |
| | | | yo, remaining ≥ 18 yo) |

Part B: MD (30 subjects ≥ age 65 years)**

| | | | |
|---|---|---|---|
| Cohort | Placebo | Placebo Normal Saline | N = up to 10 |
| 1A: | NS | from Days 1 to Day 10 | |
| Cohort | 240 mg | BGE-105 240 mg | N = up to 10 |
| 1B: | BGE-105 | for 1 h every day | |
| | | up to 10 days | |
| | | from Day 1 to Day 10 | |
| Cohort | 240 mg/ | BGE-105 (dose and | N = up to 10 |
| 1C: | 1440 mg | regimen to be | |
| | | confirmed | |
| | | from 1B cohort) | |
| | | TBD mg over TBD hrs | |
| | | every day us to 10 days | |
| | | from Day 1 to Day 10 | |

Abbreviations: h = hour;
LD = loading dose;
MD = maintenance dose;
yo = years old;
TBD = to be determined
* The total number of subjects in Part B may be decreased or increased up to 48 subjects total.
**Heavy water 3 times daily starting on Day −5 through Day 10. D3-creatine 1 capsule on Day −3, Day 6, and Day 11.

A matching BGE-105, Placebo for Infusion product is created by using saline in corresponding IV bag sizes. If a label is added to the saline bags used to create active BGE-105 for infusion, the label for the placebo saline bags match to maintain the blind.

Study Duration. Subjects in Part A (SAD) participate in this study for approximately 42 days including a Screening/Pre-Treatment Period of up to 14 days, a Treatment Period of 5 days, and a Follow-up Visit 27 days after the first administration of study drug (BGE-105 or placebo).

Subjects in the Part B (MD) participate in this study for approximately 81 days including a Screening Period of up to 16 days, an Outpatient Pre-Treatment Period of 5 days (Day −5 to Day −1) with heavy water and D3-creatine (D3-Cr), and a Treatment Period of 10 days on bed rest with heavy water/D3-Cr and BGE-105 or Placebo, and a post-dose Follow-Up Period (Days 11 to Day 60) that includes follow-up visits on Days 11, 12, 13, 14, 21, 30, and 60 days after the first administration of study drug (BGE-105 or placebo).

### Part A: Cohorts Screening and Pre-Treatment

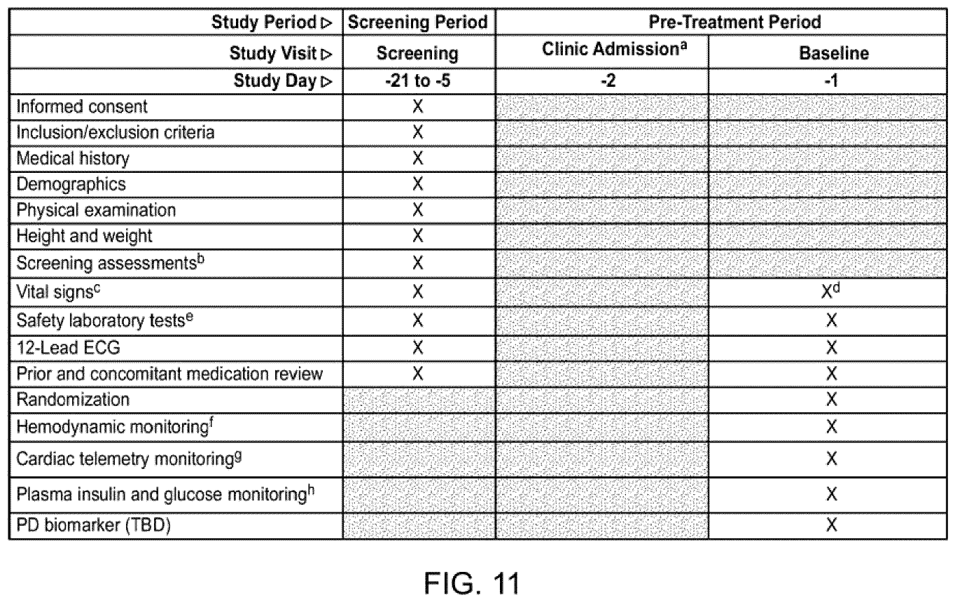

Subjects in Part A is admitted to the clinic on Day −2. On Day −1, baseline assessments is performed, and the subject is randomized to blinded treatment with study drug (BGE-105 or placebo). An outline of the protocol for Part A cohorts is outlined in FIG. 11 and is described in Table 8 below.

TABLE 8
Treatment protocol for Part A cohort

a. Subjects will be admitted to the clinic on the evening of Day −2 and will fast overnight for
Baseline procedures on Day −1.
b. Screening-specific assessments include a pregnancy test (for subjects that are pre-menopausal),
urine drug screen (dipstick test), alcohol breath test, and screening for HIV antibodies, HbsAg, and
HCV antibodies.
c. Vital signs include diastolic and systolic blood pressure and pulse. In addition, temperature and
respiratory rate should be collected on Day −1 at the timepoint that mimics Day 1 predose.
d. On Day −1, blood pressure was collected at timepoints that mimic Day 1 and Day 3 as closely as
possible, based on the anticipated dose start time (2 h, 3 h, 4 h, 6 h, 8 h, and 12 h).
e. Safety laboratory tests include complete blood count, clinical chemistries, and urinalysis. Subjects
are to be fasting for a minimum of 8 hours.
f. Continuous cardiac ECG telemetry monitoring was used, starting 24 hours before the
administration of study drug.
g. Blood samples for plasma insulin and glucose monitoring should be collected in the morning,
before breakfast and after fasting for 8 hours overnight
Abbreviations: AE = adverse event;
ECG = electrocardiogram;
HbsAg = hepatitis B surface antigen;
HCV = hepatitis C virus;
HIV = human immunodeficiency virus;
PD = pharmacodynamic;
SAD = single ascending dose
TBD = to be determined

### Part A: SAD Cohorts Treatment and Follow-Up

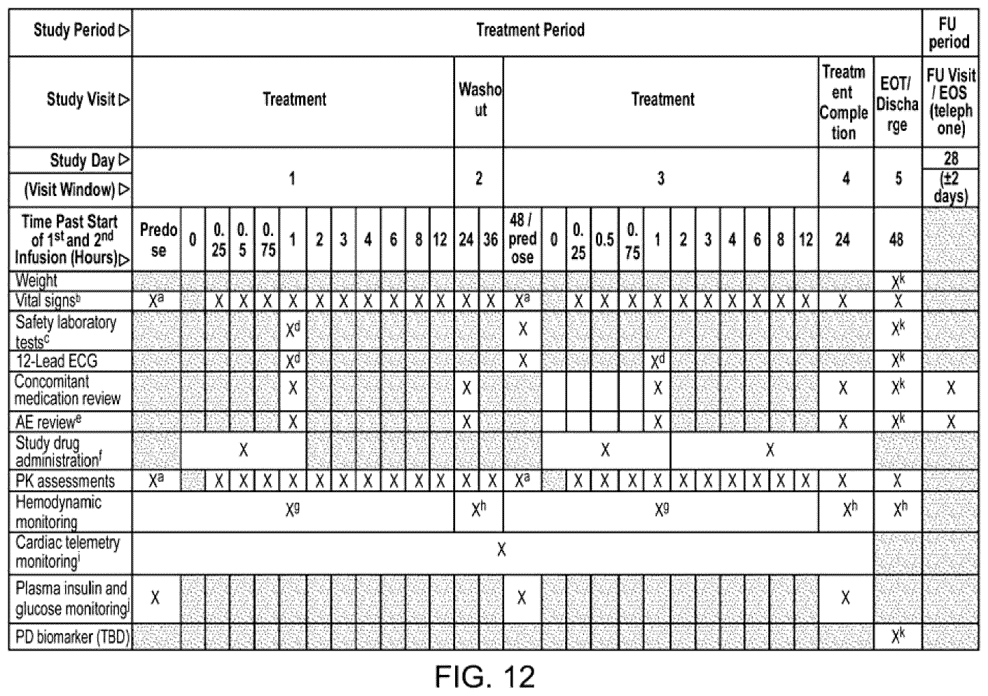

On Day 1 for Part A, subjects receive LD, 1-hour infusion. On Day 3, after a 48-hour washout period, subjects then receive a 23-hour infusion (1-hour LD followed by a 22-hour MD). No earlier than 24 hours after the end of infusion, subjects is discharged from the clinic on Day 5 (End of Treatment Period [EOTP]). A treatment protocol for the SAD cohorts of Part A is outlined in FIG. 12 and is described in Table 9 below.

TABLE 9
Treatment protocol for the SAD cohorts of Part A

a. Predose assessments (vital signs and PK assessments) should be done within 30 min of
the start of infusion.
b. Vital signs include diastolic and systolic blood pressure and pulse. In addition,
temperature and respiratory rate should be collected on predose Day 1 and EOT Day 5.
c. Safety laboratory tests include complete blood count, clinical chemistries, and
urinalysis.
d. Assessments should be done within 1 hour after the end of the LD infusions on Day 1
and Day 3.
e. AEs was recorded from the start time of the first BGE-105 or placebo study drug
infusion until the end of the subject's participation in the study. At discharge from the
unit, any ongoing AEs was documented in source and in EDC be followed until the event
is resolved or stabilized. Any new AEs occurring after discharge will clearly note the start
date and end date.
f. Study drug was administered via IV infusion as a LD, 1-hour infusion on Day 1. After a
48-hour wash-out, study drug will then be administered as a 23-hour infusion (1-hour LD
followed by a 22-hour MD).
g. Continuous cardiac ECG telemetry monitoring was used, continuing through the end of
dosing on Day 4.
h. Blood samples for plasma insulin and glucose monitoring should be collected in the
morning, before breakfast and after fasting overnight.
i. Assessments can be done any time after 24 hours after the end of infusion and before
discharge.
Abbreviations: AE = adverse event;
ECG = electrocardiogram;
EOS = end of study;
EOT = end of treatment;
FU = follow-up;
IP = investigational product;
IV = intravenous;
LD = loading dose;
MD = maintenance dose;
PD = pharmacodynamic;
PK = pharmacokinetic;
SAD = single ascending dose;
TBD = to be determined

### Part B: MD Cohorts Screening and Pre-Treatment

Subjects in Part B are admitted to the clinic on the evening of Day −2 and fast overnight from 10:00 PM for Baseline procedures on Day −1. A pre-treatment protocol for the MD cohorts of Part B is outlined in FIG. 13 and is described in Table 10 below.

TABLE 10
Pre-treatment protocol for the MD cohorts of Part B

A. Subjects was admitted to the clinic on the evening of Day −2 and will fast overnight from
10:00PM for Baseline procedures on Day −1. Subjects are to be fasting from 10:00 PM
onward (min 8 hours fast) each night but may drink as much water as requested.
B. Screening-specific assessments include a pregnancy test (for subjects that are pre-
menopausal), urine drug screen (dipstick test), alcohol breath test, and screening for HIV
antibodies, HbsAg, and HCV antibodies.
C. Vital signs include diastolic and systolic blood pressure and pulse.
D. Safety laboratory tests include complete blood count, clinical chemistries, and urinalysis.
E. Samples are to be collected in the morning while subject is fasting from 10:00PM the night
before.
F. AEs was recorded from the start time of the first consumption of heavy water until the end
of the subject's participation in the study. At discharge from the unit, any ongoing AEs was
documented in source and in EDC was followed until the event is resolved or stabilized. Any
new AEs occurring after discharge will clearly note the start date and end date.
G. Subject Bed Rest Screening Questionnaire.
H. Continuous cardiac ECG telemetry monitoring will start 24 hours before the start of the
first BGE-105 or placebo infusion.
i. Muscle circumference, cross-sectional area, color flow analysis, AP diameter, and echo
density of the vastus lateralis and gastrocnemius was measured by ultrasound. Ultrasound
imaging must be conducted by the same technician throughout the study for all subjects.
Ultrasound readings must be done by the same technician throughout the study for ALL
subjects.
J. On Day −5 through Day −1, subjects will receive 3 doses of heavy water spaced
approximately four hours apart, in the morning, afternoon, and evening. On Day −5, subjects
will check into the clinic and be discharged to home approximately 1 hour after the last
evening dose. Subject was dispensed adequate amounts of heavy water for home consumption
until the next clinic visit. The site will telephone the subjects daily and remind them to
consume heavy water as instructed.
K. Samples are to be collected before consumption of heavy water and the D3-Cr capsule, as
applicable.
L. The samples for the D3-Cr urine assessments are to be collected from the second urination
after waking up and while fasting from 10:00PM the night before. The D3-Cr urine
assessment must be done 48 h (+60m) after the consumption of the D3-Cr capsule.
M. Subjects began a BMR-based diet starting with breakfast on Day −1 and site staff will
encourage subjects to consume liquids. The total amount of meals in percentage and liquids in
mL was recorded.
N. A wearable activity device such as a FitBit was dispensed to the subjects to wear from
Day −5 through the last FU Visit on Day 60 to explore recording physical activity parameters. The
site will adequately train subjects on how to operate the wearable at home and was instructed
on charging the device and home-use.
O. Physical Function Assessment/Rehabilitation includes 1) the Sit to Stand Test, 2) Short
Physical Performance Battery (SPPB), and 3) the Tinetti Performance Oriented Mobility
Assessment (POMA). The baseline assessment for these tests was conducted by the
occupational therapist while the subject is in-clinic anytime during the Pre-Treatment Period
(Day −5, Day −3, Day −2 or Day −1). There was one occupational therapist assigned to each
subject throughout the study and they will conduct all of these assessments for the subject to
minimize bias and learning effects. These assessments will only be conducted once during the
Pre-Treatment Period.
P. Study eligibility was confirmed with review of inclusion/exclusion criteria before assigning
the subject number and the first consumption of heavy water.
Q. Weight only.
R. On Day −1, vital signs was collected at timepoints that mimic Day 1 as closely as possible,
based on the anticipated start time of the BGE-105/placebo infusion (predose [additionally
temperature and respiratory rate], 0.25 h, 0.5 h, 0.75 h, 1 h, 2 h, 3 h, 4 h, 6 h, 8 h, and 12 h).
S. Fasting plasma insulin and glucose collection was conducted at the timepoint that mimics
Day 1 as closely as possible, based on the anticipated start time of the BGE-105/placebo
infusion
T. D3-creatine capsules in Part B, is provided to subjects during the pre-treatment and
treatment periods before breakfast and after fasting overnight at timepoints described
previously. D3-creatine capsules are filled using creatine (methyl-D3) powder. Each capsule
contains 30 mg of D3-creatine and cellulose filler.
Abbreviations: AP = anterior-posterior;
BMR = basal metabolic rate;
D3-Cr = deuterated creatine;
ECG = electrocardiogram;
FSR = fractional synthetic rate;
HbsAg = hepatitis B surface antigen;
HCV = hepatitis C virus;
HIV = human immunodeficiency virus;
PD = pharmacodynamic;
TBD = to be determined.

### Part B: MD Cohorts Treatment and Follow-Up

A treatment protocol for the MID cohorts of Part B is outlined in FIG. 14 and is described in Table 11 below.

TABLE 11
Treatment protocol for the MD cohorts of Part B

A. For bed rest duration of >10 days, the Treatment Completion procedures was conducted on
the day of the last BGE-105 infusion. EOT procedures was conducted 1 day after the last
BGE-105 infusion. Day 12, 13, 14, 30 and 60 procedures was conducted 2, 3, 4, 20, and 50
days after the last BGE-105 infusion, respectively.
B. Subjects will remain in the clinic through Day 13, with the option to stay an extra day if
returning to the clinic for protocol assessments on Day 14 is not logistically feasible.
C. Vital signs was collected daily and include diastolic and systolic blood pressure and pulse.
On Days −1, 1, 5, and 10, vital signs was collected at the following timepoints: predose
(additionally temperature and respiratory rate), 0.25 h, 0.5 h, 0.75 h, 1 h, 2 h, 3 h, 4 h, 6 h, 8 h, and
12 h after the start of study drug infusion. Vital signs will also be collected on Day 10 (23 h)
and before discharge on Day 12.
D. Safety laboratory tests include complete blood count, clinical chemistries, and urinalysis.
E. Samples are to be collected in the morning. Subjects are to be fasting from 10:00 PM
onward (min 8 hours fast) each night but may drink as much water as requested.
F. ECGs are to be taken predose and immediately following the completion of the 1-hour
infusion. On Day 11, an ECG will also be taken again 23 h after the start of the Day 10 study
drug infusion.
G. AEs was recorded from the start time of the first consumption of heavy water until the end
of the subject's participation in the study. At discharge from the unit, any ongoing AEs was
documented in source and in EDC was followed until the event is resolved or stabilized. Any
new AEs occurring after discharge will clearly note the start date and end date.
H. BGE-105 study drug was administered via IV infusion as a 1-hour infusion.
I. PK samples were collected daily predose. On Days 1, 5, and 10, PK samples were collected
at the following timepoints: predose, 0.25 h, 0.5 h, 0.75 h, 1 h, 2 h, 3 h, 4 h, 6 h, 8 h, and 12 h after
the start of study drug infusion. Samples will also be collected 24 h (Day 11), 48 h (Day 12),
72 h (Day 13), and 96 h (Day 14) after the start of the last infusion. PK samples were taken
from the opposite arm used for the IV infusion.
J. Continuous cardiac ECG telemetry monitoring was used starting on Day 1 pre-dose and
continuing through the end of dosing on Day 10.
K. Subjects were asked to remain in their beds, apart from using a wheelchair to be taken to
the shower and they will shower seated. They will also use a wheelchair to get to and from the
bathroom. When not in bed, subjects were in a wheelchair.
L. Subjects will wear the pneumatic compression cuffs the entire duration of bed rest, except
for when in a wheelchair to use the bathroom and shower.
M. Muscle circumference, cross-sectional area, color flow analysis, AP diameter, and echo
density of the vastus lateralis and gastrocnemius were measured by ultrasound. Ultrasound
imaging must be conducted by the same technician throughout the study for all subjects.
Ultrasound readings must be done by the same technician throughout the study for all
subjects.
N. Ultrasound and skeletal muscle micro-biopsy were conducted on Day 6 predose and on
Day 11 at the 24 h timepoint.
O. On Day 1 through Day 10, subjects will receive 3 doses of heavy water spaced
approximately four hours apart, in the morning, afternoon, and evening.
P. Samples are to be collected before consumption of heavy water and the D3-Cr capsule, as
applicable.
Q. The samples for the D3-Cr urine assessments are to be collected after an overnight fast
starting at 10 pm the night before AND from the second urination after waking up while still
fasted On Days −1, 8, and 13, the D3-Cr urine assessments must be done 48 h (+60m) after the
consumption of the D3-Cr capsules.
R. Subjects were on a BMR-based diet ending at 24 h on Day 11 (after the ultrasound, muscle
microbiopsy, and laboratory samples are collected) and site staff will encourage subjects to
consume liquids. The total amount of meals in percentage and liquids in mL was recorded.
S. A wearable activity device such as a FitBit and corresponding electronic tablet was
dispensed to the subjects to use from Day −5 through the last follow up (FU) Visit on Day 60
to explore recording of physical activity parameters. The site will adequately train subjects on
how to operate the wearable at home and was instructed on charging the device and home-
use. Subjects were able to keep the device and tablet for personal use at the Day 60 Follow-
Up Visit.
T. Procedures are to be conducted before starting the period of bed rest.
U. Physical Function Assessment/Rehabilitation includes 1) the Sit to Stand Test, 2) Short
Physical Performance Battery (SPPB), and 3) the Tinetti Performance Oriented Mobility
Assessment (POMA). The Sit to Stand Test was conducted on Day 12, 13, 14, 30, and 60. The
SPPB was conducted on Day 12 and 30. The POMA was conducted on Day 13, 14, 21, 30,
and 60 (only if the Day 30 score was not within ±10% of the baseline score). There was an
occupational therapist assigned to each subject throughout the study and they will conduct all
of these assessments for the subject on each study day to minimize bias and learning effects.
These assessments will only be conducted once on each study day. If the Day 30 POMA score
is not within +/−10% of the baseline score, subjects may have additional telehealth
appointments between the Day 30 and Day 60 if needed. The POMA was conducted and
documented in the source.
Abbreviations: AP = anterior-posterior;
BMR = basal metabolic rate;
D3-Cr = deuterated creatine;
ECG = electrocardiogram;
FSR = fractional synthetic rate;
HbsAg = hepatitis B surface antigen;
HCV = hepatitis C virus;
HIV = human immunodeficiency virus;
PD = pharmacodynamic;
TBD = to be determined.

### Follow-Up Period

For Part A, on Day 28 (approximately 27 days after administration of the first dose of study drug), subjects are contacted by telephone for safety follow-up assessments. The date on which the subject completes the Follow-up Visit is the subject's end of study date (or EOS). For Part B, subjects remain in-clinic through Day 13 for safety follow-up assessments, laboratory sample collection, and physical function rehabilitation following 10 days of bed rest. Subjects have the option to remain in-clinic for an additional day if returning to the clinic for protocol assessments on Day 14 is not logistically feasible. Subjects have a telehealth visit on Day 21, and in-clinic visits on Days 30 and 60 for safety follow-up assessments.

### Pharmacokinetic and Pharmacodynamic Assessments

Ultrasound. For subjects in Part B only, an ultrasound is performed at timepoints described previously to measure muscle circumference, cross-sectional area, color flow analysis, AP diameter, and echo density of the vastus lateralis and gastrocnemius on one of the legs. The leg measured has to remain consistent.

Ultrasound images were collected during the following study visits: Day −1 (baseline), Day 6 (pre-dose), and day 11 (end of treatment). Ultrasounds were performed of both the vastus lateralis and gastroenemius to measure cross-sectional area, color flow doppler, antero-posterior (AP) diameter, and echo density. Muscle circumference was also measured. Ultrasound imaging was conducted by the same operator throughout the study for all subjects. Ultrasound readings were done by the same reader throughout the study for all subjects. The ultrasound operator and reader were blinded to the study treatment, whether subjects received active study drug BGE-105 or placebo. The leg measured (right vs. left) and leg location (medial vs. lateral) remained consistent for all ultrasounds.

Muscle circumference was measured in centimeters (cm) using a measuring tape and recorded on the ultrasound worksheet at the time of imaging. The targeted measurements for circumference that were acquired at the below markers: 15 cm superior of the mid patella for the Vastus Lateralis, and 3-inch inferior to the popliteal vessels for the gastrocnemius.

Ultrasound Procedure

The ultrasound images were collected before conducting the skeletal muscle micro-biopsy. Prior to testing, subjects were instructed to wear shorts on testing day to avoid compression of the upper leg musculature and to expose the upper portion of the thigh. Subjects were required to lay supine on an examination table with both legs fully extended for a minimum of 5 minutes to allow for fluid shifts to occur. Each subject was instructed to lay on their non-dominant side to obtain skeletal muscle ultrasound images of the vastus lateralis and gastrocnemius in the dominant leg. Subjects were positioned with their legs on top of one another and slightly bent at the knee. Ultrasound images of the vastus lateralis will be captured at 50% of the straight-line distance from the greater trochanter and the lateral epicondyle of the femur.

To ensure proper probe placement and consistent image capture location, a dotted line was drawn transversely and longitudinally along the surface of the skin from the aforementioned location. All measures of muscle morphology were obtained using a B-mode, 12-MHz linear probe US (General Electric vivid E9) to provide acoustic contact without depressing the dermal layer of the skin. Longitudinal B-mode and transverse field of view (FOV) images were acquired during each exam and analyzed. Ultrasound settings remained fixed for examination of each subject: image gain was set at 50 decibels (dB), dynamic range was set at 72, and image depth was set at 5 cm. Three panoramic transverse images (PTI) were captured in the transverse plane, perpendicular to the long axis of the muscle. These images utilized the extended-field-of-view ultrasonography in order to include entire area of the vastus lateralis in a single panoramic image.

Additionally, three single longitudinal images (SLI) were captured in the longitudinal plane, parallel to the long axis of the muscle. Single Still Longitudinal Image (SLI) Selection includes: (i) Consistent pressure was applied by probe for Minimal muscle compression, (ii) To avoid affecting the measurement value, the probe was carefully set on the thigh using plenty of ultrasound gel so that the probe does not directly touch the skin to push the soft tissue; (iii) Superficial aponeurosis of vastus lateralis (adipose tissue/muscle interface) was as close to horizontal as possible; (iv) Entire length of image consisted of muscle fibers (no aponeuroses or inconsistencies in probe pressure or placement). This same technique was applied to ultrasound of the gastrocnemius. Either the medial head or lateral head of the gastrocnemius was used for ultrasound evaluation, but not both. The same head was used for all gastrocnemius ultrasounds throughout the study.

Heavy Water Consumption and Assessments. For subjects in Part B only, heavy water, deuterated H₂O (D₂O), is provided to subjects to drink during the pre-treatment and treatment periods before breakfast, in the afternoon, and after fasting overnight, at timepoints described previously. Blood samples (10 mL) and urine samples are collected at timepoints described previously to assess skeletal muscle protein fractional synthetic rate (FSR).

Plasma Insulin and Glucose. Blood samples (4 mL) for plasma insulin and glucose monitoring are collected in the morning, before breakfast and after fasting overnight at timepoints described previously.

Proteomics. For subjects in Part B only, blood samples (4 mL) for proteomic analysis are collected at timepoints described previously.

Bioenergetics. For subjects in Part B only, blood samples (8 mL) for bioenergetic assessments are collected at timepoints described previously.

### Statistical Analyses

The statistical analysis plan (SAP) provide the statistical methods and definitions for the analysis of the safety, PK, PD, and untargeted metabolome and proteome data, as well as describe the approaches to be taken for summarizing other study information such as subject disposition, demographics and baseline characteristics, investigational product exposure, and prior and concomitant medications. The SAP also includes a description of how missing, unused, and spurious data are addressed. Descriptive statistics are supplied according to the nature of the criteria:

Quantitative variable: sample size, arithmetic mean, standard deviation (SD), standard error of the mean (SEM), minimum, median and maximum, and quartiles, if necessary (with geometric mean, arithmetic and geometric coefficients of variation (CV), and quartiles for pharmacokinetic (PK) parameters.

Qualitative variable include: sample size, absolute and relative frequencies per class. All listings are presented by cohort and treatment. Details of the statistical analysis are described in a SAP, which is finalized before database lock.

Unless specified otherwise, all calculations is performed using SAS statistical software, version 9.3 or later, and/or Phoenix WinNonlin. All safety, PK, PD, and PK/PD data is displayed in tables and listings separately for each treatment group. Post-Text TLFs are provided in collated electronic MS Word .rtf files (i.e., table columns and rows appear in MS Word Table format). The statistical analyses of the study is conducted in a GCP environment (ICH E6).

Sample Size:

No formal sample size calculation has been done. Cohort and overall study size are based on practical considerations. The study plan is to enroll up to 72 volunteer subjects to receive at least one dose of BGE-105 or placebo.

In the SAD (Part A), 18 subjects received BGE-105 and 6 subjects received placebo.

For the MD (Part B), 30 subjects receive BGE-105 or placebo. All Part B subjects are ≥65 years of age. A subject may be replaced on a case-by-case basis at the discretion of the sponsor. The replacement subject is assigned the same treatment as the subject being replaced.

Analysis Populations:

The Safety Analysis Set include all subjects who had received ≥1 administration of study drug (either BGE-105 or placebo). The Safety Analysis Set is used for safety analysis. Subjects is analyzed based on the actual treatment received.

The Pharmacokinetic (PK) Set include enrolled subjects who had received ≥1 administration of study drug without any event and/or major protocol deviation affecting the PK evaluation and with completed PK profile(s). The inclusion/exclusion of subjects with incomplete PK profile(s) in this set is agreed upon between the Sponsor and the CRO before the PK concentration dataset is locked.

The Pharmacodynamic (PD) Set include all enrolled subjects who have completed the study without any protocol deviation affecting the PD evaluation with a baseline sample and ≥1 postbaseline sample for PD evaluation. The inclusion/exclusion of subjects with incomplete PD profile(s) in this set is agreed upon between the Sponsor and the CRO before the PD concentration dataset is locked.

The Pharmacokinetic/Pharmacodynamics (PK/PD) Set include all subjects who are in both the PD Set and the PK Set.

### Safety Analyses

Safety data is summarized using descriptive statistics (number of subjects, mean, median, standard deviation, minimum, and maximum) for continuous variables and using frequency and percentages for discrete variables.

Adverse events are coded using the Medical Dictionary for Regulatory Activities. The number of events, incidence, and percentage of TEAEs are calculated overall by system organ class, preferred term, and treatment group for each cohort and treatment group. The number and percentage of subjects with TEAEs are further summarized by severity and relationship to study drug. AEs related to study drug, AEs leading to withdrawal, SAEs, and deaths are also similarly summarized and/or listed in a similar manner.

Clinical laboratory tests, vital signs, and ECG findings are summarized by treatment group and study visit. Descriptive statistics are calculated for quantitative safety data as well as for the difference from baseline, if applicable. Frequency counts are compiled for the classification of qualitative safety data. The baseline for safety data is defined as the last value before administration of the first dose of IP. Potentially clinically important findings are also summarized and/or listed.

### Pharmacokinetic Analysis:

Individual BGE-105 plasma concentrations is listed and descriptive statistics including means, geometric means, medians, ranges, standard deviations, and coefficients of variation is provided. Corresponding concentration-time profiles (individual and means) is displayed in plots.

Relevant plasma PK parameters are derived for BGE-105 by standard noncompartmental methods and tabulated along with descriptive statistics and graphs.

The linear dose-proportionality of Cmax, AUC0-t, and AUC0-inf (if applicable) are investigated for BGE-105 using an exponential regression model (“power model”).

### Pharmacodynamic Analysis:

Data is analyzed as change from baseline within each dose cohort and treatment group. Treatment comparisons are made as a linear contrast of all treated compared to all placebo and for each individual dose cohort versus all placebo based on time-matched samples acquired during the 24-hour period on Day −1. P-values are reported uncorrected for multiplicity.

For Part B, changes in leg circumference, cross-sectional area, color flow analysis, AP diameter, and echo density of the vastus lateralis and gastrocnemius, as measured by ultrasound, are analyzed first as a linear contrast comparing all treated to placebo and then as individual dose comparisons to placebo. P-values are reported uncorrected for multiplicity.

Skeletal muscle protein synthesis rate (%/time) for individual muscle proteins are compared first as a linear contrast including all doses versus placebo and then as individual dose comparisons to placebo.

The Benjamini-Hochberg procedure are used to control the false positive discovery rate for the multiplicity of proteins assessed.

D3-creatine total muscle mass is analyzed as change from baseline within each cohort and treatment group. Treatment comparisons is made as a linear contrast of all treated compared to all placebo and for each individual dose cohort versus all placebo based on time-matched samples acquired.

The micro needle biopsy is analyzed by three different measures including target biomarkers, proteomics, and changes in protein (exploratory).

Targeted Biomarkers: The means of targeted biomarkers (creatine kinase-muscle [CK-M], etc.) for each MD cohort is compared using analysis of variance (ANOVA).

Proteomics: The proportion of proteins with high vs. low FSR is compared against baseline values.

Changes in Protein (Exploratory): the detection of individual proteins with significant changes over time, after correction for multiple comparisons.

The Sit to Stand Test and the SPPB are analyzed as a change from within subject and as applicable by each dose cohort and treatment group. The POMA assessment is an exploratory measure. Any changes from baseline are summarized within subject and as applicable by each dose cohort and treatment group.

### Pharmacokinetic/Pharmacodynamic Analysis:

Individual biomarker plasma concentrations were listed and descriptive statistics including means, geometric means, medians, ranges, standard deviations, and coefficients of variation are provided. Corresponding concentration-time profiles (individual and means) are displayed in plots.

Relevant PD parameters are listed and tabulated along with descriptive statistics and graphs.

The relationship between BGE-105 and plasma and other PD parameters were investigated using a graphical exploratory and simple modelling approach.

Unblinded Data Review. There are unblinded data reviews conducted by the Sponsor after the completion of each cohort. An unblinded data review takes place after the completion of Part A to confirm the study design and dose for Part B. A Sponsor unblinded data review is also conducted after the completion of each of the MD cohorts.

### Result

BGE-105 is shown to prevent frailty and to improve muscle function in the elderly using single or multiple doses.

BGE-105 given to healthy adult subjects (≥18 years of age in Part A, ≥65 years of age in Part B) with an emphasis on older subjects (≥65 years of age in both Parts A and B) after administration of BGE-105 by constant intravenous (IV) infusion in single ascending doses and multiple ascending doses, is shown to be safe and tolerable.

### Results for 3 SAD Cohorts in Part A—BGE-105-101 Study

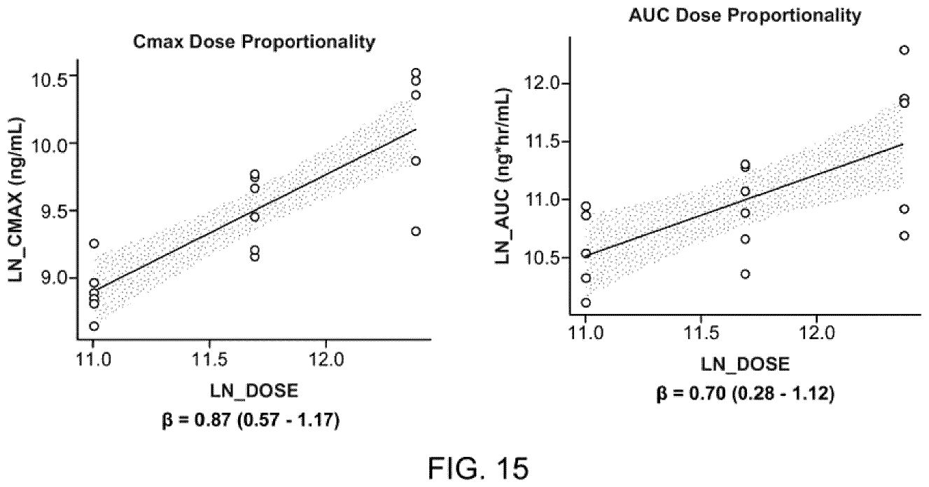

Pharmacokinetic (PK) data from the 3 SAD cohorts demonstrated dose proportionality displayed in FIG. 15. The Cₘₐₓ remained within the expected range and for the highest dose (240 mg/1440 mg) the AUCₗₐₛₜ was 1062 μg*hr/mL (Table 4 below):

For the SAD cohorts in Part A, all doses were well tolerated including the highest dose of 240 mg/1440 mg. There were no emerging safety concerns or trends and there were no serious adverse events. A maximum tolerated dose from the 3 SAD cohorts was not determined. A summary of pharmacokinetic parameters of Part A (single ascending dose) of Phase 1 study BGE-105-101 is provided in Table 4.

TABLE 4
Summary of Pharmacokinetic Parameters of Part A (Single Ascending Dose)
of Ongoing Phase 1 Study BGE-105-101
Mean (% CV)

| | SAD1 | | SAD2 | | SAD3 | |
|---|---|---|---|---|---|---|
| | (60 mg/360 mg) | | (120 mg/720 mg) | | (240 mg/1440 mg) | |
| | Day 1 | Day 3 | Day 1 | Day 3 | Day 1 | Day 3 |
| Cmax (μg/mL) | 7.430 | 10.147 | 13.662 | 14.808 | 26.720 | 35.620 |
| | (22%) | (16%) | (25%) | (32%) | (41%) | (44%) |
| Dose- | 0.12 | — | 0.11 | — | 0.11 | — |
| normalized | (22%) | | (25%) | | (41%) | |
| Cmax | | | | | | |
| AUC0-24 | 34.701 | 173.033 | 53.736 | 266.900 | 107.216 | 612.045 |
| (μg · hr/mL) | (28%) | (19%) | (32%) | (31%) | (57%) | (44%) |
| AUClast | 37.384 | 306.409 | 56.810 | 449.858 | 113.960 | 1062.000 |
| (μg · hr/mL) | (29%) | (19%) | (33%) | (32%) | (58%) | (45%) |
| AUCinf | 39.582 | — | 58.814 | — | 119.315 | — |
| (μg · hr/mL) | (31%) | | (34%) | | (60%) | |
| Dose- | 0.66 | — | 0.49 | — | 0.50 | — |
| normalized | (31%) | | (34%) | | (60%) | |
| AUCinf | | | | | | |
| Half-life (hr) | 9.1 | — | 8.6 | — | 8.9 | — |
| | (20%) | | (12%) | | (17%) | |

FIG. 15 demonstrated the dose proportionality for the 3 SAD cohorts. The 95% confidence interval for the slope of dose vs Cmax (A) and AUC(B) includes 1, suggesting that BGE-105 PK was linear across the dose range. Low point estimates and wide confidence interval were likely due to between-subject variability.

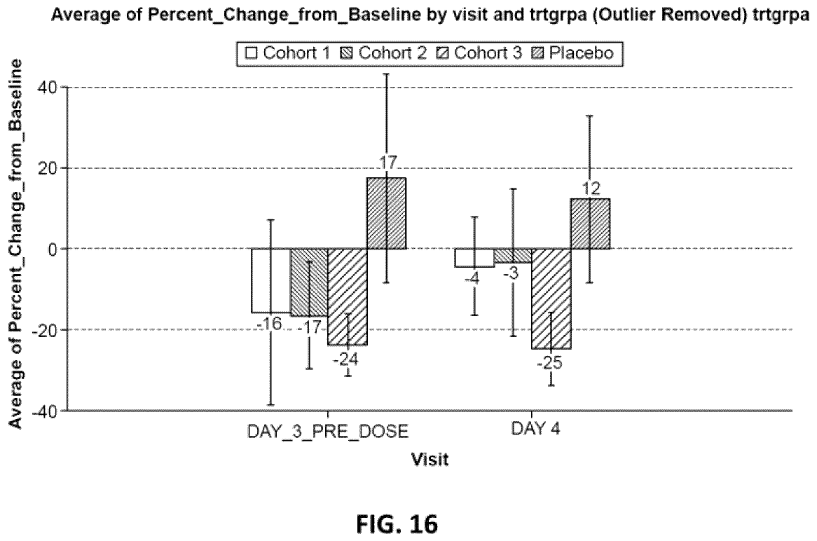

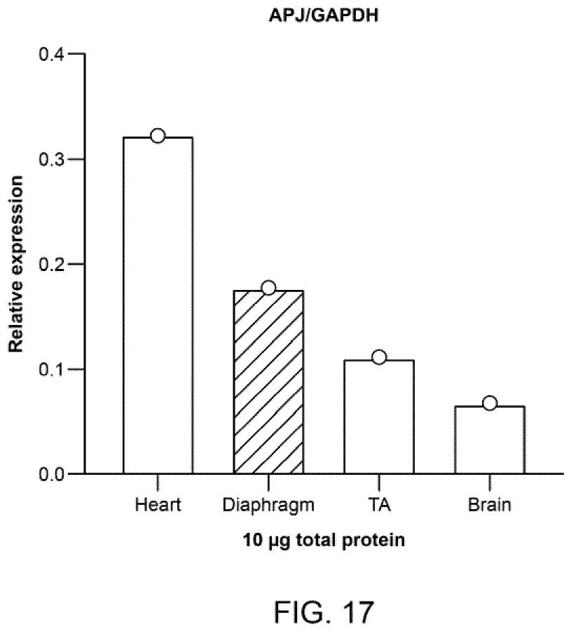

FIG. 16 shows preliminary results for SAD cohorts 1-3 (study Part A), where there that there was a 17% increase from baseline in HOMA-IR on the Day 3 Predose visit and 12% increase from baseline in HOMA-IR on the Day 4 visit for the Placebo group of the SAD study (Part A) of Example 9. In the BGE-105 treated patients (cohorts 1,2, and 3) of the SAD study (Part A) of Example 9, there was a decrease in the percent change from baseline in HOMA-IR for both visits with the largest decrease in the highest dosing group (cohort 3) indicating a positive effect on insulin sensitivity and an improvement in insulin resistance.

### Results for 3 MD Cohorts in Part B BGE-105 Study

In the double-blind, placebo-controlled study, 21 healthy volunteers aged ≥65 years underwent strict bed rest for 10 days while receiving daily IV infusions of placebo (n=10) or a fixed dosage of BGE-105 (n=11). One day before (baseline, D-1) and 5 (D5) and 10 (D10) days after initiation of bed rest, key muscle atrophy endpoints were measured: thigh circumference; cross-sectional area (CSA) and A-P diameter of vastus lateralis (ultrasound); ultrasound muscle quality grade, an index that quantifies fatty degeneration in muscle (ultrasound echo density); and muscle protein synthesis rate (biopsy). Parameters measured during the 10-day bed rest period include thigh circumference, muscle size, muscle quality (e.g., fatty degeneration), and muscle protein synthetic rate. Table 5 summarizes the results.

TABLE 5
Key aspects of muscle atrophy outcomes after 10-day bed resting in older adults

| | | | Cross- | | | |
|---|---|---|---|---|---|---|
| | | | sectional | AP | Muscle | Cumulative |
| | | Thigh | area | diameter | grade | protein |
| | | circumference | (CSA) | (thickness) | (progression) | synthesis |
| % | Placebo | −6.4% | −21.2% | −19.5% | −80% (8 out | 15.9% |
| change: | | | | | of 10) | |
| baseline | BGE-105 | +0.8% | −5.7% | −8.0% | −9% (1 out | 22% |
| to D 10 | Single | | | | of 11) | |
| | dosage of | | | | | |
| | 240 mg | | | | | |
| | daily | | | | | |
| | (cohort | | | | | |
| | MD 1B) | | | | | |
| p value | | <0.0001 | <0.01 | <0.05 | <0.005 | <0.005 |

*Effect size based on Cramer's V. All percentages are relative to baseline values measured 1 day before initiation of dosing.

Measurements of thigh circumference and the vastus lateralis are among the gold standard markers of skeletal muscle atrophy. The vastus lateralis, the largest muscle in the quads, is among the commonly studied muscles given (a) its functional importance in mobility and disability, (b) location and architecture of the muscle, which results in ease of ultrasound measurement and biopsy, and (c) atrophy is more profound in older patients than the muscles in the lower leg. Endpoint metrics were measured at baseline, after 5 days of bedrest, and after 10 days of bedrest, including endpoints such as limb circumference, muscle area via ultrasound, and measurement of muscle quality via ultrasound (measured normal muscle vs fat), fractional synthetic rates. A number of endpoint metrics correlated with muscle size and muscle function biochemically, such as the thigh circumference, size of the muscle via ultrasound, to calculate diameter, thickness, and cross-sectional area of the muscle (calculated after ultrasound).

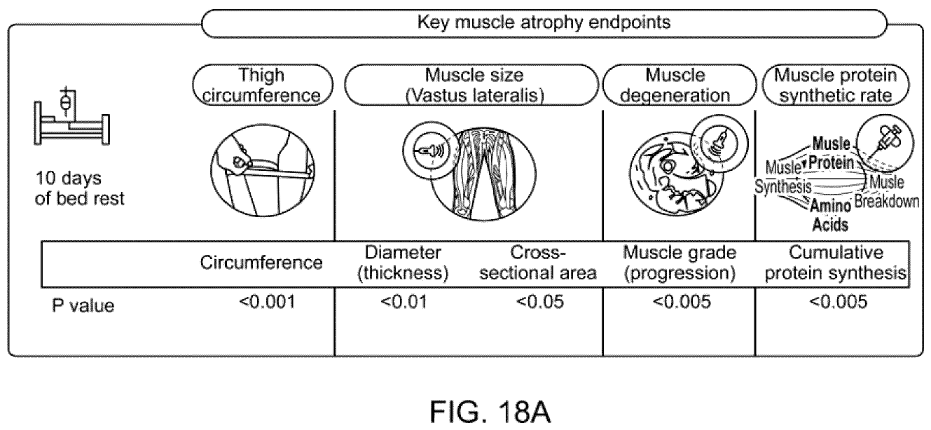

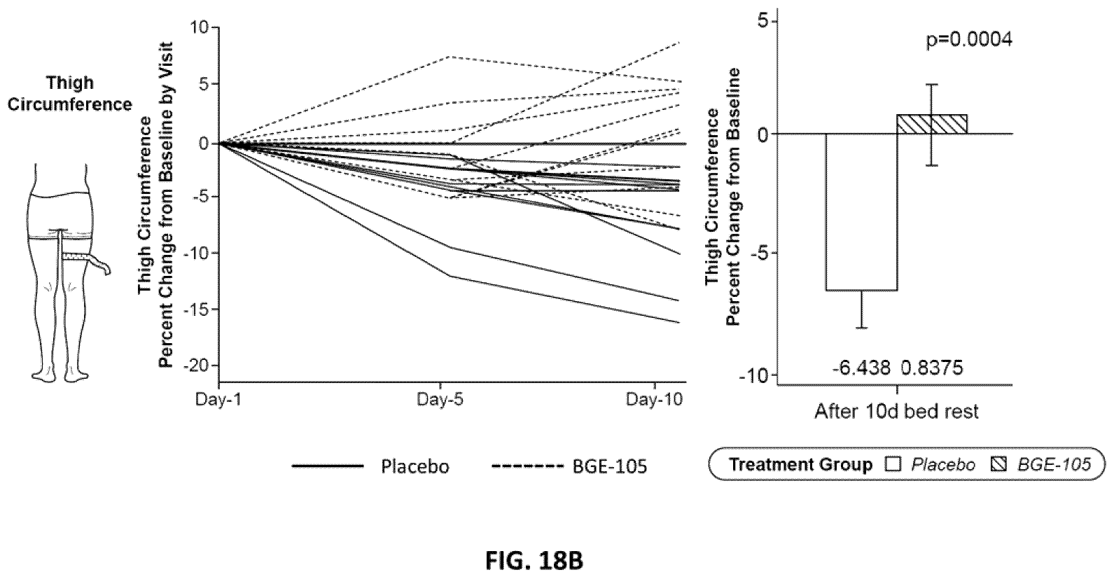

FIGS. 18A-18B show the effects of BGE-105 on rest-induced reduction in thigh circumference, Vastus Lateralis diameter % (thickness), Vastus Lateralis cross sectional area (CSA) %, muscle degeneration, and cumulative protein synthesis rate % (as measured from a biopsy) as summarized in Table 5. Measurements were made 15 cm superior of the mid patella. BGE-105 significantly reduced muscle atrophy across multiple key endpoints, in healthy volunteers aged ≥65 years. The higher rate of muscle protein synthesis in BGE-105 treated patients vs. placebo group provides a potential mechanistic basis for BGE-105's protective effect on muscle dimensions. The findings of the Phase 1b trial support investigation of BGE-105 as a treatment of a wide range of age-related syndromes driven by loss of muscle. These conditions include acute myopathies in hospitalized patients on mechanical ventilation, as well as chronic medical conditions. BGE-105 significantly prevented muscle atrophy across multiple endpoints: (circumference (p<0.001), Diameter (p<0.01), cross sectional area (p<0.05), muscle grade (progression) (p<0.005), and cumulative protein synthesis (p<0.005).

FIG. 18A shows that patients treated with BGE-105 (Cohort 1B) had an increase in thigh circumference, muscle size, muscle quality (e.g., fatty degeneration), and muscle protein synthesis rate as compared to the placebo group (Cohort 1A).

FIG. 18B shows thigh circumference of patients received placebo (Cohort 1A of MD study of Example 9) and BGE-105 treatment (Cohort 1B of MD study of Example 9) period is measured and presented as percent change from baseline over the course (middle panel) or after (left panel) of the 10-day bed rest duration. p=0.0004.

FIG. 18B shows BGE-105 significantly prevented bed rest-induced reduction in thigh circumference (middle panel). The results show that thigh circumference is correlated with thickness and cross-sectional area of constituent muscles via ultrasound. Older patients (>65 years old) experience substantial muscle atrophy with 10 days of bed rest, particularly in the leg. In general, older patients experience about 3.2-4.7% loss of total lean body mass, and about 6-7% loss of lean leg mass (cohort 1A, MD1A). After the 10-day bed rest period, older patients in the placebo group show a 6.438% reduction of thigh circumference while older patients in the BGE-105 treated group show a 0.8% increase in thigh circumference, <0.0001. Measurements are made 15 cm superior of the mid patella (left panel). Solid lines represent the treated patients, vs the dotted lines show the placebo. FIG. 18B shows that treated patients with BGE-105 show higher thigh circumference as compared to the placebo (cohort 1A). All percentages are relative to baseline values measured 1 day before initiation of dosing.

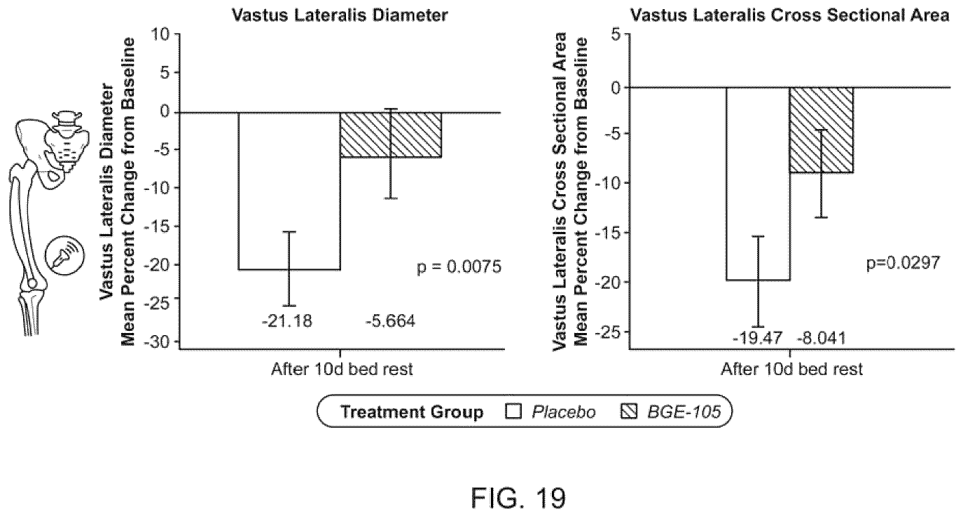

Muscle size was measured as a function of ultrasound, and the results showed that there was a 21% decrease in the placebo group (Cohort 1A) as compared to 5.664% decrease in the treatment group (Cohort 1B), showing about a 75% improvement in muscle dimension. FIG. 19 shows BGE-105 significantly prevented bed rest-induced reduction in thickness and cross-sectional area of the vastus lateralis via ultrasound (left panel). After 10-day bed rest, patients with BGE-105 treatment only had a 5.664% reduction of vastus lateralis diameter—as compared to patients in the placebo group showing a 21.18% reduction of the vastus lateralis diameter (p=0.0075) (middle panel). Similar results were seen in the measurements of vastus lateralis cross sectional area (right panel). BGE-105 treatment showed a 8.041% reduction of the cross-sectional area of the Vastus Lateralis, which was a significantly different reduction than the 19.47% reduction of the cross-sectional area of the Vastus Lateralis observed in the placebo group (p=0.0297). Percentages are shown as the mean percent change from baseline.

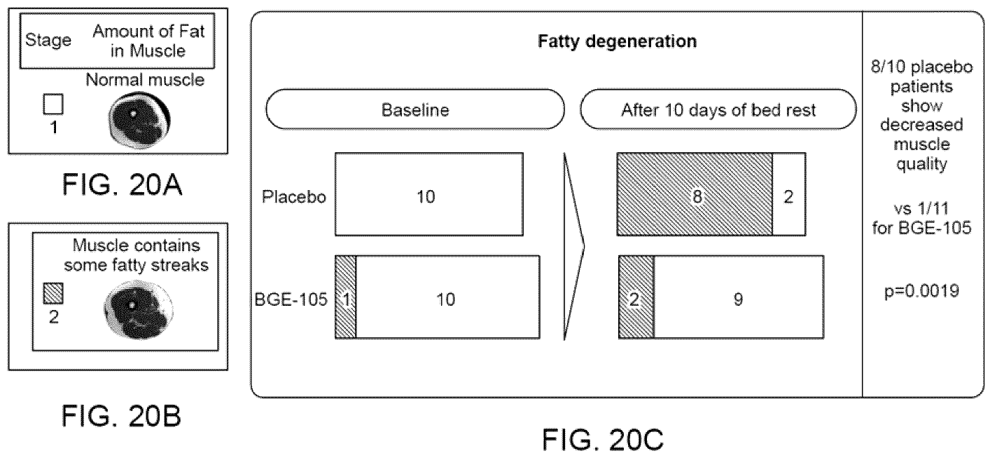

FIGS. 20A-20C show that BGE-105 significantly prevented bed rest-induced fatty degeneration of the vastus lateralis via echo density. Muscle grading is performed according to panels A-B. An echo density measurement determines the muscle quality. The echo density measurements showed that the placebo group (Cohort 1A) resulted in a 6.4% decrease in the placebo group (Cohort 1A), as compared to a 0.8% increase in the treatment group (Cohort 1B, 240 mg daily).

The echo density measurements showing the number of patients receiving an ultrasound muscle quality grading scale using a numerical grading system of grade 1 or a grade 2 classifying fatty muscle atrophy by measuring fatty infiltration of the muscle fibers of a biopsy from the patient. FIG. 20A illustrates amount of fat in normal muscle (1), FIG. 20B illustrates muscle containing some fatty streaks (2), FIG. 20C illustrates fatty degeneration changes from baseline to after 10 days of bed rest in placebo and BGE-105 groups. Open box represents normal muscle (grade 1), and striped box represents muscle that contains some fatty streaks (grade 2). After 10 days of bed rest, eight (8) out of 10 patients in the placebo group advanced to a grade 2, whereas only one (1) out of 11 BGE-105 treated patients moved to a grade 2 after 10 days of bed rest. (p=0.0019). The echo density measurements showed that 80% of patients went from an ultrasound muscle quality grade of 1 (normal muscle tissue) to a 2 (muscle tissue with fatty streaks), whereas in the BGE-105 treatment group (Cohort 1B), only 1 patient went from a grade of 1 to a 2, showing statistical significance.

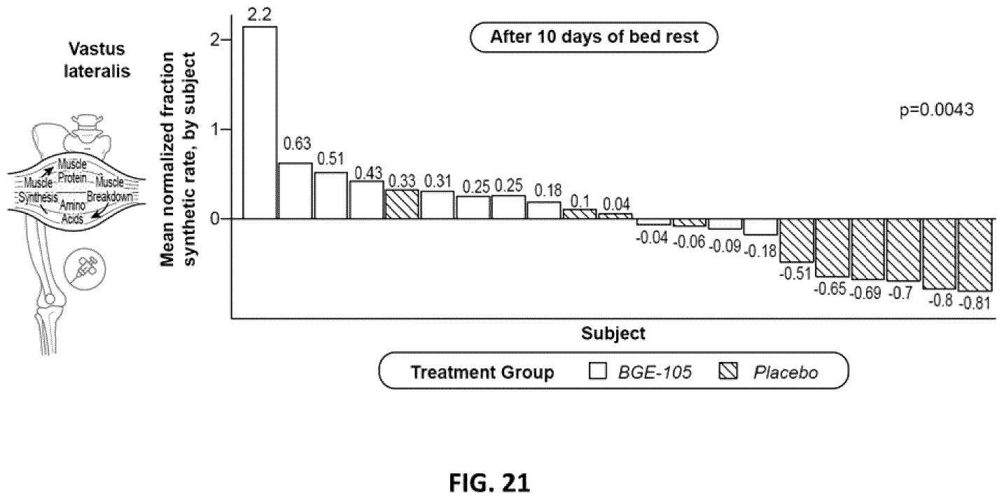

FIG. 21 shows rate of muscle protein synthesis in the vastus lateralis, measured via microbiopsy in individual subjects in the BGE-105 treated group and the placebo group. Muscle protein synthesis is measured after 10 days bed rest and normalized fraction synthetic rate by subject is presented. p=0.0043.

FIG. 21 shows BGE-105 resulted in an elevated rate of muscle protein synthesis in the vastus lateralis, measured via microbiopsy. The graph represents a ratio of the BGE-105 treated group versus the placebo group. The bards above the x-axis represent a higher level of muscle protein synthesis versus the placebo group after 10 days of bedrest. The muscle protein synthetic rate showed the cumulative muscle synthesis rate over the course of 10 days of bed rest. The muscle cumulative protein synthesis rate significantly increased in the BGE-105 treatment group (Cohort 1A) as compared to the placebo group (Cohort 1A). After 10 days of bed rest, 8 out of 11 (about 72.7%) of BGE-105 treated patients experience elevated rate of muscle protein synthesis, and have a mean normalized fraction synthetic rate ranging from 0.18-2.2. In comparison, only 3 out of 10 (30%) of placebo patients experienced elevated rate of muscle protein synthesis. The mean normalized fraction of synthetic rate ranges from 0.04-0.33. The results demonstrate that BGE-105 is efficient in inducing or maintaining muscle protein synthesis in the vastus lateralis (p=0.0043).

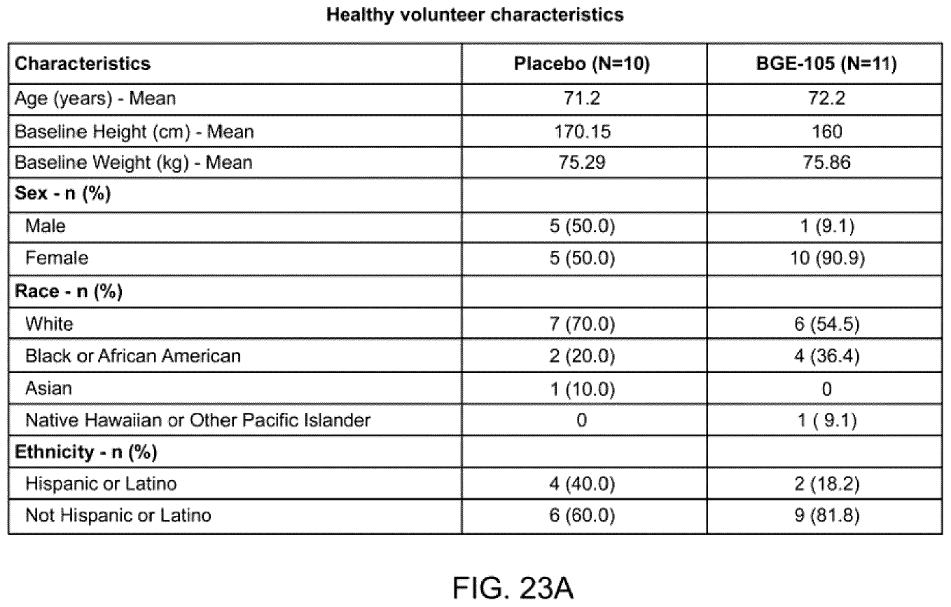

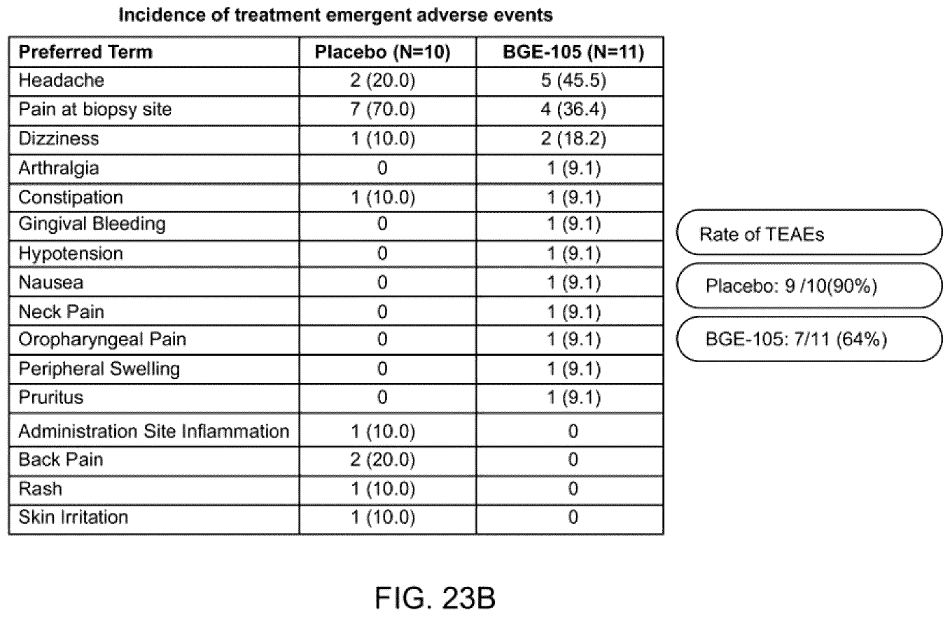

FIGS. 23A-23B shows healthy volunteer characteristics of the Phase 1b bed rest atrophy of the MD study and incidence of treatment emergent adverse events (Part B of Example 9). The Placebo group is represented as Cohort 1A of the MD, Part B study, and the BGE-105 treated group is represented as Cohort 1B of the MD, Part B study of Example 9. FIG. 23B shows incidence of treatment emergent adverse events (TEAEs). Nine (9) out of 10 (90%) of volunteers in the placebo group exhibited TEAEs, whereas seven (7) out of 11 (64%) of volunteers in the BGE-105 group exhibited TEAEs.

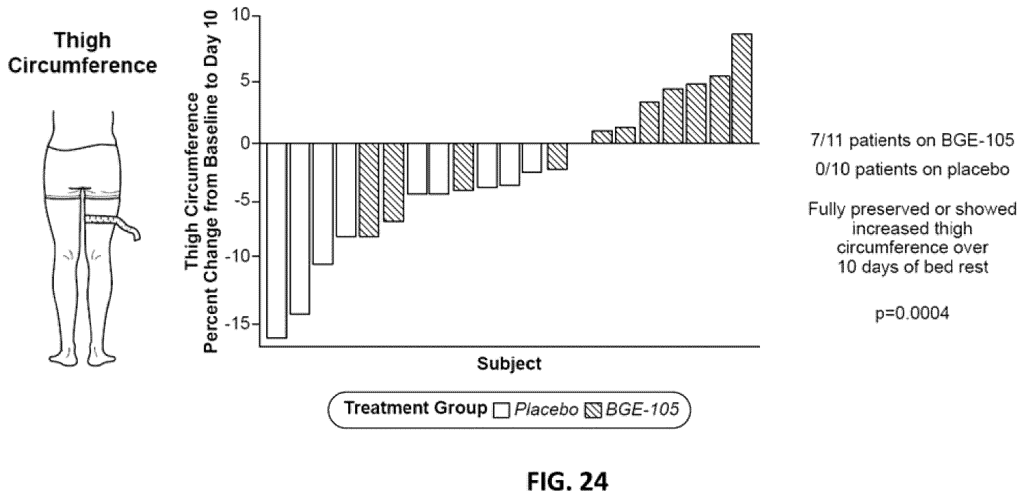

FIG. 24 shows effect of BGE-105 on rest-induced reduction in thigh circumference of subjects participating in Phase 1b, MD, Part B clinical trial of Example 9. Thigh circumference was measured in patients receiving placebo or BGE-105 treatment and presented as percent change from baseline. Subjects received BGE-105 fully preserve or show increased thigh circumference over 10 days of bed rest. Measurements are made 15 cm superior of the mid patella. P=0.0004. After 10 days of treatment, seven (7) out of 11 (64%) subjects on BGE-105 exhibited fully preserved or showed increased thigh circumference over 10 days of bed rest. P=0.0004. No (0%) subjects on placebo showed thigh circumference increase from baseline.

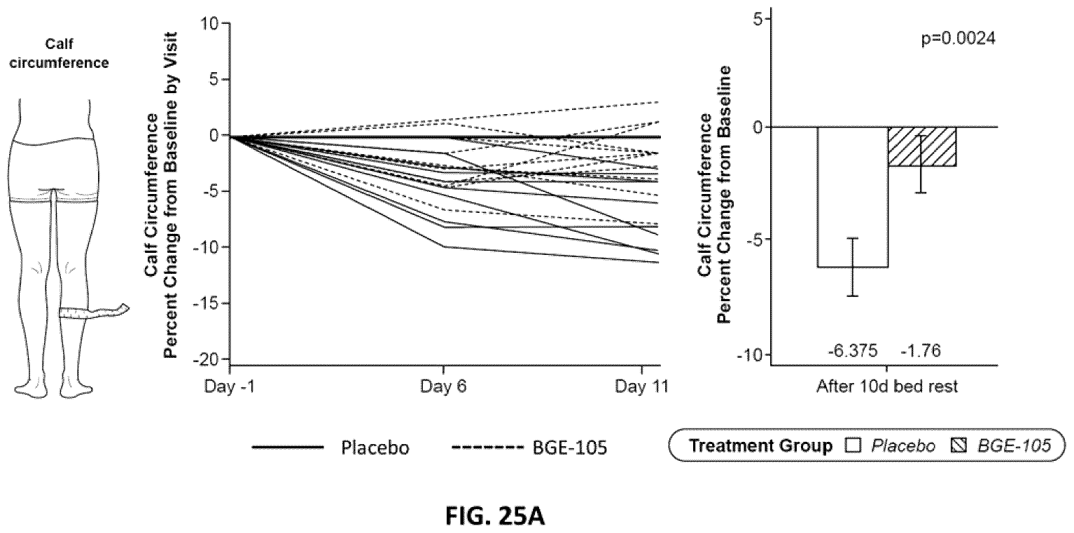

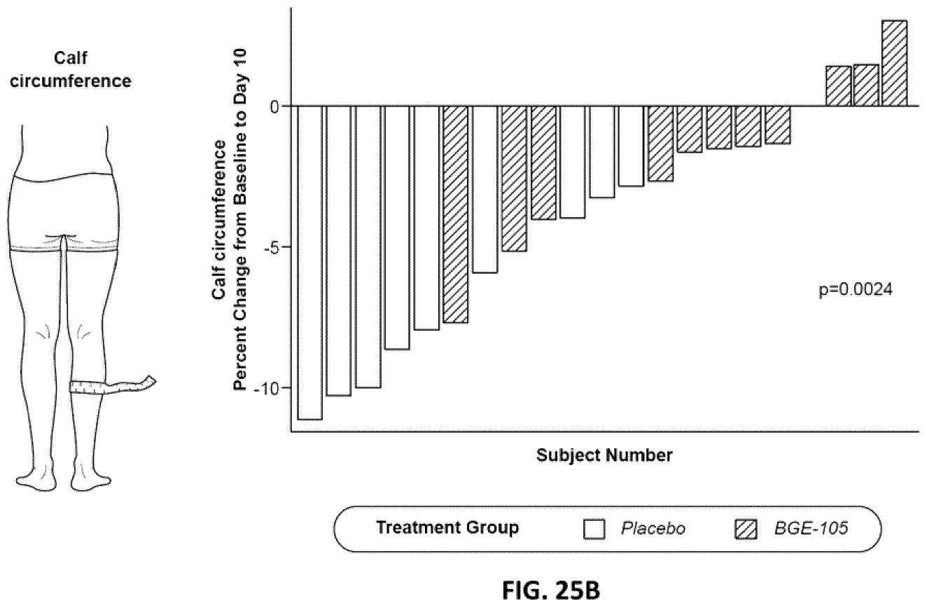

FIGS. 25A-25C show effect of BGE-105 on rest-induced reduction in calf circumference of subjects participating in Phase 1b, MD, Part B clinical trial of Example 9. Calf circumference of patients receiving placebo or BGE-105 treatment was measured and presented as percent change from baseline over the course (middle panel) or after (left panel) the 10-day bed rest duration (panel A). Calf circumferences are presented as percent change from baseline to day 10 (panel B). p=0.0024. Measurements were made 15 cm superior of the mid patella. FIG. 25C shows number of subjects exhibiting calf circumferences percentage change from baseline to 10 days.

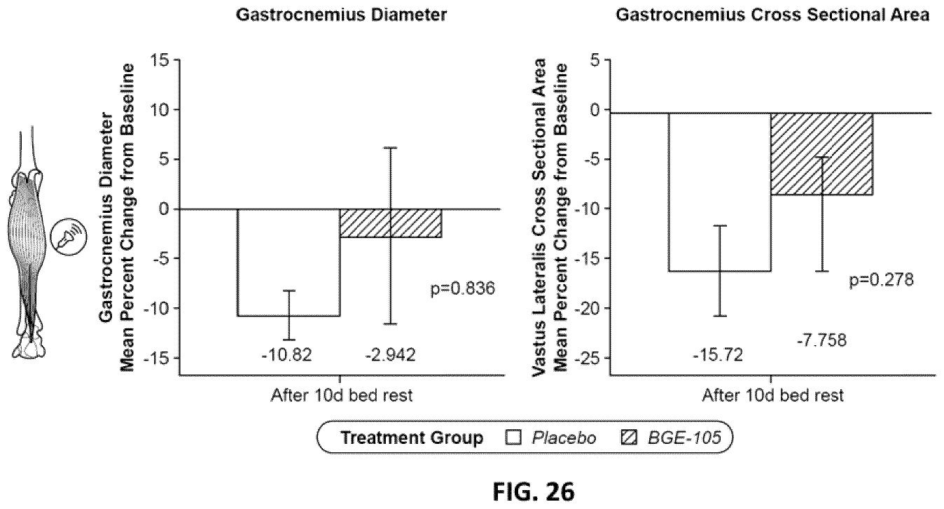

FIG. 26 shows the effects of BGE-105 on gastrocnemius diameter and gastrocnemius cross-sectional area via ultrasound after 10-days of bed rest. The gastrocnemius diameter (middle panel, p=0.836) or gastrocnemius cross sectional area (left panel, p=0.278) of patients treated with BGE-105 or placebo was presented as a mean percent change from baseline. As shown, there was a larger decrease in gastrocnemius diameter (10.982% decrease) and cross-sectional area (15.72% decrease) in the placebo group as compared to the diameter (2.942 decrease) and cross-sectional area (7.758% decrease) in the BGE-105 treated group.

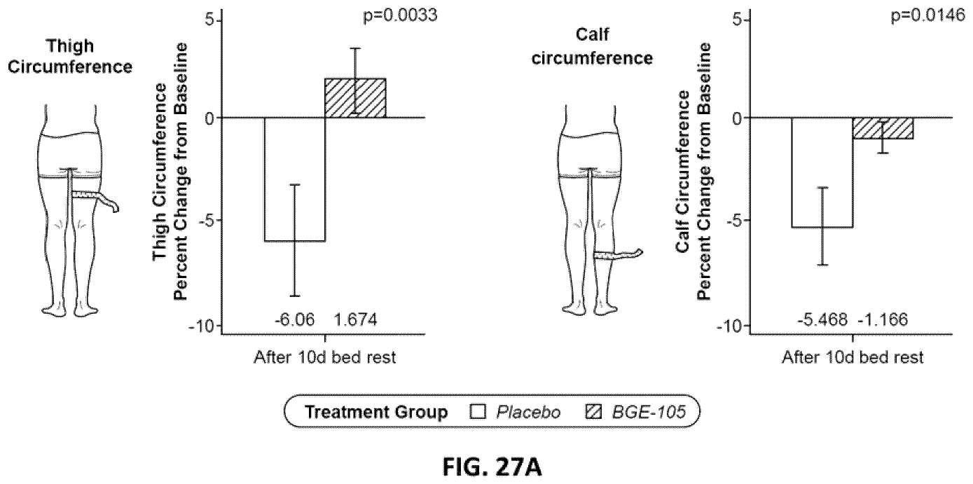

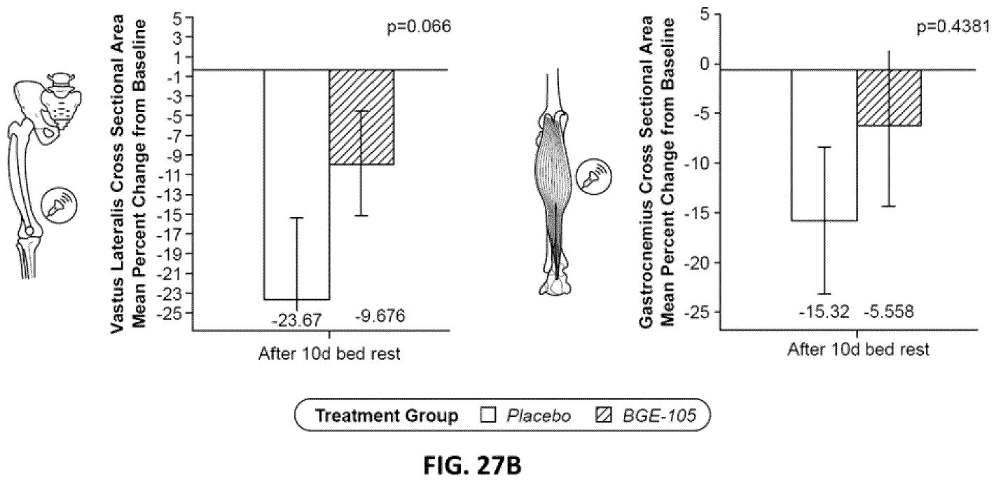

FIGS. 27A-27B show effects of BGE-105 in thigh, calf circumference, vastus lateralis cross sectional area, and gastroenemius cross sectional area measurements in female subjects. Thigh circumference (p=0.0033), calf circumference (p=0.0146), vastus lateralis cross sectional area (p=0.066), and gastrocnemius cross sectional area (p=0.4381) in subjects receiving placebo or BGE-105 were measured and presented as percent change from baseline. As shown, there is a larger decrease in thigh circumference (6.06% decrease) and calf circumference (5.486% decrease) in the placebo group as compared to the thigh circumference (1.674% decrease) and calf circumference (1.166% decrease) in the BGE-105 treated group (FIG. 27A). As shown, there is a larger decrease in vastus lateralis cross sectional area (23.67% decrease) and gastrocnemius cross sectional area (15.32% decrease) in the placebo group as compared to the vastus lateralis cross sectional area (9.676% decrease) and gastrocnemius cross sectional area (5.558% decrease) in the BGE-105 treated group (FIG. 27B).

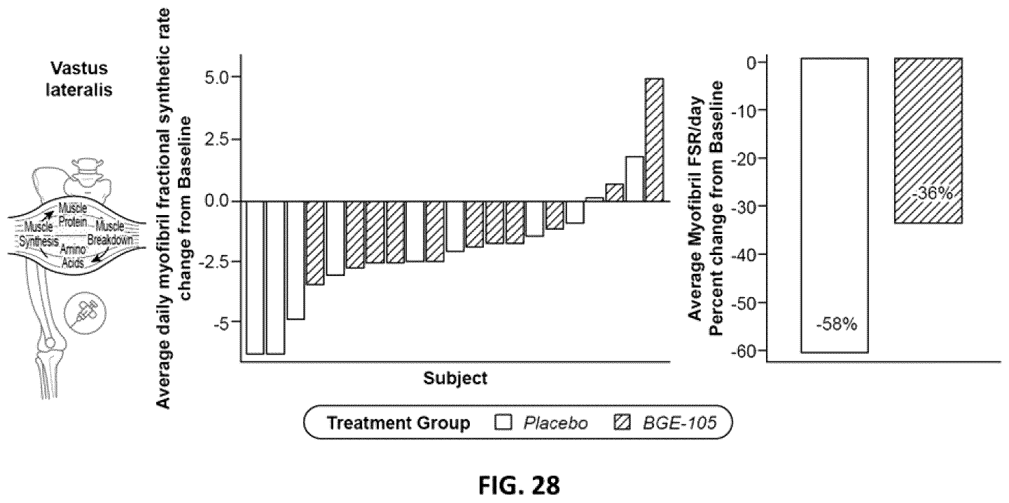

FIG. 28 shows synthetic rate of muscle myofibril proteins in the vastus lateralis in BGE-105 treated and placebo treated patients. Average vastus lateralis muscle myofibril synthesis rate is measured and presented as change from baseline (left panel). Average myofibril FSR/day is presented as percent change from baseline (right panel). As shown, there is a larger decrease in the synthetic rate of muscle myofibril proteins in placebo (58% decrease) vs BGE-105 treated (36% decrease) group.

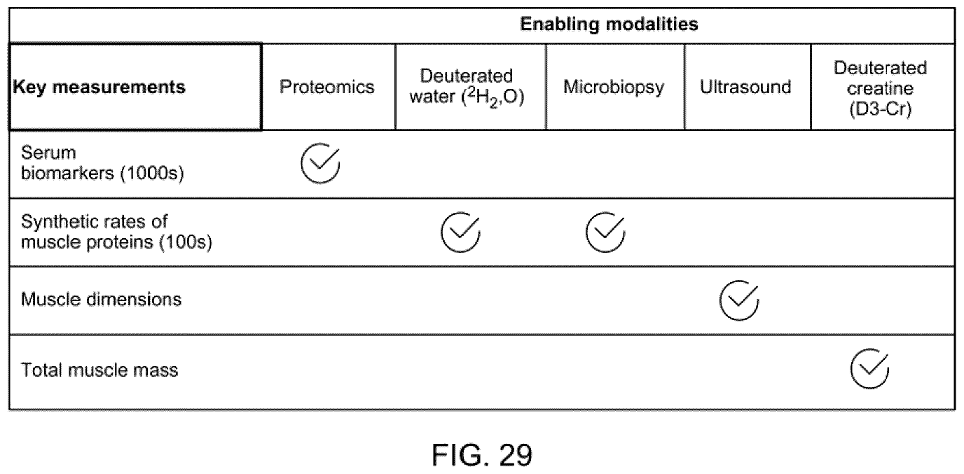

FIG. 29 illustrates measurements taken during the Phase 1b bed rest atrophy study used for assessment of several muscle dynamics as shown in Example 9. Key measurements include serum biomarkers, synthetic rates of muscle proteins, muscle dimensions, and total muscle mass.

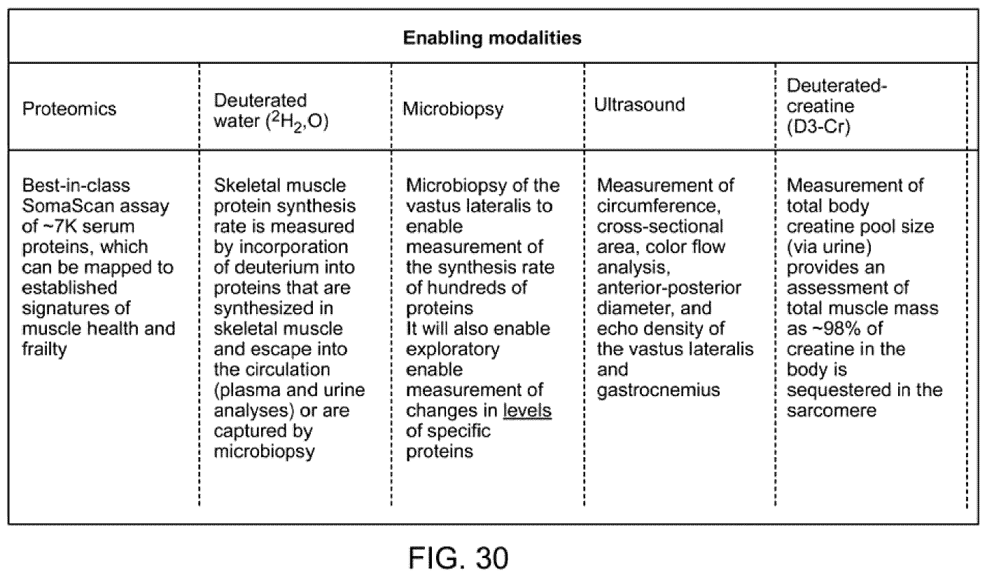

FIG. 30 illustrates description of modalities used to assess muscle dynamics in Phase 1b beds rest atrophy study of Example 9. Proteomics analysis involves SomaScan assay of ˜7k serum proteins, which can be mapped to established signatures of muscle health and frailty. Skeletal muscle protein synthesis rate is measured by incorporation of deuterium into proteins that are synthesized in skeletal muscle and escape into the circulation (plasma and urine analyses) or are captured by microbiopsy. Microbiopsy of the vastus lateralis is used to enable measurement of the synthesis rate of hundreds of proteins. It will also enable exploratory measurement of changes in levels of specific proteins. Untrasound measures circumference, cross-sectional area, color flow analysis, anterior-posterior diameter, and echo density of the vastus lateralis and gastrocnemius. Measurement of total body creatine pool size (via urine) provides an assessment of total muscle mass as ˜98% of creatine in the body is sequestered in the sarcomere.

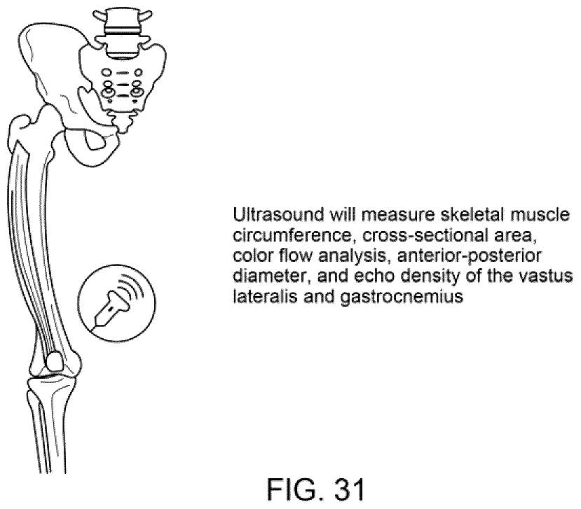

FIG. 31 illustrates the site and method of ultrasound measurements of leg muscle. Ultrasound measures skeletal muscle circumference, cross-sectional area, color flow analysis, anterior-posterior diameter, and echo density of the vastus lateralis and gastrocnemius.

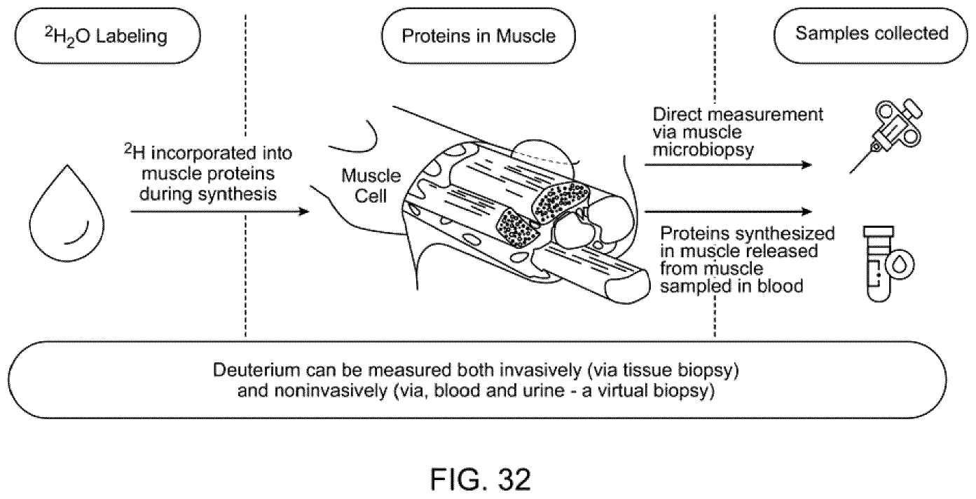

FIG. 32 illustrates incorporation of deuterated water into muscle proteins for calculation of the fractional synthetic rate. Deuterium can be measured both invasively (via tissue biopsy) and noninvasively (via, blood and urine—a virtual biopsy).

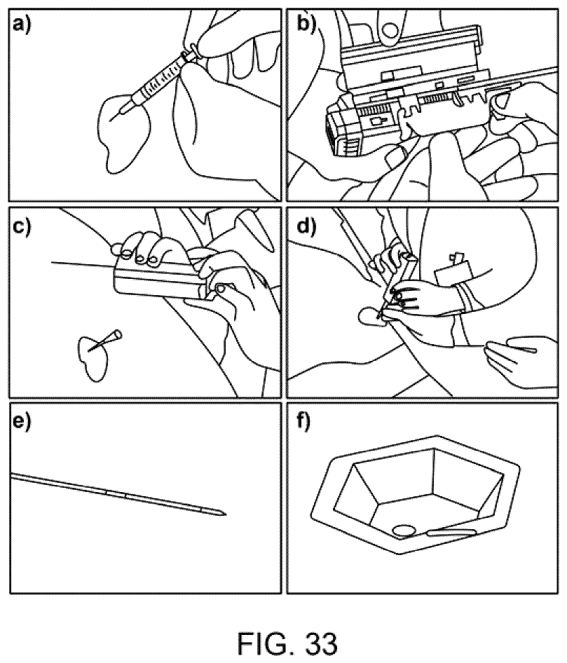

FIG. 33 shows microbiopsy of the vastus lateralis performed at several time points (at baseline day zero of bedrest, at day 5 of bedrest, and at the end of the 10-day bed rest period) for measuring the fractional synthetic rate of hundreds of muscle proteins. Biopsy performed with microneedle was collected at three timepoints: baseline, midway through the treatment period, and end of the treatment period.

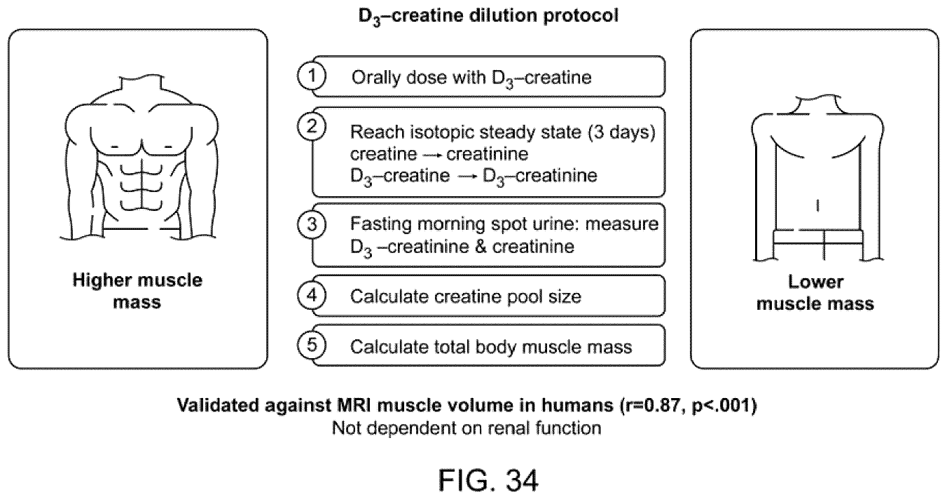

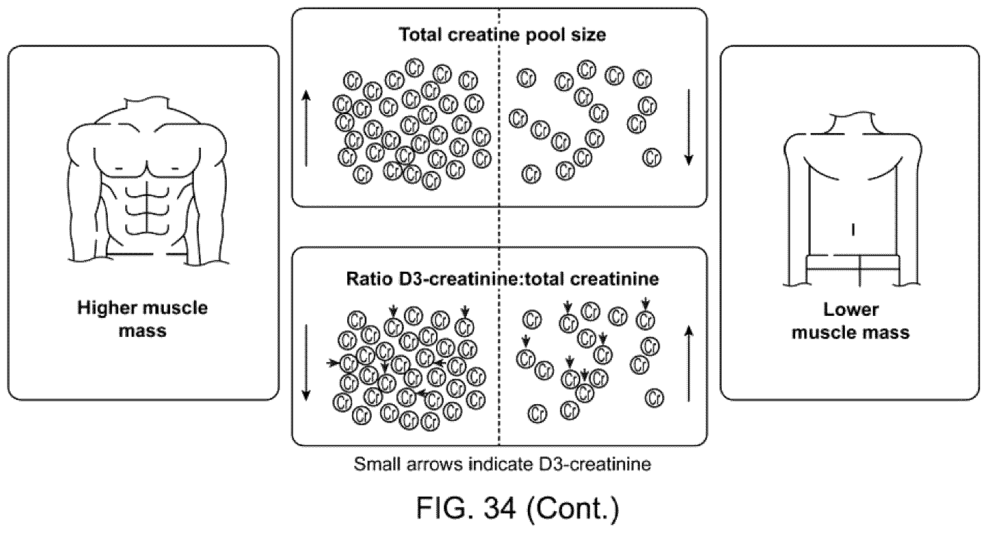

FIG. 34 depicts a flowchart for assessing total muscle mass using a D3-creatine tracer during the phase 1b study of Example 9. The data is validated against MRI muscle volume in humans (r=0.87, p<0.01). Not dependent on renal function. Briefly, subjects were given orally dose with D3-creatine. Subjects reached isotopic steady state (about 3 days). D3-creatinine and creatinine were measured in fasting morning spot urine. Creatine pool size and total body muscle mass were calculated. Higher creatine pool size and higher ratio of D3-creatinine:total creatinine indicates higher muscle mass. Lower creatine pool size and lower ratio of D3-creatinine:total creatinine indicates lower muscle mass.

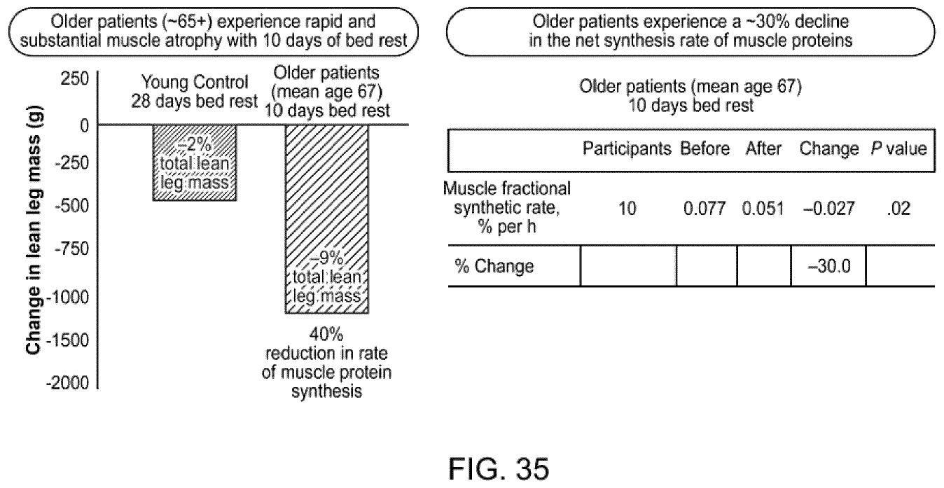

FIG. 35 shows correlation of atrophy of skeletal muscle and decrease in muscle protein synthesis in older patients (>65 years old). Muscle atrophy was presented as change in lean leg mass (g) (left panel). Muscle fractional synthetic rate is measured before and after the 10 day bed rest period in patients treated with BGE-105 or placebo, and is presented as percent change (right panel). Data demonstrate that older patients (65+) experienced rapid and substantial muscle atrophy with 10 days of bed rest. A reduction of 9% of total lean leg mass was observed in older patients after 10 days bed rest, whereas only 2% reduction of total lean leg mass in young control subjects after 28 days of bed rest. About 40% of old patients showed reduction in rate of muscle protein synthesis. Older patients also experienced about 30% decline in the net synthesis rate of muscle proteins. P=0.02.

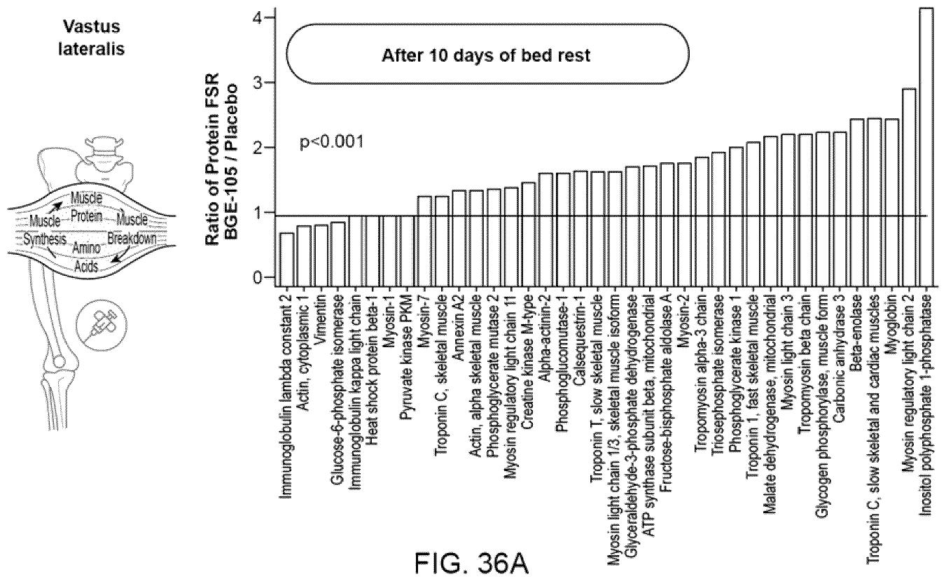

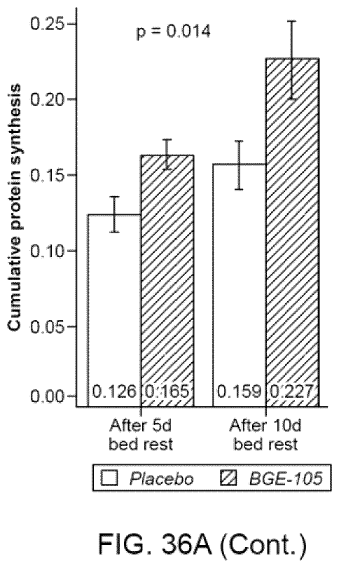

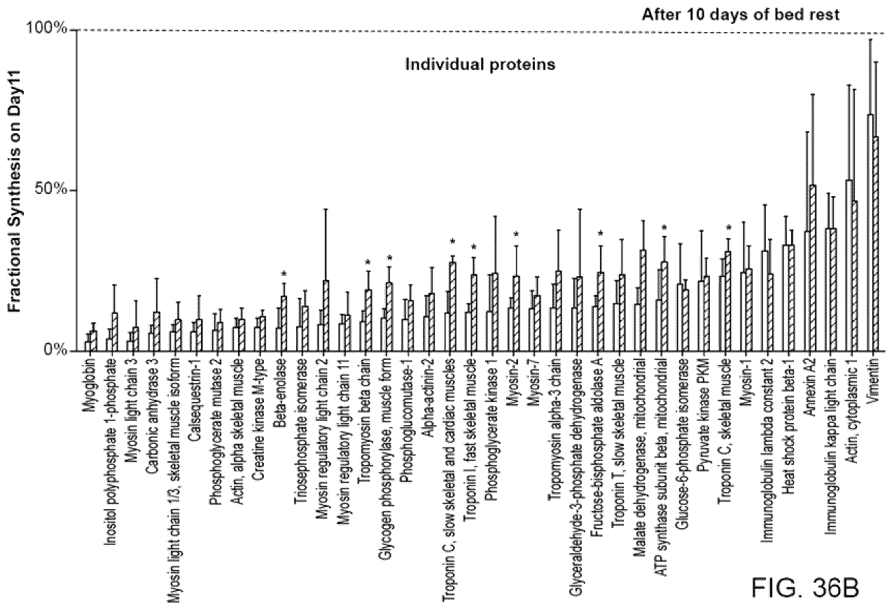

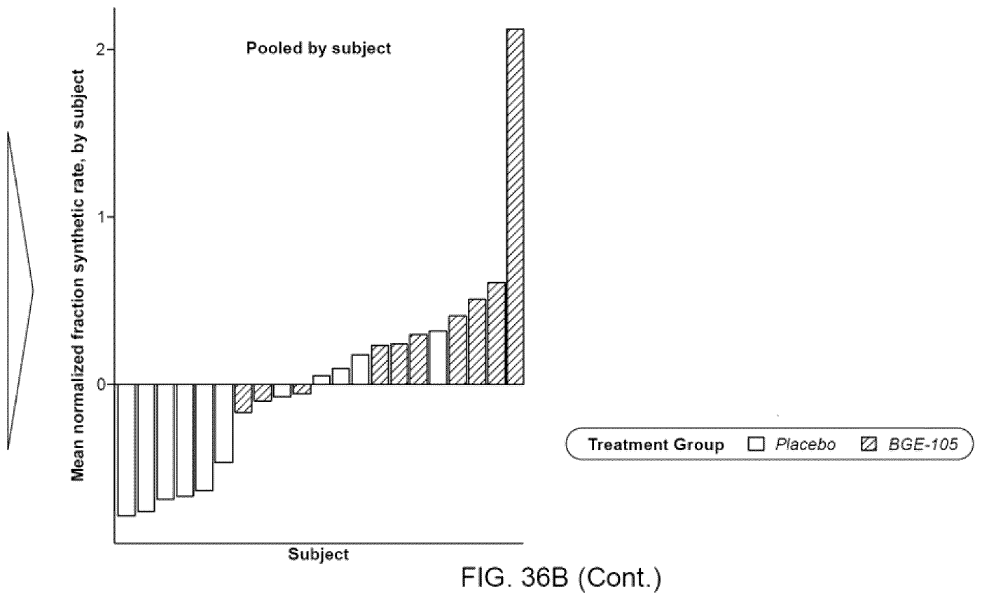

FIG. 36A shows the effect of BGE-105 on muscle protein synthesis in vastus lateralis microbiopsies in subjects treated with BGE-105 or placebo. Shown is ratio of protein FSR BGE-105 over placebo after 10 days of bed rest (middle panel). Shown is comparison of cumulative protein synthesis in placebo and BGE-105 treated subjects at after 5 day bed rest and after 10 day bed rest (right panel).

FIG. 36B shows the fractional synthesis of BGE-105 treated patients and placebo treated patients on day 11 of bedrest. As shown, BGE-105 resulted in higher muscle protein synthesis in the vastus lateralis compared to placebo-treated patients, measured via microbiopsy.

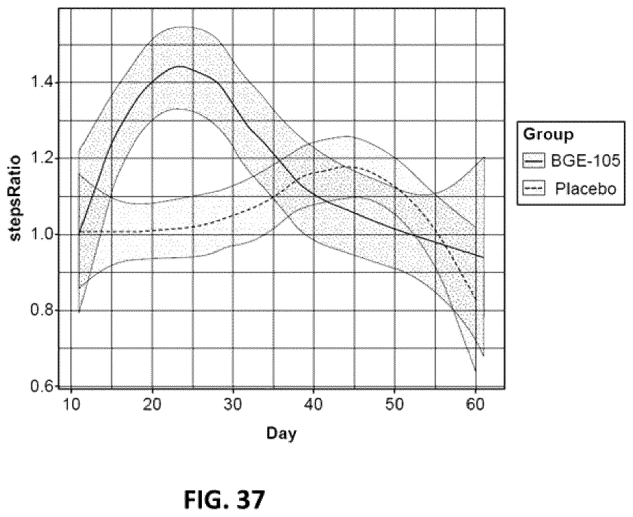

FIG. 37 shows the step ratio of patients who wore a wearable activity device during Day 10 through Day 60 (post-bedrest time period) of the Phase 1b, MD, Part B clinical trial as described above. The ratio is calculated by dividing eat subjects later step counts by their baseline step count, which is the mean daily steps seen over the pre-bedrest period. Patients treated with BGE-105 showed an increase in physical activity by determining the number of steps taken by the patient using the wearable activity device as compared to the patients who received a placebo. The activity levels start off near the baseline and then increase for the BGE-105 treated group for several weeks before the curve converges again.

Muscle atrophy, loss of muscle mass and strength, is a universal feature of human aging that increases the risk of multiple morbidities, shortens lifespan, and diminishes quality of life. Hospitalization and periods of forced inactivity greatly accelerate this loss in older people.

The analysis of the inventors' unique human aging cohorts revealed that the apelin pathway is a strong predictor of healthy longevity and muscle function, and translated directly into the clinical finding of this study that apelin pathway activation with BGE-105 improved muscle physiology in older adults.

Analysis of proprietary human biobanks showed that apelin pathway activity, which declines with age, was positively associated with longevity, mobility, and cognitive function. Apelin, the natural ligand of APJ, is secreted by skeletal muscle in response to exercise and regulates multiple aspects of muscle metabolism, growth, and repair.

The double-blind, placebo-controlled clinical trial evaluated the safety and pharmacodynamics of BGE-105. Twenty-one volunteers underwent 10 days of bed rest while receiving infusions of BGE-105 or placebo.

Volunteers on placebo (n=10) exhibited muscle atrophy, reflected by statistically significant reductions in thigh circumference and ultrasound measurement of vastus lateralis muscle dimensions (cross sectional area and thickness) and muscle quality (fatty degeneration).

Treatment with BGE-105 (n=11) significantly ameliorated muscle atrophy relative to placebo:

Muscle dimensions: Volunteers receiving BGE-105 showed a 100% improvement in thigh circumference (p<0.001) relative to placebo-treated volunteers, and ultrasound measurements showed a 58% improvement in vastus lateralis cross-sectional area (p<0.05) and a 73% improvement in vastus lateralis thickness (p<0.01).

Muscle quality: Ultrasound echo density measurements revealed that the muscle quality grading scale, an index that quantifies degeneration in muscle, worsened in 8 of 10 volunteers on placebo vs. only 1 of 11 volunteers receiving BGE-105 (p<0.005).

Muscle protein synthesis: Proteomic analysis of muscle microbiopsy samples revealed that bed rest decreased production of muscle proteins, and this effect was significantly ameliorated by BGE-105 (p<0.005). The higher rate of muscle protein synthesis in the drug vs. placebo group provides a potential mechanistic basis for BGE-105's protective effect on muscle dimensions.

Apelin agonist BGE-105 resulted in statistically significant improvement vs. placebo in muscle size, quality, and protein synthesis in volunteers ≥65 years old during 10 days of bed rest, with no serious adverse effects. BGE-105 treatment resulted in statistically significant prevention of muscle atrophy relative to placebo in healthy volunteers aged 65 or older after 10 days of strict bed rest.

On day 10, volunteers receiving BGE-105 showed improvement in bed rest-induced atrophy relative to placebo-treated volunteers, reflected in multiple metrics (Table). BGE-105 was well tolerated in the study, with no severe adverse effects reported. The result is shown in Table 5 above.

BGE-105 was well tolerated in the study in terms of safety. BGE-105 significantly reduced muscle atrophy across multiple key endpoints, in healthy volunteers aged ≥65 years. The higher rate of muscle protein synthesis in BGE-105 treated vs. placebo group provided a mechanistic basis for BGE-105's protective effect on muscle dimensions. The findings of the Phase 1b clinical trial support investigation of BGE-105 as a treatment of a wide range of age-related syndromes driven by loss of muscle. These conditions include acute myopathies in hospitalized patients on mechanical ventilation, as well as chronic medical conditions that are common among millions of older people but lack approved therapeutics for prevention or treatment, representing an enormous unmet clinical need.

### Proteomic Signatures of Physical Function and Mortality

Proteomic profiling and analysis were performed on serum collected from the phase 1B clinical trial subjects (treated vs placebo) as described above. Eleven (11) treated and 10 placebo subject's serum levels were profiled for their proteomics collected at day −1 (baseline), day 5 and day 11. A linear regression model was implemented with an interaction term between treatment group and day to identify proteins whose differential abundance between the treatment groups influenced the average rate of change per day of a given protein. This model was fit separately for all proteins measured, and the resulting coefficient (on the interaction term) for each protein was used to rank order all proteins from most positive to most negative coefficient.

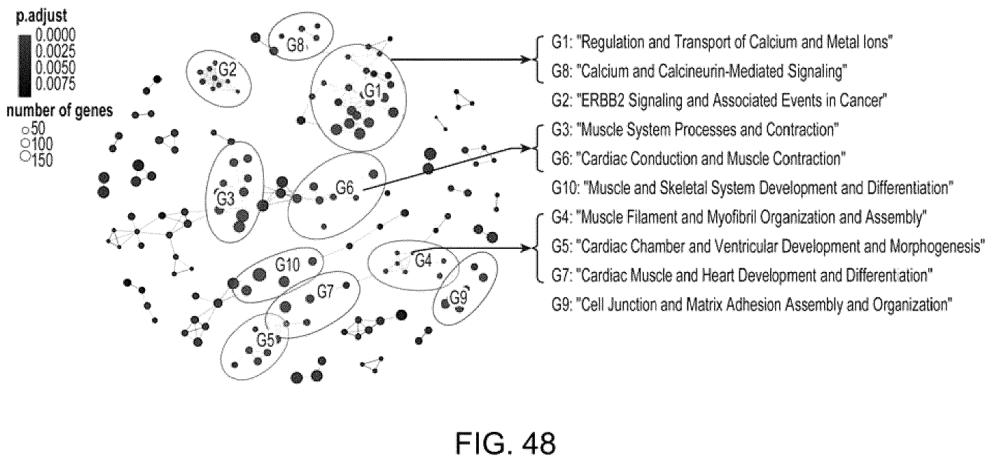

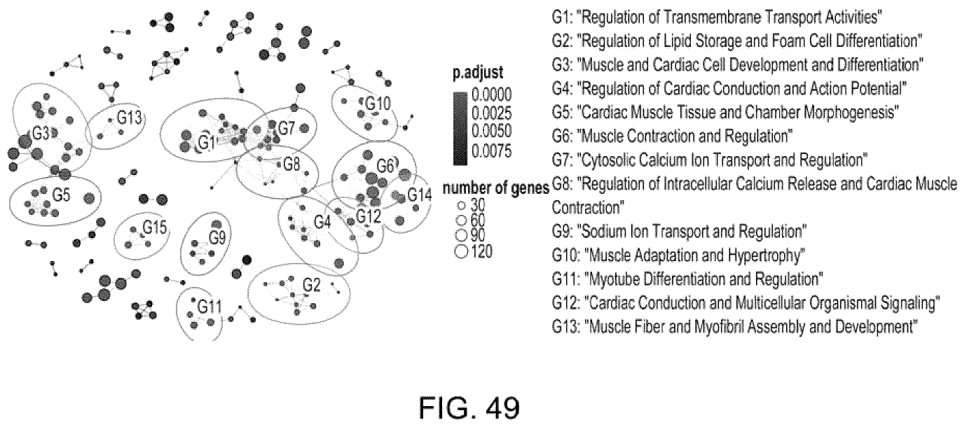

To test if the effect of BGE-105 on the plasma proteome significantly affected proteomic signatures of physical function and mortality, an enrichment analysis was performed using GSEA method, using (1) the aforementioned ranked list of proteins affected by BGE-105 and (2) various protein sets. For each human aging cohort phenotype (various physical function phenotypes and mortality), two protein sets consisting respectively (to preserve directionality) of the proteins positively and negatively associated (p<0.05) with that phenotype. Significant phenotypes in the right-hand graph of FIG. 48 and FIG. 49 with their GSEA plots and p-values (significant phenotypes were not shown when their protein sets overlapped substantially with those of already shown phenotypes).

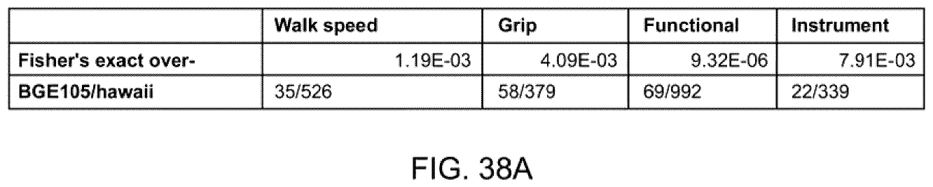

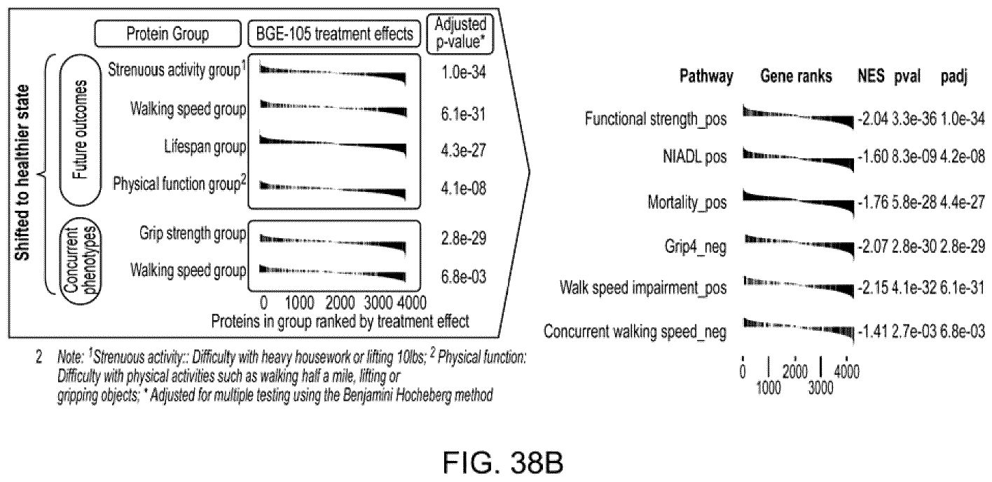

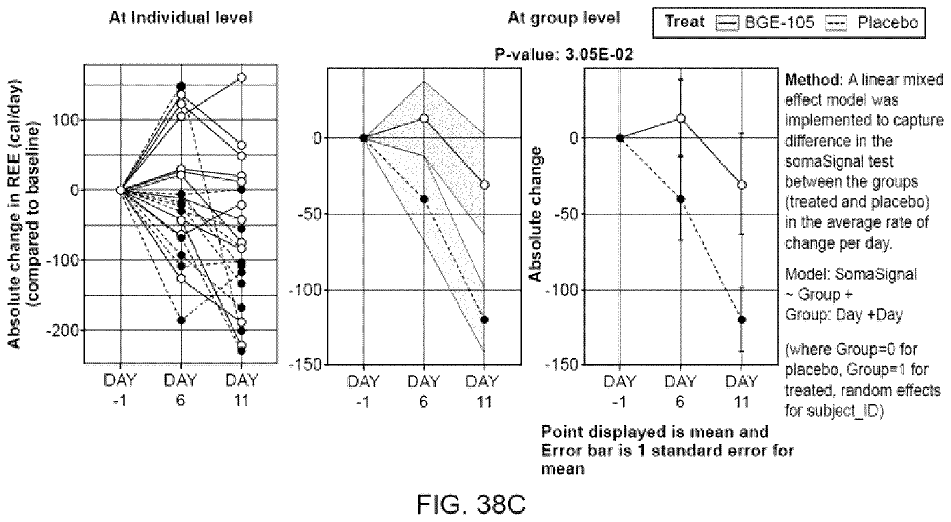

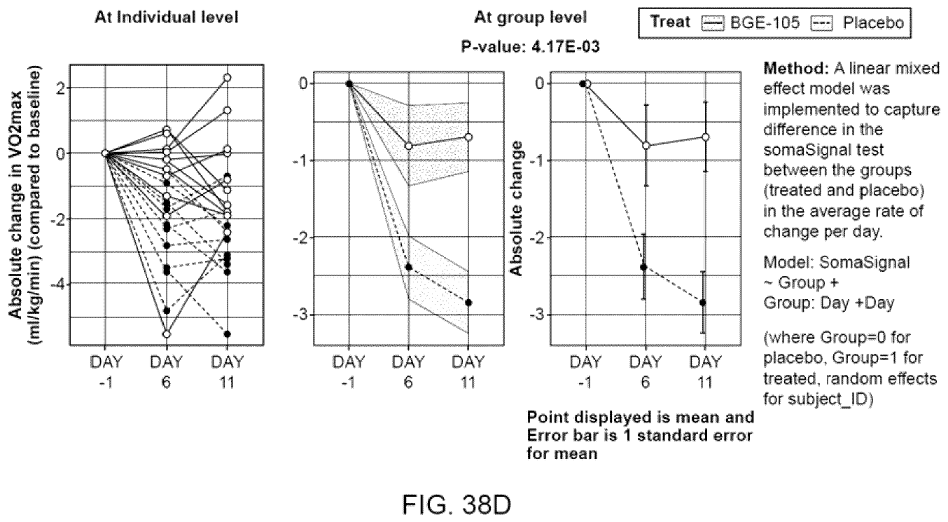

The proteomics data showed significant enrichment of proteins that were associated with concurrent and future decline in muscle function; i.e. the clinical trial data analysis showed a trend in the proteome shifted towards a more healthy functional outcomes for individuals that were treated with BGE-105 under bedrest condition. This observation reinforced the initial observations of the importance of apelin in preservation of grip strength with aging suggesting potential long-term benefits of treatment with BGE-105. FIGS. 38A-38D show proteomic profiling analysis performed on serum collected from subjects of the phase 1B clinical trial of Example 9. 11 treated and 11 placebo subject's serum levels were profiled for their proteomics collected at day −1 (baseline), day 5 and day 11. FIG. 38A shows proteomics data from patients in the phase 1B clinical trial, providing the number of proteins associated with frailty (functionality), walk speed, instrumental activities of daily living (IADL) (functional instrument), and grip strength that are changed in patients treated with BGE-105 in the phase 1b clinical trial as described above. IADL is a specific class of functional activities being measured. For frailty (functionality), 69 out of 992 proteins associated with frailty in patients of the phase 1b clinical trial were changed by treatment with BGE-105 (p<0.05). For walk speed, 35 out of 526 proteins associated with walk speed in patients of the phase 1b clinical trial were changed by treatment with BGE-105. For grip strength, 58 out of 379 proteins associated with grip strength were changed by treatment with BGE-105. FIG. 38B shows that BGE-105 shifted the serum proteome towards a healthier state, recapitulating the benefits of naturally high apelin levels in subjects treated with BGE-105. Protein groups include strenuous activity group, walking speed group, lifespan group, physical function group, grip strength group, and walking speed group. Strenuous activity refers to difficulty with heavy housework or lifting 10 lbs. Physical function refers to difficulty with physical activities such as walking half a mile, lifting or gripping objects. P-value was adjusted for multiple testing using the Benjamini Hocheberg method.

Protein group definitions for the proteomics data shown in FIGS. 38A-38B is provided in the table below:

### Protein Group Definitions

| Technical | Presentation | Association | |
|---|---|---|---|
| name | name | Directional | Definition |
| Concurrent | Walking speed | negative | Proteins negatively associated with walk speed |
| walking | (concurrent | | (m/s) measured at exam 4 in the Hawaii |
| speed | outcomes) | | cohort (p-value < 0.05). The model |
| | | | implemented was a linear regression with walk speed |
| | | | variable (m/s), corrected for confounders: Age, |
| | | | pack years smoked, and monthly alcohol consumption. |
| NIADL | Physical | positive | Proteins positively associated with difficulty |
| | function | | with aspects of basic function or general |
| | (future | | aspects of physical strength and mobility, such |
| | outcomes) | | as walking half a mile, lifting 10 lbs, or |
| | | | gripping small objects (N = 896 with 463 events; |
| | | | p-value < 0.05). The model implemented |
| | | | was a Cox regression and corrected for |
| | | | confounders: age, pack years smoked, and |
| | | | monthly alcohol consumption. |
| Mortality | Mortality | positive | Proteins positively associated with all-cause |
| | (future | | mortality in Hawaii cohort (N = 933 with 897 |
| | outcomes) | | events; p-value < 0.05). The model |
| | | | implemented was a Cox regression and |
| | | | corrected for confounders: age, pack years |
| | | | smoked, and monthly alcohol consumption |
| Grip4 | Grip strength | negative | Proteins negatively associated with grip |
| | (concurrent | | strength measured at exam 4. The model |
| | outcomes) | | implemented was a linear regression with grip |
| | | | strength (kg), corrected for confounders: Age, |
| | | | pack years smoked, and monthly alcohol consumption. |
| Walk speed | Walking speed | positive | Proteins positively associated with walk speed |
| impairment | (future | | declining below 0.8 m/s (N = 884 with 243 |
| | outcomes) | | events; p-value < 0.05). The model |
| | | | implemented was a Cox regression and |
| | | | corrected for confounders: age, pack years |
| | | | smoked, and monthly alcohol consumption. |
| Functional | Strenuous | positive | Proteins positively associated with increased |
| strength | activity | | difficulty with heavy housework or lifting 10 |
| | (future | | lbs (N = 933 with 379 events; p-value < 0.05). he |
| | outcomes) | | model implemented was a Cox |
| | | | regression and corrected for confounders: age, |
| | | | pack years smoked, and monthly alcohol |
| | | | consumption. |

SomaSignal Test Using Proteomics Data

Next, a linear mixed effect model was implemented in the proteomics data to capture difference in a SomaSignal test between the two groups (treated and placebo) in the average rate of change per day.

When assessing resting energy expenditure, the Somasignal test was used to predict an individual's resting energy expenditure in calories per day (cal/day) using 122 aptamers. Population used: UK-based study of 9,022 individuals (aged 29-64 yrs), Model performance: CCC=0.66, R2=0.46 (CI: 0.42-0.49). FIG. 38C shows changes in baseline energy expenditure in BGE-105 treated and placebo treated subjects using a SomaSignal test on the proteomic data. FIG. 38C shows the absolute change in REE (cal/day) compared to baseline (end of treatment, p=0.03). As shown, changes in baseline energy expenditure were largely driven by changes in muscle mass. As shown in FIG. 38C, the proteomics data predicted that the resting energy rate was “rescued” by BGE-105 treatment.

When assessing cardiorespiratory fitness (VO₂) max, a SomaSignal test was used to predict estimated peak exercise capacity using 52 aptamers. North-american-based study of 743 individuals (aged 15-65 yrs), Model performance: CCC=0.85, R2=0.75 (CI: 0.68-0.81). FIG. 38D shows changes in cardiorespiratory fitness (VO₂) max and basal metabolic rate in BGE-105 treated and placebo treated subjects using a SomaSignal test on the proteomic data. As shown in FIG. 38D, VO₂ max was driven by maximal cardiac output and uptake of oxygen by skeletal muscle. BGE-105 treatment shifted the proteome towards predicted higher basal metablic rate and VO₂ max by the SomaSignal test (end of treatment, p=0.004).

BGE-105 significantly changed the serum levels of some of the same proteins that are associated in BioAge longitudinal aging cohort data with future decline in physical function as assessed by walk speed, activities of daily living (functional instrument) and grip strength. These proteins can then be used to try to identify patients who are responding to BGE-105 treatment and are likely to have reduced muscle atrophy.

The result also demonstrate that clinical multi-omics provide predicted benefits for muscle strength, metabolism, and aerobic capacity.

Results of Disuse Atrophy Clinical Study

The study demonstrated that compared to placebo, BGE-105 significantly reduced muscle loss as measured by vastus lateralis muscle thickness (75% reduction in loss) and cross-sectional area (>50% reduction in loss). This preservation of muscle mass was supported by the maintenance of skeletal muscle fractional protein synthetic rates (as measured by D₂O method and muscle biopsy). Crucially, the synthesis of myofibrillar structural proteins such as myosin and troponin was preserved compared to placebo.

### 7.10. Example 10: Phase 2 Study of BGE-105 for the Prevention of Diaphragmatic Atrophy

This is a clinical Phase 2 study of BGE-105 for the prevention of muscle atrophy leading to physical dysfunction in older patients on prolonged bed rest due to severe illness, surgery, or trauma. This example investigates the efficacy of BGE-105 for the prevention of diaphragmatic atrophy (DA).

Patients undergoing mechanical ventilation (MV) undergo rapid diaphragmatic atrophy (DA) given muscle disuse. Diaphragmatic atrophy is highly prevalent in the critical illness setting. It has high impact on morbidity and mortality, with no standardized treatments. Prevention of diaphragmatic atrophy is among the highest unmet needs in critical illness medicine given impact on patient ventilator weaning. Intensive care unit (ICU) diaphragm atrophy results in poor clinical outcomes and significant resource utilization. About 40-75% of mechanical ventilation (MV) patients develop significant diaphragmatic atrophy given muscle disuse. DA is the leading cause of difficulty weaning from MV and leads to poor clinical outcomes and increased resources. Generally, patients with DA have 2× longer time on MV (7 vs 4 days), 2× longer time in the ICU (12 vs. 6 days), and/or 4× higher in-hospital mortality (27% vs. 7%). There is no approved therapeutic for prevention or treatment of DA.

This Phase 2 clinical trial assesses the effect of BGE-105 in preventing adverse outcomes in older patients under mechanical ventilation in the intensive care unit (ICU). This study assesses the ability of BGE-105 to prevent: ICU diaphragmatic atrophy. These conditions, which affect millions of patients every year, are both associated with poor clinical outcomes and substantially increased mortality. No effective treatments are currently available, representing a high unmet medical need.

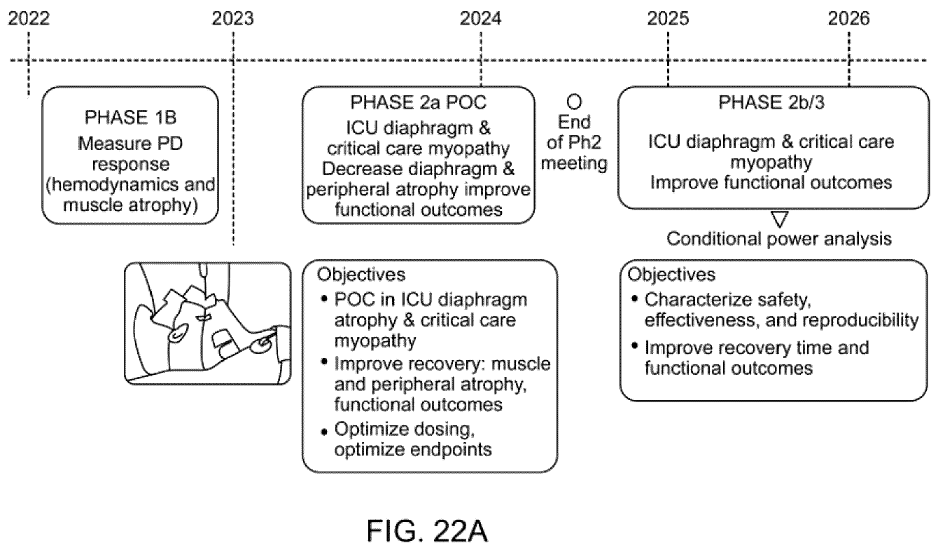

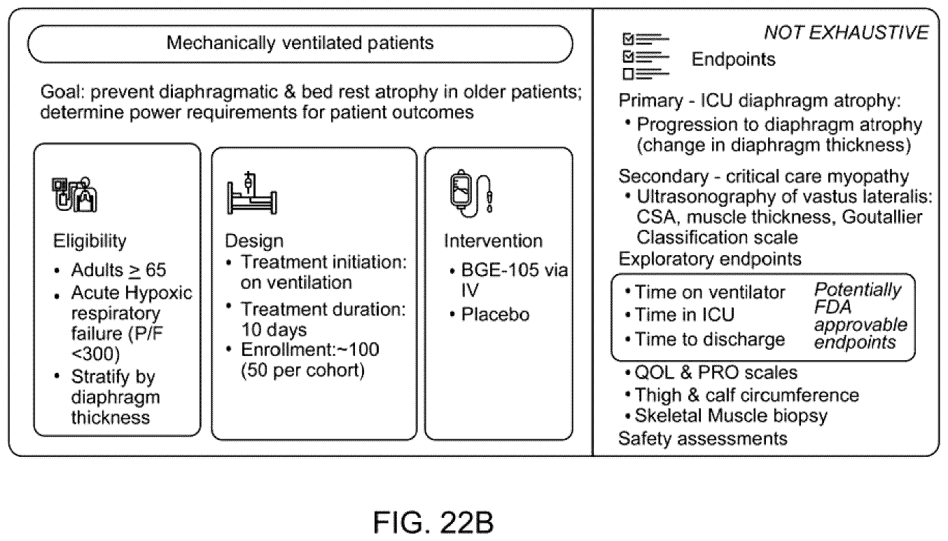

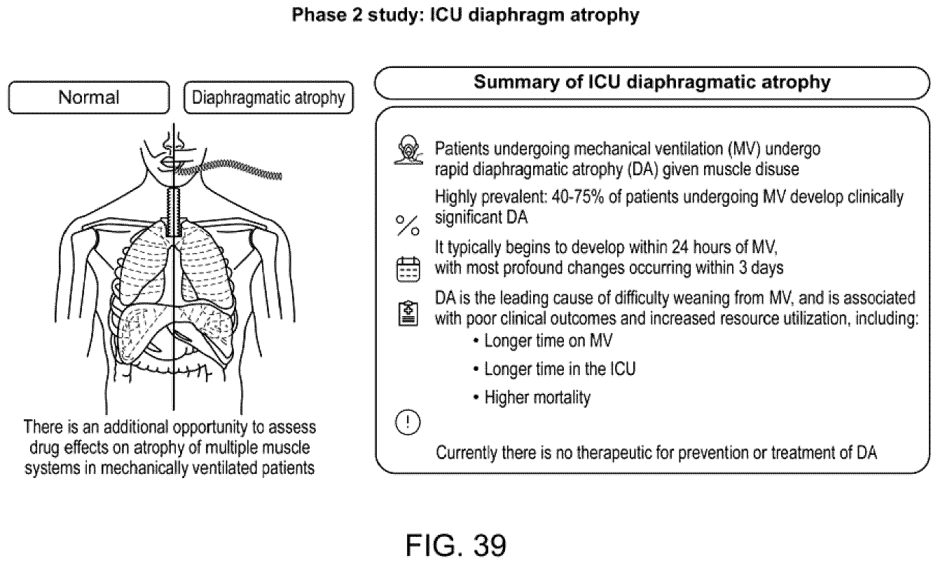

FIGS. 22A-22B and FIG. 39 illustrate an outline of the phase 2 point of care (POC) trial in ICU diaphragm. FIG. 22A illustrates a timeline of BGE-105 clinical trials. FIG. 22B illustrates design of the phase 2 POC trial in mechanically ventilated patients with ICU diaphragm atrophy. The study investigates POC in ICU diaphragm atrophy for improve recovery: decrease of muscle and peripheral atrophy, and/or improve functional outcomes. Other objectives include optimize dosing and optimize endpoints, to characterize safety, effectiveness, and reproducibility of the treatment.

The goal of the trail is to prevent diaphragmatic and bed rest atrophy in older mechanically ventilated patients and to determine power requirements for patient outcomes.

Adults mechanically ventilated patients ≥65 years old who have acute hypoxic respiratory failure (P/F<300) and stratified by diaphragm thickness are eligible for enrolment. Treatment is initiated on ventilation for a duration of 10 days. Approximately 100 patients are enrolled: 50 in placebo and 50 in BGE-105 treated. BGE-105/placebo is administered via IV.

Primary endpoint—ICU diaphragm atrophy: evaluate progression to diaphragm atrophy as indicated by change in diaphragm thickness.

Secondary endpoint-critical care myopathy: ultrasonography of vastus lateralis as indicated by cross sectional area (CSA), muscle thickness, Goutallier Classification scale.

Exploratory endpoints include time on ventilator, time in ICU, time to discharge, quality of life (QOL) and PRO scales, thigh and calf circumference, and skeletal muscle biopsy.

### 7.11. Example 11: Phase 2 Study of BGE-105 for the Prevention of Critical Care Myopathy

This is a clinical Phase 2 study of BGE-105 for the prevention of muscle atrophy leading to physical dysfunction in older patients on prolonged bed rest due to severe illness, surgery, or trauma. This example investigates the efficacy of BGE-105 for the prevention of critical illness myopathy (CIM).

Critical illness myopathy is highly prevalent in the critical illness setting. It has high impact on morbidity and mortality, with no standardized treatments. Critical illness myopathy is significantly de-risked by phase 1b study in bed rest atrophy. About 40-90% of ICU patients develop critical illness myopathy, a condition of proximal muscle weakness and atrophy. CIM is also associated with poor clinical outcomes and increased resources as DA (Example 11). About 65% of CIM patients have disabling and prolonged weakness post-ICU discharge, including 15% with continued weakness at 1 year. CIM increases about 30% increase in ICU-related costs, about 15%-25% increase in mortality, both in-hospital and at 5-years.

FIG. 22A illustrates a timeline of the phase 2 point of care (POC) trial in critical illness myopathy. FIG. 22B illustrates design of the phase 2 POC trial in mechanically ventilated patients with critical illness myopathy. The study investigates POC in critical illness myopathy for improve recovery time and functional outcomes, optimizes dosing and optimizes endpoints, to characterize safety, effectiveness, and reproducibility of the treatment.

### 7.12. Example 12: BGE-105 Efficacy in Improving Muscle Functions

Novel apelin receptor agonist. BGE-105 is a potent, oral small molecule agonist of the apelin APJ receptor currently in Phase 1 with safety, PK & PD data in 220+ people. There are currently no approved APJ receptor agonists, highly novel target. Involved in regulation of many cardiac, vascular, metabolic, muscle (e.g., such as skeletal muscle), and gastrointestinal functions. Organ dysfunction associated with age and disease-related dysregulation of apelin signaling is expected to respond to treatment with APJ receptor agonists.

BGE-105 improves old mouse muscle function and frailty in multiple preclinical models testing elderly mice.

BGE-105 improves frailty (running wheel activity, grid hang time). Protects mouse muscles from atrophy in hindlimb immobilization. Improves muscle regeneration after cardiotoxin challenge.

BioAge's informatics platform revealed strong link to longevity and healthspan. Older adults with increased apelin levels live longer, with improved physical and mental function.

BGE-105 is studied in a Phase 1b bed rest trial to explore potential of BGE-105 to decrease rate of muscle loss.

Generally healthy men and women aged >65 years old. Single-blind, placebo-controlled 10-day bed rest model with standardized diet with composition based on age/sex/weight. Assessing safety, pharmacokinetics, changes in insulin sensitivity (HOMA-IR), skeletal muscle protein fractional synthetic rates using proteomic evaluation following enrichment of body water with deuterated water, total body muscle mass evaluation using deuterated creatine supplementation, vastus lateralis and gastrocnemius muscle volume and cross-sectional area assessed by ultrasound, and physical performance measured by 30 second sit-to-stand test.

Frailty can be measured by detecting the serum proteins that are associated in BioAge longitudinal aging cohort data with future decline in physical function as assessed by walk speed, activities of daily living (functional instrument) and grip strength. These proteins can then be used to try to identify patients who are at risk for future sarcopenia, muscle atrophy or loss of muscle strength who are in need of treatment with BGE-105. BGE-105 significantly changed the serum levels of some of the same proteins that are associated in BioAge longitudinal aging cohort data with future decline in physical function as assessed by walk speed, activities of daily living (functional instrument) and grip strength. These proteins can then be used to try to identify patients who are responding to BGE-105 treatment and are likely to have reduced muscle atrophy.

### 7.13. Example 13: Phase 2 Clinical Study in Older Mechanically Ventilated Patients to Prevent ICU Diaphragmatic Atrophy & Critical Illness Myopathy

This example is an update with clinical protocol for the clinical trials of BGE-105 in preventing ICU diaphragmatic atrophy or critical illness myopathy in older mechanically ventilated patients as described in Examples 10 and 11.

Patients undergoing mechanical ventilation (MV) undergo rapid diaphragmatic atrophy (DA) given muscle disease. This is highly prevalent in 40-75% of patients undergoing MV that develop clinically significant DA. DA typically begins to develop within 24 hours of MV, with most profound changes occurring within 3 days. DA is the leading cause of difficulty weaning from MV, and is associated with poor clinical outcomes and increased resource utilization, including longer time on MV, longer time in ICU, and higher mortality. This study as outlined in FIG. 39 assesses the effects of BGE-105 on elderly patients that are mechanically ventilated in preventing ICU diaphragm atrophy and/or critical illness myopathy to determine the effects of BGE-105 on atrophy of multiple muscle systems in mechanically ventilated patients.

TABLE 12
Phase 2 clinical study in older mechanically ventilated patients to prevent ICU
diaphragmatic atrophy and critical illness myopathy protocol.
Eligibility:
100 Patients that ≥65 years old, and have acute hypoxemic respiratory failure (P/F < 300),
stratified by diaphragm thickness are included in this study. 50 patients receive IV
administration of BGE-105, and 50 patients receive a placebo (IV saline). There is an 8-week
follow up following treatment.
Treatment initiation:
Patients are on mechanical ventilation for <36 hours
Treatment duration:
BGE-105 or placebo is administered daily for 10 days
Primary endpoint:
ICU diaphragm atrophy is evaluated for proportion of patients with ≥10% decrease in
diaphragm thickness.
The primary endpoint is the progression of patients to ≥10% diaphragmatic atrophy given
correlation with poor patient outcomes: Primary endpoint includes ≥10% decrease in
diaphragmatic thickness which has been shown to strongly predict clinical trajectory of
intubated patients, including: 31% lower daily chance of being weaned from the ventilator
71% increased average duration of ICU admission, 3-fold higher odds of an ICU
complication.
Outcomes:
Time on ventilator, Rate of reintubation, Time in ICU, Time to discharge, Mortality, Day
alive at home/days, alive out of hospital, Physiological, Airway occlusion pressure, Maximal
inspiratory pressure, Skeletal muscle biopsy, Functional performance, QOL scales, Medical
Research Council Score, (muscle strength), Montreal Cognitive Assessment, Change in
ambulation status, Daily activity level (wearable).
Secondary endpoints:
Muscle measurements, diaphragm thickening fraction, ultrasonography: vastus lateralis,
abdominal muscles; thigh circumference

Result: In adults over 65 years of age receiving invasive mechanical ventilation for acute hypoxemic respiratory failure, BGE-105 prevents diaphragm muscle atrophy during the early course of mechanical ventilation. Patients who are responding to BGE-105 treatment and are likely to have reduced muscle atrophy.

### 7.14. Example 14: sn RNAseq Analyses of Human Muscle Tissue Samples from Healthy Adult Patients (Phase Lb Clinical Study)

This example presents updated proteomic analysis data from biosamples collected from healthy adult patients in Phase 1b clinical study as described in Example 9.

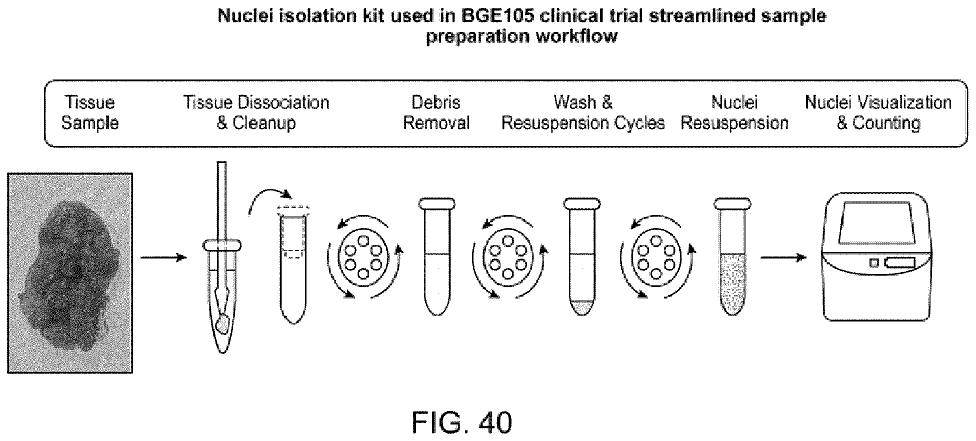

FIGS. 40-62 show details and results of snRNAseq analyses of human muscle tissue samples from the above Phase 1b clinical study of BGE-105 for treating muscle atrophy. These results are consistent with demonstration of efficacy in the clinical studies described herein.

FIG. 40 illustrates nuclei isolation for 10× Genomics single cell transcriptome gene expression technology used to assess tissue samples. FIG. 40 illustrates workflow of a nucleic isolation kit used in BGE-105 clinical trial streamlined sample preparation. Tissue samples were collected from patients and processed in a laboratory. Tissues were dissociated and cleaned up. The debris were removed and the sample (e.g., nuclei) was washed, resuspended for multiple cycles according to routine tissue processing procedures. Cell nuclei were visualized and counted using standard transcriptome gene expression technologies 10× Genomics single cell library technology is used for assessing tissue samples.

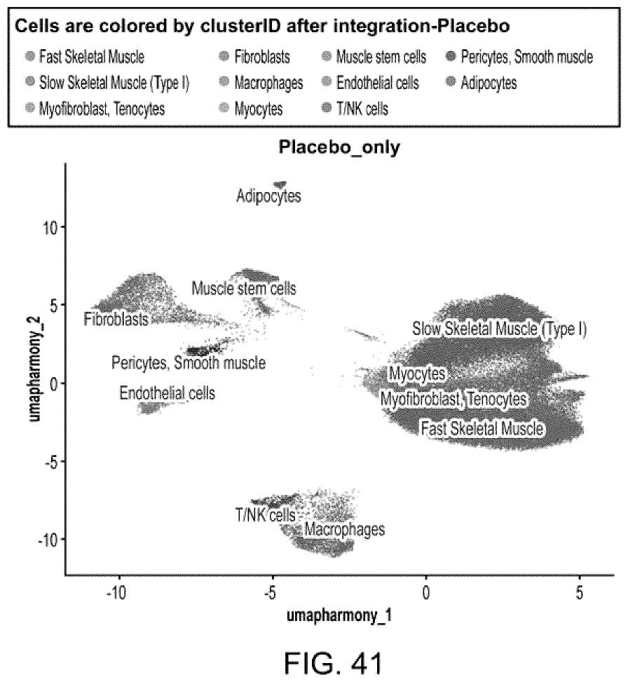

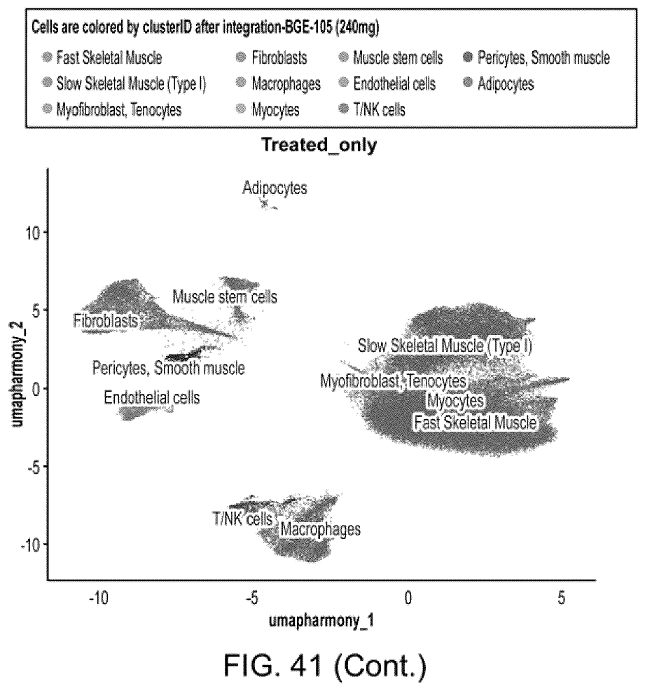

FIG. 41 shows 11 cell types were identified, consistent with published muscle atlas (see e.g., Perez et al., 2022, Single nuclei profiling identifies cell specific markers of skeletal muscle aging, frailty, and senescence. Aging (Albany NY). 2022 Dec. 13; 14(23):9393-9422. doi: 10.18632/aging.204435. Epub 2022 Dec. 13. PMID: 36516485; PMCID: PMC9792217). Cell type clustered include fast skeletal muscle, slow skeletal muscle (type 1), myofibroblast, tenocytes, fibroblasts, muscle stem cells, pericytes, smooth muscle, endothelial cells, T/NK cells, and adipocytes. Two methods and two annotations were used. Top 20 variable genes within clusters were used as markers for cell type annotation in placebo and BGE-105 treated groups.

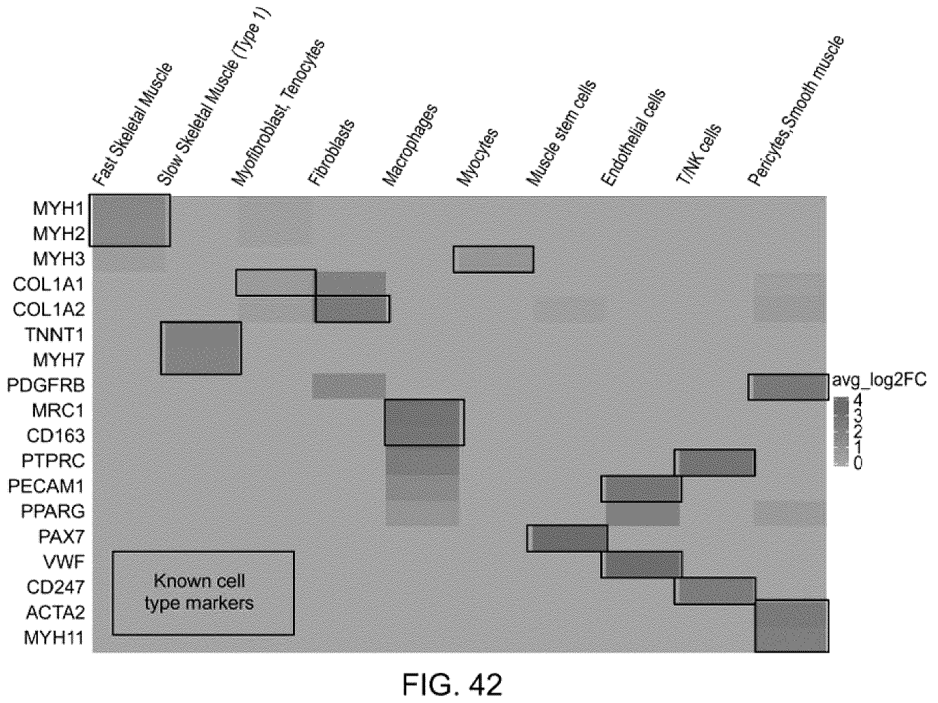

FIG. 42 shows that there is consistency between the top 20 cell-type specific expressed genes and known cell type markers. Plot shows log 2(fold change) in expression in a specific cell-type compared to rest for the top 20 cell-type specific expressed genes. Analysis was performed using all samples. Biomarkers tested include MYH1, MYH2, MYH3, COL1A1, COL1A2, TNT1, MYH7, PDGFRB, MRC1, CD163, PTPRC, PECAM1, PPARG, PAX7, VWF, CD247, ACTA2, and MYH11. For example, MYH1 and MYH2 gene expression were predominantly detected in fast skeletal muscles, which TNT1 and MYH7 gene expression were predominantly detected in slow skeletal muscles.

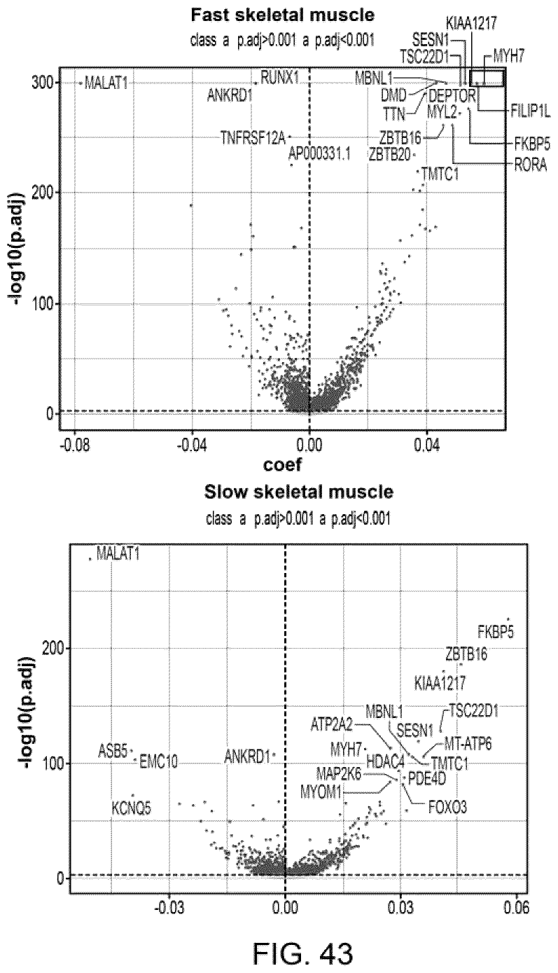

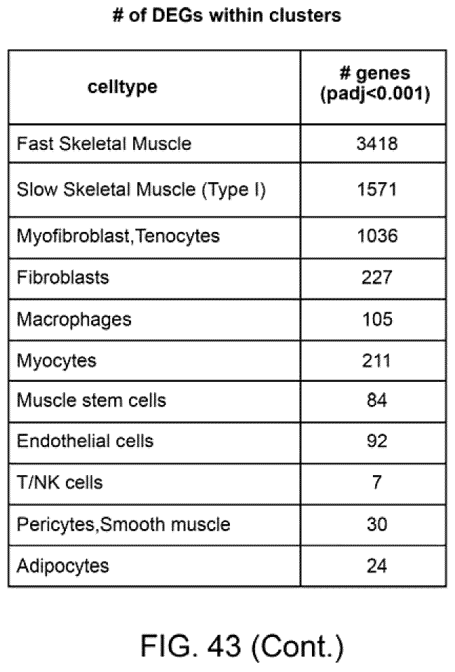

FIG. 43 shows that differentially expressed genes (DEG) associated with BGE105 were identified for each cell types including fast skeletal muscle and slow skeletal muscle.

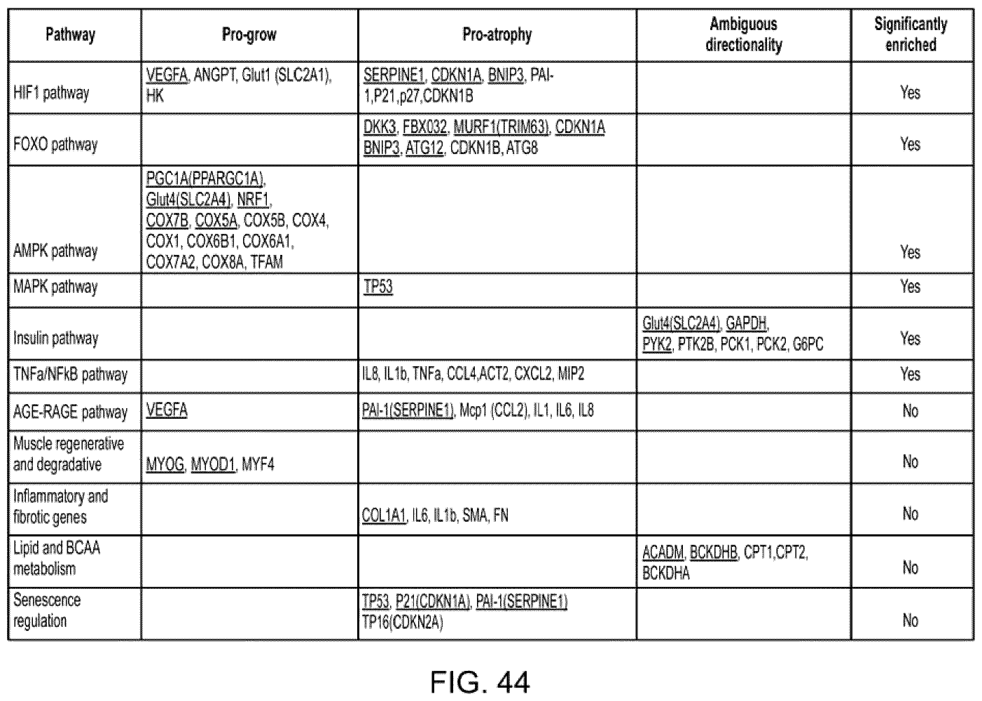

FIG. 44 shows that signaling pathways that control muscle loss and promote muscle growth were enriched in BGE105 treatment associated genes in fast skeletal muscle. Genes with notable significance (padj<0.1) were underscored.

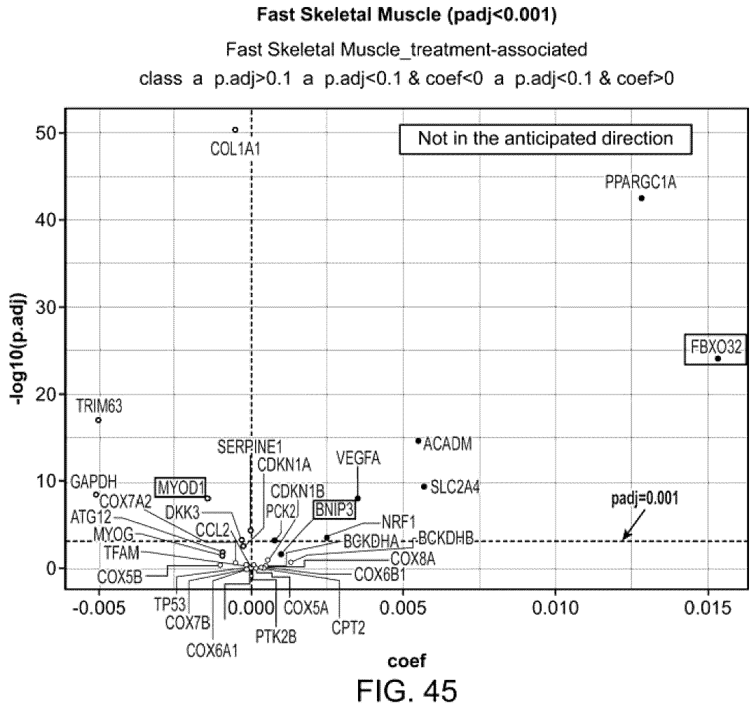

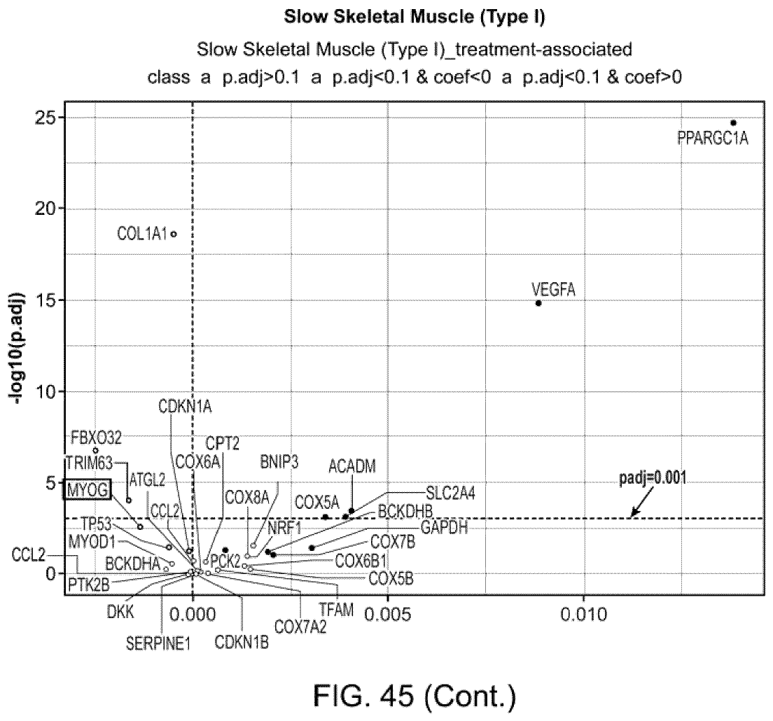

FIG. 45 shows that treatment association of most of significant genes (padj<0.1) in muscle growth/loss related signaling pathways were in the anticipated direction. Shown are detection of genes associated with fast skeletal muscle treatment (padj<0.001) and slow skeletal muscle (Type I) treatment.

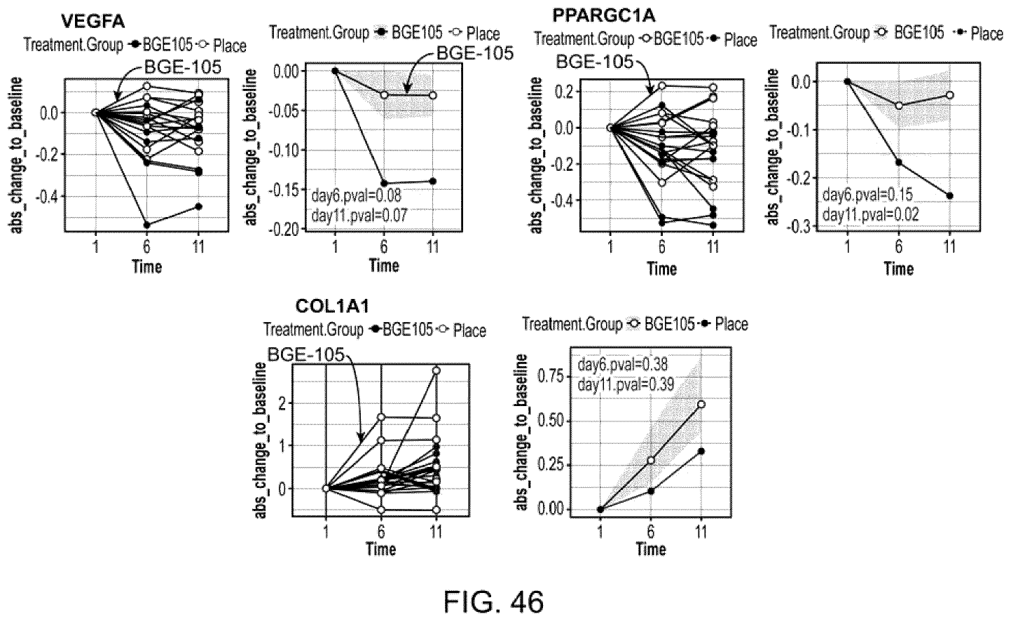

FIG. 46 shows that bulk expression level of VEGFA, PPRGC1A and COL1A1 were higher in treated group than that in placebo group on day 11.

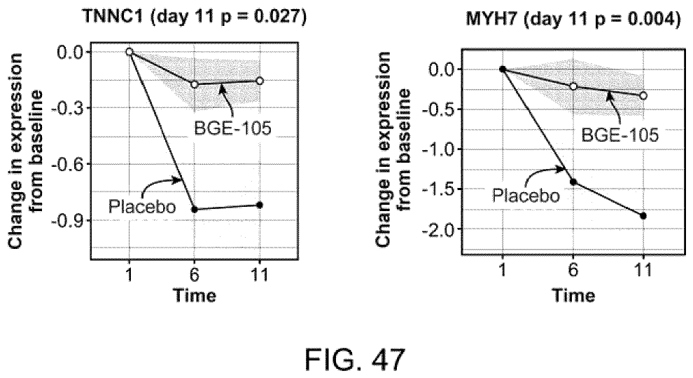

FIG. 47 shows that bulk expression level of TNNC1 and MYH7 were higher in treated compared to placebo on day 11. Shown is bulk expression level: mean of gene across all cells for each patient. Cell types were ignored. TNNC1 and MYH7 are both slow skeletal muscle cell markers.

FIG. 48 shows that for fast skeletal muscle: 10 groups of 5+ enriched pathways were identified. These include pathways involve in: regulation and transport of calcium and metal ions, calcium and calcinenurin-mediated signaling, ERBB2 signaling and associated events in cancer, muscle system processes and contraction, cardiac conduction and muscle contraction, muscle and skeletal system development and differentiation, muscle filament and myofibril organization and assembly, cardiac chamber and ventricular development and morphogenesis, cardiac muscle and heart development and differentiation, and/or cell junction and matric adhesion assembly and organization.

FIG. 49 shows that for slow skeletal muscle: 13 groups of 5+ enriched pathways were identified. These include pathways involve in: regulation of transmembrane transport activities, regulation of lipid storage and foam cell differentiation, muscle and cardiac cell development and differentiation, regulation of cardiac conduction and action potential, cardiac muscle tissue and chamber morphogenesis, muscle contraction and regulation, cytosolic calcium ion transport and regulation, regulation of intracellular calcium release and cardiac muscle contraction, sodium ion transport and regulation, muscle adaption and hypertrophy, myotube adaptation and hypertrophy, cardiac conduction and multicellular organismal signaling, and/or muscle fiber and myofibril assembly and development.

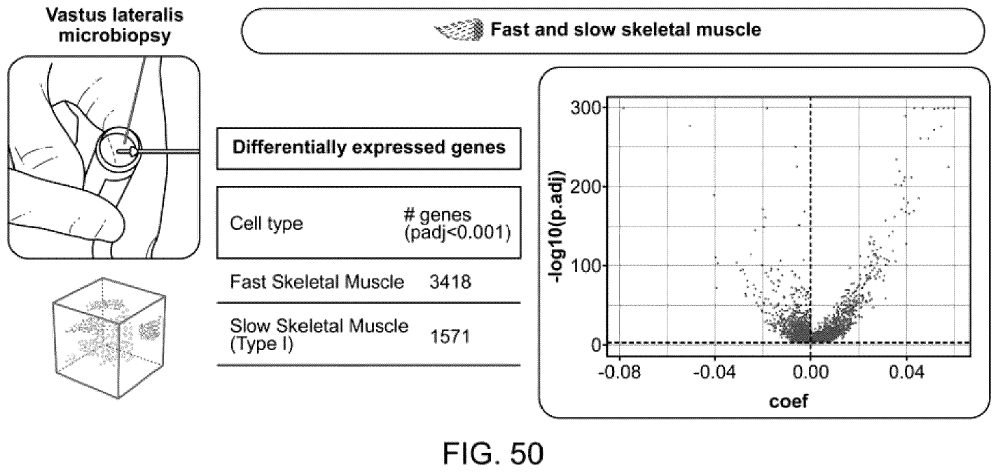

FIG. 50 shows cell type-specific patterns of differential gene expression associated with BGE-105 treatment identified in muscle biopsies collected from vastus lateralis. Shown are differentially expressed genes detected in fast skeletal muscle (3418) and slow skeletal muscle (Type 1) (1571).

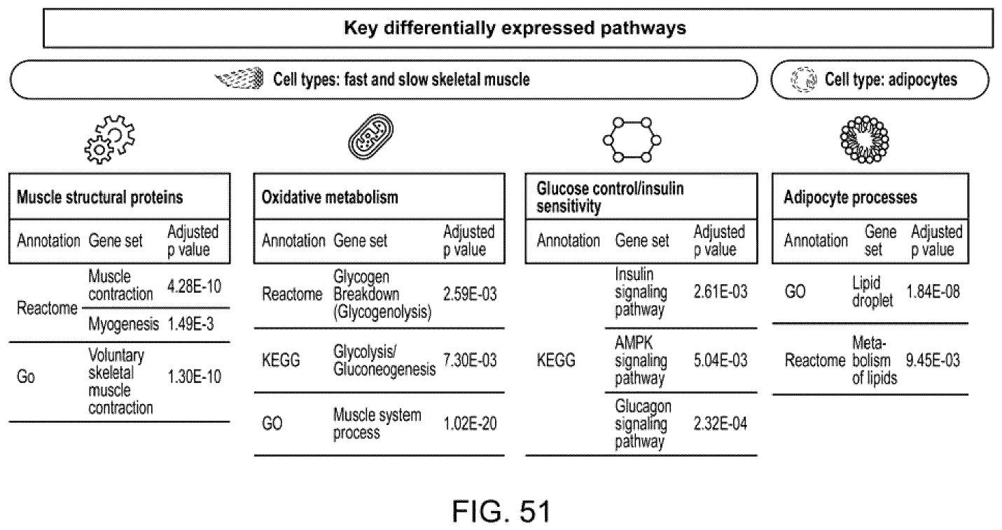

FIG. 51 shows that differentially regulated pathways suggest BGE-105 beneficial effects on key muscle and adipocyte processes.

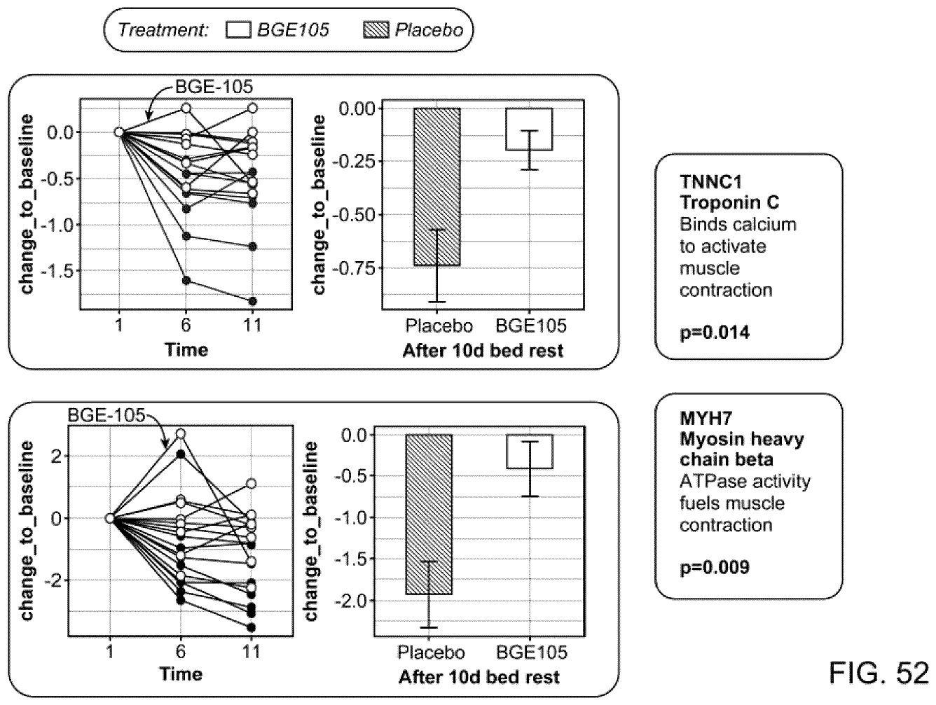

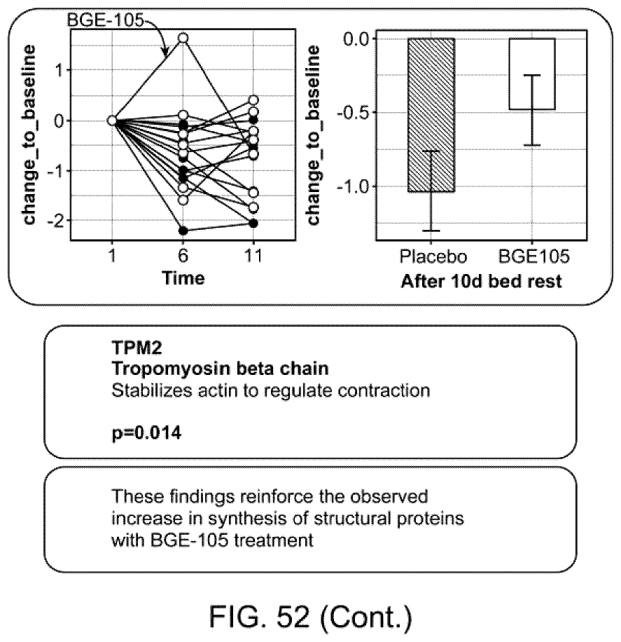

FIG. 52 shows BGE-105 prevents bed rest-induced downregulation of muscle contractile proteins in fast and slow skeletal muscles. p values reflect change after 10 days of bedrest, compared to baseline. BGE-105 prevents reduction of troponin C (TNNC1), which binds calcium to activate muscle contraction (p=0.014); myosin heavy chain beta (MYH7), which is involved in ATPase activity fuels muscle contraction (p=0.009); and tropomyosin beta chain (TPM2), which stabilizes actin to regulate contraction (p=0.014). These findings reinforce the observed increase in synthesis of structural proteins with BG5-105 treatment.

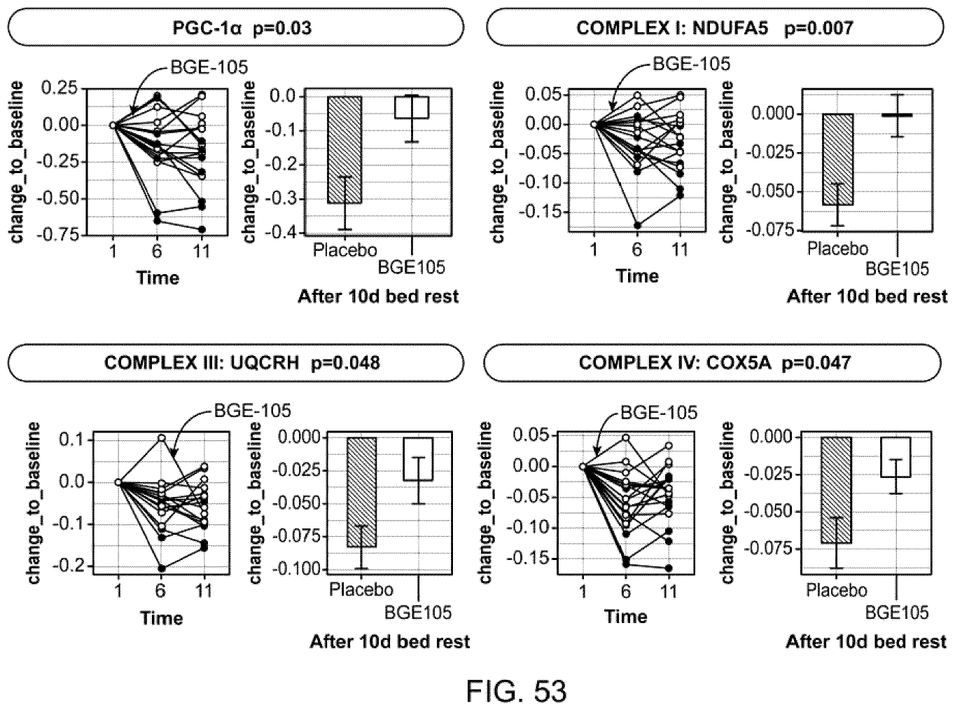

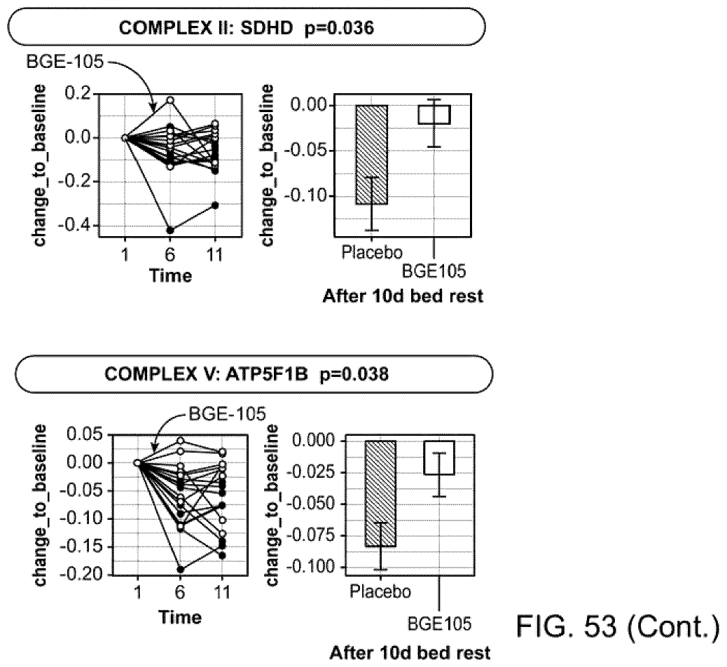

FIG. 53 shows BGE-105 prevents bed rest-induced downregulation of mitochondrial biogenic regulator PGC-1α and all respiratory complexes. Shown are representative genes including PGC-1α (p=0.03), COMPLEX 1: NDUFA (p=0.007), COMPLEX II: SDHD (p=0.036), COMPLEX III: UQCRH (p=0.048), COMPLEXT IV: COX5A (p=0.047), COMPLEX V: ATP5F1B (p=0.038).

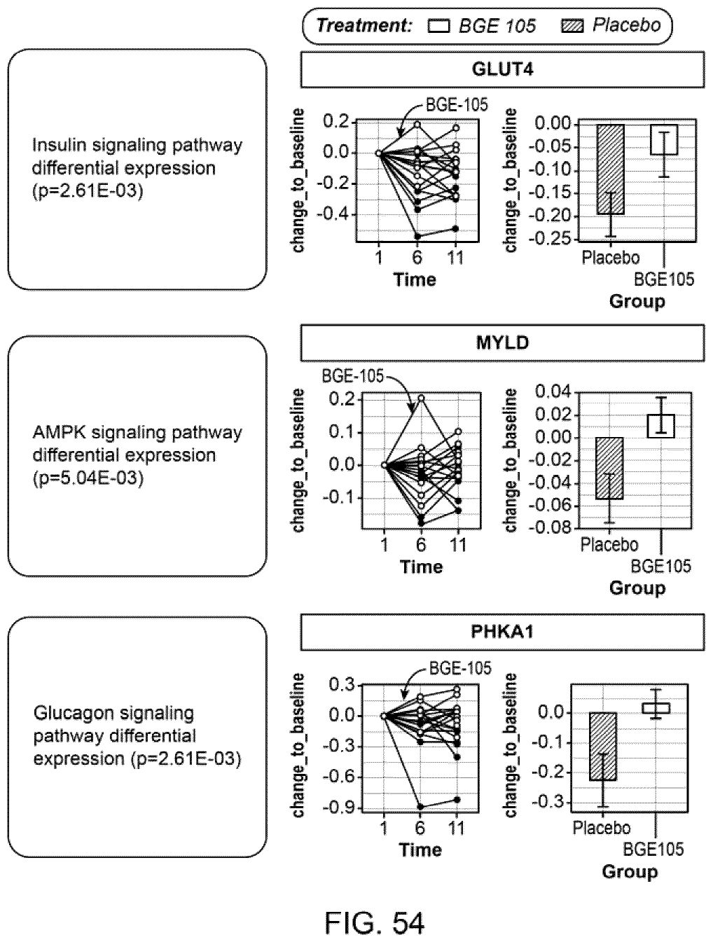

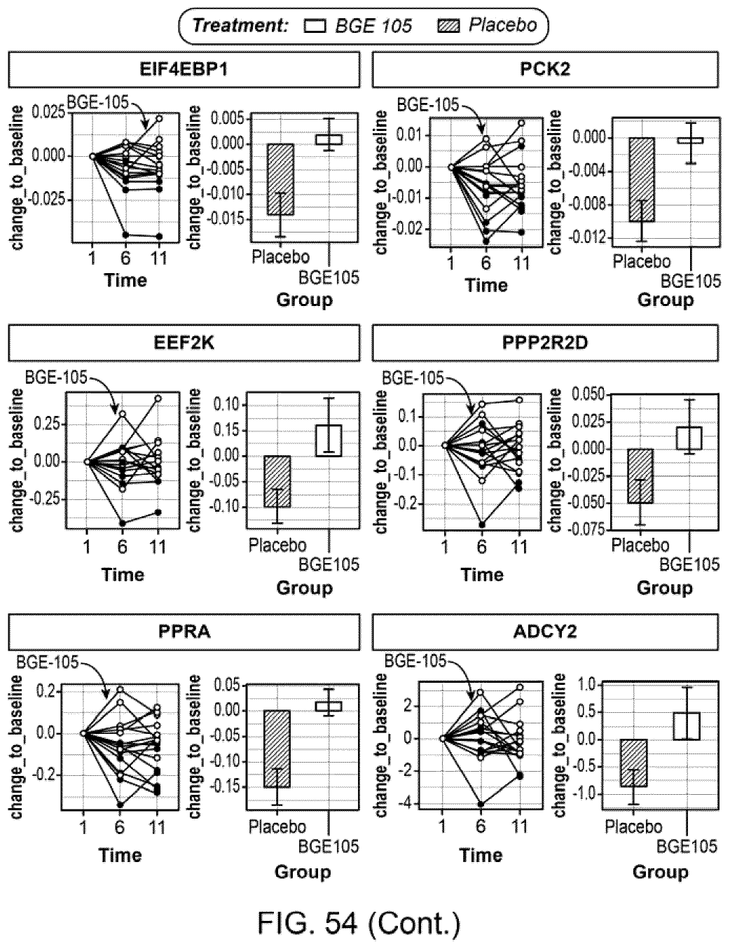

FIG. 54 shows BGE-105 prevented detrimental expression level of genes involved in muscle metabolic processes. Shown are differential gene expression of insulin signaling pathway (p=2.61E-03), AMPK signaling pathway (p=5.04E-03), and glucagon signaling (p=2.61E-03). Exemplary genes are GLUT4, EIF4EBP1, PCK2 (insulin signaling pathway); MYLD, EEF2K, PPP2R2D (AMPK signaling pathway); and PHKAA1, PPRA, ADCY2 (glucagon signaling pathway).

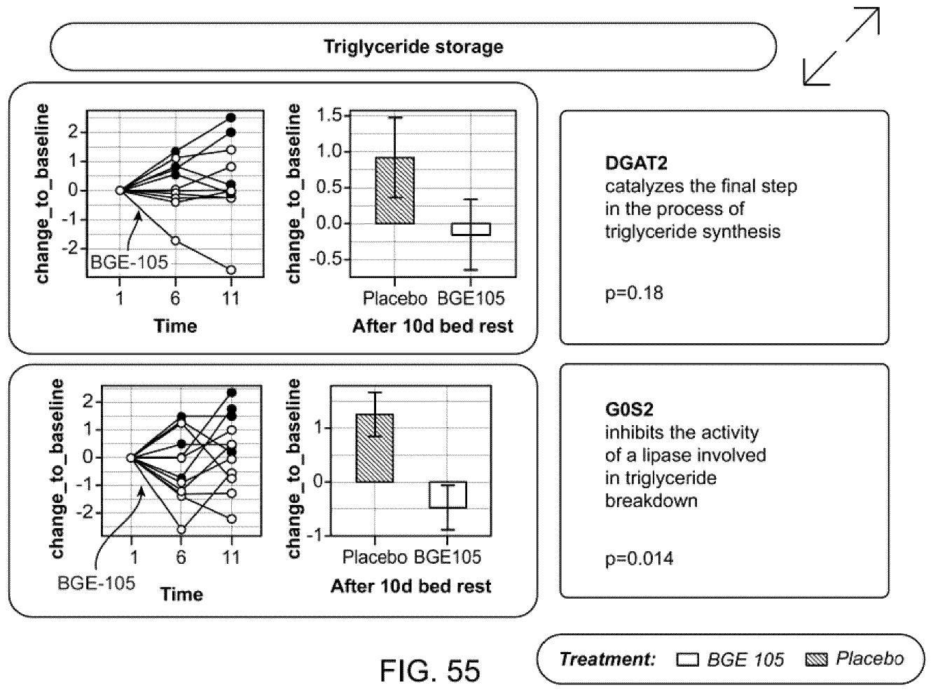

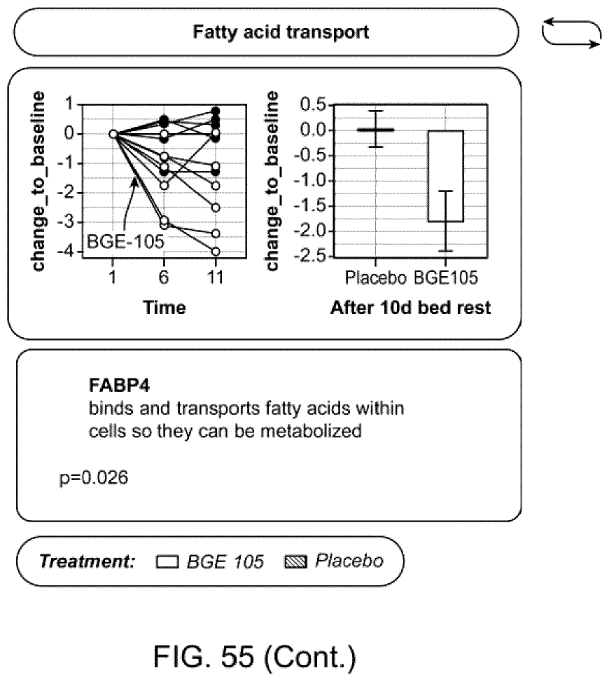

FIG. 55 shows BGE-105 prevents bed rest-induced upregulation of genes involved in triglyceride storage and fatty acid metabolism, potential mechanism for promoting fat loss. Exemplary genes include DGAT2, which catalyzes the final step in the process of triglyceride synthesis (p=0.18); GOS2, which inhibits the activity of a lipase involved in triglyceride breakdown (p=0.014); and/or FABP4, which binds and transports fatty acids within cells so they can be metabolized (p=0.026).

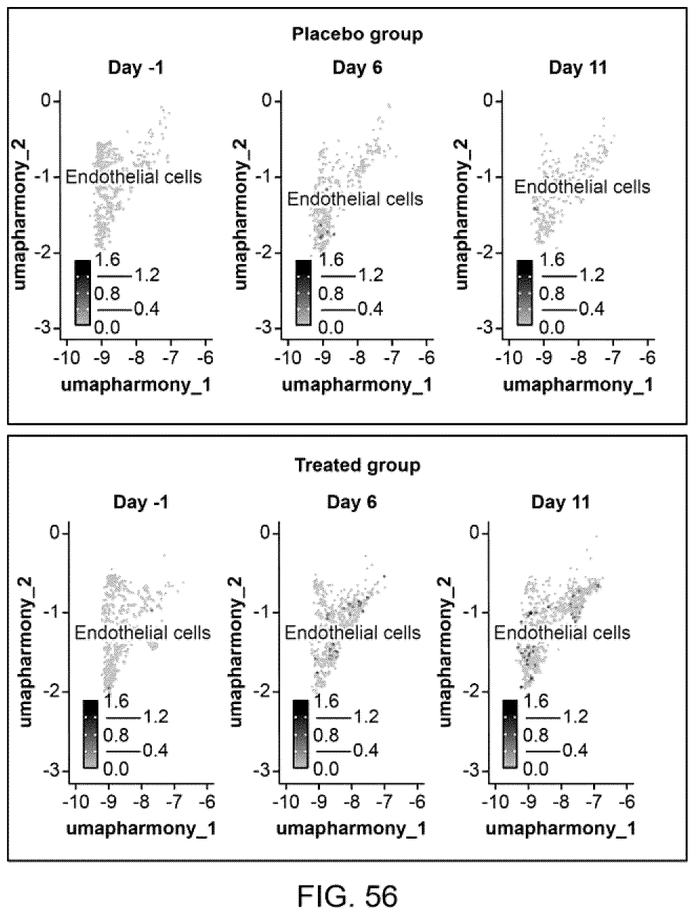

FIG. 56 shows APLNR was expressed in more Endothelial cells in the treated group. A small proportion of cells expressed APLNR (272 cells). 46% of these cells were within the endothelial cells. T-test of APLNR expression level in detected cells between treated and placebo groups: Day6_t.stat=2.02, Day6_p.val=0.04, Day11_t.stat=2.33, Day11p.val=0.02.

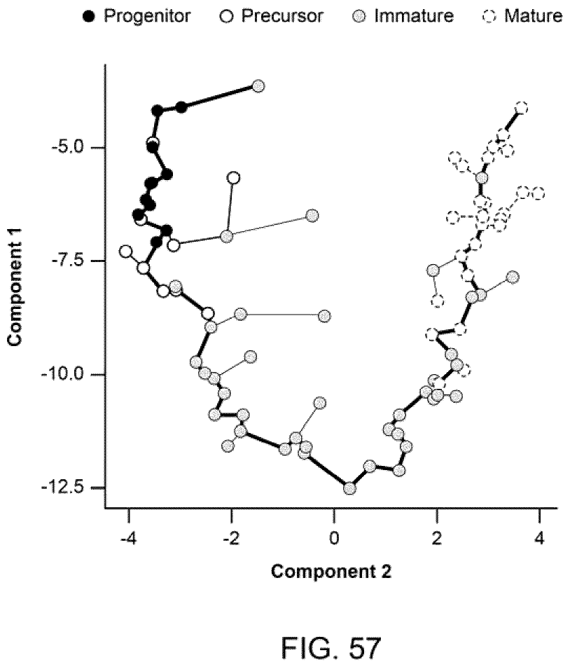

FIG. 57 Cell differentiation trajectory and pseudotime inference. Pseudotime inference for cell differentiation is a computational approach to model dynamic changes and transitions between different cellular states such as transition between progenitor, precursor, immature, and mature cell states. It allows for the distinction between early and late stages of a biological process. Pseudotime analysis can help uncover the chronological sequence of gene expression changes during a biological process, thereby allowing us to understand the underlying molecular mechanisms.

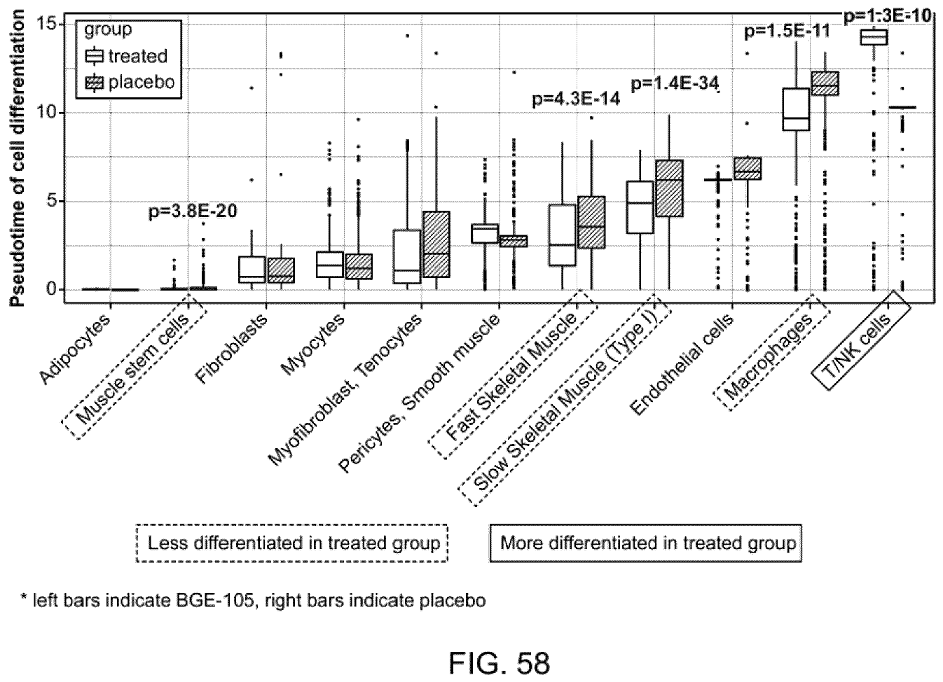

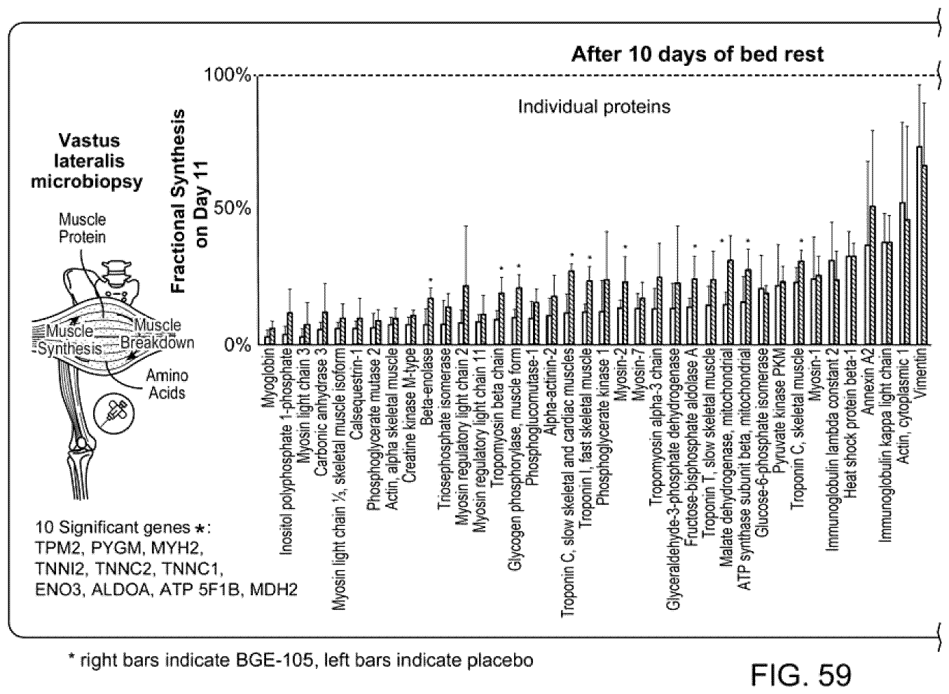

FIG. 58 shows a plot indicating cells were less differentiated with treatment for Fast/Slow skeletal muscle, macrophages, T/NK cells and muscle stem cell. Baseline muscle stem cells (treated and placebo) were set as the root. As shown, muscle stem cells, fast skeletal muscle, slow skeletal muscle (Type I), and macrophages are less differentiated in treated group, whereas T/NK cells are more differentiated in treated group.

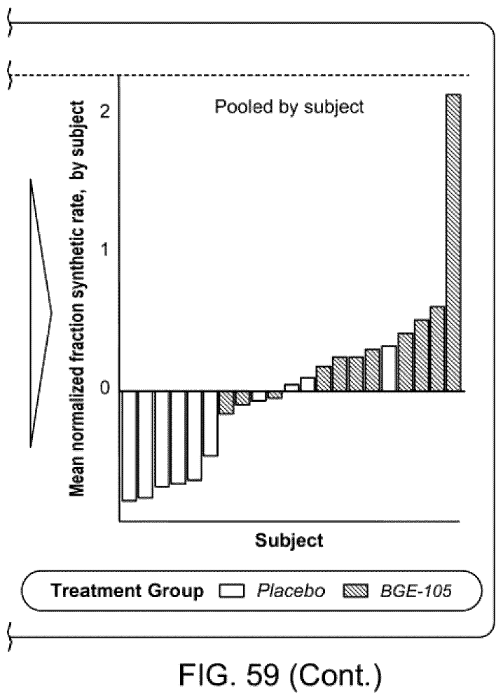

FIGS. 59-61 illustrate the results of secondary analyses. Evaluation of signal from protein synthesis rate analysis. Investigation of aging and muscle signatures from published research.

FIG. 59 shows BGE-105 resulted in relatively higher muscle protein synthesis in the vastus lateralis, measured via microbiopsy (p<0.005). Shown is muscle protein synthetic rate after 10 days of bed rest. Asterisks indicate 10 significant genes: TPM2, PYGM, MYH2, TNNI2, TNNC2, TNNC1, ENO3, ALDOA, ATP 5F1B, MDH2.

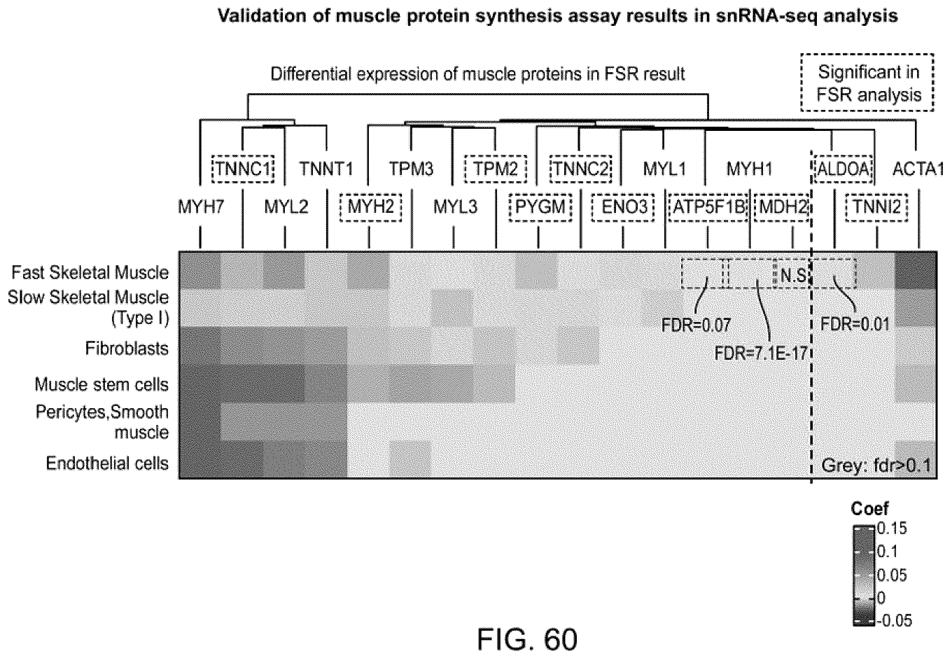

FIG. 60 shows the validation of muscle protein synthesis assay results in snRNA-seq analysis. 15 out of 18 muscle proteins show statistically highly expressed in fast skeletal muscle in treated compared to the placebo group. 13 myofibril proteins (5 proteins were significant in FSR analysis: TPM2, TNNC1, TNNC2, TNNI2, MYH2) and 5 significant non-myofibril proteins in FSR analysis (5 proteins: ENO3, PYGM, ALDOA, MDH2 ATP5F1B). All 38 proteins in the heavy-water were also measured in snRNA-seq data.

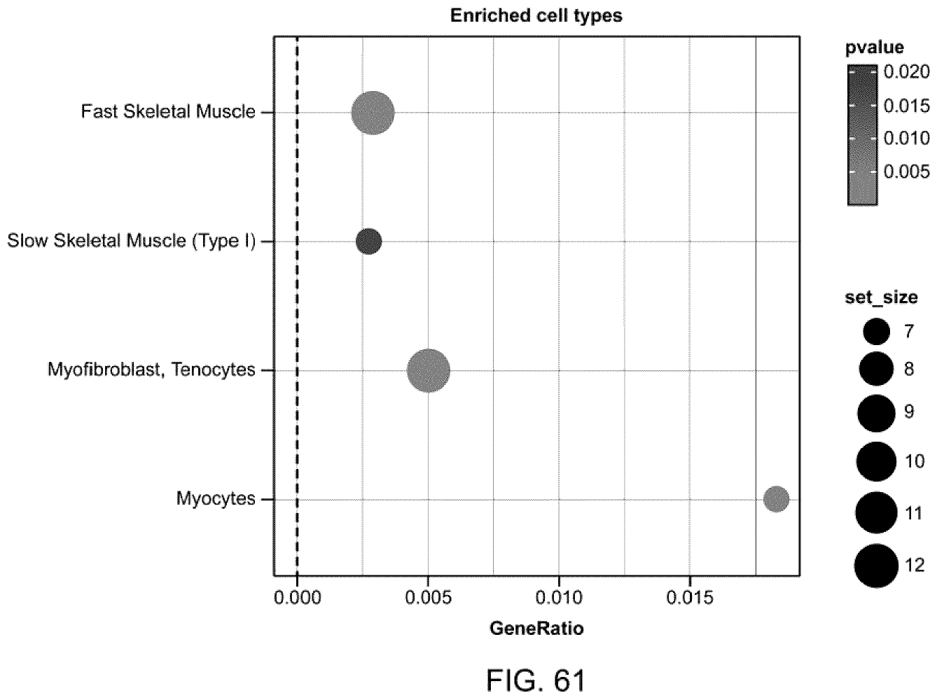

FIG. 61 shows that BGE-105 treatment shifts the transcriptome of fast/slow skeletal muscles towards a state linked to younger muscle. Transcripts negatively associated with BGE-105 treatment showed enrichment for the muscle aging signature. The result correlates with report on gene expression changes in old vs young muscle (see e.g., Perez et al., 2022).

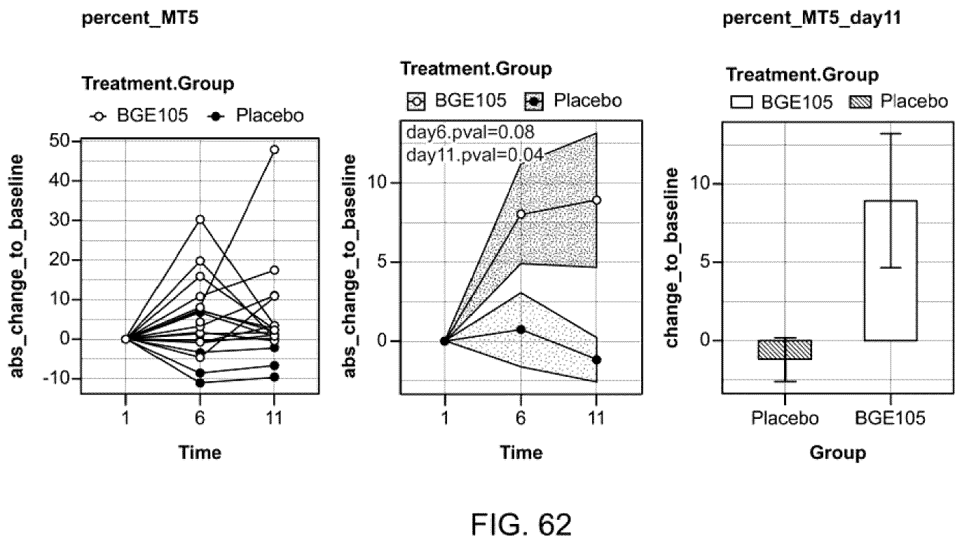

FIG. 62 shows graph that indicate percentage of cells that have Mitochondria reads more than 5% within a sample. Y-axis is the percentage of cells that MT reads >5% within a sample (62 samples=21 patients*3 time points) compared to that in baseline. P values of T-test are day 6, p=0.08; day 11, p=0.04. Cells that have low qualities have been removed before calculating the proportion of cells that have more than 5% Mitochondrial reads for each patient (cells with less than 200 genes or less than 500 reads were removed, total remaining MT cells (>5%).

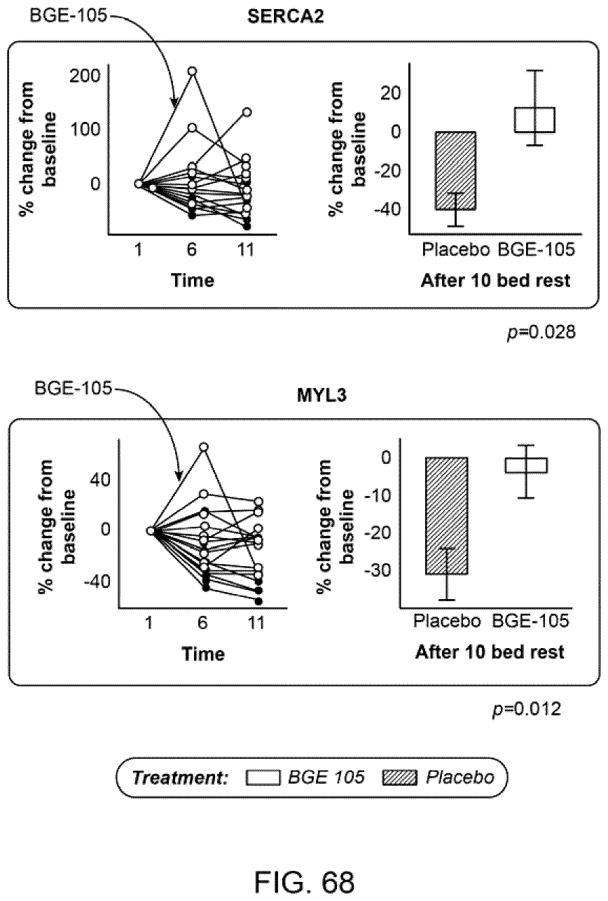

FIG. 68, left and right panels, show BGE-105 prevented bed rest-induced downregulation of muscle contractile proteins in fast and slow skeletal muscle. Shown are single-nucleic transcriptomics data of percentage change from baseline for calcium-ATPase type 2 in the sarco-/endoplasmic reticulum (SERCA2) and myosin light chain 3 (MYL3). As shown, BGE-105 treatment prevented downregulation of SERCA2 (p=0.028) or MYL3 (p=0.012) after 10 days bed rest when compared to placebo. p values reflect change after 10 days of bedrest, compared to baseline.

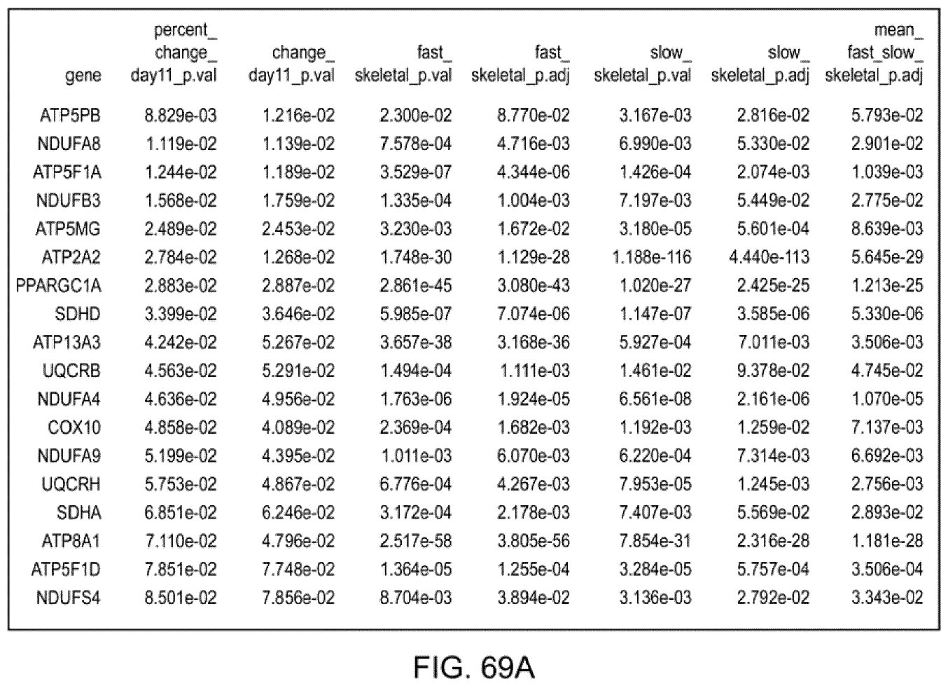

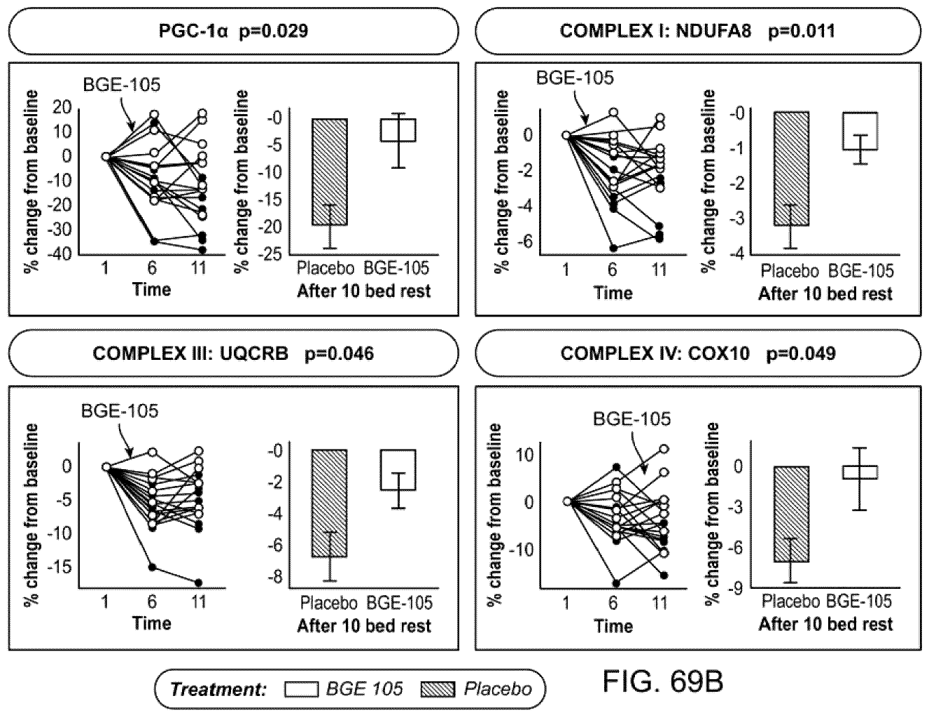

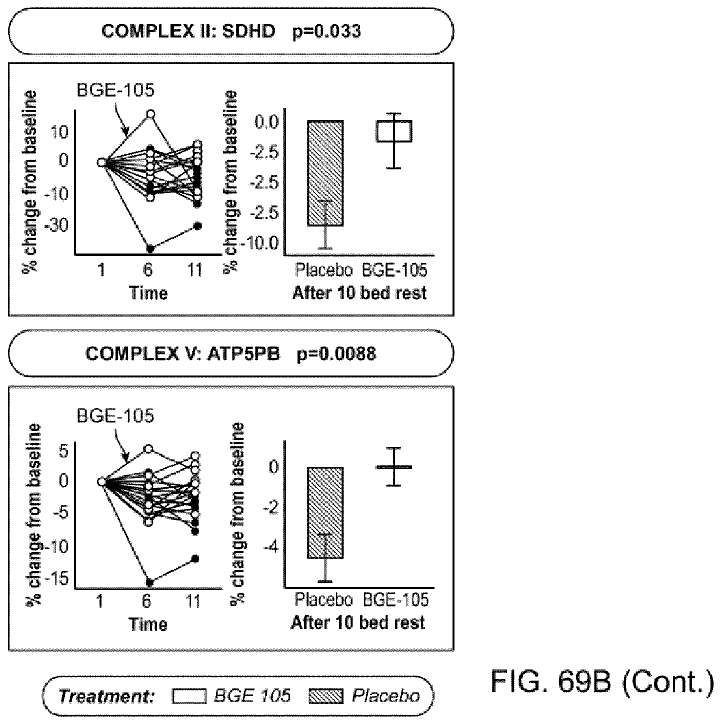

FIGS. 69A-69B show BGE-105 prevented bed rest-induced downregulation of mitochondrial biogenic regulator PGC-1α and all respiratory complexes. FIG. 69A shows a summary of downregulation of respiratory complexes. Exemplary genes include ATP5PB, NDUFA8, ATP5F1A, NDUFB3, ATP5MG, ATP2A2, PPARGC1A, SDHD, ATP13A3, UQCRB, NDUFA4, COX10, NDUFA9, SDHA, ATP8A1, ATP5F1D, and/or NDUFS4. FIG. 69B shows expression of representative genes in BGE-105 treated and placebo subjects. For example, BGE-105 prevented downregulation of PGC-1α (p=0.029), NDUFA8 (p=0.011), SDHD (p=0.033), UQCRB (p=0.046), COX10 (p=0.049), and/or ATP5PB (p=0.0088). p values reflect change after 10 days of bedrest, compared to baseline.

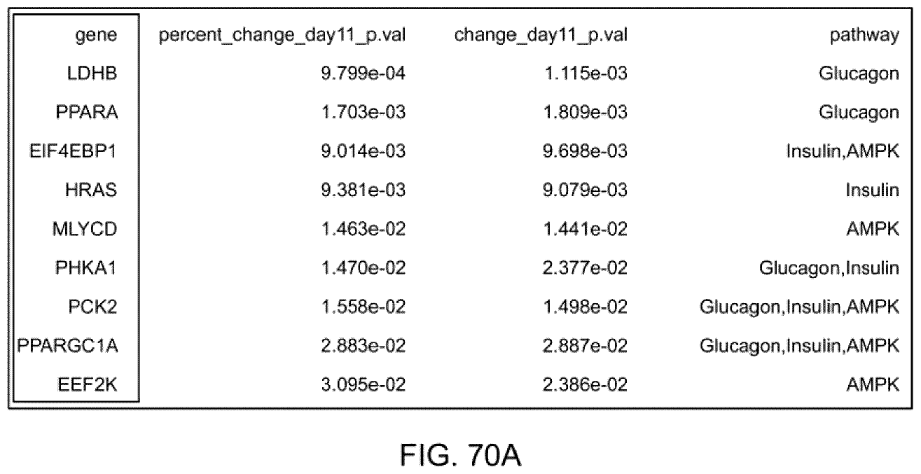

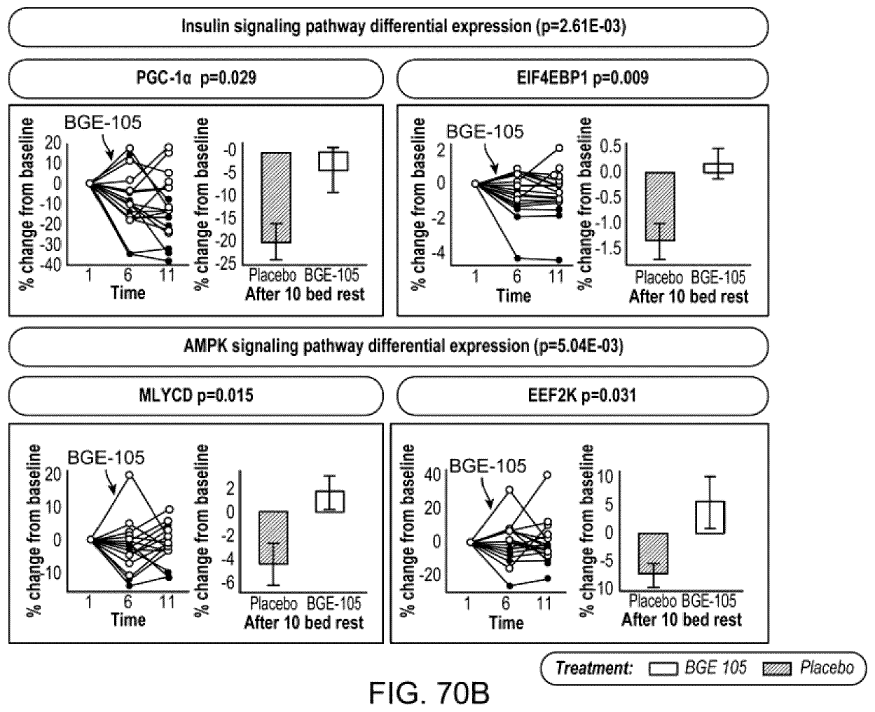

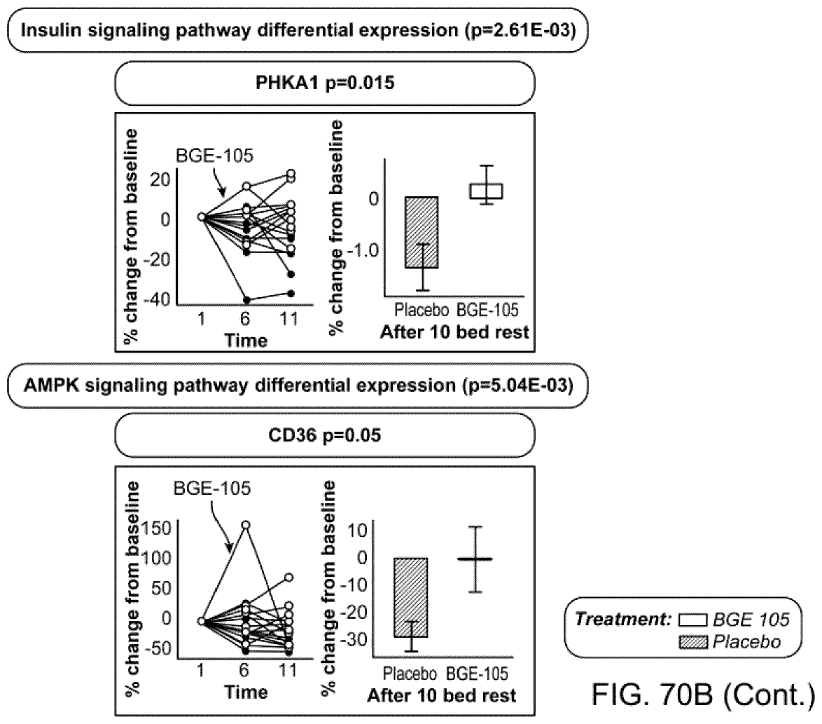

FIGS. 70A-70B show BGE-105 preserved gene expression involved in glucose metabolism. p values reflect change after 10 days of bedrest, compared to baseline. Exemplary genes include LDHB, PPARA, EIF4EBP1, HRAS, MLYCD, PHKA1, PCK2, PPARGC1A, EEF2K. FIG. 70A shows a summary of preserved gene expression in glucagon, insulin and AMPK pathways. FIG. 70B shows insulin signaling pathway differential expression (p=2.61E-03) and AMPK signaling pathway differential expression (p=5.04E-03). For example, BGE-105 prevented downregulation of PGC-1α (p=0.029), EIF4BP1 (p=0.009), PHKA1 (p=0.015), MLYCD (p=0.015), EEF2K (p=0.031), and/or CD36 (p=0.05).

In conclusion, patients who are responding to BGE-105 treatment and are likely to have reduced muscle atrophy.

### 7.15. Example 15: Enhancing Resilience in Hospitalised Elderly COPD Patients Through Prevention of Muscle Atrophy

COPD Patients are at Risk for Loss of Resilience

Acute exacerbation of chronic obstructive pulmonary disease (AECOPD) is the second most common cause of emergency admission to hospital in the UK with over 100,000 admissions per year. Of these admissions, 20% of patients are readmitted within 30 days, making AECOPD the leading cause of hospital readmission. A key factor driving these readmissions is COPD-associated muscle dysfunction, which increases skeletal muscle atrophy and mortality, and prevents a return to baseline function.

The important impact of skeletal muscle atrophy associated with repeated AECOPD is clear: muscle atrophy leads to a deterioration of resilience with an even higher occurrence of muscle atrophy developing as a direct result of immobility due to hospitalisation for acute exacerbations of COPD.

Pulmonary Rehabilitation is Insufficient for AECOPD Recovery

Despite receiving standard-of-care rehabilitation during hospitalisation and post-hospital discharge, older COPD patients experience significant muscle loss, often associated with longer length of hospital stay, and with increased short-term and long-term morbidity and mortality (12% mortality rate at 90 days post-hospitalisation compared to 4.9% for myocardial infarct mortality).

Although clinical trials have demonstrated that post-exacerbation pulmonary rehabilitation (PEPR) has the potential to increase physical function and reduce the risk of re-hospitalisation through exercise programs, education, and behaviour interventions, PEPR is less effective in practice because fewer than 10% of AECOPD patients complete PEPR in this context. Even if PEPR is completed, this program may not prevent acute loss of resilience because it is initiated after the most acute and proinflammatory phase of the illness. Earlier intervention might be more successful in preventing acute muscle wasting; however, in the largest trial conducted in this population, an exercise intervention could not be delivered with sufficient intensity to confer long term reduction in hospital readmission or increments in physical performance and health status. The development of acute muscle wasting, therefore, represents an important unmet need in the patient with AECOPD and a potential trait treatable by muscle-targeted drug therapies.

Apelin Pathway Activation Attenuates Muscle Loss and Improves Frailty in Nonclinical and Clinical Studies

Apelin is an endogenous peptide exerkine that primarily targets skeletal muscle tissue. Apelin has been implicated in improving muscle regeneration and stem cell activation while reducing muscle atrophy and inflammation. In BioAge's human aging cohort, apelin module activation predicted human longevity, demonstrating that apelin levels are strongly associated with lifespan and healthspan (e.g., muscle strength). Additionally, apelin levels typically decrease with age, further highlighting the potential importance of apelin agonists in older populations. BGE-105 (also known as azelaprag) is a small-molecule apelin receptor agonist licensed for development in muscle aging.

BGE-105 in Nonclinical Models

Nonclinical studies in rodents demonstrated the ability of BGE-105 to mitigate the muscle and strength loss seen in various models of muscle loss.

BioAge evaluated treatment with BGE-105 in a muscle disuse atrophy mouse model. Loss of muscle mass was induced by hindlimb casting: the right hind leg was immobilized by casting for 14 days. The left hind leg remained mobile and served as an intra-individual control. Treatment with BGE-105 prevented the loss of muscle mass in the tibialis anterior of the casted limb relative to the non-casted limb compared to the muscle loss in vehicle treated animals (p<0.0001).

BioAge evaluated daily treatment with BGE-105 versus vehicle only in aged mice (23-24 months old) over 2 months. This study demonstrated statistically significant improvements for the azelaprag-treated animals in total daily distance run (exercise wheel) and grid hang times (upside down grip strength measure).

BGE-105 in Disuse Atrophy Clinical Study

BioAge completed a double-blind, non-randomized Ph1b trial in healthy older volunteers (≥65 years old) who underwent 10 days of continuous bedrest. BGE-105 (240 mg) or placebo was administered once daily intravenously for 10 days, and normal activities were resumed on Day 11. Muscle atrophy was accessed at one day before (D-1) initiation of bedrest (baseline), 5 days (D5) and 10 days (D10) after bedrest.

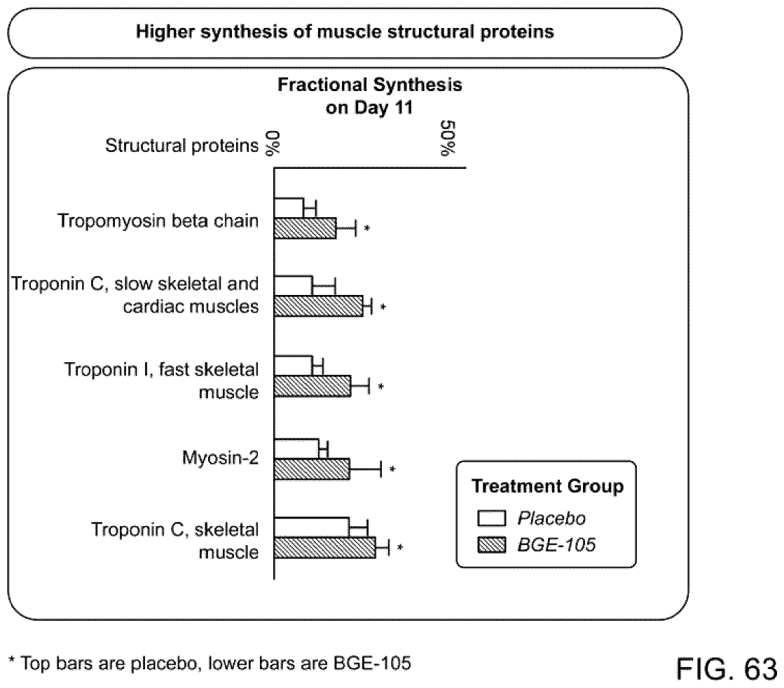

The study demonstrated that compared to placebo, BGE-105 significantly reduced muscle loss as measured by vastus lateralis muscle thickness (75% reduction in loss) and cross-sectional area (>50% reduction in loss) (FIG. 19). This preservation of muscle mass was supported by the maintenance of skeletal muscle fractional protein synthetic rates (as measured by D2O method and muscle biopsy). Crucially, the synthesis of myofibrillar structural proteins such as myosin and troponin was preserved compared to placebo (FIG. 63).

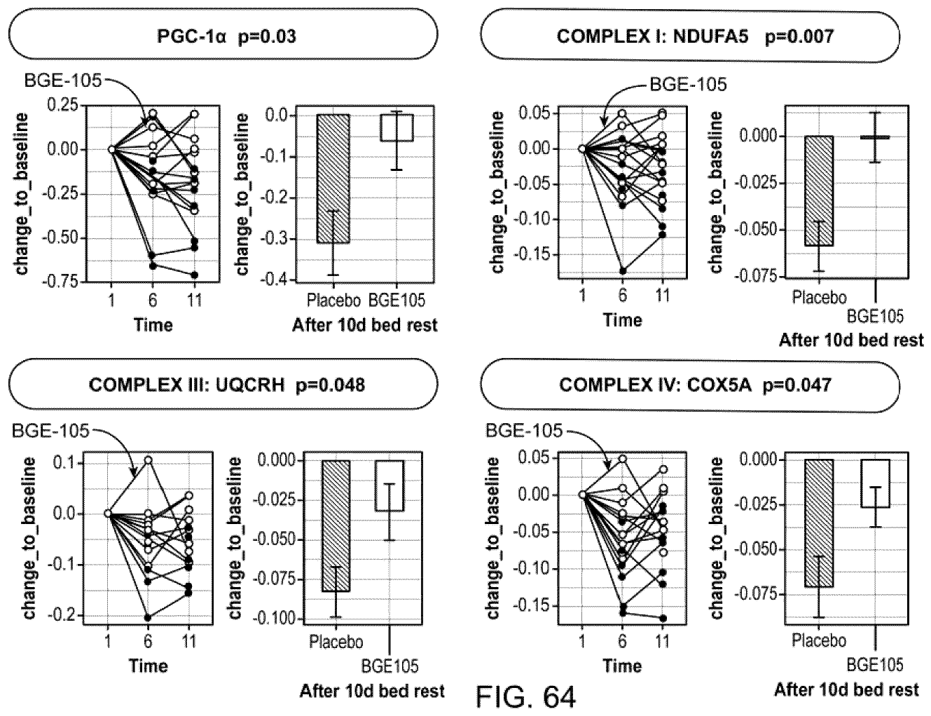

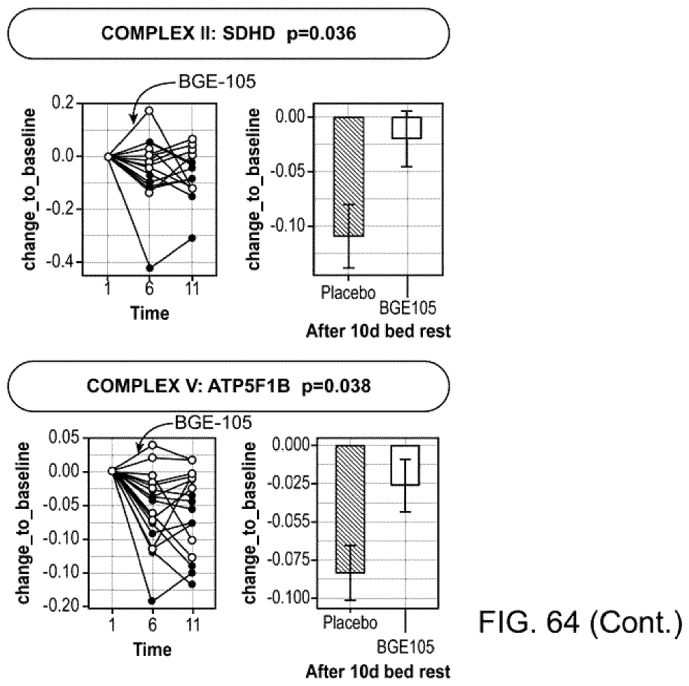

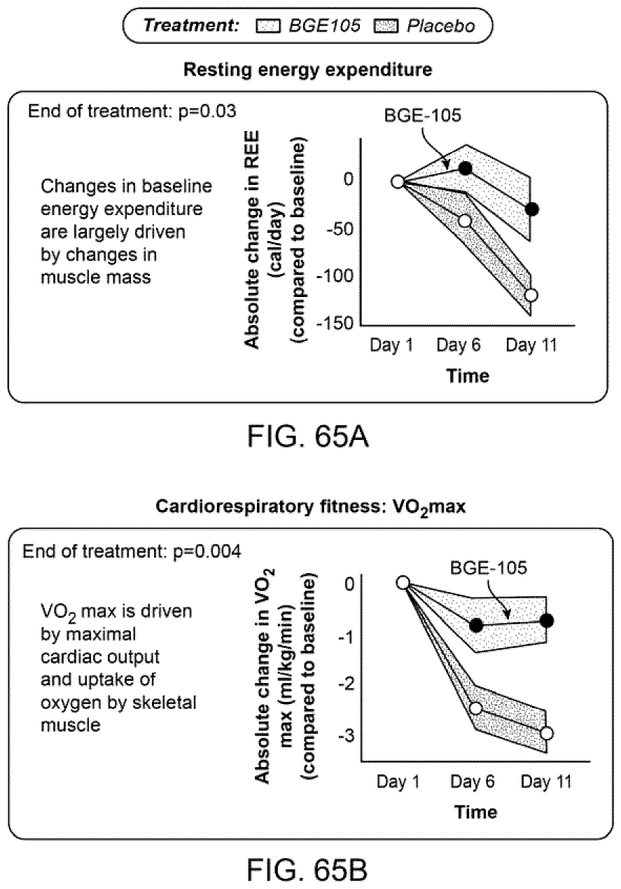

Transcriptomics (by single-nucleus RNAseq) of muscle tissue from study subjects demonstrated that BGE-105 prevented bed rest-induced downregulation of mitochondrial biogenic regulator PGC-1α and all respiratory complexes (FIG. 64). At the proteomic level, BGE-105 treatment shifted the plasma proteome towards an estimated higher basal metabolic rate and VO₂ max by SomaSignal test (FIG. 65). Previous studies in hospitalised patients documented the challenge of overcoming the anabolic resistance in this patient population, wherein nutritional supplement fails to maintain muscle protein synthesis and exercise training is a barrier for older, frail patients.

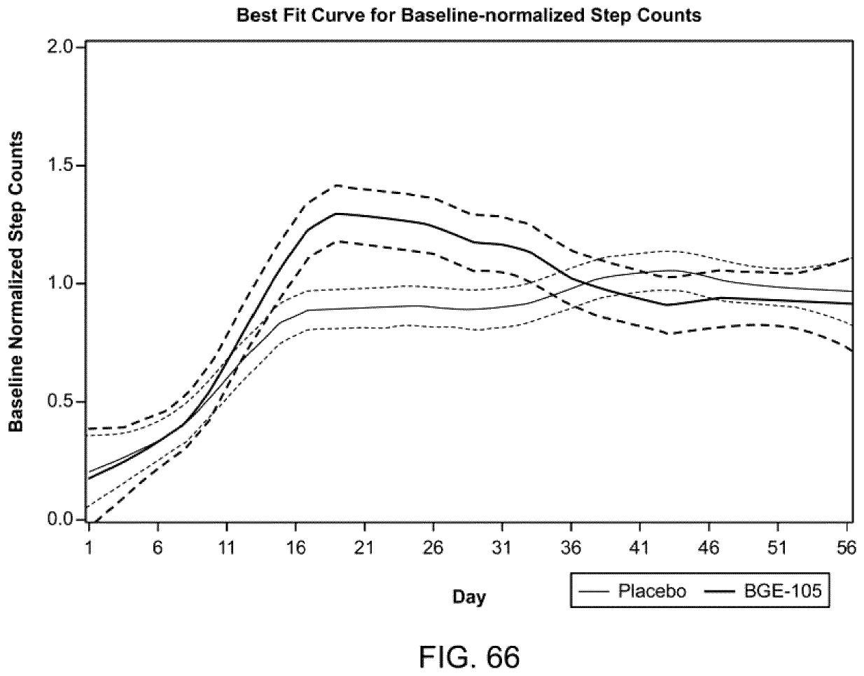

This study piloted wearable activity monitoring; encouraging accelerometry results demonstrated that post-bedrest recovery of activity (starting on Day 11; bedrest and BGE-105/placebo treatment occurred from Day 1 to 10) was more pronounced in the BGE-105 group (FIG. 66).

This provides a human model akin to acute hospitalisation in COPD, and serves as proof-of-concept that acute muscle loss can be ameliorated by BGE-105 regardless of nutritional and exercise capacity.

In summary, previous preclinical and clinical studies demonstrate target engagement, efficacy, including improvements in both overall activity and muscle atrophy, favorable pharmacokinetics, and human safety in over 200 subjects shown in a series of Phase 1a/1b trials. BGE-105 is a highly selective and potent agonist of the apelin receptor. BGE-105 has been shown to preserve muscle size and quality in older volunteers (≥65 years old) compared to placebo in a 10-day bed rest study.

BGE-105 can mitigate acute muscle loss and promote functional improvements by multiple mechanisms including prevention of diminishing muscle protein synthesis and improved metabolic function. Improved resilience may also impact long-term rehospitalisation risk in this population because of the preservation of muscle mass and strength associated with use of BGE-105 during prior hospitalisation. Therefore, addressing frailty and muscle loss is critical to improve outcomes for these patients. This disclosure provide methods to intervene and prevent muscle atrophy to decrease frailty risk, improve resilience and support recovery.

Study

To address this devastating disease and the cycle of resilience deterioration, this disclosure provide methods to prevent acute skeletal muscle wasting effects during hospitalisation for AECOPD by treating subjects with an apelin receptor APJ agonist (BGE-105) started while in hospital.

Target Population

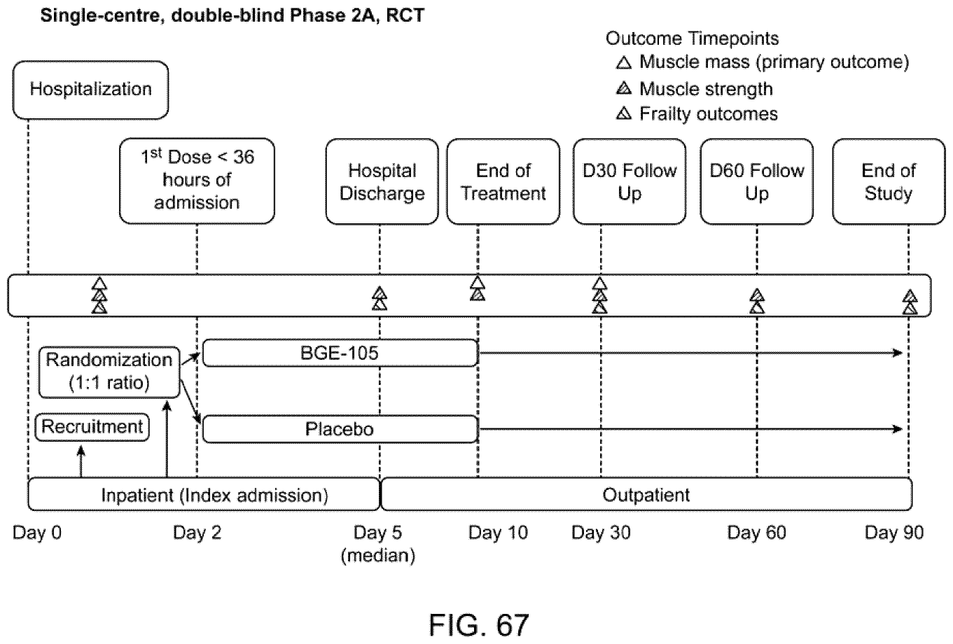

This randomized, double blind, Phase 2a study will treat 60 older subjects (30 each, BGE-105 and placebo) who are hospitalised for AECOPD and at risk for prolonged hospitalisation (>5 days). Subjects at risk for prolonged hospitalisation is defined as: ≥60 years old, previously hospitalized for AECOPD, and/or have Modified Medical Research Council (mMRC) Dyspnea Scale ≥2. FIG. 67 shows a flow chart of the single-center, doubled-blinded Phase 2a, RCT clinical trial.

Intervention and Control

BGE-105 (or placebo) is administered via daily one-hour IV infusion for 10 days. Subjects are followed for 90 days for muscle mass, function, readmission, and mortality. The primary objective is to assess whether BGE-105 (240 mg, 1440 mg) results in the preservation of muscle mass following admission to hospital with an exacerbation of COPD compared with usual care.

First dose (BGE-10 or placebo) is administered on Day 2, less than 36 hours of admission. Patients are discharged on Day 5 and the treatment ends on Day 10. Beginning Day 5 until Day 90 post initiation of the treatment, patients are required to return for follow up visits at Day 30 and Day 60 until end of study on Day 90.

Outcomes

The primary outcome is muscle mass. Other outcomes are muscle strength and/or frailty outcomes. Based on a difference of 50% reduction of muscle loss (supported by the phase 1b bed rest study), 30 subjects per study arm are required for a proof-of-concept study.

This study correlates measurements to understand the treatment with BGE-105 and determine if resilience is maintained or improved in subjects who are hospitalised for exacerbation of COPD. These measurements include but are not limited to grip strength before and after treatment, functional/performance measurement (e.g., Short Physical Performance Battery [SPPB]), muscle mass by D3-creatine, muscle protein synthesis by D2O by microbiopsy, composition and change in limb muscle by biopsy, ultrasound, DEXA, and CT, mortality and readmission at Days 30, 60, and 90, and quality of life assessments. Success is achieved through maintenance of skeletal muscle strength, mass, and quality due to improved protein synthesis resulting in lower rates of readmission and possibly shorter duration of initial hospital stay.

Study Aim

The purpose of this proposed study is to intervene and prevent muscle atrophy to improve frailty risk, resilience, and recovery.

COPD patients hospitalised with an exacerbation are a vulnerable group with more than half (56%) fulfilling the criteria of physical frailty (by Fried's frailty criteria, see e.g., Fried et al., 2001. Biol Sci Med Sci. 2001 March; 56(3):M146-56. doi: 10.1093/gerona/56.3.m146. PMID: 11253156), and only 5% being physically robust. This frail population has both a lower muscle mass at time of admission and a larger loss in muscle mass during their hospital admission, compounding the effect of the event. Data suggests that this loss occurs early in the hospital admission, with the majority experiencing a clinical significant loss of more than 5% muscle mass (quadriceps).

In this study, loss of muscle mass is used as a precursor to loss of resilience (manifested as longer hospital stay, poor regain of function, rehospitalisation, and mortality), decreased physical performance, and increased frailty. Muscle mass is well established as measured by various methods and correlates with muscle performance/strength and frailty (e.g., SPPB, grip strength, HR-QoL), and ultimately, risk of hospitalisation and mortality in patients with COPD. The SPPB is a widely used clinical assessment tool designed to evaluate physical functioning and performance in older adults, and is supported by the European Working Group on Sarcopenia in Older People (EWGSOP) for assessing physical performance, resilience, and frailty in older individuals. SPPB is commonly used to assess mobility, balance, and lower extremity strength through the balance, gait speed, and chair stand tests. The SPPB provides a quantitative measure of physical performance and can help identify those who may be at risk of functional challenges and decline. For patients with COPD, poorer performance on the SPPB (i.e., every 1 point decrease) is associated with higher risk for AECOPD hospitalisation and longer length of stay. Conversely, improved performance on the SPPB (i.e., every 1 point increase) is associated with improved risk of mortality and hospital readmission in patients admitted with AECOPD.

Muscle mass, muscle strength and endurance, physical performance, frailty, and resilience outcomes is assessed at 5 timepoints from baseline (within 36 hours of hospital admission) to up to 90 days. BGE-105 has shown up to 75% reduction in muscle loss from disuse atrophy in the healthy volunteer bed rest study, as well as the preservation of muscle metabolism and physical function. In this proposed study, we aim to reduce the muscle loss in hospitalised COPD patients by 50%, which should translate to maintenance of muscle function, and potentially a greater than 25% reduction in frailty/improvement in resilience. Decrease in frailty is measured by various outcomes, including improvement on the SPPB and its component test, the chair stand test (driven mainly by the quadricep muscles), where the goal is to demonstrate a greater than 1-point difference in the SPPB or the chair stand test in the subjects treated with BGE-105. This study also aims to better the understanding from loss of muscle mass to loss of muscle strength, to decreased physical performance and resilience, especially when combined with the exploration of underlying molecular mechanisms, will help inform future clinical studies in enhancing healthspan in the most vulnerable populations. Table 12 summarizes Constituent Research Thrusts and Activities and Key Intermediate Assessments and Milestones.

TABLE 12
Constituent research thrusts and activities and key intermediate
assessments and milestones

| | |
|---|---|
| Primary | Thigh muscle mass by DEXA (dual-energy x-ray |
| outcome | absorptiometry) at baseline, Days 10 and 30 |
| Other muscle | Muscle thickness, volume, and composition by quadriceps |
| outcomes | ultrasound (baseline, discharge, Day 10, and follow up |
| (mass and | visits) and lower limb CT (discharge and Day 90) |
| metabolism) | Total lean mass by DEXA (baseline, Days 10 and 30) |
| | Total body muscle mass by urine D3-creatine (baseline |
| | and follow-up visits) |
| | Muscle protein synthesis by D₂O with muscle |
| | microbiopsy analysis (baseline, discharge, and Day 10) |
| | Body composition by multi-frequency bioelectrical |
| | impedance analysis (BIA) (baseline, Day10, discharge, |
| | and follow-up visits) |
| Physical | Grip strength (baseline, discharge, and follow-up visits) |
| function and | Quadriceps muscle strength and endurance (baseline, |
| resilience/ | Day 10, discharge, and follow up visits) |
| frailty | Short physical performance battery (SPPB; baseline, |
| outcomes | discharge, and follow-up visits) |
| | Daily activity level as measured by wearable |
| | activity monitor |
| | Fried Frailty Phenotype: weight loss, exhaustion, |
| | low physical activity, slowness, |
| | weakness (baseline, discharge, and follow-up visits) |
| Patient- | Mortality at Days 30, 60, and 90 |
| centered | Hospital readmission at Days 30, 60, and 90 |
| outcomes and | Days in hospital at Days 30, 60, and 90 |
| healthcare | Days Alive and at Home (DAH) Days 30, 60, and 90 |
| utilization | |
| Health related | EQ-5D (retroactive pre-AECOPD baseline, |
| quality of | discharge, Days 30, 60, and 90) |
| life (HR-QoL) | Clinical global impression (CGI), patient global |
| | impression of change |
| Mechanism | Blood biomarkers and proteomics (baseline, Day 10, and |
| exploration and | follow-up visits) |
| biosampling | Single-nucleus RNAseq with muscle microbiopsy |
| | Biosample banking for future analyses of blood, |
| | saliva, and urine |

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of treating a muscle condition in a subject, the method comprising administering to a subject in need thereof an effective dose of an apelin receptor agonist.

2. The method of claim 1, wherein the muscle condition is an age-related muscle condition.

3. The method of claim 1, wherein the subject is human and at least 40-years-old.

4.-9. (canceled)

10. The method of claim 1, wherein the muscle condition is a skeletal muscle condition.

11.-12. (canceled)

13. The method of claim 1, wherein the subject is not suffering from, or at risk of, a heart failure.

14. The method of claim 1, wherein the muscle condition is associated with:
inflammation and/or impairment of mitochondrial function;
loss-of-function of muscle;
decrease in the ability to regenerate muscle;
decrease in the ability to heal after injury of muscle;
the loss-of-function of muscle stem cells;
insulin insensitivity or resistance; or
Type 2 diabetes mellitus.

15.-16. (canceled)

17. The method of claim 1, wherein the muscle condition is selected from sarcopenia, frailty, hip fracture, ICU associated muscle weakness, mechanical ventilation-related muscle weakness, immobilization associated muscle weakness, recovery from muscle injury, muscle atrophy, diaphragm atrophy, critical illness myopathy, and muscle wasting.

18. (canceled)

19. The method of claim 1, wherein the subject has, or is identified as having:
low muscle strength or low muscle force;
chronic obstructive pulmonary disease (COPD);
low lower limb muscle mass;
low upper limb muscle mass; or
low muscle volume.

20.-23. (canceled)

24. The method of claim 19, wherein the muscle volume is skeletal muscle volume.

25. The method of claim 24, wherein the muscle is tibialis anterior, tibialis posterior, gastrocnemius, sartorius, vastus intermedius, vastus laterals, vastus medialis, soleus, rectus femorus, extensor digitorum longus, or diaphragm.

26.-29. (canceled)

30. The method of claim 1, wherein the dose is 200 mg.

31.-35. (canceled)

36. A method for maintaining and/or increasing muscle mass and/or muscle strength in an human subject, the method comprising administering to a subject in need thereof an effective dose of an apelin receptor agonist.

37. The method of claim 36, wherein the subject is at least 60-years-old.

38.-41. (canceled)

42. The method of claim 36, wherein the human subject has, or is identified as having, low muscle strength, low muscle force, low lower limb muscle mass, low upper limb muscle mass, or low muscle volume.

43.-47. (canceled)

48. The method of claim 42, wherein the muscle is diaphragm, tibialis anterior, tibialis posterior, gastrocnemius, sartorius, vastus intermedius, vastus laterals, vastus medialis, soleus, rectus femorus, or extensor digitorum longus.

49. (canceled)

50. The method of claim 42, wherein the human subject is mechanically ventilated.

51. The method of claim 36, wherein the human subject has, or is identified as having;
reduced diaphragm thickness as compared to a human subject that is not mechanically ventilated;
diaphragm atrophy;
ventilator-induced diaphragmatic dysfunction (VIDD), or
hypoxic respiratory failure.

52.-90. (canceled)

91. The method of claim 1, wherein the apelin receptor agonist is of formula (I) or (II): 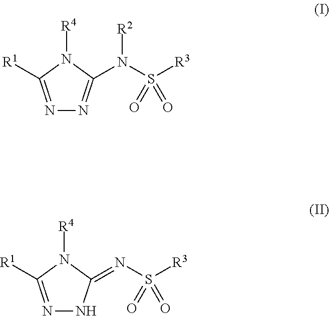
or a pharmaceutically acceptable salt thereof, a tautomer thereof, a pharmaceutically acceptable salt of the tautomer, a stereoisomer of any of the foregoing, or a mixture thereof,
wherein:
R¹ is an unsubstituted pyridyl, pyridonyl, or pyridine N-oxide, or is a pyridyl, pyridonyl, or pyridine N-oxide substituted with 1, 2, 3, or 4 R^{1a} substituents;
R^{1a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —C₂-C₆ alkenyl, —O—(C₁-C₆ alkyl)-OH, —O—(C₁-C₆ alkyl)-O—(C₁-C₆ alkyl), —O—(C₁-C₆ haloalkyl)-OH, —O—(C₁-C₆ haloalkyl)-O—(C₁-C₆ alkyl), —O—(C₁-C₆ perhaloalkyl)-OH, —O—(C₁-C₆ perhaloalkyl)-O—(C₁-C₆ alkyl), —NH₂, —NH(C₁-C₆ alkyl), —N(C₁-C₆ alkyl)₂, —C(═O)—(C₁-C₆ alkyl), —C(═O)OH, —(C═O)—O—(C₁-C₆ alkyl), —C(═O)NH₂, —C(═O)NH(C₁-C₆ alkyl), —C(═O)N(C₁-C₆ alkyl)₂, phenyl, —C(═O)-(heterocyclyl), or a heterocyclyl group, wherein the heterocyclyl group of the —C(═O)-(heterocyclyl) or heterocyclyl group is a 3 to 7 membered ring containing 1, 2, or 3 heteroatoms selected from N, O, and S;
R² is selected from —H, and C₁-C₄ alkyl or is absent in the compounds of Formula II;
R³ is selected from an unsubstituted C₁-C₁₀ alkyl, a C₁-C₁₀ alkyl substituted with 1, 2, or 3 R^{1a} substituents, a group of formula —(CR^{3b}R^{3c})-Q, a group of formula —NH—(CR^{3b}R^{3c})-Q, a group of formula —(CR^{3b}R^{3c})—C(═O)-Q, a group of formula —(CR^{3d}R^{3e})—(CR^{3f}R^{3g})-Q, a group of formula —(CR^{3h}═CR^{3c})-Q, and a group of formula -(heterocyclyl)-Q, wherein the heterocyclyl of the -(heterocyclyl)-Q has 5 to 7 ring members of which 1, 2, or 3 are heteroatoms selected from N, O, and S and is unsubstituted or is substituted with 1, 2, or 3 R^{3h} substituents;
R^{1a} in each instance is independently selected from —F, —Cl, —CN, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), C₂-C₆ alkenyl, C₂-C₆ alkynyl, —NH₂, —NH(C₁-C₆ alkyl), and —N(C₁-C₆ alkyl)₂;
R^{3b} and R^{3c} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3d} and R^{3e} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3f} and R^{3g} are independently selected from —H, —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), and —N(C₁-C₆ alkyl)₂;
R^{3h} in each instance is independently selected from —F, —Cl, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —O—(C₁-C₆ alkyl)-OH, —O—(C₁-C₆ alkyl)-O—(C₁-C₆ alkyl), —NH₂, —NH(C₁-C₆ alkyl), —N(C₁-C₆ alkyl)₂, and oxo;
Q is a monocyclic or bicyclic C₆-C₁₀ aryl group, a monocyclic or bicyclic heteroaryl group with 5 to 10 ring members containing 1, 2, or 3 heteroatoms selected from N, O, or S, a C₃-C₈ cycloalkyl group, or a 3 to 7 membered heterocyclyl group containing 1, 2, or 3 heteroatoms selected from N, O, or S, wherein the C₆-C₁₀ aryl group, the heteroaryl group, the cycloalkyl group, and the heterocyclyl group are unsubstituted or are substituted with 1, 2, 3, or 4 R^{Q} substituent;
R^{Q} in each instance is independently selected from —F, —Cl, —Br, —I, —CN, —C₁-C₆ alkyl, —C₁-C₆ haloalkyl, —C₁-C₆ perhaloalkyl, —C₂-C₆ alkenyl, —C₂-C₆ alkynyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), —C(═O)N(C₁-C₆ alkyl)₂, —S(═O)₂—(C₁-C₆ alkyl), phenyl, and a heteroaryl group, and the Q heterocyclyl group may be substituted with 1 oxo R^{Q} substituent;
R⁴ is selected from a monocyclic or bicyclic C₆-C₁₀ aryl group, a monocyclic or bicyclic heteroaryl group with 5 to 10 ring members containing 1, 2, or 3 heteroatoms independently selected from N, O, and S, and a monocyclic or bicyclic heterocyclyl group with 5 to 10 ring members containing 1, 2, 3, or 4 heteroatoms independently selected from N, O, and S, wherein the C₆-C₁₀ aryl group, the heteroaryl group, or the heterocyclyl group are unsubstituted or are substituted with 1, 2, or 3 R^{4a} substituents;
R^{4a} in each instance is independently selected from —F, —Cl, —Br, —I, —CN, —C₁-C₆ alkyl, —C₁-C₆ haloalkyl, —C₁-C₆ perhaloalkyl, —OH, —O—(C₁-C₆ alkyl), —O—(C₁-C₆ haloalkyl), —O—(C₁-C₆ perhaloalkyl), —NH₂, —NH(C₁-C₆ alkyl), —N(C₁-C₆ alkyl)₂, —C(═O)—(C₁-C₆ alkyl), —C(═O)OH, —C(═O)—O—(C₁-C₆ alkyl), —C(═O)NH₂, —C(═O)NH(C₁-C₆ alkyl), and —C(═O)N(C₁-C₆ alkyl)₂, and the heterocyclyl R⁴ group may be further substituted with 1 oxo substituent; and
further wherein:
if R⁴ is an unsubstituted or substituted phenyl ring and R³ is a group of formula —(CR^{3b}═CR^{3c})-Q, then at least one of the following is true:
a) R⁴ is substituted with at least one —O—(C₁-C₆ alkyl) group;
b) Q is not an oxadiazole;
c) R^{3b} is not —H;
d) R^{3c} is not —H;
e) R¹ is not a 2-pyridyl group; or
f) R⁴ is substituted with two or more —O—(C₁-C₆ alkyl) groups.

92.-105. (canceled)

106. The method of claim 90, wherein the apelin receptor agonist is (2S,3R)—N-(4-(2,6-dimethoxyphenyl)-5-(5-methyl-3-pyridinyl)-4H-1,2,4-triazol-3-yl)-3-(5-methyl-2-pyrimidinyl)-2-butanesulfonamide or a pharmaceutically acceptable salt thereof.
